(19) United States
(12) Patent Application Publication
(10) Pub. No.: US 2025/0074919 A1
(43) Pub. Date: Mar. 6, 2025

(54) Heteroaryl compounds as inhibitors of irak4, compositions and applications thereof

(71) Applicant: Accro Bioscience (HK) Limited, Wan Chai (HK)

(72) Inventors: Xiaohu Zhang, Suzhou (CN); Sudan He, Suzhou (CN); Haikuo Ma, Suzhou (CN)

(21) Appl. No.: 18/720,657

(22) PCT Filed: Dec. 30, 2022
(86) PCT No.: PCT/US2022/082600

§ 371 Date: Jun. 16, 2024

(30) Foreign Application Priority Data
Dec. 31, 2021 (CN) 202111661982.5
Oct. 24, 2022 (CN) 202211305127.5

(51) Int. Cl. C07D 498/04; A61K 31/444; A61K 31/4545; A61K 31/496; A61K 31/5377; A61K 31/675; A61K 45/06; C07D 491/048; C07D 519/00; C07F 9/6561

(52) U.S. Cl. CPC C07D 498/04; A61K 31/444; A61K 31/4545; A61K 31/496; A61K 31/5377; A61K 31/675; A61K 45/06; C07D 491/048; C07D 519/00; C07F 9/6561

(57) ABSTRACT

The present disclosure relates to the field of medicinal chemistry, and specifically relates to a compound with interleukin-1 receptor-associated kinase 4 (IRAK4) inhibitory activity, and pharmaceutical compositions and applications thereof. The present disclosure provides a compound of Formula (I) as an effective IRAK4 inhibitor, which can be used for the prevention and/or treatment of IRAK4-related diseases and/or conditions.

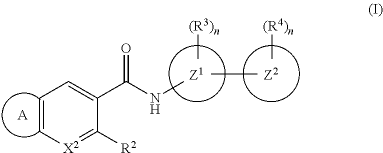

### CROSS REFERENCE TO RELATED APPLICATIONS

This application claims the benefit of Chinese Patent Applications 202111661982.5, filed on Dec. 31, 2021; and 202211305127.5, filed on Oct. 24, 2022; all of which are hereby incorporated by reference in their entirety.

### FIELD OF INVENTION

The present disclosure is in the field of medical technology and relates to amide compounds having inhibitory activity against the MyD88/IRAK4 pathway, compositions comprising such compounds, and methods of their use. The present disclosure further pertains to pharmaceutical compositions comprising at least one compound according to the present disclosure that are useful for the treatment of conditions related to kinase modulation and methods of inhibiting the activity of kinases, including IRAK-4 in a mammal (various diseases including tumors, inflammatory diseases, autoimmune diseases, neurodegenerative diseases, metabolic diseases, and genetic diseases).

### BACKGROUND OF THE INVENTION

Interleukin-1 receptor-associated kinase 4 (IRAK4) is a serine/threonine protein kinase that is involved in the interleukin (IL) receptor family (e.g., IL-1R, IL-18R, or IL-33R) and Toll-like receptors (TLRs) family (except TLR3). It plays an important role in the inflammatory signaling pathway. See Henderson and Goldbach-Mansky, Clin. Immunol. 2010, 135, 210-222.

Animal experiments have shown that knocking out IRAK4 protein in mice could prevent interleukin-1 receptor (IL-1R)-mediated or TLR-mediated signaling pathway transmissions and the release of related cytokines. See Suzuki et al., Nature 2002, 416, 750-756. More and more evidence shows that IRAK4 is an attractive target for treating inflammatory diseases (e.g., rheumatoid arthritis, inflammatory bowel disease, systemic lupus erythematosus, etc.)

As a key regulator in the Toll-like signal transduction pathway, IRAK4 can form a myddosome complex through its death domain (DD) with myeloid differentiation primary response 88 (MyD88) and downstream IRAK1/2. See Lin et al., Nature 2010, 465, 885-890. In this complex, IRAK4 is first activated by trans-autophosphorylation, subsequently IRAK1/2 is activated, followed by the activation of the downstream nuclear factor-κB (NF-κB) and mitogen-activated protein kinase (MAPK) signal transduction pathways. See Ferrao et al., Mol. Cell 2014, 55, 891-903. Therefore, both the kinase activity and the scaffolding function of IRAK4 play an important role in the transduction of inflammatory signals. More and more experiments have demonstrated that inhibiting the kinase activity of IRAK4 by gene editing or small molecule compounds can reduce the expression of inflammatory cytokines in vitro and in vivo, and relieve the symptoms of the related inflammatory phenotypes. See Chaudhary et al., J. Med. Chem. 2015, 58, 96-110. In addition, it has been reported that in some malignant blood cancers (29% of activated B-cell-like diffuse large B-cell lymphoma (ABC-DLBCL), Waldenstrand's macroglobulinemia (WM), etc.) MyD88 has acquired functional mutations, which indicates that the regulation of IRAK4 may have therapeutic effects on some blood cancers. See Ondrejka et al., Am. J. Clin. Pathol. 2013, 140, 387-394.

Currently there is no marketed drug targeting IRAK4. Many pharmaceutical companies have worked on this target. Among them, PF-06650833 developed by Pfizer was the first IRAK4 inhibitor to enter the clinic. See Lee et al., J. Med. Chem. 2017, 60, 5521-5542. This IRAK4 inhibitor has entered Phase II clinical trials for treating rheumatoid arthritis and hidradenitis suppurativa. Recently Bayer has launched two IRAK4 inhibitors (BAY-1830839 and BAY-1834845) which are currently in Phase I clinical trials. R-835 by Rigel also has entered Phase I clinical trials. The indications for the IRAK4 inhibitors by Bayer and Rigel are mainly in the fields of autoimmune diseases, inflammation, and ABC-DLBCL. CA4948, a small molecule inhibitor of IRAK4 developed by Curis, has been reported to exhibit positive effect on six patients with relapsed or refractory myelodysplastic syndrome (MDS) and acute myeloid leukemia (AML). See Gummadi et al., ACS Med. Chem. Lett. 2020, 11, 2374-2381. Accordingly, developing small molecule inhibitors of IRAK4 is a research direction of great clinical values.

### SUMMARY OF THE INVENTION

The present disclosure provides compounds that can inhibit IRAK4, and compositions and applications thereof. These disclosed IRAK4 inhibitors, and compositions and applications thereof, may effectively prevent or treat diseases and disorders related to IRAK4 activation.

One goal of the present disclosure is to provide IRAK4 inhibitors, and compositions and applications thereof.

The first aspect of the present disclosure provides a compound of Formula (I):

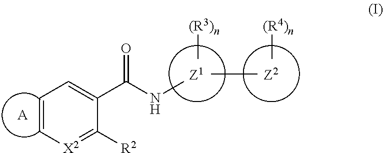

or a pharmaceutically acceptable salt, ester, solvate, prodrug, isotope-labeled derivative, or isomer thereof, wherein:
ring A is selected from the group consisting of:

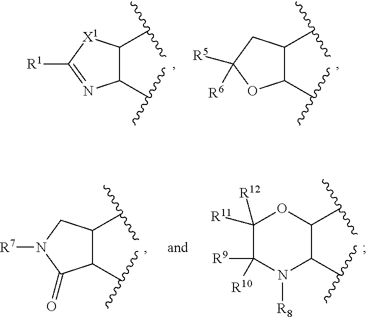

each n is independently 0, 1, 2, or 3;
X¹ is O or S;
X² is N or CH;
if present, R¹ is hydrogen, C₁₋₆ alkyl, C₃₋₆ cycloalkyl, C₁₋₂ alkylene-C₃₋₆ cycloalkyl, —NH(C₁₋₆ alkyl), —N(C₁₋₆ alkyl)₂, —NH(C₃₋₆ cycloalkyl), —N(C₃₋₆ cycloalkyl)₂, —OC₁₋₆ alkyl, 3-8 membered heterocyclyl, 6-10 membered bridged biheterocyclyl, 5-12 membered spiro biheterocyclyl, 6-12 membered fused biheterocyclyl, C₆₋₁₀ aryl, or 5-10 membered heteroaryl, wherein each of C₁₋₆ alkyl, C₃₋₆ cycloalkyl, aryl, heteroaryl, heterocyclyl, bridged biheterocyclyl, spiro biheterocyclyl, and fused biheterocyclyl are substituted with 1, 2 or 3 R^{a} independently; if present, each of heterocyclyl, bridged biheterocyclyl, spiro biheterocyclyl, fused biheterocyclyl, and heteroaryl comprises one or more heteroatoms independently selected from the group consisting of O, S, NH, N, P(═O), S(═O) and S(═O)₂;
R² is hydrogen, C₁₋₆ alkyl, C₃₋₆ cycloalkyl, C₁₋₂ alkylene-C₃₋₆ cycloalkyl, —NH(C₁₋₆ alkyl), —N(C₁₋₆ alkyl)₂, —NH(C₃₋₆ cycloalkyl), —N(C₃₋₆ cycloalkyl)₂, hydroxy, —OC₁₋₆ alkyl, —OC₃₋₆ cycloalkyl, O-heterocyclyl wherein the heterocyclyl is a 3-8 membered heterocyclyl, 3-8 membered heterocyclyl, 6-10 membered bridged biheterocyclyl, 5-12 membered spiro biheterocyclyl, 6-12 membered fused biheterocyclyl, C₆₋₁₀ aryl, or 5-10 membered heteroaryl, wherein each of C₁₋₆ alkyl, C₃₋₆ cycloalkyl, aryl, heteroaryl, heterocyclyl, bridged biheterocyclyl, spiro biheterocyclyl, and fused biheterocyclyl are substituted with 1, 2 or 3 R^{b} independently; if present, each of heterocyclyl, bridged biheterocyclyl, spiro biheterocyclyl, fused biheterocyclyl, and heteroaryl comprises one or more heteroatoms independently selected from the group consisting of O, S, NH, N, P(═O), S(═O) and S(═O)₂; preferably, R² is hydrogen, C₁₋₆ alkyl, C₃₋₆ cycloalkyl, C₁₋₂ alkylene-C₃₋₆ cycloalkyl, —NH(C₁₋₆ alkyl), —N(C₁₋₆ alkyl)₂, —NH(C₃₋₆ cycloalkyl), —N(C₃₋₆ cycloalkyl)₂, —OC₁₋₆ alkyl, 3-8 membered heterocyclyl, 6-10 membered bridged biheterocyclyl, 5-12 membered spiro biheterocyclyl, 6-12 membered fused biheterocyclyl, C₆₋₁₀ aryl, or 5-10 membered heteroaryl, wherein each of C₁₋₆ alkyl, C₃₋₆ cycloalkyl, aryl, heteroaryl, heterocyclyl, bridged biheterocyclyl, spiro biheterocyclyl, and fused biheterocyclyl are substituted with 1, 2 or 3 R^{b} independently; if present, each of heterocyclyl, bridged biheterocyclyl, spiro biheterocyclyl, fused biheterocyclyl, and heteroaryl comprises one or more heteroatoms independently selected from the group consisting of O, S, NH, N, P(═O), S(═O) and S(═O)₂;
if present, each R⁵ and R⁶ is independently hydrogen, deuterium, C₁₋₆ alkyl, C₃₋₆ cycloalkyl, or C₁₋₂ alkylene-C₃₋₆ cycloalkyl, wherein each C₁₋₆ alkyl and C₃₋₆ cycloalkyl is substituted with 1, 2, or 3 R^{c} independently; or R⁵ and R⁶, together with the carbon atoms they bound with, form a saturated 5- or 6-membered spiro heterocycle comprising one or more heteroatoms independently selected from the group consisting of oxygen, nitrogen, and sulfur, wherein the saturated 5- or 6-membered spiro heterocycle is substituted with 1, 2, or 3 R^{c};
if present, R⁷ is hydrogen, C₁₋₆ alkyl, C₃₋₆ cycloalkyl, C₁₋₂ alkylene-C₃₋₆ cycloalkyl, or 5- or 6-membered heterocyclyl comprising one or more heteroatoms independently selected from the group consisting of oxygen, nitrogen, and sulfur, wherein each C₁₋₆ alkyl and C₃₋₆ cycloalkyl is substituted with 1, 2, or 3 R^{c} independently; if present, R⁸ is hydrogen, C₁₋₆ alkyl, C₃₋₆ cycloalkyl, C₁₋₂ alkylene-C₃₋₆ cycloalkyl, or 5-6 membered heterocyclyl comprising one or more heteroatoms independently selected from the group consisting of oxygen, nitrogen, and sulfur; each R⁹, R, R¹¹, and R¹² is independently hydrogen, deuterium, C₁₋₆ alkyl, C₃₋₆ cycloalkyl, C₁₋₂ alkylene-C₃₋₆ cycloalkyl, or 5-6 membered heterocyclyl comprising one or more heteroatoms independently selected from the group consisting of oxygen, nitrogen, and sulfur; wherein each C₁₋₆ alkyl, C₃₋₆ cycloalkyl, and heterocyclyl is substituted with 1, 2, or 3 R^{c} independently; or R⁹ and R¹⁰, together with the carbon they bound with, form a carbonyl; ring Z¹ is selected from the group consisting of:
   (1) 5-6 membered heteroaryl comprising 1, 2, or 3 heteroatoms independently selected from the group consisting of oxygen, nitrogen, and sulfur;
   (2) phenyl;
   (3) 5-6 membered unsaturated or saturated heterocyclyl comprising 1 or 2 heteroatoms independently selected from the group consisting of oxygen and nitrogen;
   (4) 7-10 membered fused bicyclic heterocyclyl comprising 1, 2, or 3 heteroatoms independently selected from the group consisting of oxygen, nitrogen, and sulfur; and
   (5) unsaturated or saturated C₃₋₆ cycloalkyl;
ring Z² is absent, or ring Z² is selected from the group consisting of:
   (1) 5-6 membered heteroaryl comprising 1 to 4 heteroatoms independently selected from the group consisting of oxygen, nitrogen, and sulfur;
   (2) phenyl;
   (3) 5-6 membered unsaturated or saturated heterocyclyl comprising 1 or 2 heteroatoms independently selected from the group consisting of oxygen and nitrogen; and
   (4) unsaturated or saturated C₃₋₆ cycloalkyl;
   wherein when ring Z² is absent, R^{d} replaces ring Z² and R^{d} connected with ring Z¹;
if present, each R^{a} is independently hydrogen, deuterium, fluorine, chlorine, bromine, iodine, hydroxy, amino, methylamino, dimethylamino, cyano, methyl, deuterated methyl, methoxy, deuterated methoxy, ethyl, cyclopropyl, tert-butoxycarbonyl, carbamoyl, C₁₋₂ alkylene-hydroxy, C₁₋₂ alkylene-methoxy, or C₁₋₂ alkylene-deuterated methoxy; if present, each R^{b} is independently hydrogen, deuterium, fluorine, chlorine, bromine, iodine, hydroxy, amino, methylamino, dimethylamino, cyano, C₁₋₃ alkyl, deuterated methyl, methoxy, deuterated methoxy, cyclopropyl, C₁₋₂ alkylene-hydroxy, C₁₋₂ alkylene-methoxy, or C₁₋₂ alkylene-deuterated methoxy; or two R^{b} on any non-adjacent carbons, together with atoms attached thereto, form a ring; or two R^{b} on the same carbon, together with the carbon attached thereto, form a carbonyl if present, each R^{c} is independently hydrogen, deuterium, fluorine, chlorine, bromine, iodine, hydroxy, amino, methylamino, dimethylamino, cyano, methyl, deuterated methyl, methoxy, or deuterated methoxy;
if present, each R^{d} is independently hydrogen, deuterium, methyl, deuterated methyl, ethyl, cyclopropyl, C₁₋₂ alkylene-hydroxy, C₁₋₂ alkylene-methoxy, C₁₋₂ alkylene-deuterated methoxy, trifluoromethyl, trifluoromethoxy, difluoromethyl, or difluoromethoxy; and if present, each R³ and R⁴ is independently hydrogen, deuterium, fluorine, chlorine, bromine, iodine, hydroxy, amino, methylamino, dimethylamino, nitro, cyano, methyl, deuterated methyl, methoxy, deuterated methoxy, ethyl, cyclopropyl, trifluoromethyl, trifluoromethoxy, difluoromethyl, difluoromethoxy, or dimethylphosphinyl.

In some embodiments of aspects provided herein, including any one of the hitherto described embodiments, the compound is formula (I-A):

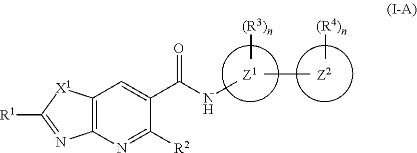

wherein n, X¹, Z¹, Z², R¹, R², R³, and R⁴ are define as above.

In some embodiments of aspects provided herein, including any one of the hitherto described embodiments, the compound is formula (I-A1):

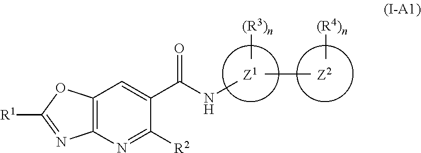

wherein n, Z¹, Z², R¹, R², R³, and R⁴ are define as above.

In some embodiments of aspects provided herein, including any one of the hitherto described embodiments, the compound is formula (I-C):

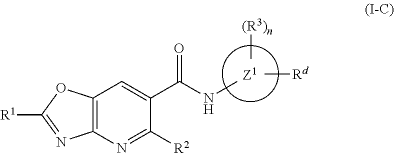

wherein n, R¹, R², R³, R^{d}, and Z¹ are defined as above.

In some embodiments of aspects provided herein, including any one of the hitherto described embodiments, the compound is formula (I-A2):

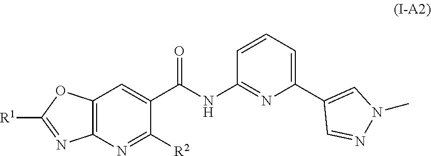

wherein n, R¹, and R² are define as above.

In some embodiments of aspects provided herein, including any one of the hitherto described embodiments, the compound is formula (I-B):

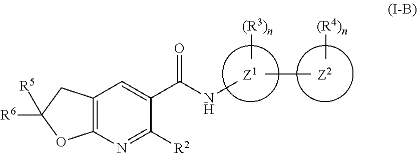

wherein n, Z¹, Z², R², R³, R⁴, R⁵, and R⁶ are define as above.

In some embodiments of aspects provided herein, including any one of the hitherto described embodiments, the compound is formula (I-B1):

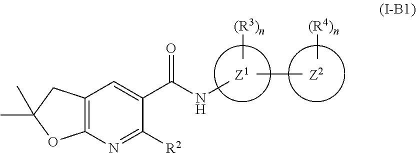

wherein n, Z¹, Z², R², R³, and R⁴ are define as above.

In some embodiments, including any one of the hitherto described embodiments, the compound is Formula (I-D):

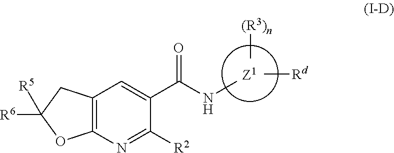

wherein n, R², R³, R⁵, R⁶, R^{d}, and Z¹ are defined as above.

In some embodiments, including any one of the hitherto described embodiments, the compound is Formula (I-B2):

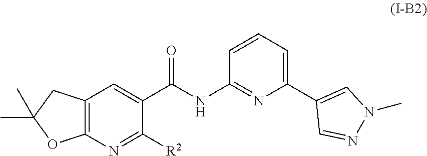

wherein R² is defined as above.

In some embodiments, including any one of the hitherto described embodiments, including compounds of Formulas (I), (I-A), (I-A1), (I-B), (I-B1), (I-C), and (I-D), ring Z¹ is selected from the group consisting of: (1) 5-6 membered heteroaryl comprising 1, 2, or 3 heteroatoms independently selected from the group consisting of oxygen, nitrogen, and sulfur; (2) phenyl; and (3) 7-10 membered fused bicyclic heterocyclyl comprising 1, 2, or 3 heteroatoms independently selected from the group consisting of oxygen, nitrogen, and sulfur.

In some embodiments, including any one of the hitherto described embodiments, (1) when ring Z² is present,

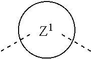

is selected from the group consisting of:

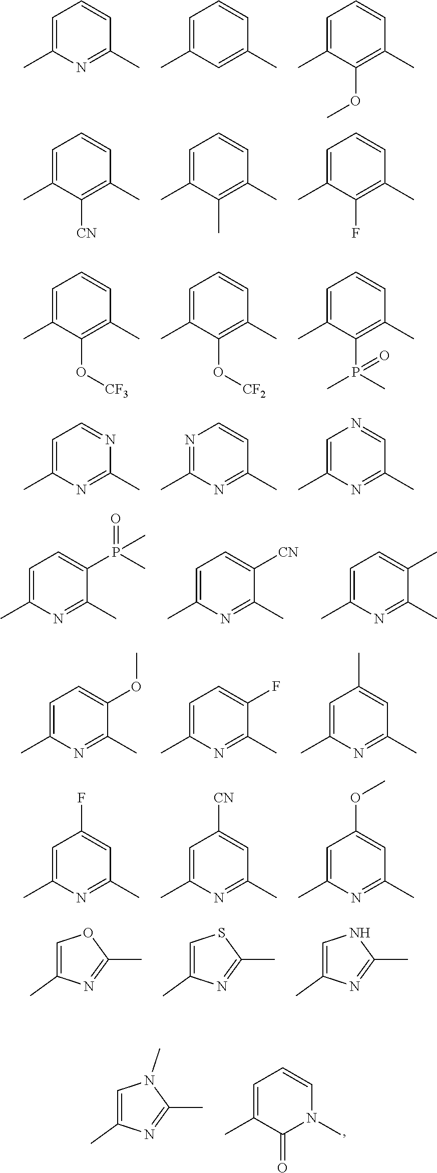

preferably selected from the croup consisting of:

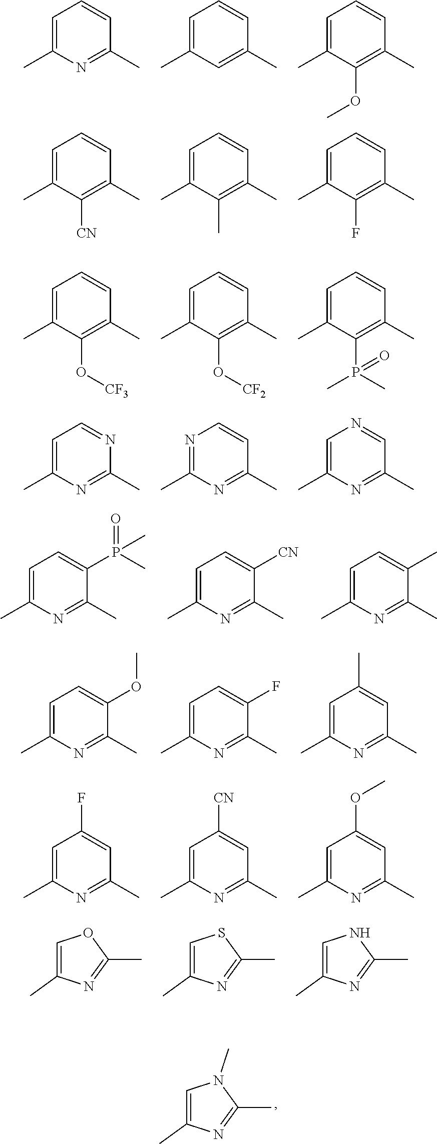

or preferably selected from the group consisting of:

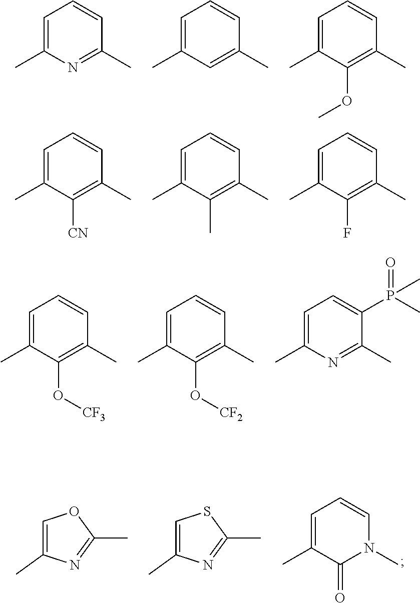

(2) when Z² is absent,

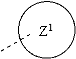

is from the group consisting of:

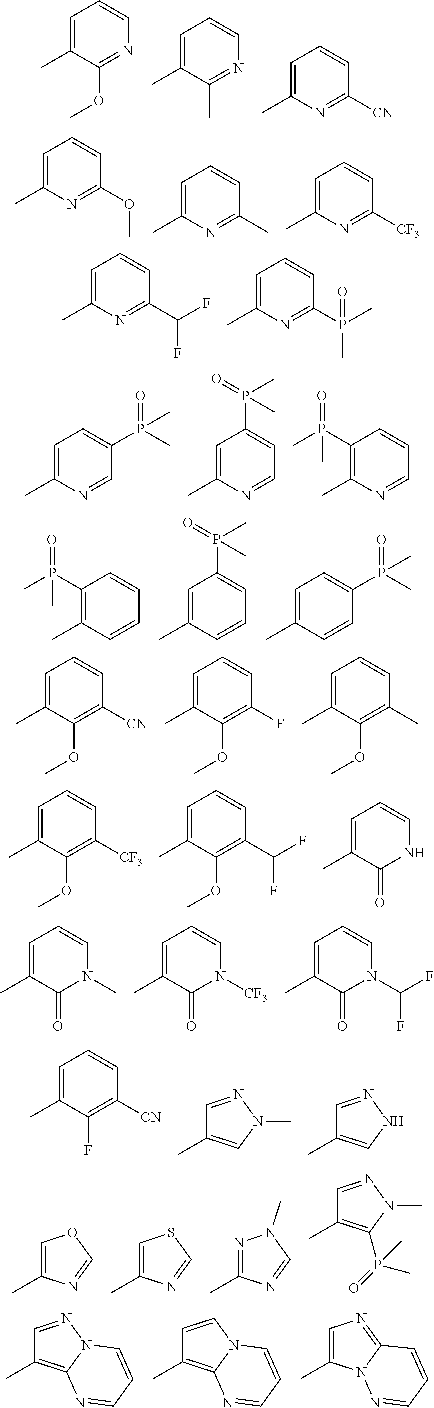

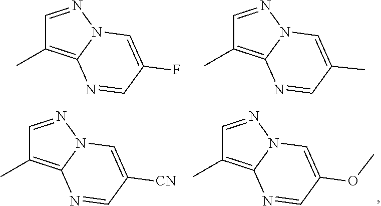

preferably selected from the group consisting of:

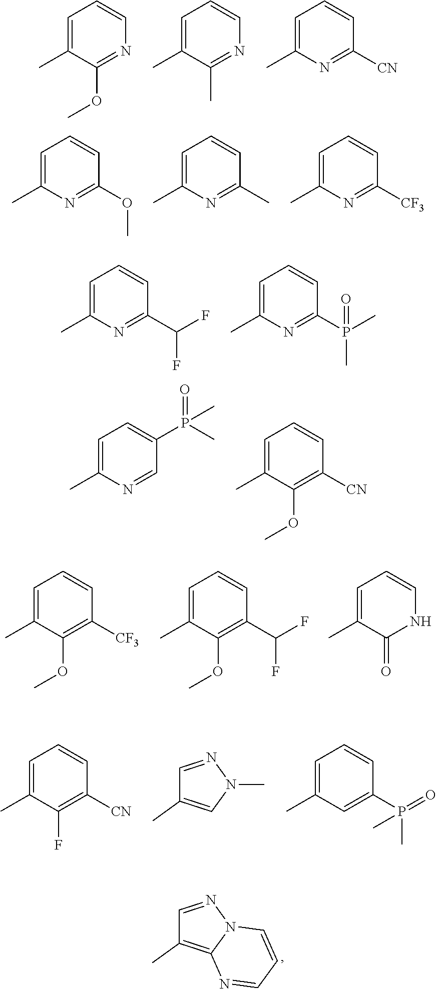

and more preferably selected from the group consisting of:

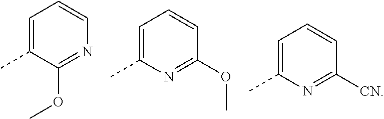

In some embodiments, including any one of the hitherto described embodiments, including compounds of Formulas (I), (I-A), (I-A1), (I-B) and (I-B1), wherein:
(1) when ring Z² is present,

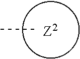

is selected from the group consisting of:

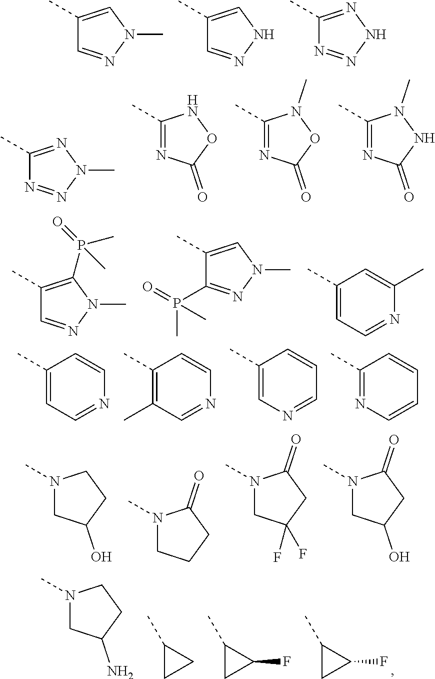

preferably selected from the group consisting of:

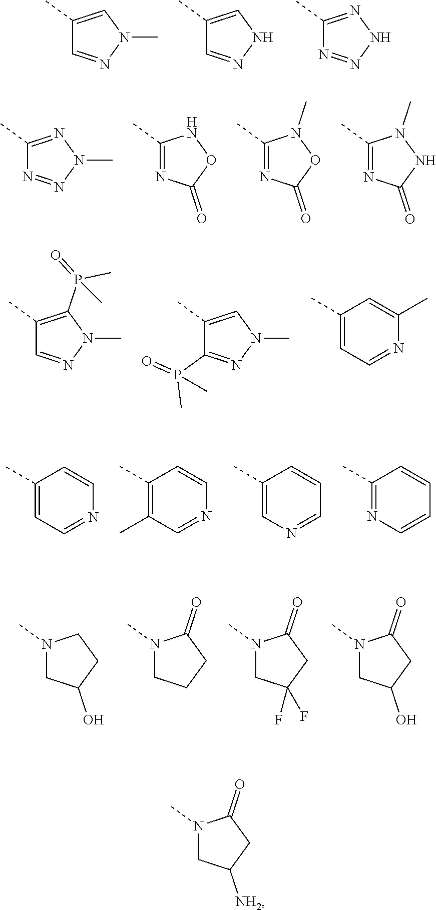

or preferably selected from the group consisting of:

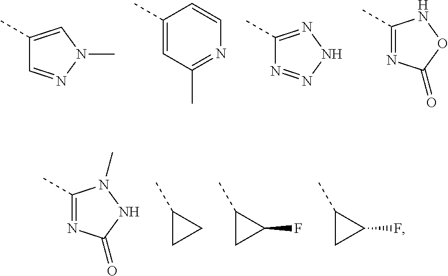

more preferably selected from the group consisting of:

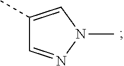

(2) when ring Z² is absent, R^{d} replaces ring Z², and R^{d} connected with ring Z¹, and R^{d} is described above.

In some embodiments, including any one of the hitherto described embodiments, including compounds of Formulas (I), (I-A), (I-A1), (I-B), (I-B1), (I-C), and (I-D), R¹ is selected from the group consisting of:

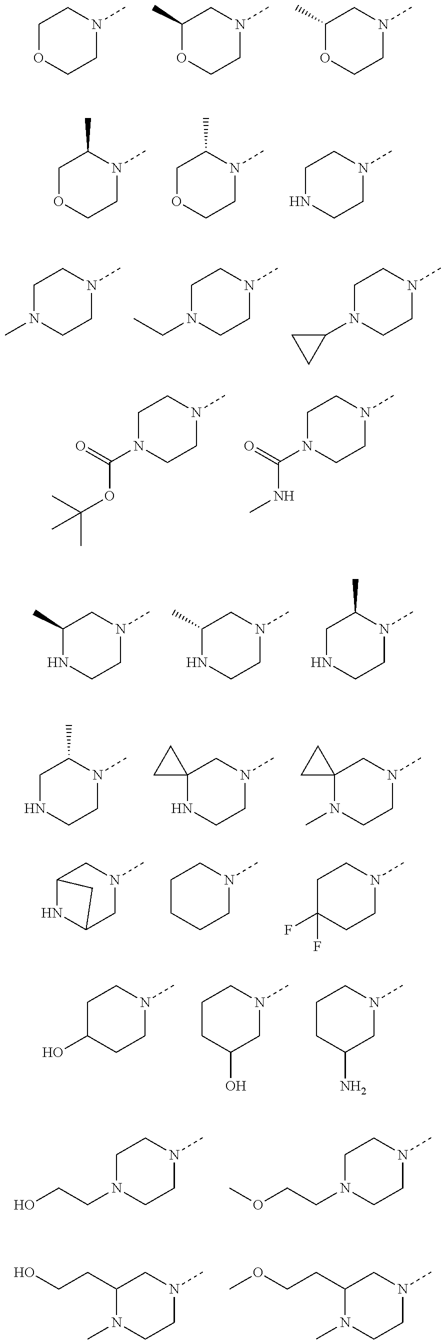

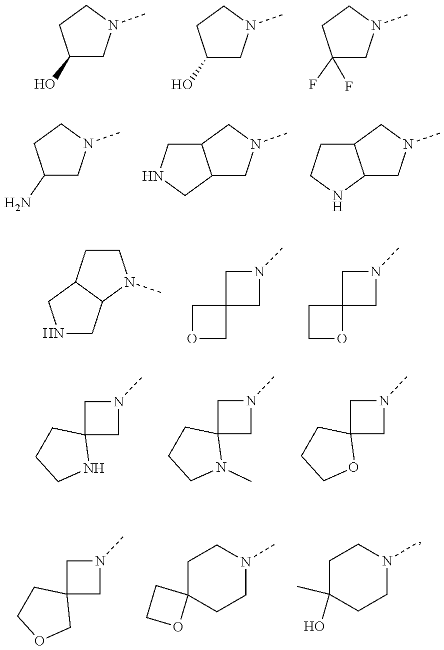

preferably selected from the group consisting of:

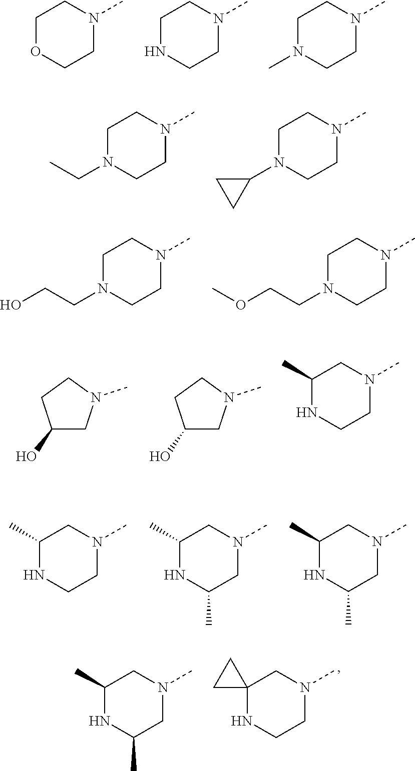

more preferably selected from the group consisting:

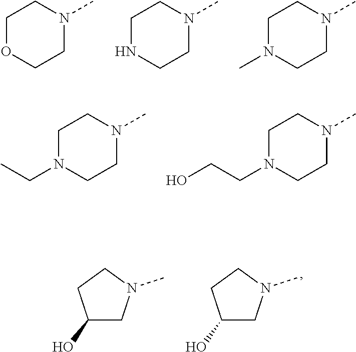

further more preferably selected from the group consisting:

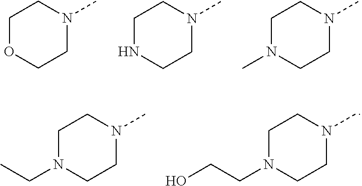

In some embodiments, including any one of the hitherto described embodiments, including compounds of Formulas (I), (I-A), (I-A1), (I-B), (I-B1), (I-C), and (I-D), R² is selected from the group consisting of —NH(C₁₋₆ alkyl), —N(C₁₋₆ alkyl)₂, —NH(C₃₋₆ cycloalkyl), —N(C₃₋₆ cycloalkyl)₂, hydroxy, —OC₁₋₆ alkyl, —OC₃₋₆ cycloalkyl, O-heterocyclyl wherein the heterocyclyl is a 3-8 membered heterocyclyl, 3-8 membered heterocyclyl, 6-10 membered bridged biheterocyclyl, 5-12 membered spiro biheterocyclyl, 6-12 membered fused biheterocyclyl, wherein each of C₁₋₆ alkyl, C₃₋₆ cycloalkyl, heterocyclyl, spiro biheterocyclyl, and fused biheterocyclyl are substituted with 1, 2 or 3 R^{b} independently, and R^{b} is described above; preferably R² is —NH(C₁₋₆ alkyl), —N(C₁₋₆ alkyl)₂, —NH(C₃₋₆ cycloalkyl), —N(C₃₋₆ cycloalkyl)₂, —OC₁₋₆ alkyl, 3-8 membered heterocyclyl, 5-12 membered spiro biheterocyclyl, and 6-12 membered fused biheterocyclyl, wherein each of C₁₋₆ alkyl, C₃₋₆ cycloalkyl, heterocyclyl, spiro biheterocyclyl, and fused biheterocyclyl are substituted with 1, 2 or 3 R^{b} independently, and R^{b} is described above.

In some embodiments, including any one of the hitherto described embodiments, R² is selected from the group consisting of:

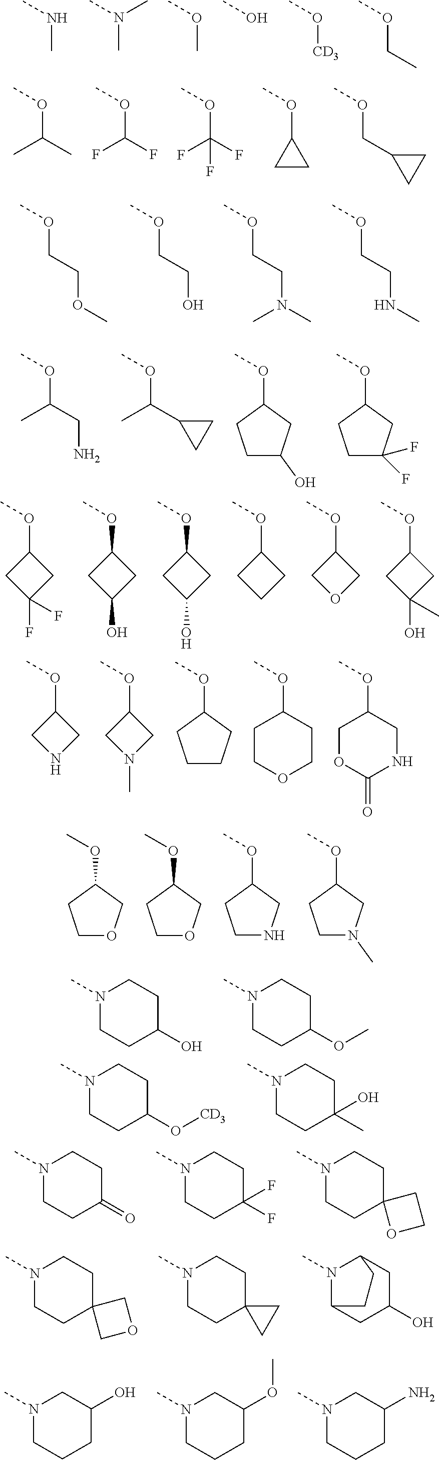

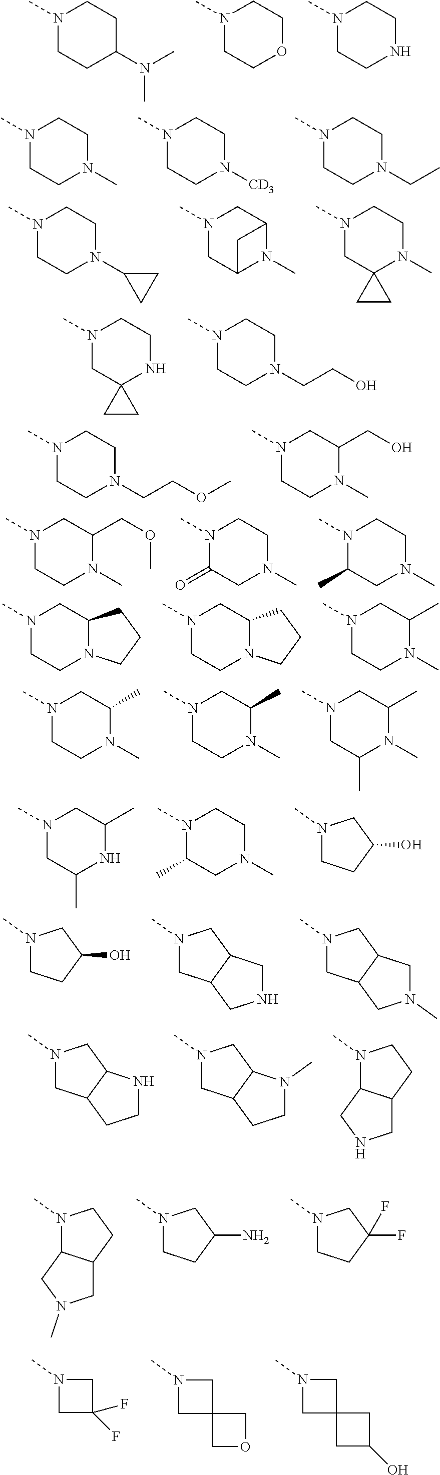

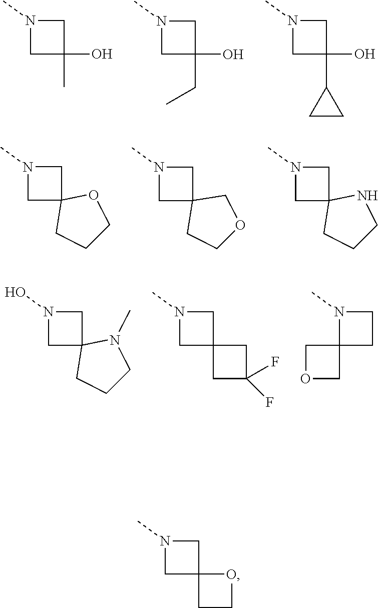

preferably selected from the group consisting of:

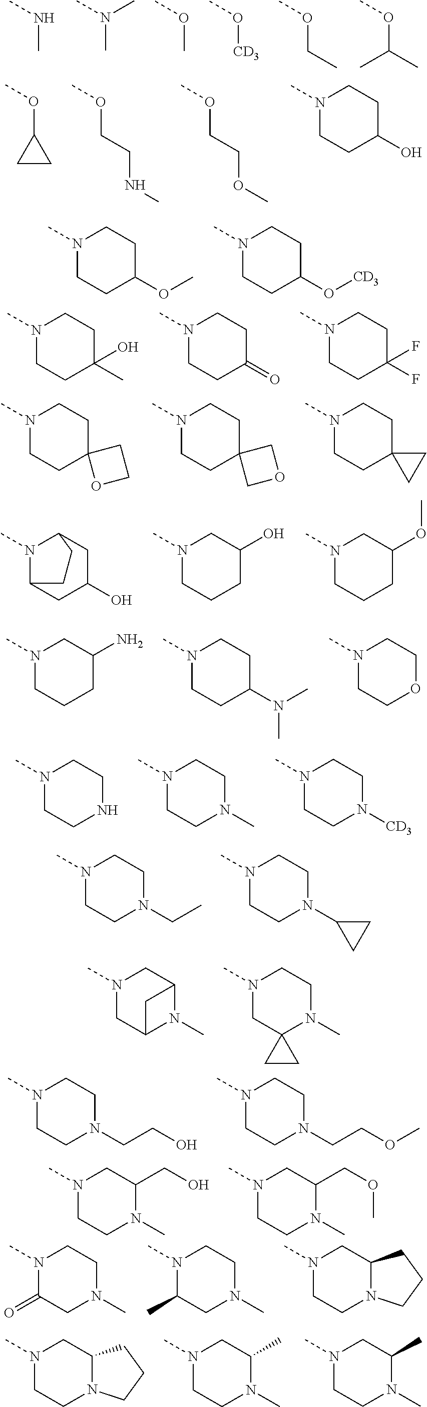

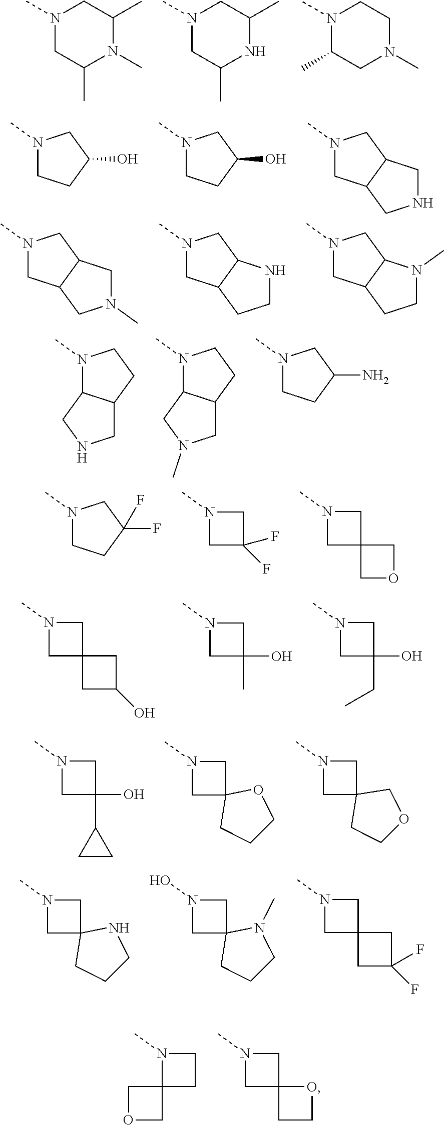

or preferably selected from the group consisting of:

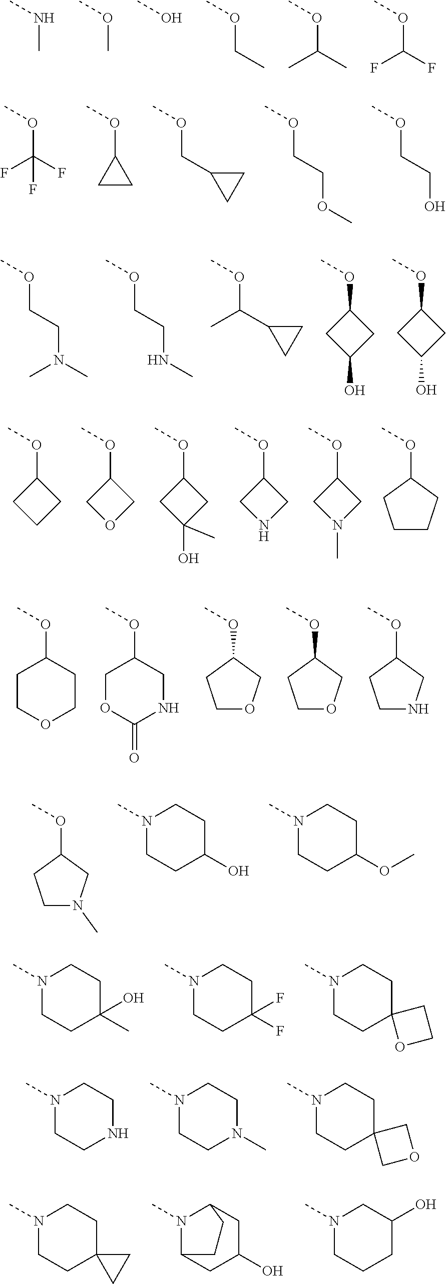

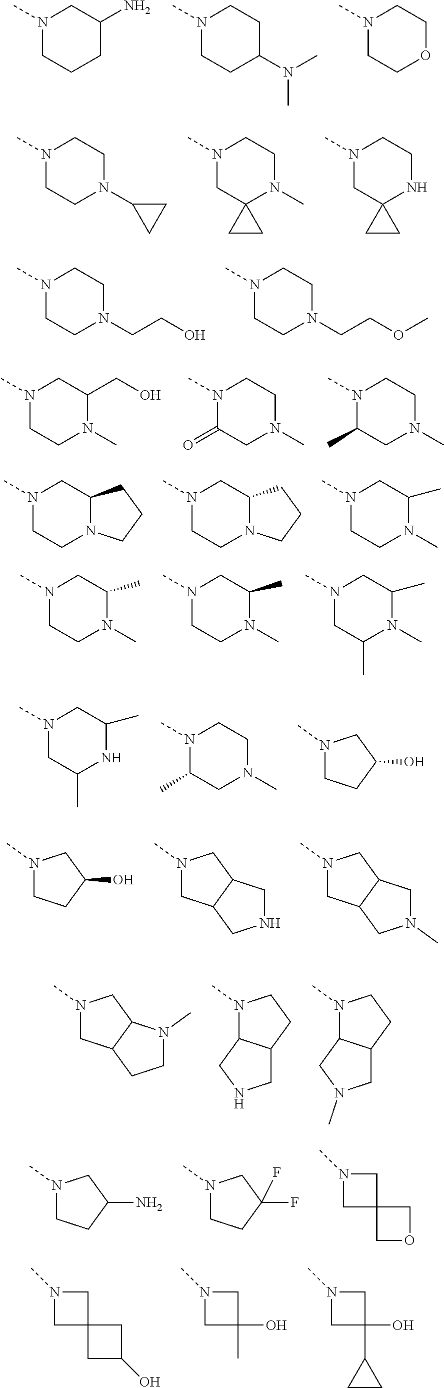

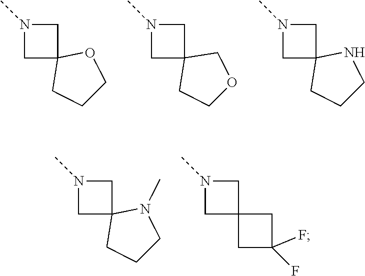

more preferably selected from the group consisting of:

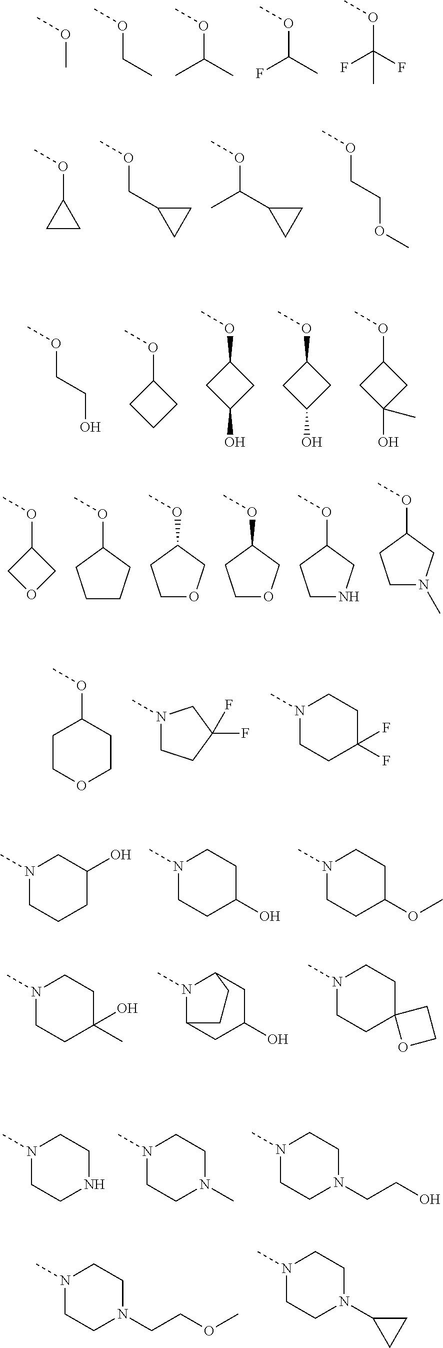

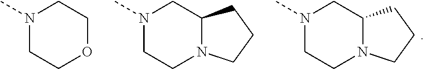

Another aspect of the present disclosure provides a compound of Formula (II):

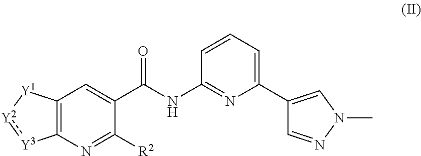

or a pharmaceutically acceptable salt, ester, solvate, prodrug, isotope-labeled derivative, or isomer thereof, wherein:
[symbol] is a single bond or a double bond;
Y₁ is O, S or CH₂;
Y₂ is CR¹ or C(R⁵)R⁶.
Y₃ is N or O;
R¹, R², R⁵ and R⁶ are defined above regarding Formula (I).

In some embodiments, including any one of the hitherto described embodiments, for a compound of Formula (II):

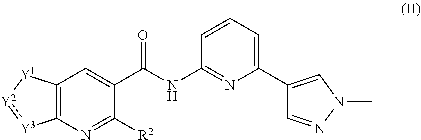

or a pharmaceutically acceptable salt, ester, solvate, prodrug, isotope-labeled derivative, or isomer thereof, wherein:

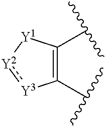

is selected from the group consisting of:

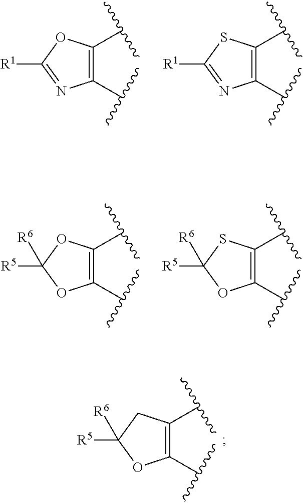

preferably selected from the group consisting of:

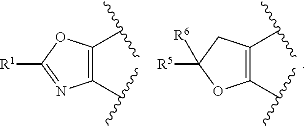

In some embodiments, including any one of the hitherto described embodiments, for a compound of Formula (II):
R¹ is selected from the group consisting of:

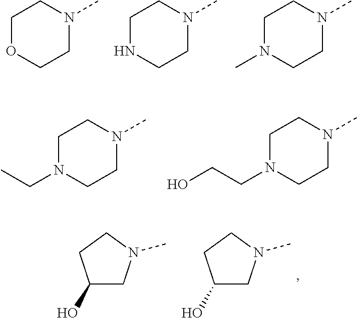

preferably selected from the group consisting of:

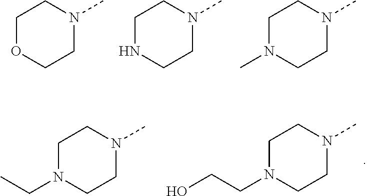

In some embodiments, including any one of the hitherto described embodiments, for a compound of Formula (II):
R² is selected from the group consisting of:

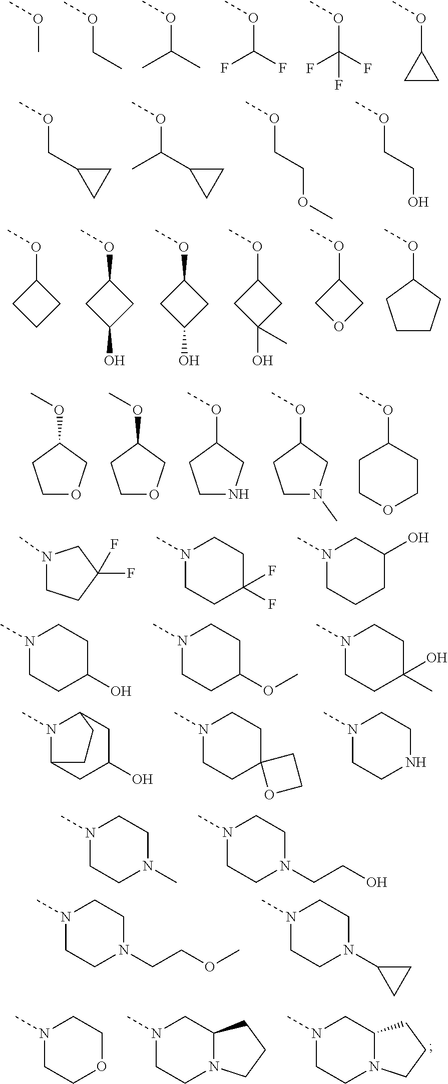

preferably selected from the group consisting of:

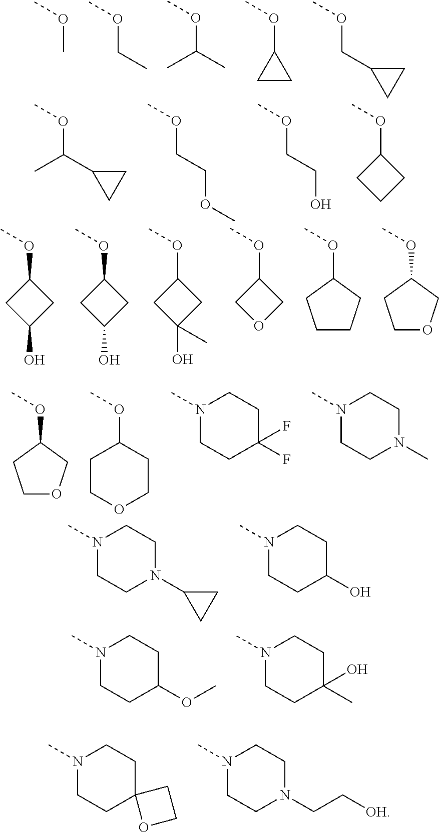

In some embodiments, including any one of the hitherto described embodiments, for a compound of Formula (II):each of R⁵ and R⁶ is independently C₁₋₆ alkyl, preferably methyl.

The second aspect of the present disclosure provides a compound or a pharmaceutically acceptable salt, ester, solvate, prodrug, isotope-labeled derivative, or isomer thereof, wherein the compound is selected from the group consisting of:

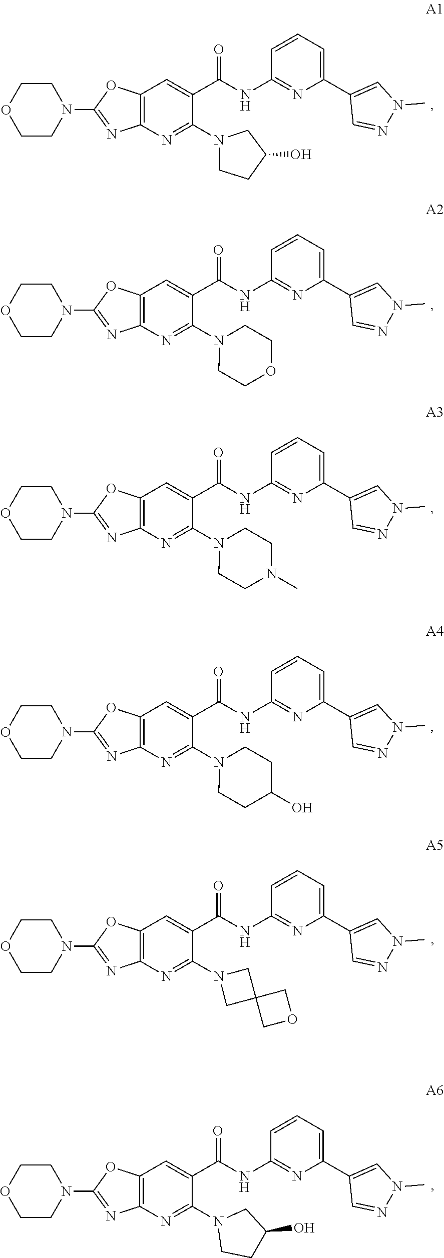 [SMILES: Cn1cc(-c2cccc(NC(=O)c3cc4oc(N5CCOCC5)nc4nc3N3CC[C@@H](O)C3)n2)cn1 ; Cn1cc(-c2cccc(NC(=O)c3cc4oc(N5CCOCC5)nc4nc3N3CCOCC3)n2)cn1 ; CN1CCN(c2nc3nc(N4CCOCC4)oc3cc2C(=O)Nc2cccc(-c3cnn(C)c3)n2)CC1 ; Cn1cc(-c2cccc(NC(=O)c3cc4oc(N5CCOCC5)nc4nc3N3CCC(O)CC3)n2)cn1 ; Cn1cc(-c2cccc(NC(=O)c3cc4oc(N5CCOCC5)nc4nc3N3CC4(COC4)C3)n2)cn1 ; Cn1cc(-c2cccc(NC(=O)c3cc4oc(N5CCOCC5)nc4nc3N3CC[C@H](O)C3)n2)cn1]

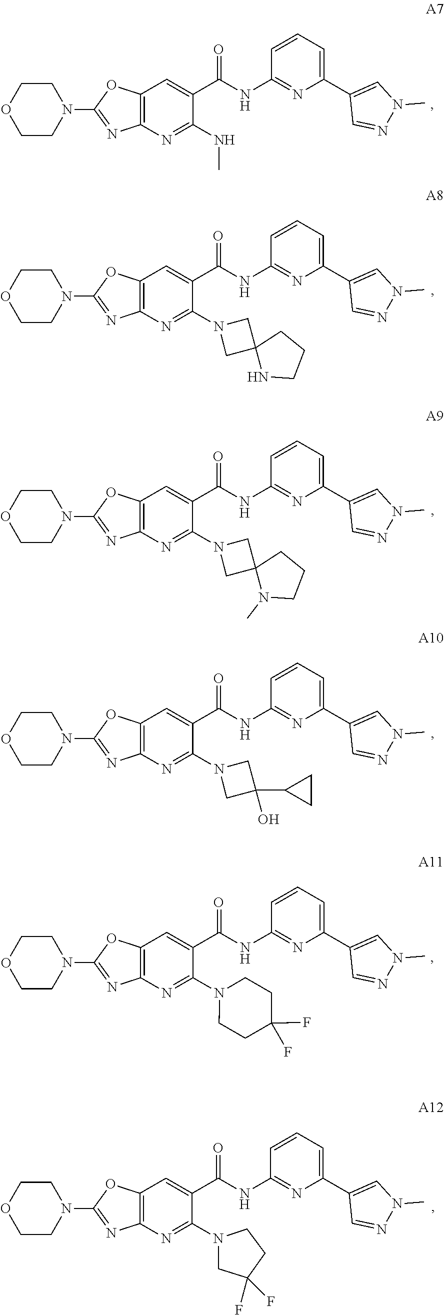 [SMILES: CNc1nc2nc(N3CCOCC3)oc2cc1C(=O)Nc1cccc(-c2cnn(C)c2)n1 ; Cn1cc(-c2cccc(NC(=O)c3cc4oc(N5CCOCC5)nc4nc3N3CC4(CCCN4)C3)n2)cn1 ; CN1CCCC12CN(c1nc3nc(N4CCOCC4)oc3cc1C(=O)Nc1cccc(-c3cnn(C)c3)n1)C2 ; Cn1cc(-c2cccc(NC(=O)c3cc4oc(N5CCOCC5)nc4nc3N3CC(O)(C4CC4)C3)n2)cn1 ; Cn1cc(-c2cccc(NC(=O)c3cc4oc(N5CCOCC5)nc4nc3N3CCC(F)(F)CC3)n2)cn1 ; Cn1cc(-c2cccc(NC(=O)c3cc4oc(N5CCOCC5)nc4nc3N3CCC(F)(F)C3)n2)cn1]

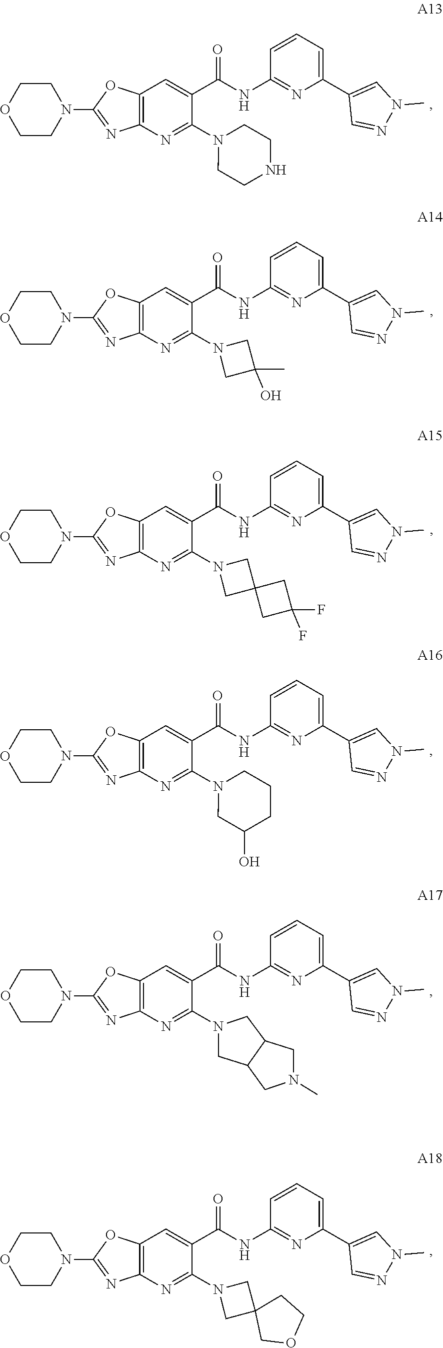 [SMILES: Cn1cc(-c2cccc(NC(=O)c3cc4oc(N5CCOCC5)nc4nc3N3CCNCC3)n2)cn1 ; Cn1cc(-c2cccc(NC(=O)c3cc4oc(N5CCOCC5)nc4nc3N3CC(C)(O)C3)n2)cn1 ; Cn1cc(-c2cccc(NC(=O)c3cc4oc(N5CCOCC5)nc4nc3N3CC4(C3)CC(F)(F)C4)n2)cn1 ; Cn1cc(-c2cccc(NC(=O)c3cc4oc(N5CCOCC5)nc4nc3N3CCCC(O)C3)n2)cn1 ; CN1CC2CN(c3nc4nc(N5CCOCC5)oc4cc3C(=O)Nc3cccc(-c4cnn(C)c4)n3)CC2C1 ; Cn1cc(-c2cccc(NC(=O)c3cc4oc(N5CCOCC5)nc4nc3N3CC4(CCOC4)C3)n2)cn1]

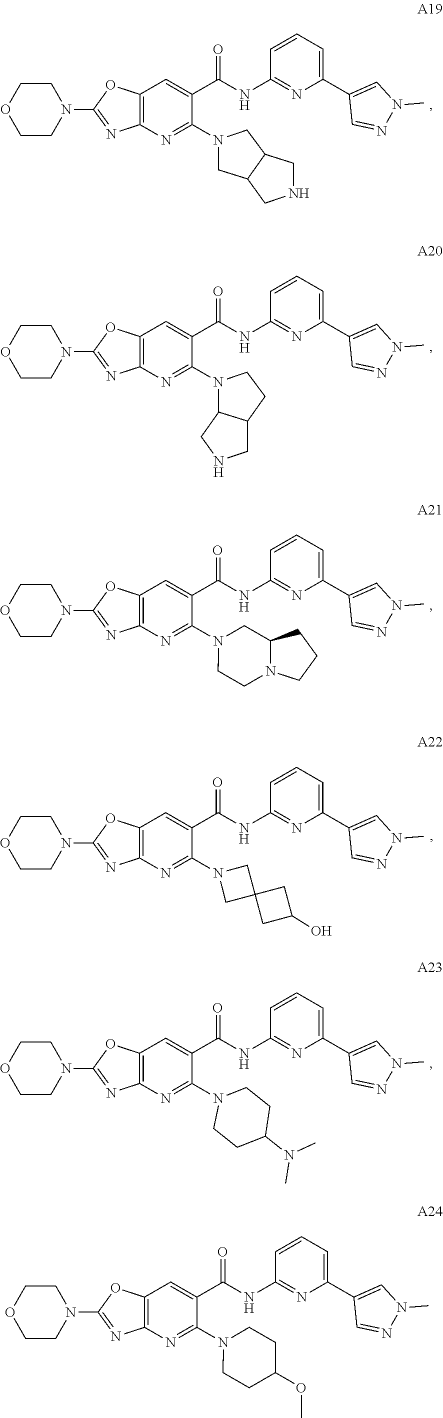 [SMILES: Cn1cc(-c2cccc(NC(=O)c3cc4oc(N5CCOCC5)nc4nc3N3CC4CNCC4C3)n2)cn1 ; Cn1cc(-c2cccc(NC(=O)c3cc4oc(N5CCOCC5)nc4nc3N3CCC4CNCC43)n2)cn1 ; Cn1cc(-c2cccc(NC(=O)c3cc4oc(N5CCOCC5)nc4nc3N3CCN4CCC[C@@H]4C3)n2)cn1 ; Cn1cc(-c2cccc(NC(=O)c3cc4oc(N5CCOCC5)nc4nc3N3CC4(CC(O)C4)C3)n2)cn1 ; CN(C)C1CCN(c2nc3nc(N4CCOCC4)oc3cc2C(=O)Nc2cccc(-c3cnn(C)c3)n2)CC1 ; COC1CCN(c2nc3nc(N4CCOCC4)oc3cc2C(=O)Nc2cccc(-c3cnn(C)c3)n2)CC1]

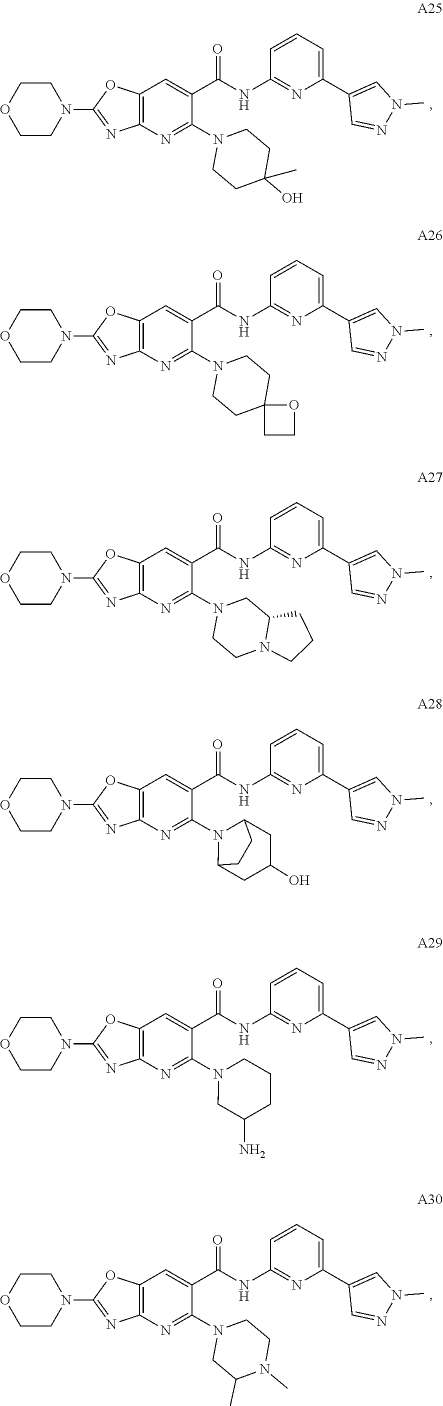 [SMILES: Cn1cc(-c2cccc(NC(=O)c3cc4oc(N5CCOCC5)nc4nc3N3CCC(C)(O)CC3)n2)cn1 ; Cn1cc(-c2cccc(NC(=O)c3cc4oc(N5CCOCC5)nc4nc3N3CCC4(CCO4)CC3)n2)cn1 ; Cn1cc(-c2cccc(NC(=O)c3cc4oc(N5CCOCC5)nc4nc3N3CCN4CCC[C@H]4C3)n2)cn1 ; Cn1cc(-c2cccc(NC(=O)c3cc4oc(N5CCOCC5)nc4nc3N3C4CCC3CC(O)C4)n2)cn1 ; Cn1cc(-c2cccc(NC(=O)c3cc4oc(N5CCOCC5)nc4nc3N3CCCC(N)C3)n2)cn1 ; CC1CN(c2nc3nc(N4CCOCC4)oc3cc2C(=O)Nc2cccc(-c3cnn(C)c3)n2)CCN1C]

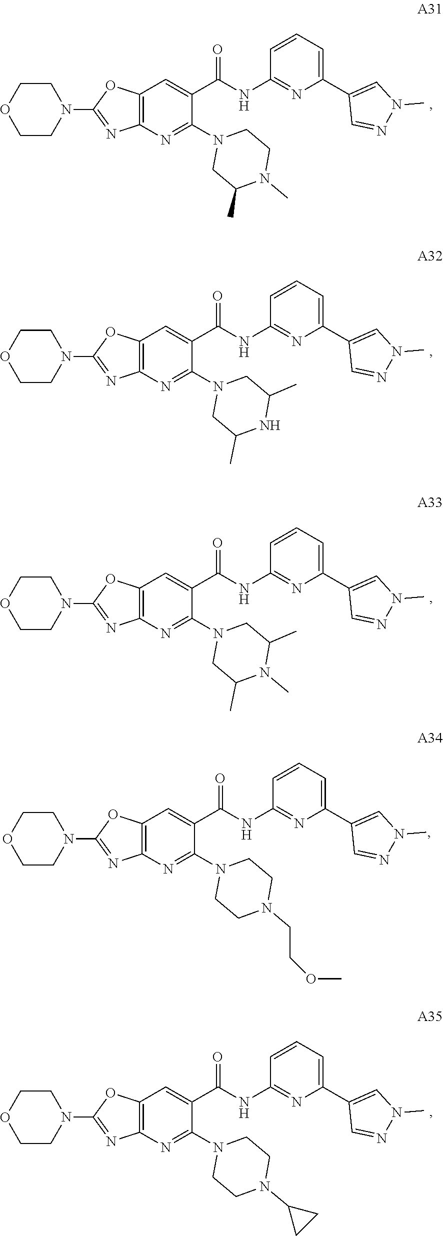 [SMILES: C[C@H]1CN(c2nc3nc(N4CCOCC4)oc3cc2C(=O)Nc2cccc(-c3cnn(C)c3)n2)CCN1C ; CC1CN(c2nc3nc(N4CCOCC4)oc3cc2C(=O)Nc2cccc(-c3cnn(C)c3)n2)CC(C)N1 ; CC1CN(c2nc3nc(N4CCOCC4)oc3cc2C(=O)Nc2cccc(-c3cnn(C)c3)n2)CC(C)N1C ; COCCN1CCN(c2nc3nc(N4CCOCC4)oc3cc2C(=O)Nc2cccc(-c3cnn(C)c3)n2)CC1 ; Cn1cc(-c2cccc(NC(=O)c3cc4oc(N5CCOCC5)nc4nc3N3CCN(C4CC4)CC3)n2)cn1]

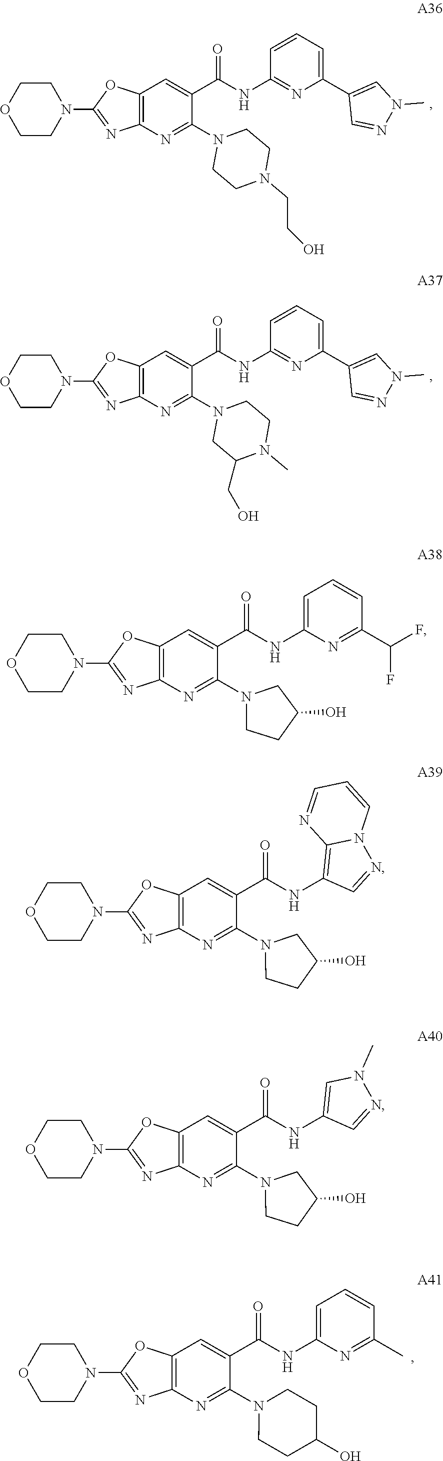 [SMILES: Cn1cc(-c2cccc(NC(=O)c3cc4oc(N5CCOCC5)nc4nc3N3CCN(CCO)CC3)n2)cn1 ; CN1CCN(c2nc3nc(N4CCOCC4)oc3cc2C(=O)Nc2cccc(-c3cnn(C)c3)n2)CC1CO ; O=C(Nc1cccc(C(F)F)n1)c1cc2oc(N3CCOCC3)nc2nc1N1CC[C@@H](O)C1 ; O=C(Nc1cnn2cccnc12)c1cc2oc(N3CCOCC3)nc2nc1N1CC[C@@H](O)C1 ; Cn1cc(NC(=O)c2cc3oc(N4CCOCC4)nc3nc2N2CC[C@@H](O)C2)cn1 ; Cc1cccc(NC(=O)c2cc3oc(N4CCOCC4)nc3nc2N2CCC(O)CC2)n1]

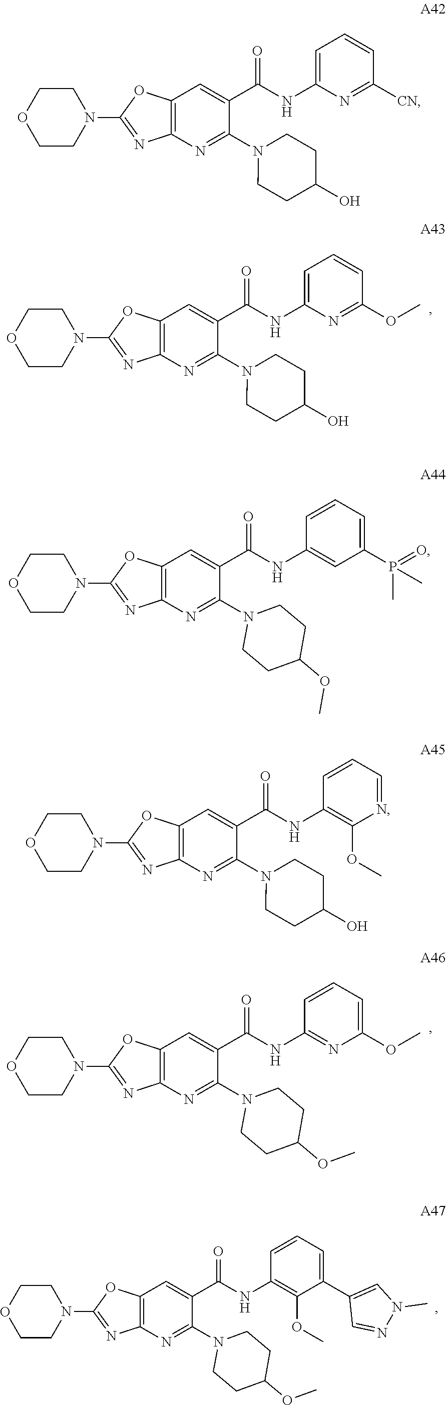 [SMILES: N#Cc1cccc(NC(=O)c2cc3oc(N4CCOCC4)nc3nc2N2CCC(O)CC2)n1 ; COc1cccc(NC(=O)c2cc3oc(N4CCOCC4)nc3nc2N2CCC(O)CC2)n1 ; COC1CCN(c2nc3nc(N4CCOCC4)oc3cc2C(=O)Nc2cccc(P(C)(C)=O)c2)CC1 ; COc1ncccc1NC(=O)c1cc2oc(N3CCOCC3)nc2nc1N1CCC(O)CC1 ; COc1cccc(NC(=O)c2cc3oc(N4CCOCC4)nc3nc2N2CCC(OC)CC2)n1 ; COc1c(NC(=O)c2cc3oc(N4CCOCC4)nc3nc2N2CCC(OC)CC2)cccc1-c1cnn(C)c1]

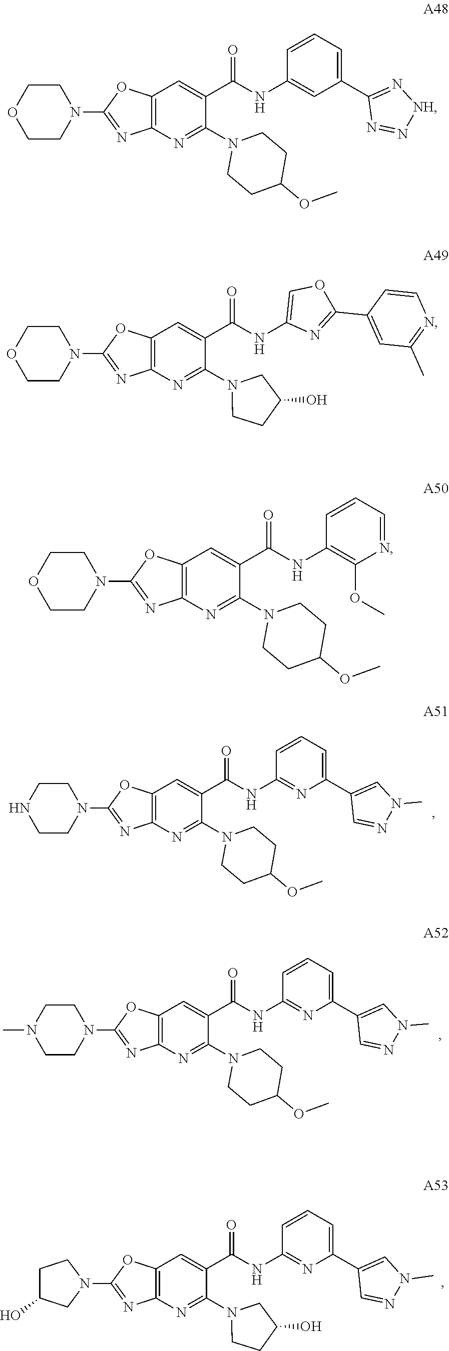

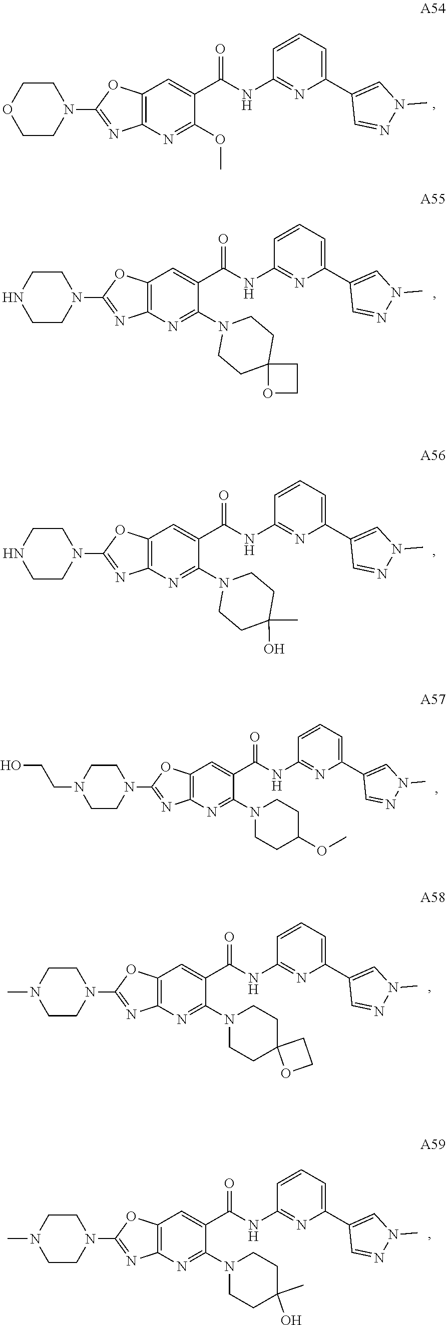

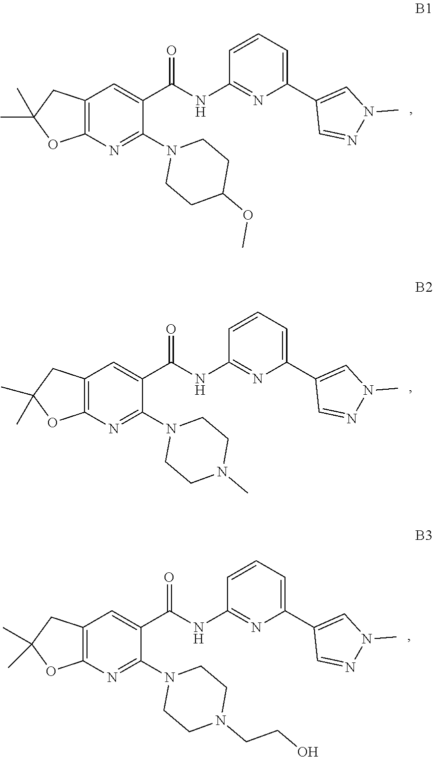

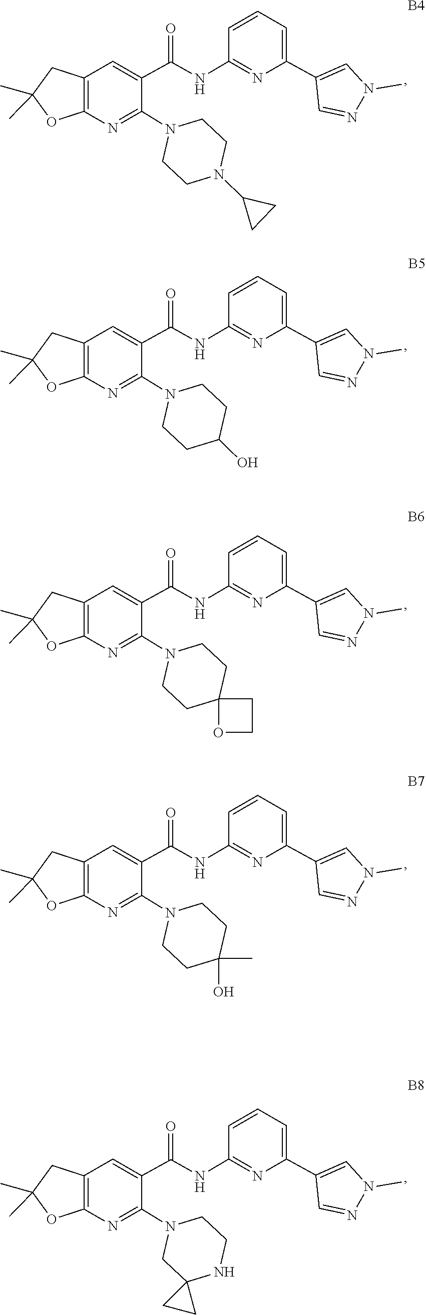

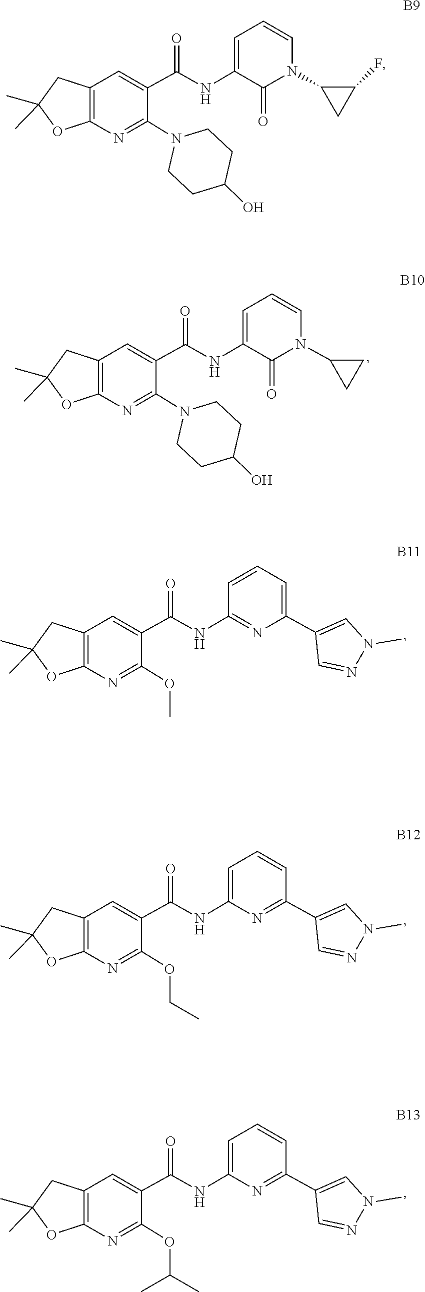

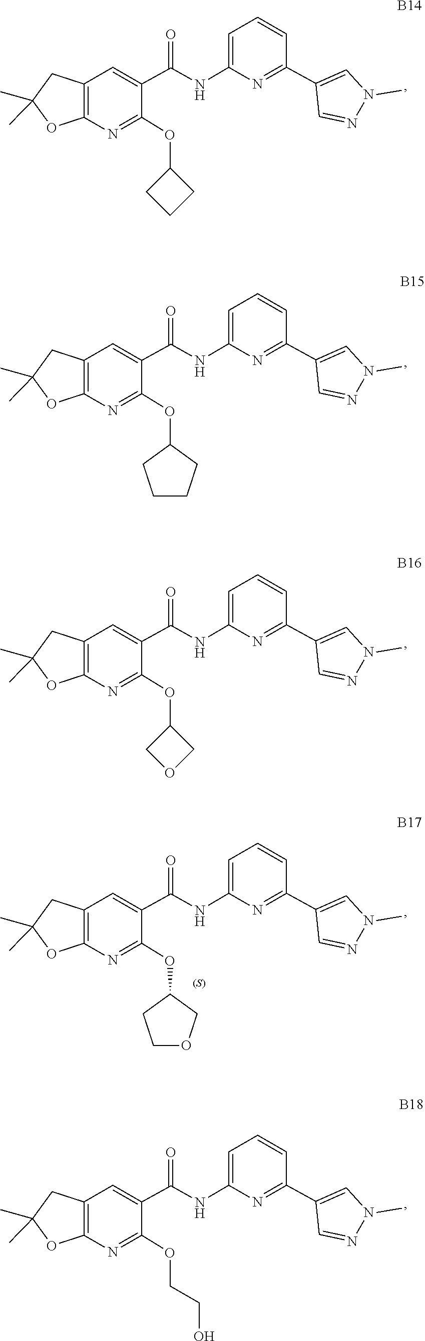

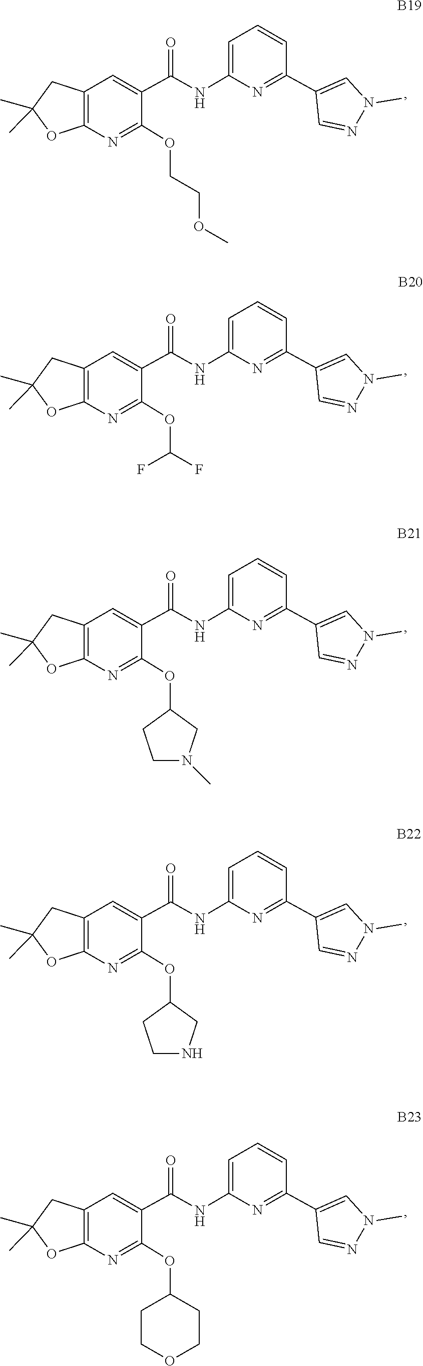

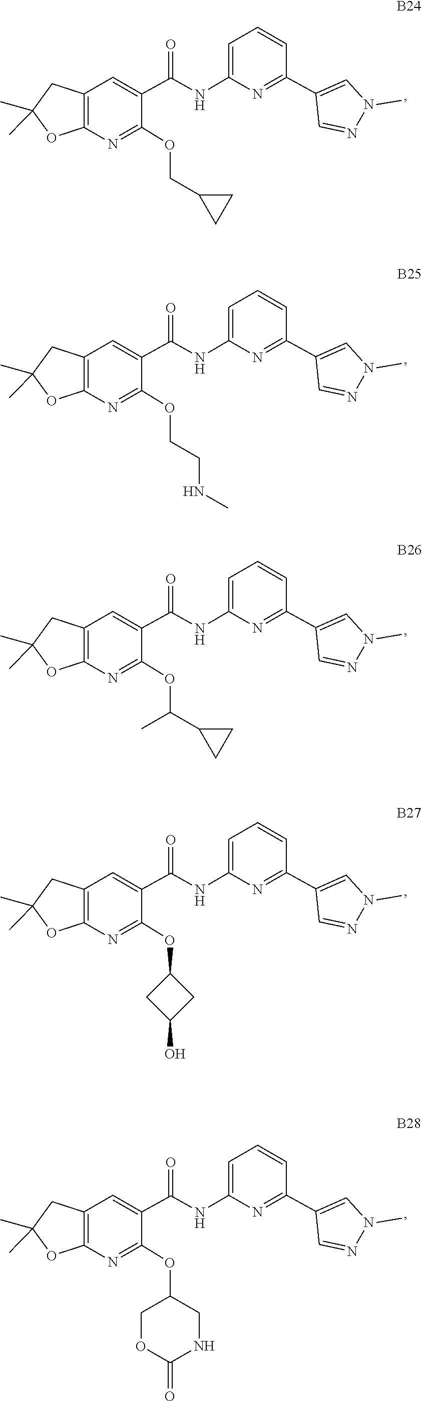

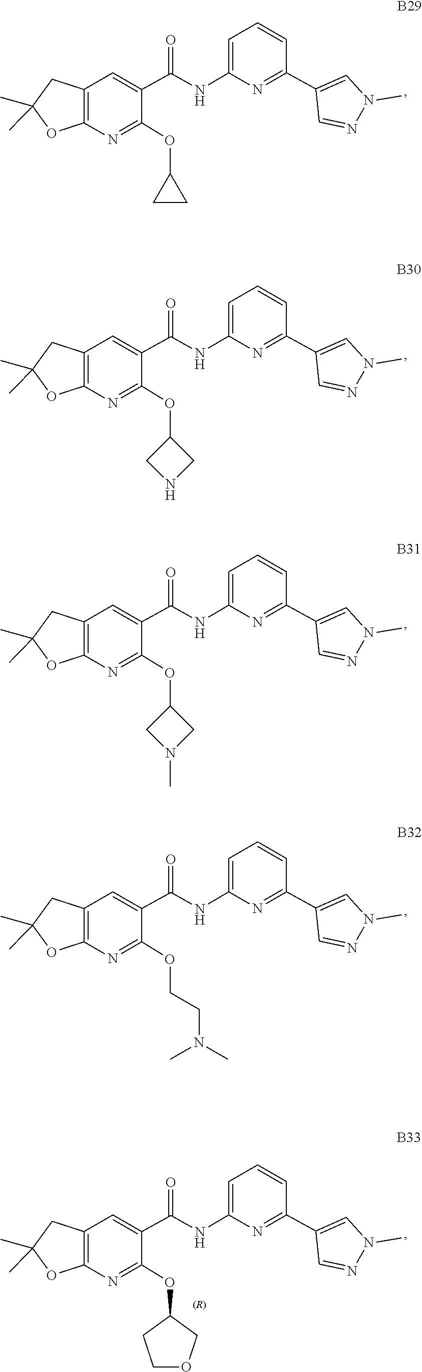

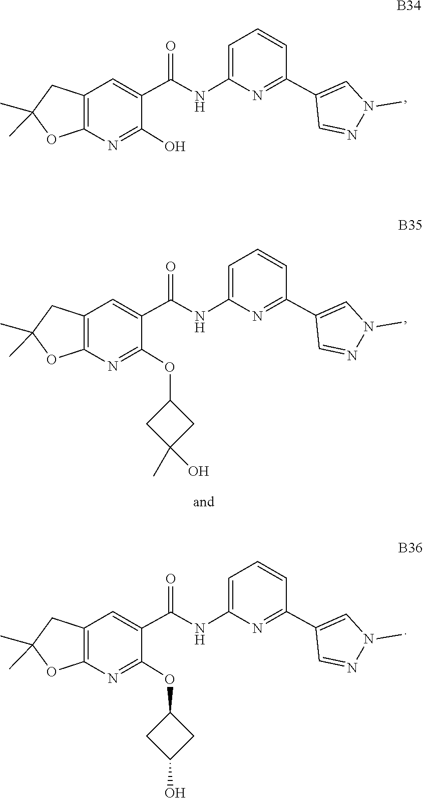

The third aspect of the present disclosure provides a pharmaceutical composition comprising a therapeutically effective amount of the compound of any one of the hitherto described embodiments, or a pharmaceutically acceptable salt, ester, solvate, prodrug, isotope-labeled derivative, or isomer thereof, and a pharmaceutically acceptable carrier.

The fourth aspect of the present disclosure provides a pharmaceutical formulation comprising the compound of any one of the hitherto described embodiments including the first and second aspects, or a pharmaceutically acceptable salt, ester, solvate, prodrug, isotope-labeled derivative or isomer thereof, or the pharmaceutical composition of the third aspect, wherein the pharmaceutical formulation is tablet, capsule, injection, granule, powder, suppository, pill, gel, dispersion, oral solution, inhalant, suspension, or solid suspension, or a combination thereof.

The fifth aspect of the present disclosure provides a composition comprising (i) the compound of any one of the hitherto described embodiments including those of the first and second aspects, or a pharmaceutically acceptable salt, ester, solvate, prodrug, isotope-labeled derivative or isomer thereof, or the pharmaceutical composition described herein according to the third aspect, or the pharmaceutical formulation according to the fourth aspect; and (ii) one or more additional therapeutic agent, the one or more additional therapeutic agent is an anti-neurodegenerative agent, an anti-inflammatory agent, and/or an anti-cancer agent.

The sixth aspect of the present disclosure provides the compound of any one of embodiments disclosed herein including those according to the first and second aspects, or a pharmaceutically acceptable salt, ester, solvate, prodrug, isotope-labeled derivative, or isomer thereof, or the pharmaceutical composition disclosed herein according to the third aspects, or the pharmaceutical formulation according to the fourth aspect, or the composition disclosed herein accordingly the fifth aspect, for use as a medicament in the treatment of the disease or disorder associated with interleukin-1 receptor-associated kinase 4 (IRAK4).

The seventh aspect of the present disclosure provides a use of the compound of any one of embodiments disclosed herein including those according to the first and second aspects, or a pharmaceutically acceptable salt, ester, solvate, prodrug, isotope-labeled derivative, or isomer thereof, or the pharmaceutical composition disclosed herein according to the third aspects, or the pharmaceutical formulation according to the fourth aspect, or the composition disclosed herein accordingly the fifth aspect, to treat and/or prevent the disease or disorder associated with IRAK4.

The eighth aspect of the present disclosure provides a method for treating a disease or disorder in a subject, the method comprising administering to the subject a therapeutically effective amount of the compound of any one of embodiments disclosed herein including those according to the first and second aspects, or a pharmaceutically acceptable salt, ester, solvate, prodrug, isotope-labeled derivative, or isomer thereof, or the pharmaceutical composition disclosed herein according to the third aspects, or the pharmaceutical formulation according to the fourth aspect, or the composition disclosed herein accordingly the fifth aspect, wherein the disease or disorder is associated with IRAK4.

In some embodiments, including any one of the hitherto described embodiments, the disease or disorder is selected from the group consisting of the disease or disorder is uveitis, dermatitis, acute lung injury, type II diabetes, arthritis, ulcerative colitis, Crohn's disease, early-onset inflammatory bowel disease, extraintestinal inflammatory bowel disease, ischemia/reperfusion injury in organ transplant, nonalcoholic fatty liver disease, autoimmune hepatitis, asthma, endometriosis, psoriasis, systemic lupus erythematosus, sarcoidosis, Wegener's granulomatosis, pulmonary fibrosis, renal fibrosis, hepatic fibrosis, myocardiale infarction, hypersensitivity pneumonitis, interstitial lung disease, ankylosing spondylitis, sclerosis, systemic sclerosis, polymyositis, rheumatoid arthritis, myasthenia gravis, juvenile onset diabetes mellitus, glomerulonephritis, autoimmune thyroiditis, graft rejection, Blau syndrome, scleroderma, stomatitis, retinitis pigmentosa, proliferative vitreoretinopathy, Best's yolk macula degeneration, eczema, urticaria, vasculitis, eosinophilic fasciitis, wet and dry age-related macular degeneration, diabetic retinopathy, retinopathy of prematurity, diabetic macular inflammation, retinal vein occlusion, cystic macular edema, glaucoma, Parkinson's disease, Alzheimer's disease, Huntington's disease, breast cancer, lung cancer, bladder cancer, pancreatic cancer, liver cancer, head and neck squamous cell carcinoma, thyroid carcinoma, sarcoma, osteosarcoma, desmoid, melanoma, prostate cancer, colorectal cancer, ovarian cancer, cervical cancer, esophageal cancer, gastric cancer, myeloma, lymphoma, mantle cell lymphoma, cutaneous T-cell lymphoma, chronic and nonprogressive anemia, idiopathic or essential thrombocythemia, leukemia, acute leukemia, chronic leukemia, lymphocytic leukemia, myelogenous leukemia, myelodysplastic syndrome, myeloproliferative disorder, brain tumor, astrocytoma, medulloblastoma, Schwann cell tumor, primary neuroectodermal tumor, or pituitary tumor.

In some embodiments, including any one of the hitherto described embodiments, the disease or disorder is lymphoma, endometriosis, psoriasis, systemic lupus erythematosus, multiple sclerosis, or rheumatoid arthritis. In some embodiments, including any one of the hitherto described embodiments, the lymphoma is primary central nervous system lymphoma or diffuse large B-cell lymphoma with MYD88 L265P mutation.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure.

Accordingly, the drawings and description are to be regarded as illustrative in nature, and not as restrictive.

### BRIEF DESCRIPTION OF THE DRAWINGS

FIG. 1 depicts the experimental results of the inhibition of IRAK4 activities by Compound A4 in Example 4;

FIG. 2 depicts the experimental results of the inhibition of IRAK4 activities by Compound B5 in Example 64;

FIG. 3 depicts the experimental results of the binding to IRAK4 by Compound A4 in Example 4; and

FIG. 4 depicts the experimental results of the inhibition of binding to IRAK4 by Compound B5 in Example 64.

Before proceeding with the detailed description, it is to be appreciated that the following detailed description is merely exemplary in nature and is not intended to limit the invention or the application and uses thereof. Hence, although the present disclosure is, for convenience of explanation, depicted and described as shown in certain illustrative embodiments, it will be appreciated that it can be implemented in various other types of embodiments and equivalents, and in various other systems and environments. Furthermore, there is no intention to be bound by any theory presented in the preceding background or the following detailed description.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Effect of the Present Disclosure

The disclosed compounds having IRAK4 inhibitory activity of the present disclosure can be used as potent inhibitors of IRAK4, and can be used to prevent and/or treat diseases and/or disorders related to or responsive to IRAK4.

### Definitions

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms.

As used herein, the singular forms “a,” “an” and “the” include plural referents unless the context clearly dictates otherwise. Thus, for example, reference to “a molecule” includes a plurality of such molecules, and the like.

The term “about” or “nearly” as used herein generally refers to within +/−15%, 10%, 9%, 8%, 7%, 6%, 5%, 4%, 3%, 2%, or 1% of the designated amount.

The term “halogen” or “halide” as used herein generally refers to fluorine, chlorine, bromine, and iodine. The term “haloalkyl” or “halo-alkyl” as used herein generally refers to an alkyl group that is substituted with one or more independently chosen halogens (e.g., “C₁-C₆ haloalkyl” groups have from 1 to 6 carbon atoms and at least one halogen). Examples of haloalkyl groups include, but are not limited to, mono-, di- or tri-fluoromethyl; mono-, di- or tri-chloromethyl; mono-, di-, tri-, tetra- or penta-fluoroethyl; mono-, di-, tri-, tetra- or penta-chloroethyl; and 1,2,2,2-tetrafluoro-1-trifluoromethyl-ethyl. The term “haloalkoxy” or “halo-alkoxy” as used herein generally refers to an alkoxy group that is substituted with one or more independently chosen halogens (e.g., “C₁-C₆ haloalkoxy” or “C₁-C₆ halo-alkoxy” groups have from 1 to 6 carbon atoms and at least one halogen attached to one of the carbon atoms). Examples of haloalkoxy groups include, but are not limited to, mono- or di-fluoromethoxy; mono- or di-chloromethoxy; mono-, di-, tri-, or tetra-fluoroethoxy; and mono-, di-, tri-, or tetra-chloroethoxy.

The term “alkyl” as used herein generally refers to a straight or branched chain saturated aliphatic hydrocarbon. Alkyl groups include groups having from 1 to 8 carbon atoms (C₁₋₈ alkyl), from 1 to 6 carbon atoms (C₁₋₆ alkyl) and from 1 to 4 carbon atoms (C₁-C₄ alkyl), including, for example, methyl, ethyl, propyl, isopropyl, n-butyl, sec-butyl, isobutyl, tert-butyl, pentyl, 2-pentyl, 3-pentyl, 2-methyl-2-butyl, 3-methyl-2-butyl, 3-methyl-1-butyl, 2-methyl-1-butyl, n-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, and 3,3-dimethyl-2-butyl. Similarly, C₁₋₃ alkyl refers to an alkyl group having from 1 to 3 carbon atoms in a straight or branched chain, including, for example, methyl, ethyl, propyl, and isopropyl. In some instances, a substituent of an alkyl group is specifically indicated. For example, “cyanoalkyl” refers to an alkyl group substituted with at least one cyano substituent. In some embodiments, C₁₋₆ alkyl is, preferably, methyl, ethyl, n-propyl, isopropyl or tert-butyl.

The term “alkylene”, alone or as part of another substituent, refers to a divalent radical derived from an alkane as exemplified by (CH₂)ₙ, where N may be a number from 1 to about 10. By way of example, “C1-2 alkylene” includes but is not limited to methylene, 1,1-ethylene, and 1,2-ethylene.

The term “alkenyl” as used herein generally refers to straight or branched chain alkene groups, which comprise at least one unsaturated carbon-carbon double bond. Alkenyl groups include C₂₋₈ alkenyl, C₂₋₆ alkenyl and C₂₋₄ alkenyl groups, which have from 2 to 8, 2 to 6 or 2 to 4 carbon atoms, respectively, including, for example, ethenyl, allyl or isopropenyl. The term “alkynyl” as used herein generally refers to straight or branched chain alkyne groups, which have one or more unsaturated carbon-carbon bonds, at least one of which is a triple bond. Alkynyl groups include C₂₋₈ alkynyl, C₂₋₆ alkynyl and C₂₋₄ alkynyl groups, which have from 2 to 8, 2 to 6 or 2 to 4 carbon atoms, respectively.

The term “alkoxy” as used herein generally refers to an alkyl group as described above attached via an oxygen bridge to another chemical moiety. Alkoxy groups include different length of the alkyl groups, such as, for example, C₁₋₆ alkoxy and C₁_₄ alkoxy groups, which have from 1 to 6 or from 1 to 4 carbon atoms, respectively. The term “OC₁₋₆ alkyl” as used herein generally refers to alkoxy groups include an alkyl group (with 1 to 6 carbon atoms) attached to an oxygen atom. Methoxy, ethoxy, n-propoxy, isopropoxy, n-butoxy, sec-butoxy, isobutoxy, tert-butoxy, n-pentoxy, 2-pentoxy, 3-pentoxy, isopentoxy, neopentoxy, hexoxy, 2-hexoxy, 3-hexoxy, and 3-methylpentoxy are representative alkoxy groups.

The term “cycloalkyl” as used herein generally refers to a group that comprises one or more saturated rings in which all ring members are carbon. For example, certain cycloalkyl groups are C₃₋₆ cycloalkyl, in which the cycloalkyl group contains one or more rings having from 3 to 6 ring members, all of which are carbon, including, for example, cyclopropyl, cyclobutyl, cyclopentyl, and cyclohexyl. The term “cycloalkenyl” as used herein generally refers to a group that comprises one or more unsaturated rings in which all ring members are carbon.

The terms “heterocyclic” or “heterocycle” or “heterocyclyl” or “cycloheteroalkyl” as used herein generally refer to a ring structure (monocycle or polycycle) containing 3-12 ring atoms (3-12 membered heterocycle), 3-8 ring atoms (3-8 membered heterocycle or 3-8 membered cycloheteroalkyl), 3-6 ring atoms (3-6 membered heterocycle or 3-6 membered cycloheteroalkyl), or 5-6 ring atoms (5-6 membered heterocycle or 5-6 membered cycloheteroalkyl), in which at least one ring atom is carbon, and at least one ring atom is heteroatom selected from N, O, and S or a heteroatom group is selected from P(═O), S(═O), and S(═O)₂. A heterocyclic group may be aromatic or non-aromatic. Piperidine and oxetane are non-limiting examples of non-aromatic heterocycles. Thiazole and pyridine are non-limiting examples of aromatic heterocycles. Other examples of heterocycle include: aziridinyl, azetidinyl, oxetanyl, pyrrolidinyl, tetrahydrofuranyl, tetrahydrothienyl, piperidinyl, morpholinyl, piperazinyl, thiomorpholinyl, tetrahydropyranyl, 1,1-dioxothiomorpholinyl, butyrolactam, valerolactam, caprolactam, butyrolactone, valerolactone and caprolactone.

The terms “6-10 membered bridged biheterocyclyl” or “6-10 membered bridged heterobicyclyl” as used herein generally refer to a saturated or partially unsaturated bicyclic ring structure containing 6 to 10 carbon or heteroatoms or heteroatom groups, in which at least one ring atom is carbon, and at least one ring atom is a heteroatom selected from N, O, and S or a heteroatom group is selected from P(═O), S(═O), and S(═O)₂. Non-limiting examples include but are not limited to 3,6-diazabicyclo[3.1.1]hept-1-yl, 3,6-diazabicyclo[3.1.1]hept-3-yl, and 3,6-diazabicyclo[3.1.1]hept-6-yl, etc.

The term “5-12 membered spiro biheterocyclyl” as used herein generally refers to a saturated or partially unsaturated bicyclic ring structure containing 5 to 12 (especially 6 to 9) carbon or heteroatoms or heteroatom groups, in which the two rings are connected by a carbon, and at least one ring atom is a heteroatom selected from N, O, and S or a heteroatom group is selected from P(═O), S(═O), and S(═O)₂. Non-limiting examples include but are not limited to 4,7-diazaspiro[2.5]oct-7-yl, 1-oxa-6-azaspiro[3.3]hept-6-yl, 2-oxa-6-azaspiro[3.3]hept-6-yl, 2,5-diazaspiro[3.4]oct-2-yl, 5-oxa-2-azaspiro[3.4]oct-2-yl, 6-oxa-2-azaspiro[3.4]oct-2-yl, and 1-oxa-7-aznd aspiro[3.5]non-7-yl, etc.

The term “6-12 membered fused biheterocyclyl” as used herein generally refers to a saturated or partially unsaturated bicyclic ring structure containing 6 to 12 (especially 7 to 10) carbon or heteroatoms or heteroatom groups. The two rings share a carbon-carbon bond, carbon-heteroatom bond, or heteroatom-heteroatom bond (in other words, the two rings share two adjacent ring atoms). The 6-12 membered fused biheterocyclyl comprises at least one ring atom that is a heteroatom selected from N, O, and S or a heteroatom group is selected from P(═O), S(═O), and S(═O)₂. Non-limiting examples include but are not limited to 3,7-diazabicyclo[3.3.0]oct-3-yl, 3,6-diazabicyclo[3.3.0]oct-3-yl, and 2,7-diazabicyclo[3.3.0]oct-2-yl, etc.

The term “aryl” refers to an all-carbon monocyclic or fused-ring polycyclic groups of 6 to 12 (C₆₋₁₂ aryl) or 6 to 10 carbon atoms (C₆₋₁₀ aryl) having a completely conjugated pi-electron system. Examples, without limitation, of aryl groups are phenyl, naphthyl, tetrahydronaphthyl, 2,3-indanyl, biphenyl, and anthracenyl. The aryl group may be substituted or unsubstituted. Typical substituents include halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and —NR^{X}R^{Y}, wherein R^{X} and R^{Y} are independently selected from the group consisting of hydrogen, alkyl, cycloalkyl, aryl, carbonyl, acetyl, sulfonyl, trifluoromethanesulfonyl and, combined, a five- or six-membered heteroalicyclic ring. Illustrative substituted alkyl group include, but are not limited to, fluoromethyl, difluoromethyl, trifluoromethyl, aminomethyl, aminoethyl, hydoxymethyl, methoxymethyl, 2-fluoroethyl, and 2-methoxyethyl, etc.

The term “heteroaryl” as used herein generally refers to an aromatic group or aryl group in which at least one ring carbon of the aromatic group (aryl) is replaced by a heteroatom selected from N, O, and S or a heteroatom group is selected from P(═O), S(═O), and S(═O). Heteroaryls include, for example, 5-12 membered heteroaryls, 5-10 membered heteroaryls, 5-7 membered monocyclic structures or 7-12 membered bicyclic structures. The number of heteroatoms or heteroatom groups in a heteroaryl can be 1, 2, 3, 4, or more. Examples included but are not limited to thienyl, pyridyl, pyrimidinyl, pyrazinyl, pyridazinyl, pyrazolyl, pyridazinyl, 1,2,5-oxadiazolyl, furanyl, pyridin-2(1H)-onyl, pyridin-4(1H)-onyl, pyrrolyl, pyrazolyl, thiazolyl, 1,2,3-triazolyl, 1,2,4-triazolyl, imidazolyl, tetrazolyl, isothiazolyl, oxazolyl, isoxazolyl, thiadiazolyl, oxadiazolyl, naphthyl, benzothienyl, indolyl, benzimidazolyl, benzothiazolyl, benzofuryl, quinolinyl, isoquinolyl and quinazolinyl. The heteroaryl group may be substituted or unsubstituted. Typical substituents include halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and —NR^{X}R^{Y}, with R^{X} and R^{Y} as defined above.

The term “7-10 membered fused bicyclic heterocyclyl” as herein refers to a fused bicyclic ring structure formed by fusing an aryl group with a heteroaryl group or fusing two heteroaryl groups, i.e., the two rings have two adjacent ring atoms in common. Non-limiting examples include, but are not limited to, benzothienyl, indolyl, benzimidazolyl, pyridimidazolyl, pyridazinoimidazolyl, pyrazolopyrimidinyl, benzothiazolyl, benzofuryl, quinolinyl, isoquinolinyl and quinazolinyl.

The term “amino” as used herein generally refers to primary amino group (—NH₂), secondary amino group (—NH—), and tertiary amino group

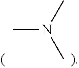

The terms “alkylamino”, “(—NH(C₁₋₆ alkyl)”, “(—N(C₁₋₆ alkyl)₂)”, “(—NH(C₃₋₆ cycloalkyl),” and “(—N(C₃₋₆ cycloalkyl)₂)” as used herein generally refer to a secondary or tertiary amine that comprises one or two alkyl or cycloalkyl groups, each of which can be independently selected.

As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. For example, Berge et al. describes pharmaceutically acceptable salts in detail in Pharmaceutical Sciences (1977) 66: 1-19. Pharmaceutically acceptable salts of the compounds provided herein include those derived from suitable inorganic and organic acids and bases. Inorganic acids from which salts can be derived include, but are not limited to, hydrochloric acid, hydrobromic acid, hydroiodic acid, sulfuric acid, nitric acid, perchloric acid, phosphoric acid, and the like. Organic acids from which salts can be derived include, but are not limited to, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, benzenesulfonic acid, p-toluenesulfonic acid, salicylic acid, succinic acid, lactic acid, malic acids, and the like.

Pharmaceutically acceptable salts derived from appropriate bases include, but are not limited to, alkali metal, alkaline earth metal, ammonium and other amine salt. Inorganic bases from which salts can be derived include, but are not limited to, sodium, potassium, lithium, ammonium, calcium, magnesium, iron, zinc, copper, manganese, aluminum, and the like. Organic bases from which salts can be derived include, but are not limited to, primary, secondary, and tertiary amines, substituted amines, including naturally occurring substituted amines, cyclic amines, basic ion exchange resins, and the like, examples include, but are not limited to, isopropylamine, trimethylamine, diethylamine, triethylamine, tripropylamine, ethanolamine, and quaternary amine N⁺(C₁₋₆ alkyl)₄. In some embodiments, the pharmaceutically acceptable base addition salt is ammonium, potassium, sodium, calcium, or magnesium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Bis salts (i.e., two counterions) and higher salts (e.g., three or more counterions) are encompassed within the meaning of pharmaceutically acceptable salts. In some embodiments, the salts are formed from reacting with LiOH, NaOH, KOH, Na₂CO₃, K₂CO₃, NaHCO₃, KHCO₃, MgCO₃, CaCO₃, NH₄OH, Et₃N, or tetrabutylammonium hydroxide.

As used herein, the term “ester” refers to organic compounds comprising an ester bond, including monoester, diester, trimester, and polyester.

As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Other solvates include, but are not limited to, methanol, ethanol, isopropanol, ethyl acetate, tetrahydrofuran, dimethyl sulfoxide, and N,N-dimethylformamide. Pharmaceutically acceptable solvates and hydrates are complexes that, for example, can include 1 to about 100, or 1 to about 10, or one to about 2, 3 or 4, solvent or water molecules.

As used herein, and unless otherwise specified, “prodrug” refers to a compound that can be converted under physiological conditions or by solvolysis to a biologically active compound described herein, e.g., Formulas (I), (I-A), (I-A1), (I-B), (I-B1), (I-C), or (I-D). Thus, the term “prodrug” refers to a precursor of a biologically active compound that is pharmaceutically acceptable. A prodrug can be inactive when administered to a subject, but is converted in vivo to an active compound, for example, by hydrolysis. A discussion of prodrugs is provided in Higuchi, T., et al, “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound described herein, e.g., Formulas (I), (I-A), (I-A1), (I-B), (I-B1), (I-C), or (I-D), in vivo when such prodrug is administered to a mammalian subject. Prodrugs of an active compound, as described herein, can be prepared by modifying functional groups present in the compounds of Formulas (I), (I-A), (I-A1), (I-B), (I-B1), (I-C), or (I-D) in such a way that the modifications are cleaved, either in routine manipulation or in vivo, to the parent active compound. Prodrugs include compounds wherein a hydroxy, amino or mercapto group is bonded to any group that, when the prodrug of the compounds of Formulas (I), (I-A), (I-A1), (I-B), (I-B1), (I-C), or (I-D) is administered to a mammalian subject, cleaves to form a free hydroxy, free amino or free mercapto group, respectively.

The terms “isotope-labeled”, “isotope label”, “isotope-labeled derivative” and “isotopically labeled” refer to unnatural proportions of atomic isotopes at one or more of atoms that constitute such compounds. For example, the compounds can be radio labeled with radioactive isotopes, such as, for example, tritium (³H), iodine-125 (¹²⁵I), carbon-14 (¹⁴C). The compounds can also be isotope-labeled with ²H, ¹¹C, ¹³C, ¹⁵N, ¹⁷, ¹⁸, ¹⁸F, ³²P ³⁵S, and ³⁶Cl. Certain isotope-labeled disclosed compounds (e.g., those labeled with ³H and ⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some embodiments, provided herein are compounds that can also contain unnatural proportions of atomic isotopes at one or more of atoms that constitute such compounds. All isotopic variations of compounds of the present disclosure, whether radioactive or not, are encompassed within the scope of the present disclosure.

The term “isomers” as used herein generally refers to different compounds that have the same molecular formula, including any and all isomeric forms including enantiomer, disastereomers, tautomers and geometric isomers (including cis- and trans-isomers). For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure, unless specified otherwise. As used herein, the term “tautomer” is a type of isomer that includes two or more interconvertible compounds resulting from at least one formal migration of a hydrogen atom and at least one change in valency (e.g., a single bond to a double bond, a triple bond to a single bond, or vice versa). Therefore, a stereochemical isomer of a compound recited in the present disclosure, and its enantiomers, diastereomers, tautomers or geometric isomers (or cis-trans isomers), or a combination thereof are all included in the scope of the present disclosure.

The term “each independently”, as used herein, means that at least two groups (or ring systems) present in a structure with the same or similar value ranges may have the same or different meanings under certain circumstances. For example, if substituent X and substituent Y are each independently hydrogen, halogen, hydroxy, cyano, alkyl or aryl, then when substituent X is hydrogen, substituent Y can be hydrogen, halogen, hydroxy, cyano, alkyl or aryl. Similarly, when the substituent Y is hydrogen, the substituent X can be hydrogen, halogen, hydroxy, cyano, alkyl or aryl.

The terms “optional” or “optionally”, as used herein, mean that the subsequently described event or circumstance may or may not occur, and that the description includes both the occurrence and the non-occurrence of the subsequent event or circumstance.

The terms “substituent” and “substituted,” as used herein, generally denote that a molecular moiety is covalently bonded to an atom within a molecule of interest. For example, a ring substituent may be a moiety such as a halogen, alkyl group, hydroxy, haloalkyl group or other group that is covalently bonded to an atom (preferably a carbon or nitrogen atom) that is a ring member. Substituents of aromatic groups are generally covalently bonded to a ring carbon atom. A straight chain substituent may be a moiety such as a halogen, alkyl group, haloalkyl group or other group that is covalently bonded to an atom (preferably a carbon or nitrogen atom) that is a member of a straight chain.

The term “pharmaceutically acceptable” as used herein generally refers to a form of the compound that is safe for administration to a subject. For example, a free base, a salt form, a solvate, a hydrate, a prodrug or derivative form of a compound described herein, which has been approved for mammalian use, via oral ingestion or any other route of administration, by a governing authority or regulatory agency, such as the Food and Drug Administration (FDA) of the United States, is pharmaceutically acceptable.

In some embodiments, the compound(s) of Formulas (I), (I-A), (I-A1), (I-B), (I-B1), (I-C), or (I-D) is used to treat a subject by administering the compound(s) as a pharmaceutical composition. To this end, the compound(s), in one embodiment, is combined with one or more pharmaceutically acceptable excipients, including carriers, diluents or adjuvants, to form a suitable composition, which is described in more detail herein.

The term “excipient” as used herein generally refers to any pharmaceutically acceptable additive, carrier, adjuvant, or other suitable ingredient, other than the active pharmaceutical ingredient (API), which is typically included for formulation and/or administration purposes.

The term “diluent” as used herein generally refers to an agent used as filler in order to achieve the desired composition volume or weight. The diluent may be present in the pharmaceutical composition within granules in the form of a single compound or in the form of a mixture of compounds. Non-limiting examples of diluent include lactose, starch, pregelatinized starch, microcrystalline cellulose, silicified microcrystalline cellulose, cellulose acetate, dextrose, mannitol, sodium phosphate, potassium phosphate, calcium phosphate, fructose, maltose, sorbitol, or sucrose.

The term “adjuvant,” as used herein generally refers to any substance or mixture of substances that increases the efficacy or potency of a compound disclosed herein on a target where the adjuvant is used together with the compound disclosed herein. However, when the adjuvant is used alone, no pharmacological effect is observed on the same target.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The terms “treat”, “treating,” “treatment,” and “therapy” as used herein generally refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals. Treatment includes the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “effective amount” or “therapeutically effective amount”, as used herein, refers to a sufficient amount of an agent or a compound being administered which will relieve one or more of the symptoms of the disease or condition being treated to some extent; achieve the goal of improvement in disorder severity and the frequency of incidence over treatment of each agent by itself, the result thereof can be reduction and/or alleviation of the signs, symptoms, or causes of a disease, or any other desired alteration of a biological system; while avoiding adverse side effects typically associated with alternative therapies. For example, an “effective amount” for therapeutic uses is the amount of the composition as disclosed herein required to provide a clinically significant decrease in disease symptoms. An appropriate “effective” amount in any individual case may be determined using techniques, such as a dose escalation study. The effective amount, in one embodiment, is administered in a single dosage form or in multiple dosage forms.

Regardless of the route of administration selected, the compounds of the present invention, which may be used in a suitable hydrated form, and/or the pharmaceutical compositions of the present invention, are formulated into pharmaceutically acceptable dosage forms or by other conventional methods known to those of skill in the art.

Actual dosage levels of the active ingredients in the pharmaceutical compositions of the present invention may be varied so as to obtain an effective amount of the active ingredient to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular hedgehog inhibitor employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day. The mode of administration can have a large effect on dosage. Higher doses may be used for localized routes of delivery.

If desired, the effective daily dose of the active compound may be administered as two, three, four, five, six or more sub-doses administered separately at appropriate intervals throughout the day, optionally, in unit dosage forms. Those of skill in the art will readily appreciate that dose levels can vary as a function of the specific compound, the severity of the symptoms and the susceptibility of the subject to side effects. Dosages for a given compound disclosed herein are readily determinable by those of skill in the art by a variety of means.

### Pharmaceutical Compositions/Formulations

One embodiment provides a pharmaceutical composition comprising a compound of Formulas (I), (I-A), (I-A1), (I-B), (I-B1), (I-C), or (I-D), or a stereoisomer, tautomer, hydrate, solvate or pharmaceutically acceptable salt thereof, and at least one pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed., Easton, Pa.: Mack Publishing Company (1995); Hoover, John E., Remington's Pharmaceutical Sciences, Mack Publishing Co., Easton, Pennsylvania (1975); Liberman, H. A. and Lachman, L., Eds., Pharmaceutical Dosage Forms, Marcel Decker, New York, N.Y. (1980); and Pharmaceutical Dosage Forms and Drug Delivery Systems, Seventh Ed., Lippincott Williams & Wilkins (1999), herein incorporated by reference for such disclosure.

A pharmaceutical composition, as used herein, refers to a mixture of a compound of Formulas (I), (I-A), (I-A1), (I-B), (I-B1), (I-C), or (I-D), with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

All formulations for oral administration are in dosages suitable for such administration. Examples of such dosage units are tablets or capsules. In some embodiments, these contain an amount of active ingredient from about 1 to 2000 mg, advantageously from about 1 to 500 mg, and typically from about 5 to 150 mg. A suitable daily dose for a human or other mammal vary widely depending on the condition of the patient and other factors, but, once again, can be determined using routine methods and practices.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

### Synthetic Methods

The examples and preparations provided below illustrated and exemplify the compounds described herein and methods of preparing such compounds. In general, the compounds described herein may be prepared by processes known in the general chemical arts.

The compounds of the present invention can be prepared using various synthetic routes, including those described below, starting from commercially available materials. Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Functional groups may be removed according to known procedures in the art.

The protection of functional groups by protecting groups, the protecting groups themselves, and their removal reactions (commonly referred to as “deprotection”) are described, for example, in standard reference works, such as J. F. W. McOmie, Protective Groups in Organic Chemistry, Plenum Press, London and New York (1973), in T. W. Greene, Protective Groups in Organic Synthesis, Wiley, New York (1981), in The Peptides, Volume 3, E. Gross and J. Meienhofer editors, Academic Press, London and New York (1981).

All synthetic procedures described herein can be carried out under known reaction conditions, advantageously under those described herein, either in the absence or in the presence (usually) of solvents or diluents.

The invention further encompasses “intermediate” compounds, including structures produced from the synthetic procedures described, whether isolated or not, prior to obtaining the finally desired compound. Structures resulting from carrying out steps from a transient starting material, structures resulting from divergence from the described method(s) at any stage, and structures forming starting materials under the reaction conditions are all “intermediates” included in the invention. Further, structures produced by using starting materials in the form of a reactive derivative or salt, or produced by a compound obtainable by means of the process according to the invention and structures resulting from processing the compounds of the invention in situ are also within the scope of the invention.

New starting materials and/or intermediates, as well as processes for the preparation thereof, are likewise the subject of this invention. In select embodiments, such starting materials are used and reaction conditions so selected as to obtain the desired compound(s).

Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Protecting groups, their introduction and removal are described above.

All reagents and solvents were obtained commercially as analytical pure or chemical pure reagents unless stated otherwise. All commercial reagents and solvent were used without purification unless stated otherwise. When required, some reagents and solvents were purified by standard techniques, such as distillation. Anyhydrous solvents were treated according to standard methods or referenced methods. For example, tetrahydrofuran may be purified by distillation from sodium. All thin-layer chromatography (TLC, GF254) analyses and column purification (100-200 mesh) were performed on silica gel (Qingdao Haiyang Chemical Co. Ltd. or Yantai Chemical Co. Ltd.), using petroleum ether (b.p. 60-90° C.)/ethyl acetate (v/v) as eluent; and spots revealed by UV visualization at 254 nm and I₂ vapor or phosphomolybdic acid. All organic layers after extraction were dried over anhydrous Na₂SO₄ unless stated otherwise. All nuclear magnetic resonance spectra (¹H NMR) were recorded using a Varian-400 spectrometer at 400 MHz using TMS as an internal standard. LC-MS was run using an Agilent 1100 system with LC-MSDTrap recorder, diode array detector (DAD) with detecting wavelength at 214 nm and 254 nm, and ESI source. The HPCL column is an AgelaDurashell C18 column (3.5 μm 4.6×50 mm). Gradients were run using 0.1 NH₄HCO₃ aqueous solution and acetonitrile with gradient 5/95 to 95/5 in the run time indicated (for example, 5 min), flow rate at 1.8 mL/min.

The size and scale of the synthetic methods will vary depending on the desired amount of end product. It is understood that while specific reactants and amounts are provided in the Examples, one of skill in the art knows other alternative and equally feasible sets of reactants that will also yield the same compounds. Thus, where general oxidizers, reducers, solvents of various nature (aprotic, apolar, polar, etc.) are utilized, equivalents will be known in the art and are herein contemplated for use in the present methods.

Many of the steps below indicate various work-ups following termination of the reaction. A work-up involves generally quenching of a reaction to terminate any remaining catalytic activity and starting reagents. This is generally followed by addition of an organic solvent and separation of the aqueous layer from the organic layer. The product is typically obtained from the organic layer and unused reactants and other spurious side products and unwanted chemicals are generally trapped in the aqueous layer and discarded. The work-up in standard organic synthetic procedures found throughout the literature is generally followed by drying the product by exposure to a drying agent, such as anhydrous Na₂SO₄, to remove any excess water or aqueous byproducts remaining partially dissolved in the organic layer and concentration of the remaining organic layer. Concentration of product dissolved in solvent may be achieved by any known means, such as evaporation under pressure, evaporation under increased temperature and pressure, and the like. Such concentrating may be achieved by use of standard laboratory equipment such as rotary-evaporator distillation, and the like. This is optionally followed by one or more purification steps which may include, but is not limited to, flash column chromatography, filtration through various media and/or other preparative methods known in the art and/or crystallization/recrystallization. (See, for instance, Addison Ault, “Techniques and Experiments for Organic Chemistry,” 6th Ed., University Science Books, Sausalito, Calif., 1998, Ann B. McGuire, Ed., pp. 45-59).

### Abbreviations

NBS is N-bromosuccinimide.
T3P is 1-propylphosphonic acid cyclic anhydride.
DMF means N,N-dimethylformamide
LDA is lithium diisopropylamide.
DCE means 1,2-dichloroethane.
DCM is dichloromethane.
TEA is triethylamine.
EtOAc or EA means ethyl acetate.
THF is tetrahydrofuran.
TFA is trifluoroacetic acid.
TKB is potassium tert-butoxide.
DAST is diethylaminosulfur trifluoride.
DMEDA is 1,2-dimethylethylenediamine.
EG is ethylene glycol.
Xantphos is 4,5-bis(diphenylphosphino)-9,9-dimethylxanthene.
HATU is 2-(7-aza-1H-benzotriazole-1-yl)-1,1,3,3-tetramethyluronium
hexafluorophosphate.
DIPEA is diisopropylethylamine.
Pd(dppf)Cl₂ is [1,1′-bis(diphenylphosphino)ferrocene]dichloropalladium(II)
Pd(OAc)₂ is palladium (II) acetate.
DPPA is diphenylphosphoryl azide.
DIAD is diisopropylazodiformate.
NMP is N-methyl pyrrolidone.
TLC means thin layer chromatography.
HPLC means high-performance liquid chromatography.
LC-MS means liquid chromatography-mass spectrometry.
NMR means nuclear magnetic resonance.

### General Synthetic Routes

The following Methods AA-BK are embodiments for some general synthetic routes leading to compounds of Formulas (I), (I-A), (I-A1), (I-B), (I-B1), (I-C), or (I-D). Detailed reaction conditions for each Method can be found in the examples shown vide infra.

### Method AA

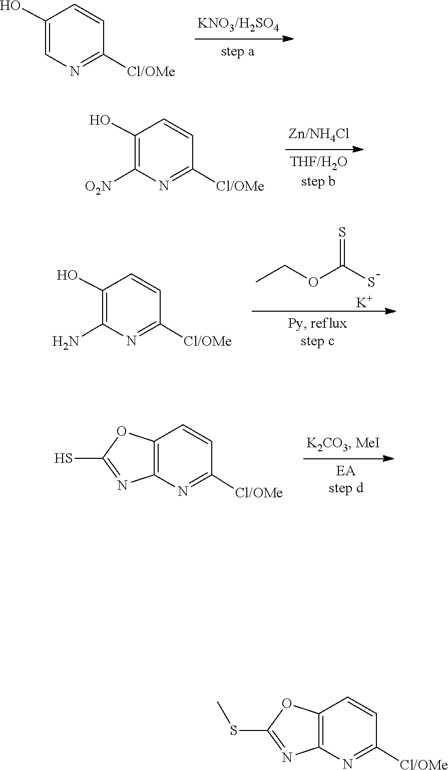

Nitration on C2 of the pyridine gave the corresponding nitro pyridinol intermediate (step a), the nitro group of which was then reduced by zinc powder to give the corresponding amino pyridinol intermediate (step b). The subsequent reaction was the cyclization of the amino pyridinol compound by treating with potassium ethylxanthate to give oxozolo [4,5-b]pyridine-2-thiol compounds (step c). The thiol group was methylated to yield the desired compound (step d).

### Method AB

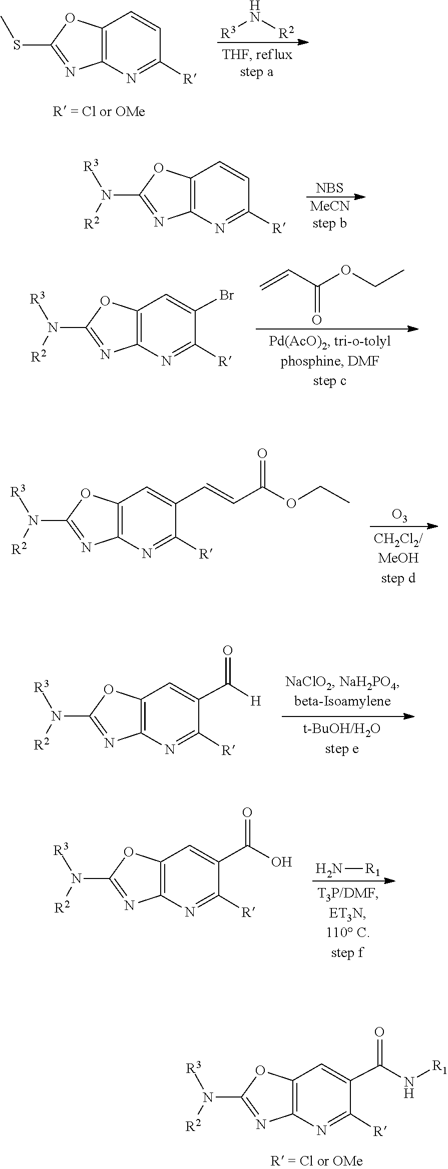

S_{N}Ar reaction of the sulfide moiety with an amine formed the corresponding amino substituted oxozolo [4,5-b]pyridine intermediate (step a). Bromination on C6 of oxozolo [4,5-b]pyridine by NBS facilitated the subsequent palladium-catalyzed Heck coupling with ethyl acrylate to give the alkene intermediate (steps b, c). Ozonization of the alkene intermediate by bubbling O₃ and Pinnick oxidation gave the carboxylic acid compound (steps d, e). Amide coupling in the presence of T₃P converted the carboxylic acid into the corresponding amine—the desired compounds (step f).

### Method AC

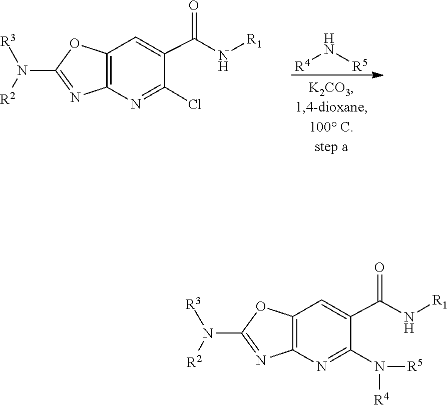

S_{N}Ar reaction of the chlorine moiety on C5 of oxozolo [4,5-b]pyridine with an amine yielded the corresponding 5-amino substituted intermediate (step a).

### Method AD

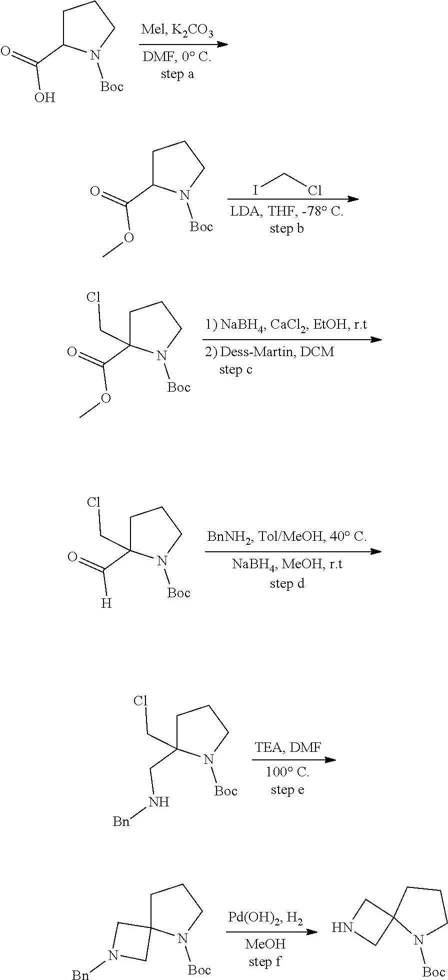

Methylation of N-(tert-butoxycarbonyl)proline was achieved by treating with iodomethane under basic condition (step a). The alkylation of proline methyl ester with chloroiodomethane yielded 2-chloromethyl substituted proline methyl ester (step b). Borohydride reduction of the ester to an alcohol followed by Dess-Martin oxidation gave an aldehyde (step c), which reacted with benzylamine via a reductive amination to afford the corresponding intermediate (step d). Intramolecular cyclization followed by benzyl-deprotection under H₂ atmosphere led to the desired compound (steps e, f).

### Method AE

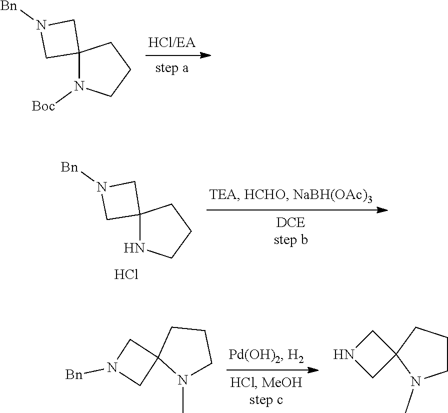

Boc-deprotection under acid condition gave a secondary amine which reacted with formaldehyde by means of reductive amination to afford a tertiary amine (steps a, b). Hydrogenation was then conducted to give 5-methyl-2,5-diazaspiro[3,4]octane (step c).

### Method AF

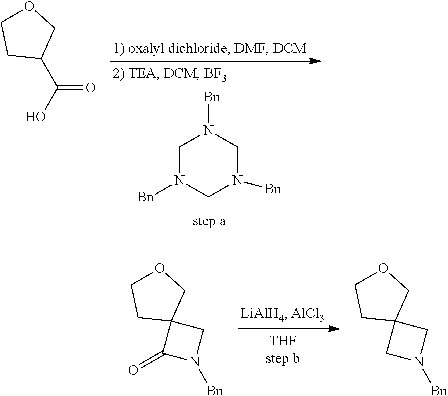 [SMILES: O=C(O)C1CCOC1 ; c1ccc(CN2CN(Cc3ccccc3)CN(Cc3ccccc3)C2)cc1 ; O=C1N(Cc2ccccc2)CC12CCOC2 ; c1ccc(CN2CC3(CCOC3)C2)cc1]

The carboxylic acid reacted with oxalyl dichloride catalyzed by DMF to yield an acyl chloride, which was treated with 1,3,5-tribenzyl-1,3,5-triazinane to afford a benzyl protected lactam (step a). Lithium aluminum hydride in the presence of a Lewis acid reduced the amide, resulting in a tertiary amine compound (step b).

### Method AG

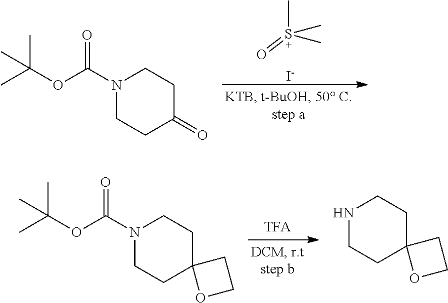

Cyclization reaction between tert-butyl 4-oxopiperidine-1-carboxylate and trimethylsulfoxonium iodide followed by Boc-deprotection reaction under acidic conditions formed the secondary amine (steps a, b).

### Method AH

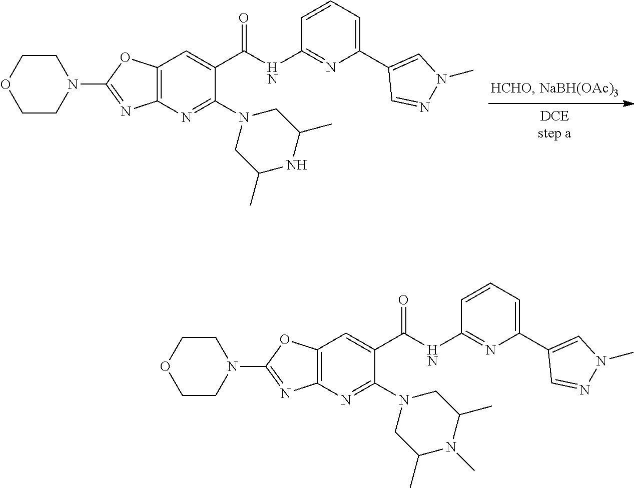 [SMILES: CC1CN(c2nc3nc(N4CCOCC4)oc3cc2C(=O)Nc2cccc(-c3cnn(C)c3)n2)CC(C)N1 ; CC1CN(c2nc3nc(N4CCOCC4)oc3cc2C(=O)Nc2cccc(-c3cnn(C)c3)n2)CC(C)N1C]

The tertiary amine compound was obtained by reductive amination between a secondary amine and paraformaldehyde together with sodium triacetoxyborohydride (step a).

### Method AI

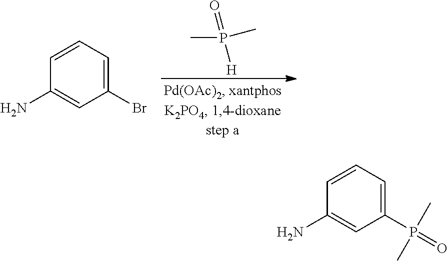

3-Bromoaniline coupling with dimethylphosphine oxide was catalyzed by palladium acetate to give the corresponding compound (step a).

### Method AJ

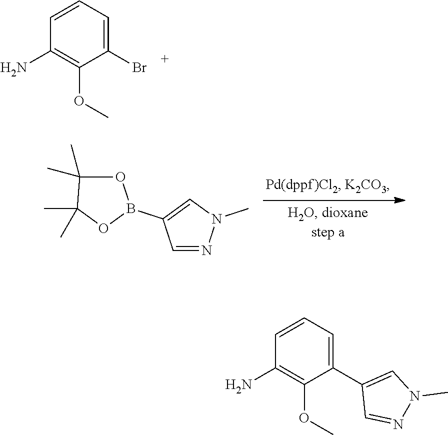

Suzuki coupling between an aryl halide and a boric acid ester gave the desired product (step a).

### Method AK

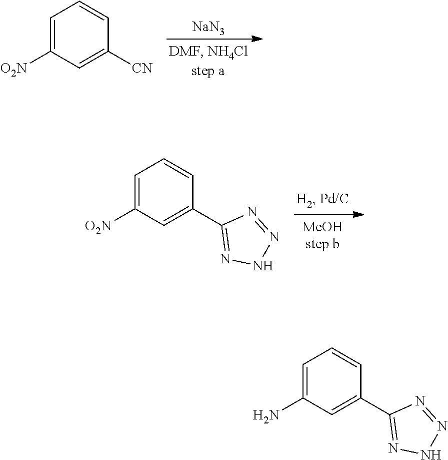 [SMILES: N#Cc1cccc([N+](=O)[O-])c1 ; O=[N+]([O-])c1cccc(-c2nn[nH]n2)c1 ; Nc1cccc(-c2nn[nH]n2)c1 ; "NaN3"]

A 2H-tetrazole intermediate was synthesized via a 1,3-dipolar cycloaddition between 3-cyano nitrobenzene and sodium azide (step a). Hydrogenation of the nitro group was then conducted to afford the aniline compound (step b).

### Method AL

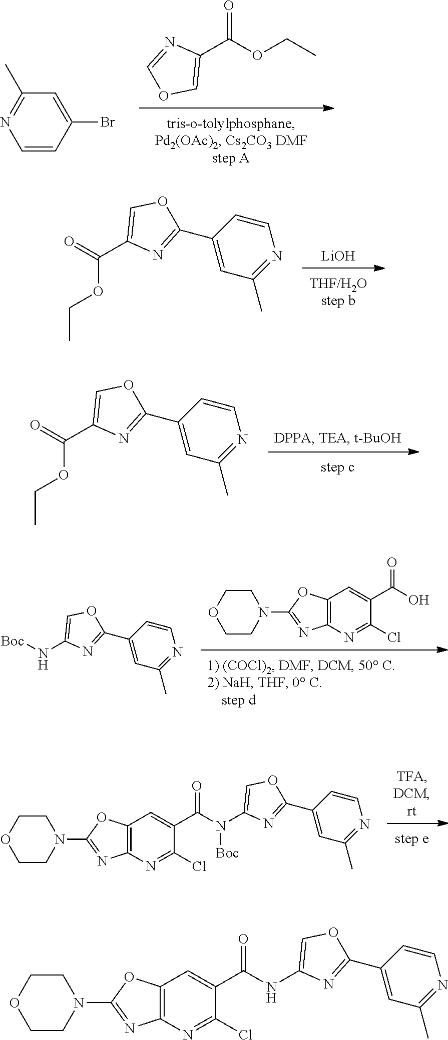

Ethyl 2-(2-methylpyridin-4-yl)oxazole-4-carboxylate was obtained by coupling an oxazole with 4-bromo-2-methyl pyridine catalyzed by palladium acetate. The ester was hydrolyzed under basic condition to afford the 4-oxazole carboxylic acid (steps a, b). The carboxylic group was transformed to Boc protected amino group by means of Curtius rearrangement (step c). The Boc protected amino group then attacked an acyl chloride prepared by another pyridine carboxylic acid under the strong basic condition, resulting in an amide, the Boc of which was then removed under strong acidic condition (steps d, e).

### Method AM

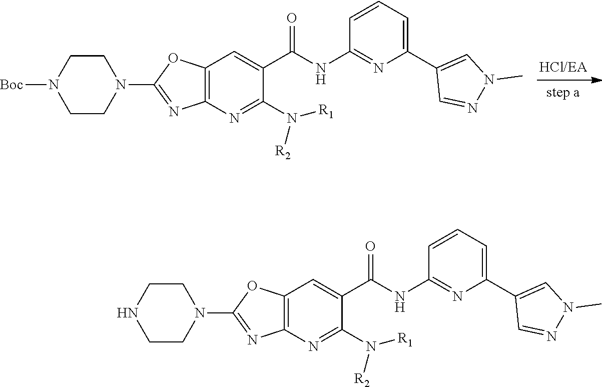

The Boc on piperazine was removed under acidic condition to afford the desired compound (step a).

### Method AN

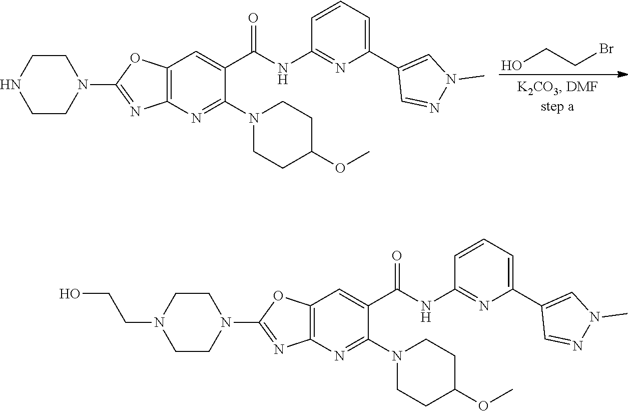

A 4-(2-hydoxyethyl) substituted piperazine was formed via an S_{N}2 reaction between 2-bromothane-1-ol and a piperazine under inorganic base condition (step a).

### Method AO

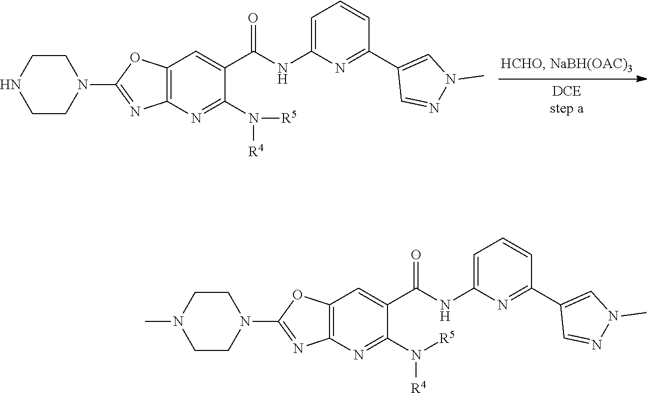

Reductive amination reaction between a piperazine and formaldehyde using sodium triacetoxyborohydride as the reducing agent led to a 4-methyl piperazine (step a).

### Method AP

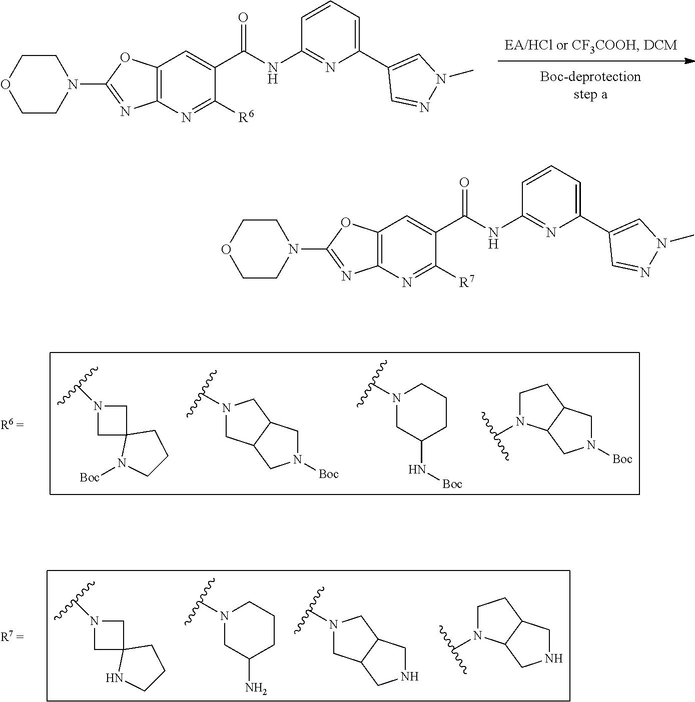

Boc-deprotection was made by the treatment of HCl/EA or CF₃COOH to obtain the corresponding compounds (step a)

### Method BA

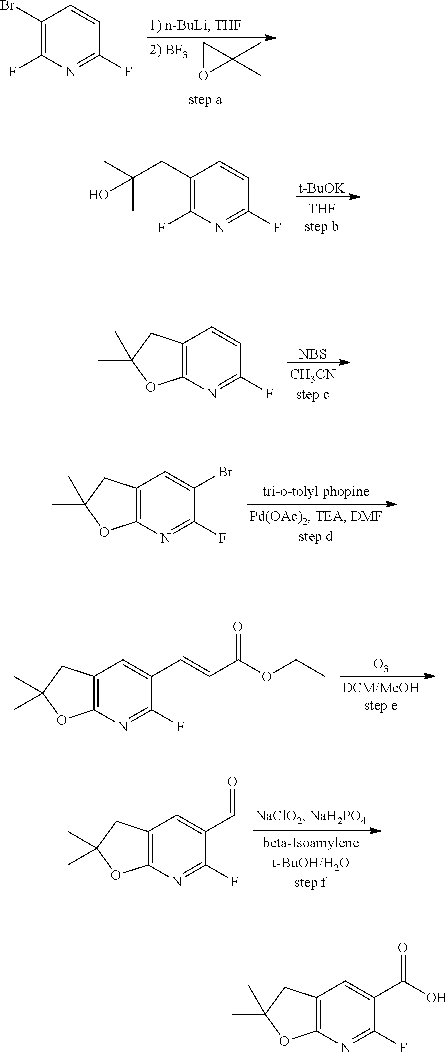

The aryl lithium reagent prepared by lithium-bromine exchange on 3-bromo pyridine with n-butyllithium attacked 2,2-dimethyloxirane to yield 3-(2,2,2-hydroxy dimethyl ethyl) pyridine (step a), which afforded 6-fluoro-2,2-dimethyl-2,3-dihydrofuro[2,3-b]pyridine via an intramolecular cyclization (step b). Bromination on C5 of 2,3-dihydrofuro[2,3-b]pyridine was conducted by NBS to afford the corresponding intermediate (step c). Heck coupling reaction between ethyl acrylate and the brominated C5 of 2,3-dihydrofuro[2,3-b]pyridine gave an alkene intermediate, which was ozonized with O₃ to yield an aldehyde, eventually oxidized by sodium chlorite via Pinnick oxidation reaction to afford a carboxylic acid (steps d, e, f).

### Method BB

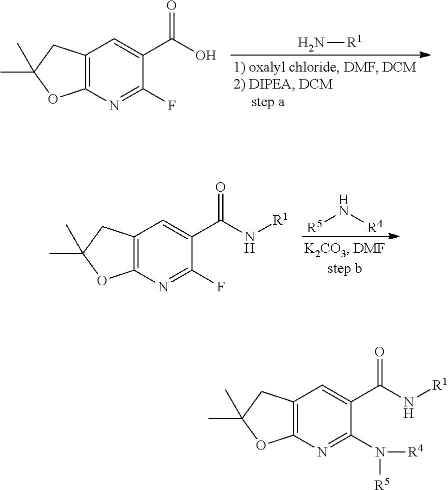

Amide coupling reaction between acyl chloride prepared by 2,3-dihydrofuro [2,3-b]pyridine-3-carboxylic acid and the corresponding amine led to the corresponding intermediate (step a). S_{N}Ar reaction on fluorinated C6 of 2,3-dihydrofuro [2,3-b]pyridine with an amine under basic conditions gave the corresponding compound (step b).

### Method BC

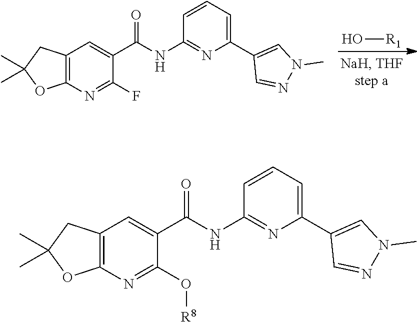

The compounds were obtained through S_{N}Ar reaction of a fluorinated aromatic ring and the corresponding alcohol under NaH basic conditions (step a).

### Method BD

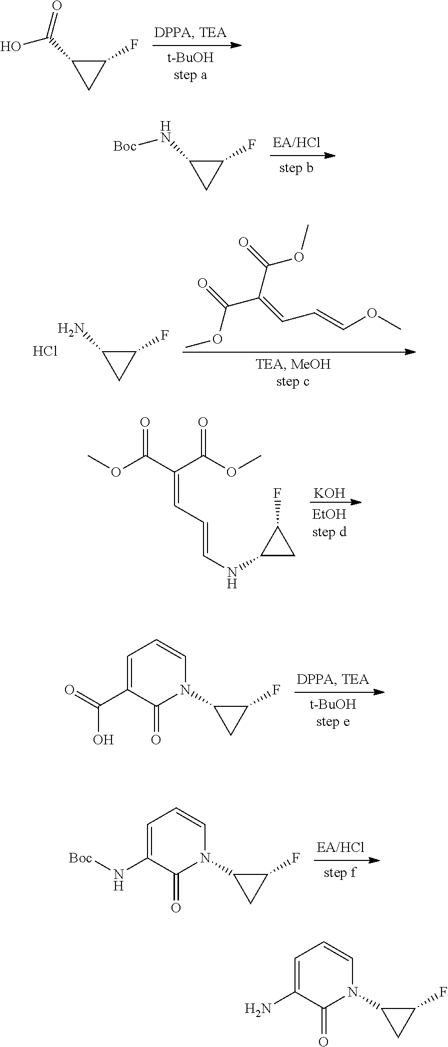

The carboxylic group of the cyclopropyl carboxylic acid was transformed to a Boc protected cyclopropyl amine via a Cuitius rearrangement, whose Boc group was removed under acidic condition to yield the primary amine hydrochloride (steps a, b). The amine reacted with dimethyl-(E)-2-(3-methoxyallylidene)malonate via a Michael addition to yield the corresponding intermediate (step c). The amino group intramolecularly attacked one of the symmetric esters and cyclized, while the other ester was hydrolyzed to a carboxylic group under strong basic conditions, resulting in 1-substituted-2-oxo pyridine-3-carboxylic acid (step d).

Another Curtius rearrangement reaction and Boc-deprotection transformed the carboxylic acid into 3-amino-1-substituted-2-pyridinone (steps e, f).

### Method BE

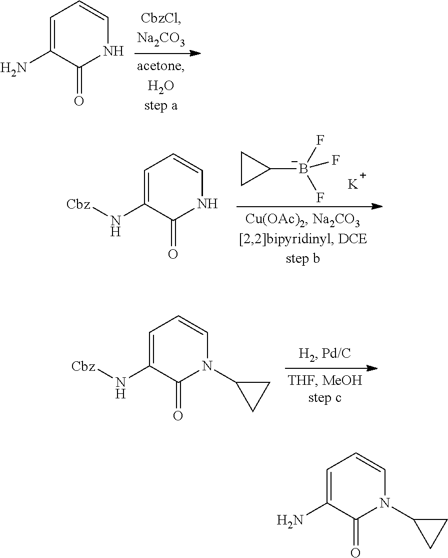 [SMILES: Nc1ccc[nH]c1=O ; O=C(Nc1ccc[nH]c1=O)OCc1ccccc1 ; F[B-](F)(F)C1CC1 ; [K+] ; O=C(Nc1cccn(C2CC2)c1=O)OCc1ccccc1 ; Nc1cccn(C2CC2)c1=O]

The amino group of 3-aminopyridin-2(1H)-one was protected by treating with CbzCl under basic conditions (step a). Cham-Lam coupling between a 2-pyridinone and potassium cyclopropyltrifluoroborate gave 3-benzyloxycarbonylamino-1-cyclopropyl-2-pyridinone. Cbz-deprotection under H₂ atmosphere yielded the product (steps b, c).

### Method BF

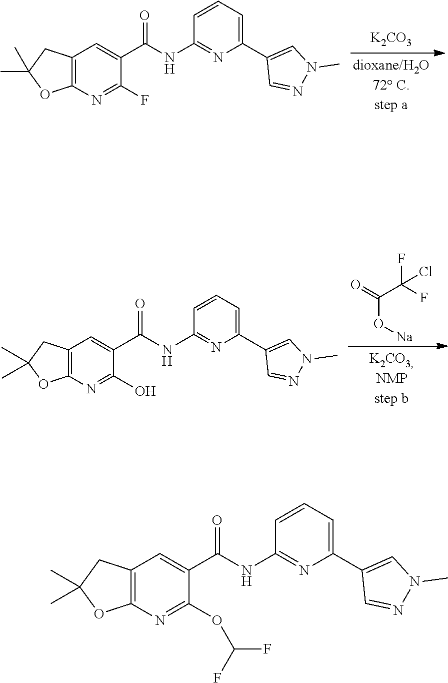

S_{N}Ar on C6 of 2,3-dihydrofuro [2,3-b]pyridine with water yielded a 2-pyridine-ol intermediate, which reacted with sodium 2-chloro-2,2-difluoroacetate to give the fluorinated compound (steps a, b).

### Method BG

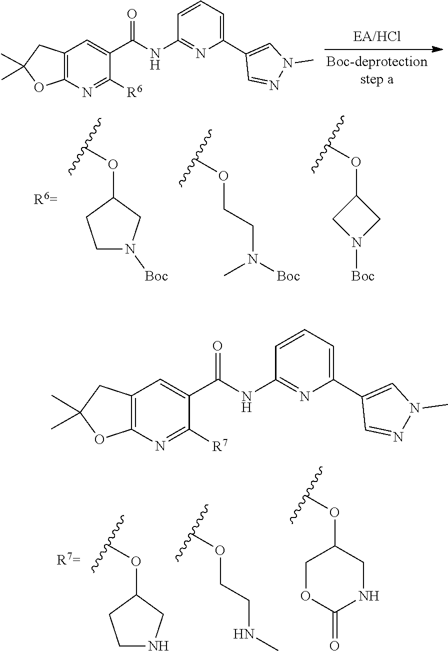

The BOC-protected amine was treated with EA/HCl to remove the BOC protecting group, affording the corresponding secondary amines (step a).

### Method BH

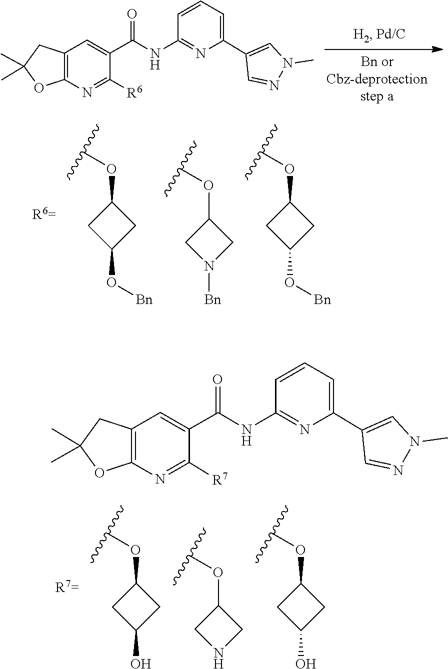

Bn- or Cbz-protecting group was removed by Pd/C under H₂ atmosphere, giving the corresponding alcohol or secondary amine.

### Method BI

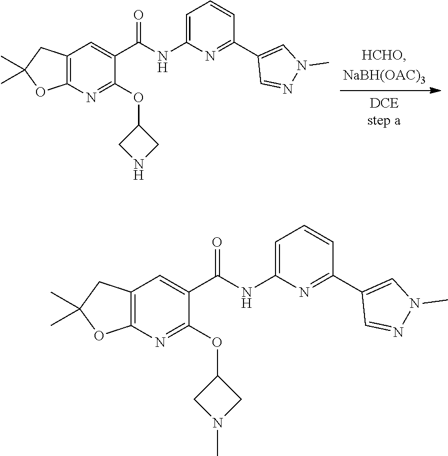

The secondary amine reacted with formaldehyde and using sodium triacetoxyborohydride as the reducing agent, to yield the tertiary amine product (step a).

### Method BJ

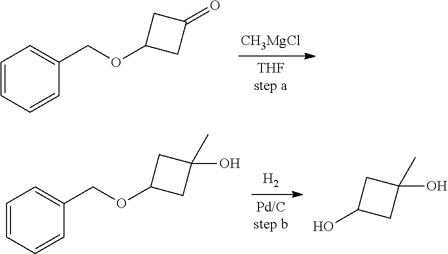

3-(Benzyloxy)cyclobutan-1-one reacted with the Grignard reagent to give a benzyl protected tetrahedral alcohol. Then the benzyl group of was removed via a hydrogenation reduction to yield 1-methyl-cyclobutane-1,3-diol (steps a, b).

### Method BK

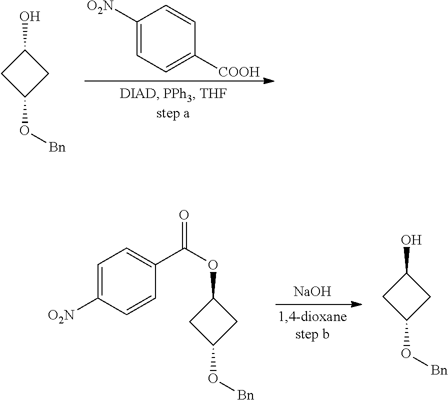 [SMILES: O[C@H]1C[C@@H](OCc2ccccc2)C1 ; O=C(O)c1ccc([N+](=O)[O-])cc1 ; O=C(O[C@H]1C[C@H](OCc2ccccc2)C1)c1ccc([N+](=O)[O-])cc1 ; O[C@H]1C[C@H](OCc2ccccc2)C1]

Mitsunobu reaction between cis-3-(benzyloxy)cyclobutan-1-ol and 4-nitrobenzoic acid gave an ester intermediate whose alcohol moiety underwent chiral inversion. Hydrolysis of the ester gave trans-3-(benzyloxy)cyclobutane-1-ol (steps a, b).

### EXAMPLES

### Example 1, Methods AA, AB, AC

### Preparation of (R)-5-(3-hydroxypyrrolidin-1-yl)-N-(6-(1-methyl-1H-pyrazol-4-yl)pyridin-2-yl)-2-morpholinooxazolo[4,5-b]pyridine-6-carboxamide: (A1)

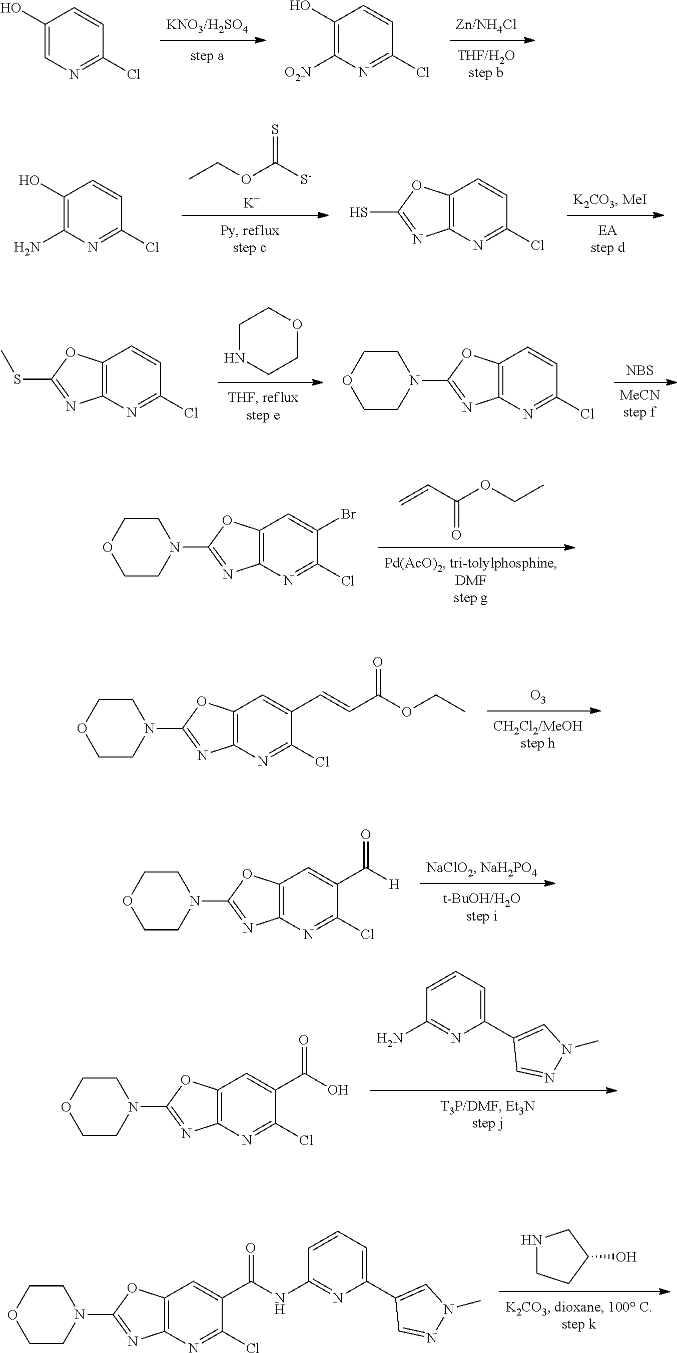

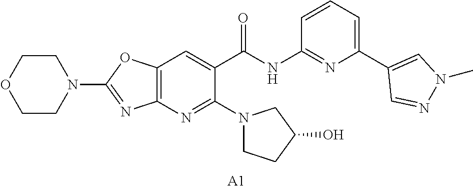

Step a. 6-Chloro-2-nitropyridin-3-ol: To a solution of 6-chloropyridin-3-ol (12 g, 96 mmol) in concentrated H₂SO₄ (150 mL) was added potassium nitrate (12 g, 116 mmol) at ice bath. After being stirred for 2 h at room temperature, the reaction mixture was poured into ice water slowly. The resulting precipitate was collected by filtration and washed with water. The filter cake was dried to give the desired product (15.4 g, 91%) as a yellow solid. ¹H NMR (300 MHz, DMSO-d₆) δ 11.94 (s, 1H), 7.76 (d, J=8.4 Hz, 1H), 7.71 (d, J=8.4 Hz, 1H).

Step b. 2-Amino-6-chloropyridin-3-ol: To a solution of 6-chloro-2-nitropyridin-3-ol (14 g, 83 mmol) in THF/water (300 mL/100 mL) was added ammonium chloride (89 g, 1673 mmol) and zinc powder (44 g, 669 mmol) in portions at 50° C. The mixture was stirred for 1 h at the same temperature. The reaction mixture was diluted with EA (200 mL) and washed with water (200 mL). The combined organic layers were dried over Na₂SO₄, filtrated and concentrated. The residue was purified by silica gel column chromatography (PE/EA=2/1) to give the desired product (11 g, 94%) as a yellow solid. ¹H NMR (300 MHz, DMSO-d₆) δ 9.72 (s, 1H), 6.82 (d, J=7.8 Hz, 1H), 6.37 (d, J=7.8 Hz, 1H), 5.88 (s, 2H). LC-MS (m/z): 145.1 [M+H]⁺.

Step c. 5-Chlorooxazolo[4,5-b]pyridine-2-thiol: A solution of 2-amino-6-chloropyridin-3-ol(23 g, 162 mmol) and potassium ethylxanthate (39 g, 243 mmol) in pyridine (100 mL) was stirred for 4 h at 120° C. After being cooled to room temperature, the solvent of mixture was removed under reduced pressure. The residue was diluted with ice water (100 mL) and the mixture was adjusted pH to 1 with 3 N hydrochloric acid. The resulting precipitate was collected by filtration and washed with water. The filter cake was dried to give the desired product (29 g, 96%) as a grey solid. ¹H NMR (300 MHz, DMSO-d₆) δ 9.86 (s, 1H), 7.87 (s, 1H), 7.31 (s, 1H). LC-MS (m/z): 187.1 [M+H]⁺.

Step d. 5-Chloro-2-(methylthio)oxazolo[4,5-b]pyridine: To a solution of 5-chlorooxazolo[4,5-b]pyridine-2-thiol (29 g, 155 mmol) in EA (300 mL) was added potassium carbonate (43 g, 313 mmol) followed with methyl iodide (20 mL, 311 mmol) at room temperature. After being stirred for 2 h, the mixture was diluted with water (200 mL) and extracted with EA (100 mL). The combined organic layers were dried over Na₂SO₄, filtrated and concentrated to give the intermediate product (28 g, 91%) as a grey solid. ¹H NMR (300 MHz, DMSO-d₆) δ 8.14 (d, J=8.4 Hz, 1H), 7.42 (d, J=8.4 Hz, 1H), 2.79 (s, 3H).

Step e. 5-Chloro-2-morpholinooxazolo[4,5-b]pyridine: To a solution of 5-chloro-2-(methylthio)oxazolo[4,5-b]pyridine (28 g, 140 mmol) in THF (250 mL) was added morpholine (46 mL) and the mixture was stirred at 90° C. overnight. Cooling to room temperature, the solvent was removed in vacuum and the residue was diluted with water (100 mL). The resulting precipitate was collected by filtration and washed with water. The filter cake was dried to give the desired product (32 g, 93%) as a grey solid. 1H NMR (300 MHz, CDCl3) δ 7.37 (d, J=8.1 Hz, 1H), 6.93 (d, J=8.1 Hz, 1H), 3.85-3.78 (m, 4H), 3.78-3.72 (m, 4H). LC-MS (m/z): 240.0 [M+H]⁺.

Step f. 6-Bromo-5-chloro-2-morpholinooxazolo[4,5-b]pyridine: To a solution of 5-chloro-2-morpholinooxazolo[4,5-b]pyridine (32 g, 133 mmol) in MeCN (330 mL) was added NBS (33 g, 186 mmol) in portions at room temperature. Upon reaction completion, the mixture was diluted with EA (1000 mL) and extracted with 1N NaOH aqueous solution. The combined organic layers were dried over Na₂SO₄, filtrated and concentrated to give the intermediate product (28 g, 66%) as a grey solid. ¹H NMR (300 MHz, DMSO-d₆) δ 8.25 (s, 1H), 3.80-3.69 (m, 4H), 3.70-3.61 (m, 4H). LC-MS (m/z): 318.0 [M+H]⁺.

Step g. Ethyl (E)-3-(5-chloro-2-morpholinooxazolo[4,5-b]pyridin-6-yl)acrylate: To a solution of 6-bromo-5-chloro-2-morpholinooxazolo[4,5-b]pyridine (11 g, 36 mmol) in dry DMF (350 mL) was added tri(o-tolyl)phosphine (3.3 g, 11 mmol), palladium acetate (0.70 g, 3.6 mmol), TEA(11 g, 107 mmol) and ethyl acrylate (18.7 mL). The mixture was refluxed at 140° C. overnight under N₂ and the solvent was removed in vacuum. The residue was purified by silica gel column chromatography (PE/EA=1/1) to give the desired product (9.1 g, 76%) as a white solid. ¹H NMR (300 MHz, DMSO-d₆) δ 8.39 (s, 1H), 7.88 (d, J=15.9 Hz, 1H), 6.72 (d, J=15.9 Hz, 1H), 4.20 (q, J=6.9 Hz, 2H), 3.77-3.72 (m, 4H), 3.71-3.67 (m, 4H), 1.26 (t, J=6.9 Hz, 3H). LC-MS (m/z): 338.1 [M+H]⁺.

Step h. 5-Chloro-2-morpholinooxazolo[4,5-b]pyridine-6-carbaldehyde: To a solution of ethyl (E)-3-(5-chloro-2-morpholinooxazolo[4,5-b]pyridin-6-yl)acrylate (4.8 g, 18 mmol) in DCM/MeOH (300 mL/100 mL) was bubbled with dry O₃ for 3 h at −70° C. The reaction was complete, which was detected by TLC, and quenched by adding dimethyl sulfide (2 mL). The solvent was removed in vacuum to give the crude product (4.0 g, 83%) as a yellow solid. ¹H NMR (300 MHz, DMSO-d₆) δ 10.22 (s, 1H), 8.04 (s, 1H), 3.77-3.72 (m, 8H).

Step i. 5-Chloro-2-morpholinooxazolo[4,5-b]pyridine-6-carboxylic acid: To a solution of 5-chloro-2-morpholinooxazolo[4,5-b]pyridine-6-carbaldehyde (5.0 g, 19 mmol) in t-BuOH (250 mL) was added 2-methyl-2-butene (13 g, 187 mmol), sodium chlorite (6.9 g, 187 mmol) and sodium dihydrogen phosphate dehydrate (17 g, 187 mmol) aqueous solution (200 mL). After stirred for 2 h at room temperature, the mixture was adjusted pH to 2 with 1N HCl aqueous solution and extracted with DCM (300 mL×3). The solvent was removed in vacuum to give the desired product (4.2 g, 79%) as a white solid. ¹H NMR (300 MHz, DMSO-d₆) δ 8.14 (s, 1H), 3.75-3.72 (m, 4H), 3.71-3.68 (m, 4H). LC-MS (m/z): 284.0 [M+H]⁺.

Step j. 5-Chloro-N-(6-(1-methyl-1H-pyrazol-4-yl)pyridin-2-yl)-2-morpholinooxazolo[4,5-b]pyridine-6-carboxamide: To a solution of 5-chloro-2-morpholinooxazolo[4,5-b]pyridine-6-carboxylic acid (1.0 g, 3.5 mmol) and 6-(1-methyl-1H-pyrazol-4-yl)pyridin-2-amine (615 mg, 3.5 mmol) in dry DMF (2 mL) was added TEA (1.8 g, 18 mmol) and 50% T₃P in DMF solution (2 mL). The reaction mixture was stirred for 2 h at 110° C. via microwave reactor. The mixture was poured into ice water (250 mL), and the resulting precipitate was collected by filtration and washed with water. The filter cake was dried to give the desired product (510 mg, 33%) as a grey solid. ¹H NMR (300 MHz, DMSO-d₆) δ 10.94 (s, 1H), 8.18 (s, 1H), 8.08 (s, 1H), 8.00-7.91 (m, 2H), 7.86-7.75 (m, 1H), 7.41 (d, J=7.5 Hz, 1H), 3.87 (s, 3H), 3.79-3.73 (m, 4H), 3.72-3.67 (m, 4H). LC-MS (m/z): 440.0 [M+H]⁺.

Step k. (R)-5-(3-Hydroxypyrrolidin-1-yl)-N-(6-(1-methyl-1H-pyrazol-4-yl)pyridin-2-yl)-2-morpholinooxazolo[4,5-b]pyridine-6-carboxamide: To a solution of 5-chloro-N-(6-(1-methyl-1H-pyrazol-4-yl)pyridin-2-yl)-2-morpholinooxazolo[4,5-b]pyridine-6-carboxamide (60 mg, 0.14 mmol) and (R)-pyrrolidin-3-ol (18 mg, 0.21 mmol) in dry 1,4-dioxane (5 mL) was added K₂CO₃ (22 mg, 0.16 mmol) and the mixture was stirred for 24 h at 100° C. The mixture was filtered and the filtrate was concentrated, purified by silica gel column chromatography (DCM/MeOH=20/1) to give the desired product (15 mg, 38%) as a white solid.

### Example 2, Method AC

### Preparation of (R)-5-(3-hydroxypyrrolidin-1-yl)-N-(6-(1-methyl-1H-pyrazol-4-yl)pyridin-2-yl)-2-morpholinooxazolo[4,5-b]pyridine-6-carboxamide: (A2)

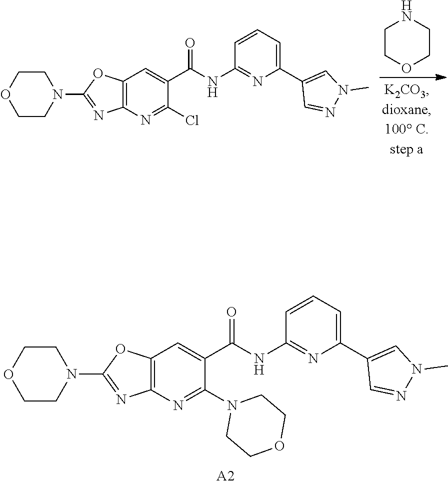

Step a. (R)-5-(3-Hydroxypyrrolidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the title compound (8 mg, 22%) as a white solid.

### Example 3, Method AC

### Preparation of N-(6-(1-methyl-1H-pyrazol-4-yl)pyridin-2-yl)-5-(4-methylpiperazin-1-yl)-2-morpholinooxazolo[4,5-b]pyridine-6-carboxamide: (A3)

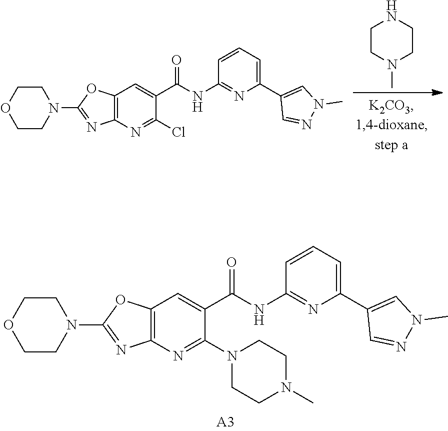

Step a. N-(6-(1-Methyl-1H-pyrazol-4-yl)pyridin-2-yl)-5-(4-methylpiperazin-1-yl)-2-morpholinooxazolo[4,5-b]pyridine-6-carboxamide: The procedure described in Example 1 was carried out to get the compound (10 mg, 29%) as a white solid.

### Example 4, Method AC

### Preparation of 5-(4-hydroxypiperidin-1-yl)-N-(6-(1-methyl-1H-pyrazol-4-yl)pyridin-2-yl)-2-morpholinooxazolo[4,5-b]pyridine-6-carboxamide: (A4)

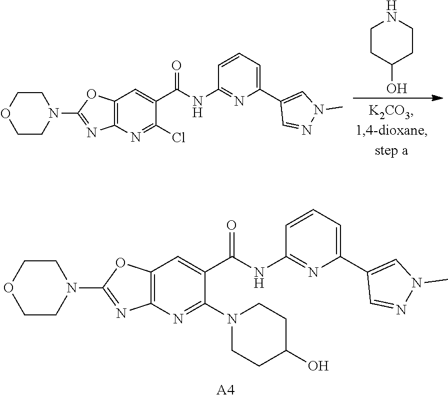 [SMILES: Cn1cc(-c2cccc(NC(=O)c3cc4oc(N5CCOCC5)nc4nc3Cl)n2)cn1 ; OC1CCNCC1 ; Cn1cc(-c2cccc(NC(=O)c3cc4oc(N5CCOCC5)nc4nc3N3CCC(O)CC3)n2)cn1]

Step a. 5-(4-Hydroxypiperidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (9 mg, 26%) as a white solid.

### Example 5, Method AC

### Preparation of N-(6-(1-methyl-1H-pyrazol-4-yl)pyridin-2-yl)-2-morpholino-5-(2-oxa-6-azaspiro[3.3]heptan-6-yl)oxazolo[4,5-b]pyridine-6-carboxamide: (A5)

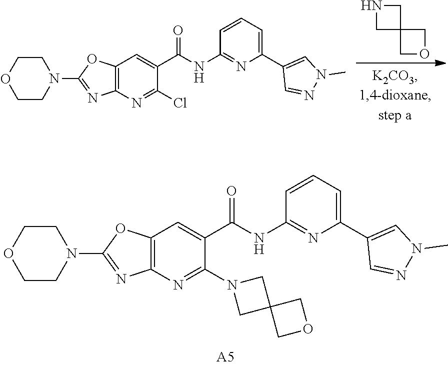 [SMILES: Cn1cc(-c2cccc(NC(=O)c3cc4oc(N5CCOCC5)nc4nc3Cl)n2)cn1 ; C1NCC12COC2 ; Cn1cc(-c2cccc(NC(=O)c3cc4oc(N5CCOCC5)nc4nc3N3CC4(COC4)C3)n2)cn1]

Step a. N-(6-(1-Methyl-1H-pyrazol-4-yl)pyridin-2-yl)-2-morpholino-5-(2-oxa-6-azaspiro[3.3]heptan-6-yl)oxazolo[4,5-b]pyridine-6-carboxamide: The procedure described in Example 1 was carried out to get the compound (8 mg, 22%) as a white solid.

### Example 6, Method AC

### Preparation of (S)-5-(3-hydroxypyrrolidin-1-yl)-N-(6-(1-methyl-1H-pyrazol-4-yl)pyridin-2-yl)-2-morpholinooxazolo[4,5-b]pyridine-6-carboxamide: (A6)

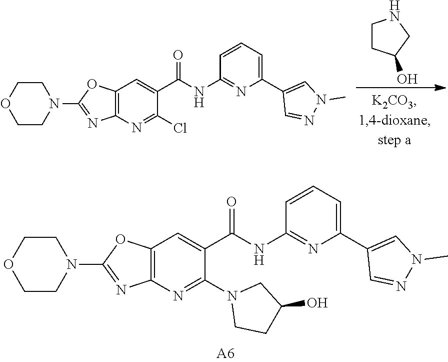 [SMILES: Cn1cc(-c2cccc(NC(=O)c3cc4oc(N5CCOCC5)nc4nc3Cl)n2)cn1 ; O[C@H]1CCNC1 ; Cn1cc(-c2cccc(NC(=O)c3cc4oc(N5CCOCC5)nc4nc3N3CC[C@H](O)C3)n2)cn1]

Step a.: (S)-5-(3-Hydroxypyrrolidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (10 mg, 30%) as a white solid.

### Example 7, Method AC

### Preparation of N-(6-(1-methyl-1H-pyrazol-4-yl)pyridin-2-yl)-5-(methylamino)-2-morpholinooxazolo[4,5-b]pyridine-6-carboxamide: (A7)

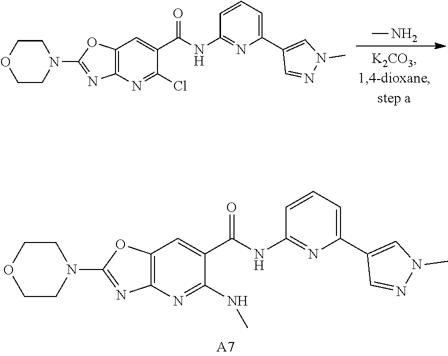

Step a. N-(6-(1-Methyl-1H-pyrazol-4-yl)pyridin-2-yl)-5-(methylamino)-2-morpholinooxazolo[4,5-b]pyridine-6-carboxamide: The procedure described in Example 1 was carried out to get the compound (5 mg, 10%) as a yellow solid.

### Example 8, Method AC, AD, AP

### Preparation of: N-(6-(1-methyl-1H-pyrazol-4-yl)pyridin-2-yl)-2-morpholino-5-(2,5-diazaspiro[3.4]octan-2-yl)oxazolo[4,5-b]pyridine-6-carboxamide: (A8)

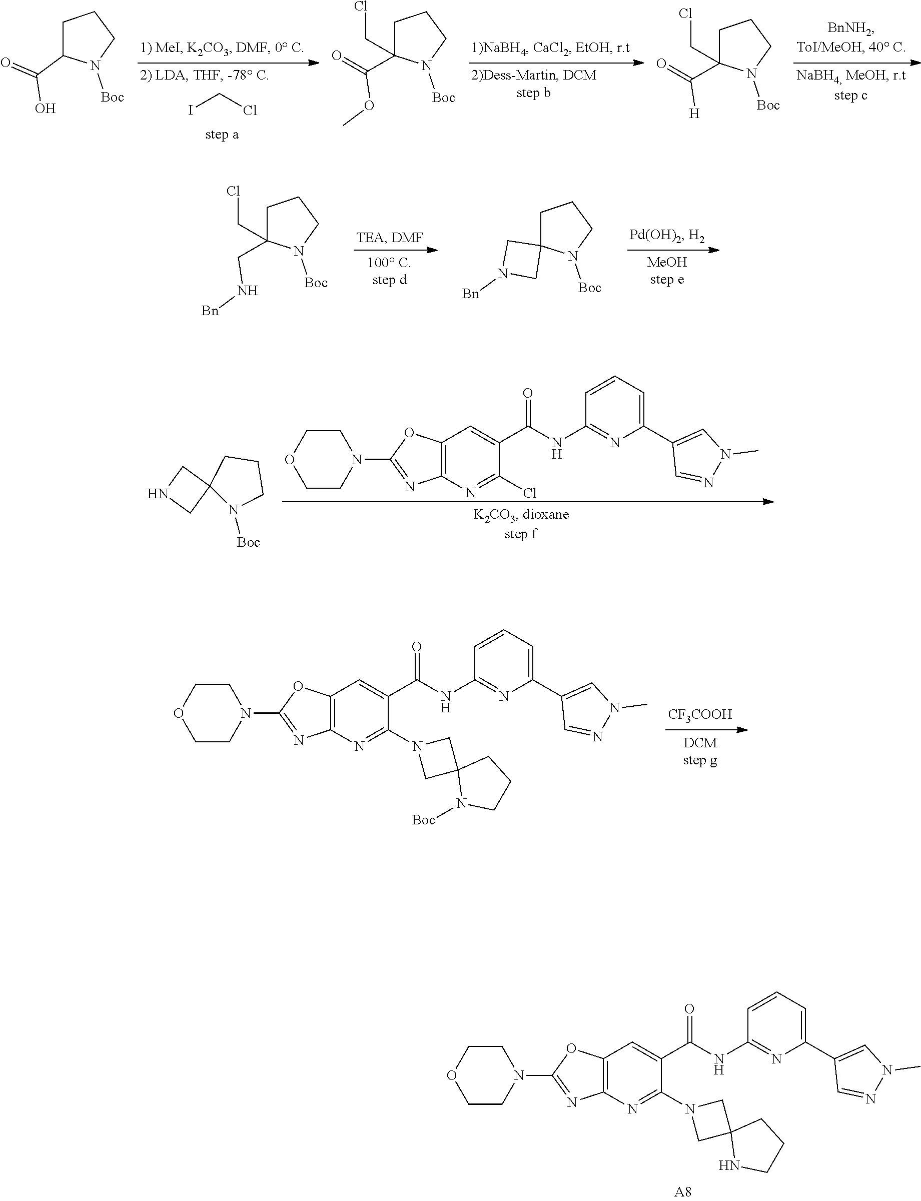

Step a.1-(tert-Butyl) 2-methyl 2-(chloromethyl)pyrrolidine-1,2-dicarboxylate: To a solution of (tert-butoxycarbonyl)proline (22 g, 100 mmol) in DMF (120 mL) was added K₂CO₃ (28 g, 200 mmol) and iodomethane slowly at room temperature. The reaction mixture was stirred at room temperature overnight, and then diluted with ethyl acetate (500 mL), washed with saturated NaCl aqueous solution (100 mL×4). The organic layer was dried over Na₂SO₄, filtered and concentrated to give an intermediate as a white solid. To a solution of LDA (150 mmol) in anhydrous THF (400 mL) was added the THF solution (30 mL) of the white solid intermediate at −78° C., and then stirred for 1 h. The THF solution (20 mL) of chloroiodomethane (21 g, 120 mmol) was added dropwise at −78° C. The reaction was stirred at room temperature overnight. The reaction mixture was quenched with saturated ammonium chloride aqueous solution (200 mL) and extracted with ethyl acetate (150 mL×3). The combined organic layers were dried over Na₂SO₄, filtered and concentrated to give the desired product (15 g, 54%) as a green oil. ¹H NMR (300 MHz, CDCl3) δ 4.55-4.14 (m, 1H), 3.99-3.84 (m, 1H), 3.84-3.56 (m, 4H), 3.55-3.36 (m, 1H), 2.50-2.26 (m, 1H), 2.22-1.81 (m, 3H), 1.51-1.33 (m, 9H). LC-MS (m/z): 299.8 [M+Na]⁺.

Step b. tert-Butyl 2-(chloromethyl)-2-formylpyrrolidine-1-carboxylate: To a solution of 1-(tert-butyl) 2-methyl 2-(chloromethyl)pyrrolidine-1,2-dicarboxylate (1.8 g, 6.6 mmol) in EtOH (50 mL) was added CaCl₂ (1.1 g, 9.9 mmol) slowly, and the mixture was stirred at room temperature for 30 min. NaBH₄ (1.0 g, 27 mmol) was added slowly at 0° C. The reaction was stirred at room temperature overnight, and then quenched with saturated ammonium chloride aqueous solution (40 mL). The resulting precipitate was filtrated. The filtrate was extracted with dichloromethane (30 mL×3). The combined organic layers were dried over Na₂SO₄, filtered and concentrated to obtain the residue. To a solution of the residue in dichloromethane (50 mL) was added Dess-Martin Periodinane (DMP) (2.8 g, 6.6 mmol) at 0° C. The reaction was stirred at room temperature for 3 h, and then quenched with saturated Na₂SO₃ aqueous solution (10 mL) and saturated NaHCO₃ aqueous solution (20 mL). The mixture was extracted with dichloromethane (20 mL×3). The combined organic layers were dried over Na₂SO₄, filtered and concentrated. The residue was purified by silica gel column chromatography (petroleum ether/ethyl acetate=10/1) to give the desired product (1.1 g, 69%) as a colorless oil. ¹H NMR (400 MHz, CDCl₃) δ 9.62-9.36 (m, 1H), 4.26-3.86 (m, 2H), 3.77-3.50 (m, 2H), 2.29-2.01 (m, 3H), 2.00-1.81 (m, 1H), 1.54-1.33 (m, 9H).

Step c. tert-Butyl 2-((benzylamino)methyl)-2-(chloromethyl)pyrrolidine-1-carboxylate: To a solution of tert-butyl 2-(chloromethyl)-2-formylpyrrolidine-1-carboxylate (9.7 g, 39 mmol) in toluene/methanol (80 mL/20 mL) was added benzylamine (4.8 g, 45 mmol), then stirred at 40° C. overnight. The reaction mixture was concentrated, and then dissolved in MeOH (100 mL). NaBH₄ (5.9 g, 156 mmol) was added slowly at 0° C. The reaction was stirred at room temperature overnight, and then quenched with saturated ammonium chloride aqueous solution (150 mL). The mixture was extracted with dichloromethane (100 mL×3). The combined organic layers were dried over Na₂SO₄, filtered and concentrated to obtain the crude product used for the next step without further purification.

Step d. tert-Butyl 2-benzyl-2,5-diazaspiro[3.4]octane-5-carboxylate: A mixture of tert-butyl 2-((benzylamino)methyl)-2-(chloromethyl)pyrrolidine-1-carboxylate and TEA (7.9 g, 78 mmol) in DMF (50 mL) was stirred at 100° C. overnight under N₂ atmosphere. After cooling to room temperature, the reaction mixture was diluted with ethyl acetate (300 mL), and then washed with saturated NaCl aqueous solution (100 mL×4). The organic layer was dried over Na₂SO₄, filtered and concentrated. The residue was purified by silica gel column chromatography (petroleum ether/ethyl acetate=10/1) to give the desired product (10 g, 85%) as a colorless oil. ¹H NMR (400 MHz, CDCl₃) δ 7.30-7.15 (m, 5H), 4.23-3.61 (m, 4H), 3.51-3.01 (m, 4H), 2.41-2.13 (m, 2H), 1.86-1.50 (m, 11H). LC-MS (m/z): 303.1 [M+H]⁺.

Step e. tert-Butyl 2,5-diazaspiro[3.4]octane-5-carboxylate: A suspension of tert-butyl 2-benzyl-2,5-diazaspirol[3.4]octane-5-carboxylate (500 mg, 1.6 mmol) and Pd(OH)₂ (34 mg, 0.25 mmol) in MeOH (50 mL) was stirred at room temperature under H₂ atmosphere for 24 h. The suspension was filtered and the filtrate was concentrated to give the desired product (300 mg, 86%) as a yellow oil. ¹H NMR (300 MHz, CDCl₃) δ 4.69-4.46 (m, 2H), 4.37 (s, 1H), 3.42-3.14 (m, 4H), 2.45-2.31 (m, 2H), 2.29-2.15 (m, 2H), 1.50 (s, 9H).

Step f. tert-Butyl 2-(6-((6-(1-methyl-1H-pyrazol-4-yl)pyridin-2-yl)carbamoyl)-2-morpholinooxazolo[4,5-b]pyridin-5-yl)-2,5-diazaspiro[3.4]octane-5-carboxylate: The procedure described in Example 1 was carried out to get the compound (50 mg, 74%) as a yellow solid. ¹H NMR (300 MHz, DMSO-d₆) δ 10.38 (s, 1H), 8.21 (s, 1H), 8.03-7.89 (m, 2H), 7.84-7.71 (m, 2H), 7.40-7.33 (m, 1H), 3.88 (s, 3H), 3.76-3.69 (m, 6H), 3.69-3.60 (m, 4H), 3.56 (s, 2H), 3.30-3.21 (m, 2H), 2.32-2.15 (m, 2H), 1.77-1.57 (m, 2H), 1.14 (s, 9H).

Step g. N-(6-(1-Methyl-1H-pyrazol-4-yl)pyridin-2-yl)-2-morpholino-5-(2,5-diazaspiro[3.4]octan-2-yl)oxazolo[4,5-b]pyridine-6-carboxamide: To a solution of tert-butyl 2-(6-((6-(1-methyl-1H-pyrazol-4-yl)pyridin-2-yl)carbamoyl)-2-morpholinooxazolo[4,5-b]pyridin-5-yl)-2,5-diazaspiro[3.4]octane-5-carboxylate (50 mg, 0.08 mmol) in dry DCM (5 mL) was added CF₃COOH (0.5 mL). The mixture was stirred at room temperature overnight. The solvent of the reaction mixture was removed in vacuum and the residue was adjusted to pH 8 with saturated NaHCO₃ aqueous solution. The aqueous phase was extracted with DCM (10 mL×3) and the organic layer was dried over Na₂SO₄, filtered and concentrated to give the product (5 mg, 12%) as a yellow solid.

### Example 9, Method AC, AE

### Preparation of: N-(6-(1-Methyl-1H-pyrazol-4-yl)pyridin-2-yl)-5-(5-methyl-2,5-diazaspiro[3.4]octan-2-yl)-2-morpholinooxazolo[4,5-b]pyridine-6-carboxamide: (A9)

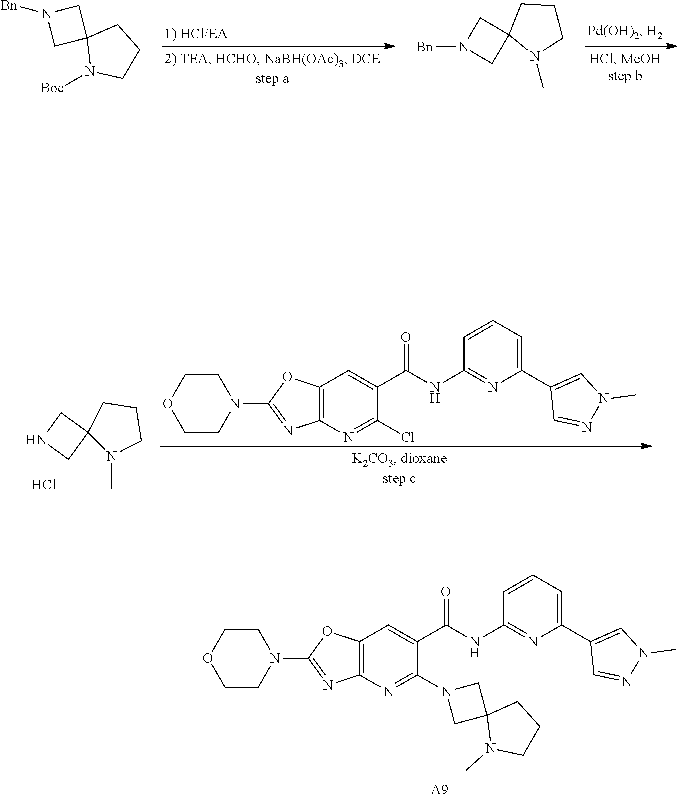

Step a. 2-Benzyl-2,5-diazaspirol[3.4]octane: To a solution of tert-butyl 2-benzyl-2,5-diazaspirol[3.4]octane-5-carboxylate (2.0 g, 6.6 mmol) in HCl/EA (15 mL, 27 mmol) was stirred at room temperature overnight. The reaction mixture was diluted with EA (100 mL), and then filtered to give an intermediate as a white solid. To a solution of the white solid and paraformaldehyde (596 mg, 20 mmol) in DCE (30 mL) was added successively TEA (3.3 g, 33 mmol) and NaBH(OAc)₃ (7.0 g, 33 mmol) at 0° C. The reaction was stirred at room temperature overnight, then quenched with saturated NaHCO₃ aqueous solution (30 mL). The mixture was extracted with dichloromethane (30 mL×3). The combined organic layers were dried over Na₂SO₄, filtered and concentrated. The residue was purified by basic Al₂O₃ column chromatography (petroleum ether/ethyl acetate=5/1) to give the desired product (680 mg, 48%) as a colorless oil. ¹H NMR (400 MHz, CDCl₃) δ 7.36-7.24 (m, 5H), 3.64 (s, 2H), 3.29 (d, J=7.2 Hz, 2H), 3.11 (d, J=7.2 Hz, 2H), 2.66 (t, J=6.8 Hz, 2H), 2.45 (s, 3H), 2.12 (t, J=7.2 Hz, 2H), 1.81-1.65 (m, 2H). LC-MS (m/z): 217.0 [M+H]⁺.

Step b. 5-Methyl-2,5-diazaspiro[3.4]octane: A suspension of 2-benzyl-2,5-diazaspiro[3.4]octane (680 mg, 3.2 mmol) and Pd(OH)₂ (68 mg, 0.48 mmol) in MeOH (50 mL) was stirred at room temperature under H₂ atmosphere for 24 h. The suspension was filtered and concentrated to give the desired product (300 mg) as a yellow oil used for the next step without further purification.

Step c. N-(6-(1-Methyl-1H-pyrazol-4-yl)pyridin-2-yl)-5-(5-methyl-2,5-diazaspiro[3.4]octan-2-yl)-2-morpholinooxazolo[4,5-b]pyridine-6-carboxamide: The procedure described in Example 1 was carried out to get the compound (10 mg, 38%) as a yellow solid.

### Example 10, Method AC

### Preparation of: 5-(3-Cyclopropyl-3-hydroxyazetidin-1-yl)-N-(6-(1-methyl-1H-pyrazol-4-yl)pyridin-2-yl)-2-morpholinooxazolo[4,5-b]pyridine-6-carboxamide: (A10)

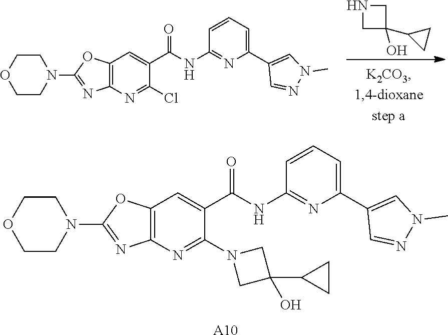 [SMILES: Cn1cc(-c2cccc(NC(=O)c3cc4oc(N5CCOCC5)nc4nc3Cl)n2)cn1 ; OC1(C2CC2)CNC1 ; Cn1cc(-c2cccc(NC(=O)c3cc4oc(N5CCOCC5)nc4nc3N3CC(O)(C4CC4)C3)n2)cn1 ; "A10"]

Step a. 5-(3-Cyclopropyl-3-hydroxyazetidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (9 mg, 26%) as a yellow solid.

### Example 11, Method AC

### Preparation of: 5-(4,4-Difluoropiperidin-1-yl)-N-(6-(1-methyl-1H-pyrazol-4-yl)pyridin-2-yl)-2-morpholinooxazolo[4,5-b]pyridine-6-carboxamide: (A11)

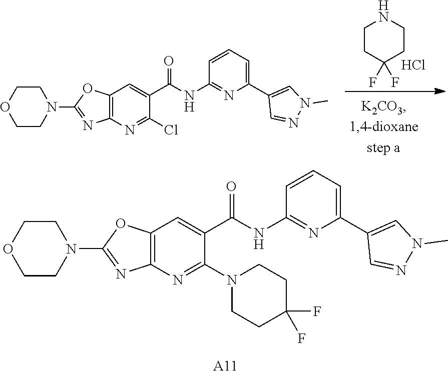 [SMILES: Cn1cc(-c2cccc(NC(=O)c3cc4oc(N5CCOCC5)nc4nc3Cl)n2)cn1 ; FC1(F)CCNCC1 ; Cl ; Cn1cc(-c2cccc(NC(=O)c3cc4oc(N5CCOCC5)nc4nc3N3CCC(F)(F)CC3)n2)cn1 ; "A11"]

Step a. 5-(4,4-Difluoropiperidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (11 mg, 23%) as a yellow solid.

### Example 12, Method AC

### Preparation of: 5-(3,3-Difluoropyrrolidin-1-yl)-N-(6-(1-methyl-1H-pyrazol-4-yl)pyridin-2-yl)-2-morpholinooxazolo[4,5-b]pyridine-6-carboxamide: (A12)

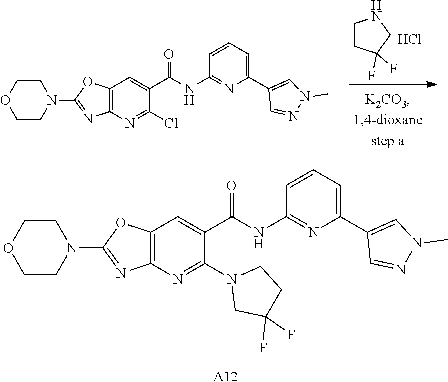 [SMILES: Cn1cc(-c2cccc(NC(=O)c3cc4oc(N5CCOCC5)nc4nc3Cl)n2)cn1 ; FC1(F)CCNC1 ; Cl ; Cn1cc(-c2cccc(NC(=O)c3cc4oc(N5CCOCC5)nc4nc3N3CCC(F)(F)C3)n2)cn1 ; "A12"]

Step a. 5-(3,3-Difluoropyrrolidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (5 mg, 11%) as a yellow solid

### Example 13, Method AC

### Preparation of: N-(6-(1-Methyl-1H-pyrazol-4-yl)pyridin-2-yl)-2-morpholino-5-(piperazin-1-yl)oxazolo[4,5-b]pyridine-6-carboxamide: (A13)

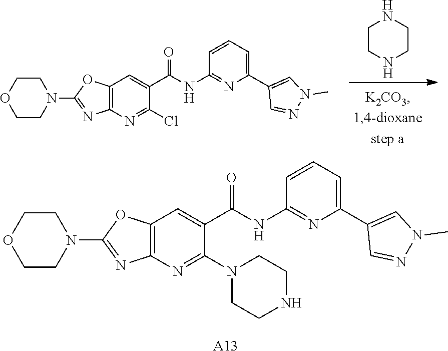 [SMILES: Cn1cc(-c2cccc(NC(=O)c3cc4oc(N5CCOCC5)nc4nc3Cl)n2)cn1 ; C1CNCCN1 ; Cn1cc(-c2cccc(NC(=O)c3cc4oc(N5CCOCC5)nc4nc3N3CCNCC3)n2)cn1 ; "A13"]

Step a. N-(6-(1-Methyl-1H-pyrazol-4-yl)pyridin-2-yl)-2-morpholino-5-(piperazin-1-yl)oxazolo[4,5-b]pyridine-6-carboxamide: The procedure described in Example 1 was carried out to get the compound (10 mg, 23%) as a yellow solid.

### Example 14, Method AC

### Preparation of: 5-(3-Hydroxy-3-methylazetidin-1-yl)-N-(6-(1-methyl-1H-pyrazol-4-yl)pyridin-2-yl)-2-morpholinooxazolo[4,5-b]pyridine-6-carboxamide: (A14)

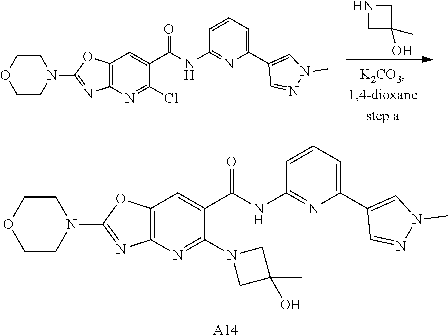

Step a. 5-(3-Hydroxy-3-methylazetidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (6 mg, 18%) as a yellow solid.

### Example 15, Method AC

### Preparation of: 5-(6,6-Difluoro-2-azaspiro[3.3]heptan-2-yl)-N-(6-(1-methyl-1H-pyrazol-4-yl)pyridin-2-yl)-2-morpholinooxazolo[4,5-b]pyridine-6-carboxamide: (A15)

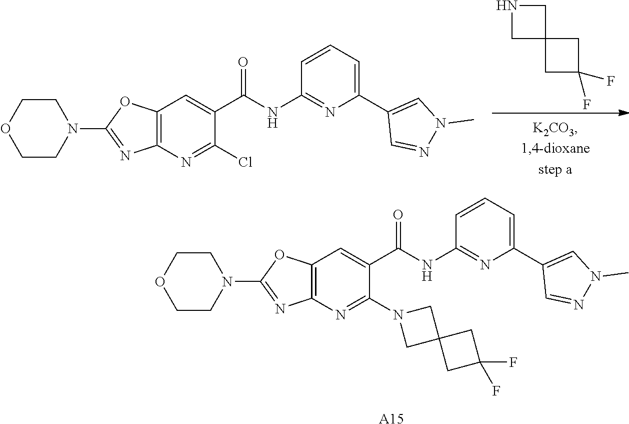 [SMILES: Cn1cc(-c2cccc(NC(=O)c3cc4oc(N5CCOCC5)nc4nc3Cl)n2)cn1 ; FC1(F)CC2(CNC2)C1 ; Cn1cc(-c2cccc(NC(=O)c3cc4oc(N5CCOCC5)nc4nc3N3CC4(C3)CC(F)(F)C4)n2)cn1 ; "A15"]

Step a. 5-(6,6-Difluoro-2-azaspiro[3.3]heptan-2-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (10 mg, 27%) as a white solid.

### Example 16, Method AC

### Preparation of: 5-(3-Hydroxypiperidin-1-yl)-N-(6-(1-methyl-1H-pyrazol-4-yl)pyridin-2-yl)-2-morpholinooxazolo[4,5-b]pyridine-6-carboxamide: (A16)

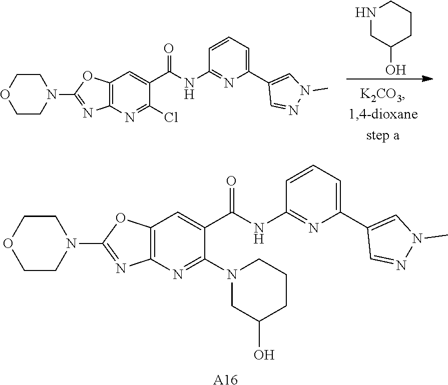 [SMILES: Cn1cc(-c2cccc(NC(=O)c3cc4oc(N5CCOCC5)nc4nc3Cl)n2)cn1 ; OC1CCCNC1 ; Cn1cc(-c2cccc(NC(=O)c3cc4oc(N5CCOCC5)nc4nc3N3CCCC(O)C3)n2)cn1 ; "A16"]

Step a. 5-(3-Hydroxypiperidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (10 mg, 17%) as a white solid.

### Example 17, Method AC

### Preparation of: N-(6-(1-Methyl-1H-pyrazol-4-yl)pyridin-2-yl)-5-(5-methylhexahydropyrrolo[3,4-c]pyrrol-2(1H)-yl)-2-morpholinooxazolo[4,5-b]pyridine-6-carboxamide: (A17)

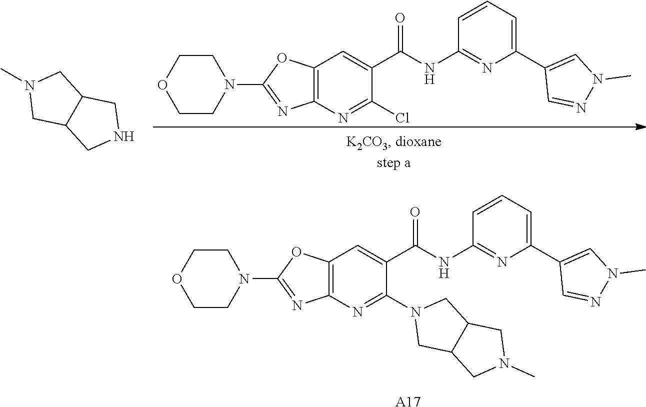

Step a. N-(6-(1-Methyl-1H-pyrazol-4-yl)pyridin-2-yl)-5-(5-methylhexahydropyrrolo[3,4-c]pyrrol-2(1H)-yl)-2-morpholinooxazolo[4,5-b]pyridine-6-carboxamide: The procedure described in Example 1 was carried out to get the compound (7 mg, 38%) as a yellow solid.

### Example 18, Method AC, AF

### Preparation of: N-(6-(1-Methyl-1H-pyrazol-4-yl)pyridin-2-yl)-2-morpholino-5-(6-oxa-2-azaspiro[3.4]octan-2-yl)oxazolo[4,5-b]pyridine-6-carboxamide: (A18)

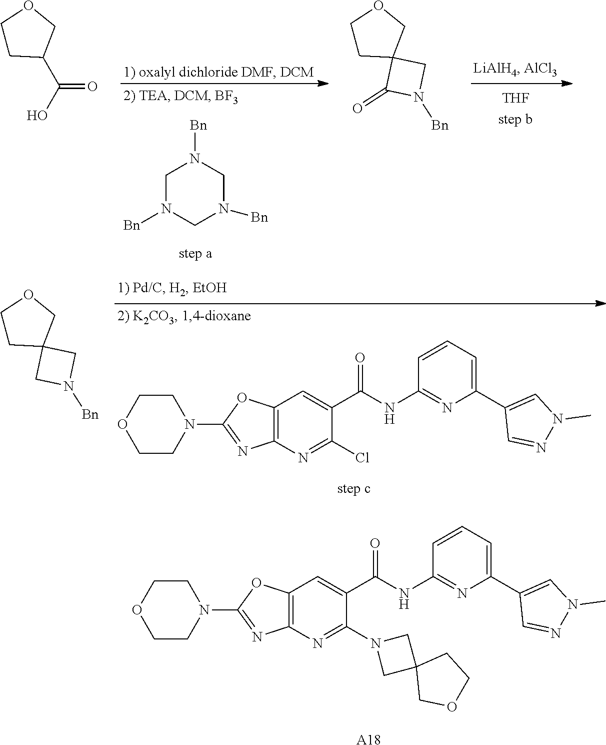

Step a. 2-Benzyl-6-oxa-2-azaspiro[3.4]octan-1-one: To a solution of tetrahydrofuran-3-carboxylic acid (500 mg, 4.3 mmol) in dry DCM (10 mL) was added catalytic amount of DMF and oxalyl chloride (1.6 g, 13 mmol). After being stirred for 1 h at room temperature, the reaction mixture was concentrated in vacuum to obtain the crude product. Meanwhile, another solution of 1,3,5-tribenzyl-1,3,5-triazinane (610 mg, 4.3 mmol) in dry DCM (10 mL) was added boron trifluoride ethyl ether (610 mg, 4.3 mmol) and stirred for 1 h under N₂ atmosphere. To a solution of acyl chloride in dry DCM (10 mL) was added successively dry TEA (1.3 g, 13 mmol) and the above-prepared solution slowly at −78° C. under N₂ atmosphere. The mixture was stirred for 10 min before removed to ice water bath overnight. The reaction mixture was quenched by adding saturated NaHCO₃ aqueous solution and extracted with DCM (30 mL×3). The organic layers were dried over Na₂SO₄, filtered and concentrated. The residue was purified by silica gel column chromatography (petroleum ether/ethyl acetate=5/1) to give the desired product (558 mg, 85%) as a yellow oil. ¹H NMR (400 MHz, CDCl₃): δ 7.33 (dd, J=14.8, 6.4 Hz, 3H), 7.23 (d, J=7.0 Hz, 2H), 4.40 (q, J=14.8 Hz, 2H), 3.99 (q, J=9.2 Hz, 2H), 3.88 (dd, J=14.0, 7.2 Hz, 2H), 3.29-3.15 (m, 2H), 2.50-2.35 (m, 1H), 2.11 (dt, J=12.4, 6.4 Hz, 1H). LC-MS (m/z): 217.9 [M+H]⁺.

Step b. 2-Benzyl-6-oxa-2-azaspiro[3.4]octane: To a solution of aluminum trichloride (366 mg, 2.8 mmol) in dry THF (20 mL) was added successively lithium aluminum hydride (460 mg, 2.1 mmol) and 2-benzyl-6-oxa-2-azaspiro[3.4]octan-1-one (460 mg, 2.1 mmol) in the ice water bath under N₂ atmosphere. After being stirred for 2.5 h at 0° C., the mixture was quenched by adding water (0.32 mL), 10% NaOH aqueous solution (0.64 mL) and another batch of water (0.96 mL). The suspension was filtered and the filtrate was diluted with water (10 mL) and extracted with EA (30 mL×3). The organic layers were dried over Na₂SO₄, filtered and concentrated to give the desired product (350 mg, 81%) as yellow oil. ¹H NMR (400 MHz, CDCl₃): δ 7.39-7.27 (m, 4H), 7.26-7.20 (m, 1H), 3.83 (s, 2H), 3.76 (t, J=6.8 Hz, 2H), 3.61 (s, 2H), 3.24 (s, 4H), 2.09 (t, J=6.8 Hz, 2H). LC-MS (m/z): 204.0 [M+H]⁺.

Step c. N-(6-(1-Methyl-1H-pyrazol-4-yl)pyridin-2-yl)-2-morpholino-5-(6-oxa-2-azaspiro[3.4]octan-2-yl)oxazolo[4,5-b]pyridine-6-carboxamide: A suspension of 2-benzyl-6-oxa-2-azaspiro[3.4]octane (200 mg, 0.10 mmol) and 10% Pd/C (50 mg) in MeOH (50 mL) was stirred at room temperature under H₂ atmosphere overnight. The suspension was filtered and concentrated to give the desired product used for the next step without further purification. The procedure described in Example 1 was carried out to get the compound (27 mg, 48%) as a yellow solid.

### Example 19, Method AC, AP

### Preparation of: 5-(Hexahydropyrrolo[3,4-c]pyrrol-2(1H)-yl)-N-(6-(1-methyl-1H-pyrazol-4-yl)pyridin-2-yl)-2-morpholinooxazolo[4,5-b]pyridine-6-carboxamide: (A19)

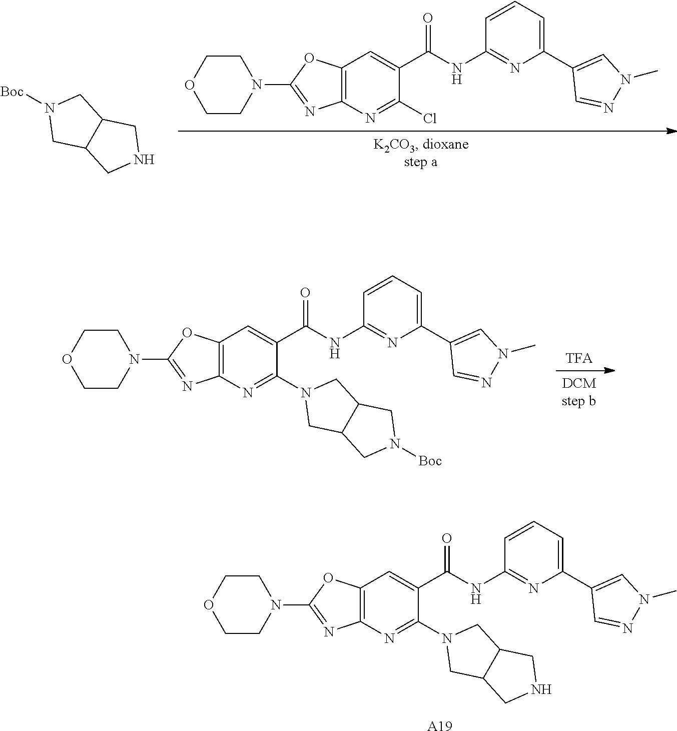

Step a. tert-Butyl 5-(6-((6-(1-methyl-1H-pyrazol-4-yl)pyridin-2-yl)carbamoyl)-2-morpholinooxazolo[4,5-b]pyridin-5-yl)hexahydropyrrolo[3,4-c]pyrrole-2(1H)-carboxylate: The procedure described in Example 8 was carried out to get the compound (50 mg, 74%) as a yellow solid. ¹H NMR (300 MHz, CDCl₃) δ 10.68 (s, 1H), 8.22-8.05 (m, 2H), 7.94-7.81 (m, 2H), 7.70 (s, 1H), 7.24-7.16 (m, 1H), 3.97 (s, 3H), 3.90-3.75 (m, 8H), 3.75-3.49 (m, 4H), 3.45-3.26 (m, 3H), 3.13-2.95 (m, 2H), 2.17 (m, 1H), 1.39 (s, 9H).

Step b. 5-(Hexahydropyrrolo[3,4-c]pyrrol-2(1H)-yl)-N-(6-(1-methyl-1H-pyrazol-4-yl)pyridin-2-yl)-2-morpholinooxazolo[4,5-b]pyridine-6-carboxamide: The procedure described in Example 8 was carried out to get the compound (27 mg, 65%) as a yellow solid.

### Example 20, Method AC, AP

### Preparation of: 5-H[3,4-b]pyrrol-1(2H)-yl)-N-(6-(1-methyl-1H-pyrazol-4-yl)pyridin-2-yl)-2-morpholinooxazolo[4,5-b]pyridine-6-carboxamide: (A20)

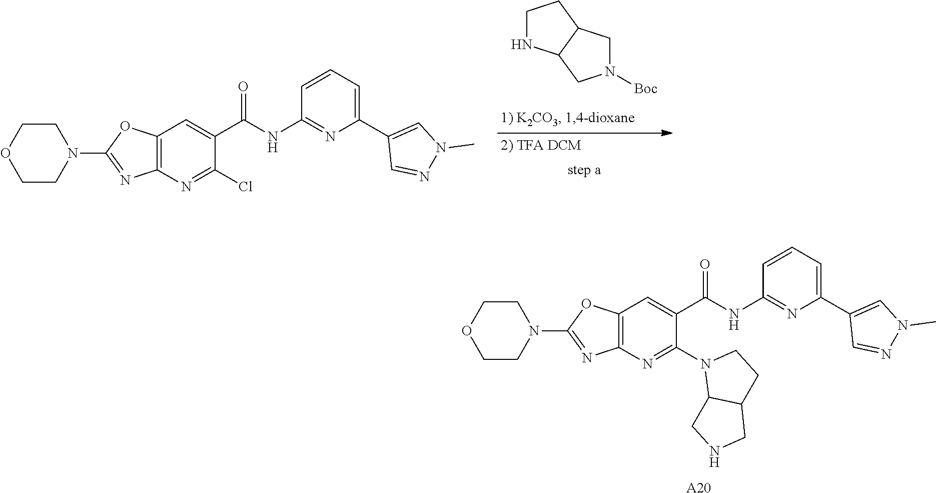

Step a. 5-(Hexahydropyrrolo[3,4-b]pyrrol-1(2H)-yl)-N-(6-(1-methyl-1H-pyrazol-4-yl)pyridin-2-yl)-2-morpholinooxazolo[4,5-b]pyridine-6-carboxamide: The procedure described in Example 8 was carried out to get the compound (11 mg, 19%) as a yellow solid.

### Example 21, Method AC

### Preparation of: (R)-5-(Hexahydropyrrolo[1,2-a]pyrazin-2(1H)-yl)-N-(6-(1-methyl-1H-pyrazol-4-yl)pyridin-2-yl)-2-morpholinooxazolo[4,5-b]pyridine-6-carboxamide: (A21)

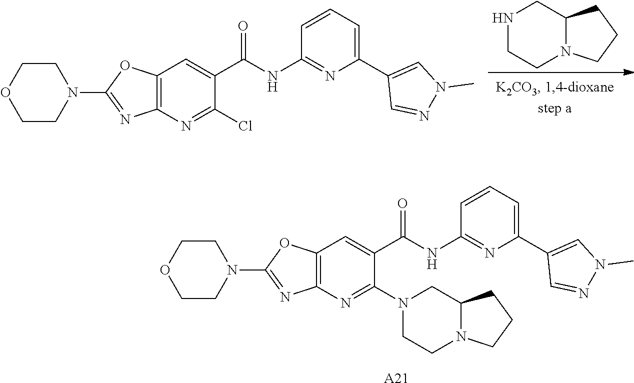

Step a. (R)-5-(Hexahydropyrrolo[1,2-a]pyrazin-2(1H)-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (10 mg, 17%) as a yellow solid.

### Example 22, Method AC

### Preparation of: 5-(6-Hydroxy-2-azaspiro[3.3]heptan-2-yl)-N-(6-(1-methyl-1H-pyrazol-4-yl)pyridin-2-yl)-2-morpholinooxazolo[4,5-b]pyridine-6-carboxamide: (A22)

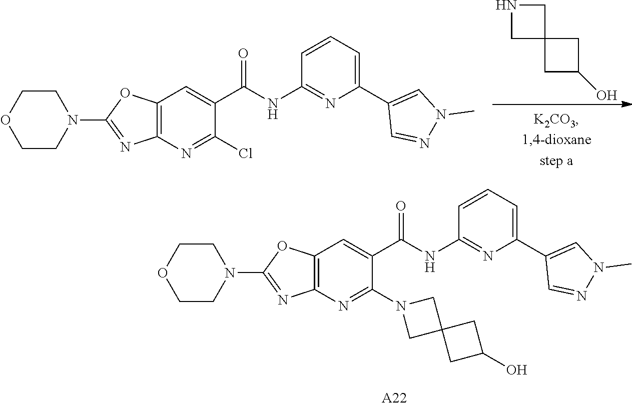 [SMILES: Cn1cc(-c2cccc(NC(=O)c3cc4oc(N5CCOCC5)nc4nc3Cl)n2)cn1 ; OC1CC2(CNC2)C1 ; Cn1cc(-c2cccc(NC(=O)c3cc4oc(N5CCOCC5)nc4nc3N3CC4(CC(O)C4)C3)n2)cn1 ; "A22"]

Step a. 5-(6-Hydroxy-2-azaspiro[3.3]heptan-2-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (10 mg, 17%) as a yellow solid.

### Example 23, Method AC

### Preparation of: 5-(4-(Dimethylamino)piperidin-1-yl)-N-(6-(1-methyl-1H-pyrazol-4-yl)pyridin-2-yl)-2-morpholinooxazolo[4,5-b]pyridine-6-carboxamide: (A23)

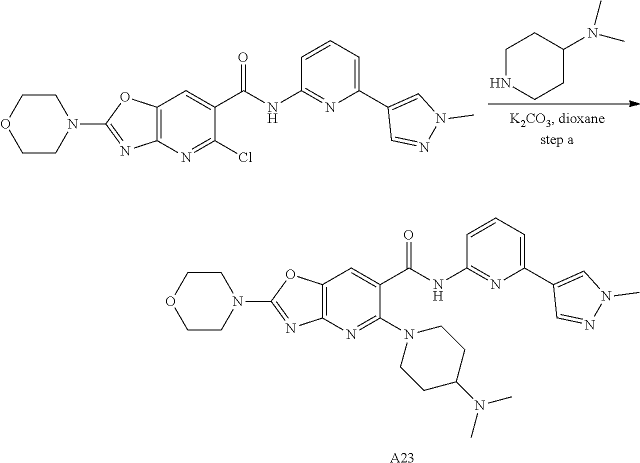

Step a. 5-(4-(Dimethylamino)piperidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (27 mg, 37%) as a yellow solid.

### Example 24, Method AC

### Preparation of: 5-(4-Methoxypiperidin-1-yl)-N-(6-(1-methyl-1H-pyrazol-4-yl)pyridin-2-yl)-2-morpholinooxazolo[4,5-b]pyridine-6-carboxamide: (A24)

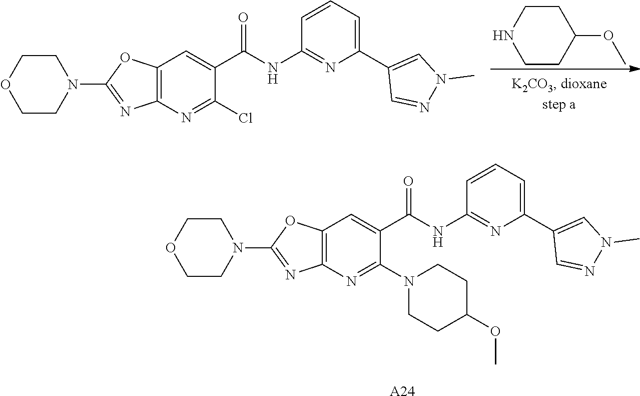

Step a. 5-(4-Methoxypiperidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (16 mg, 28%) as a white solid.

### Example 25, Method AC

### Preparation of: 5-(4-Hydroxy-4-methylpiperidin-1-yl)-N-(6-(1-methyl-1H-pyrazol-4-yl)pyridin-2-yl)-2-morpholinooxazolo[4,5-b]pyridine-6-carboxamide: (A25)

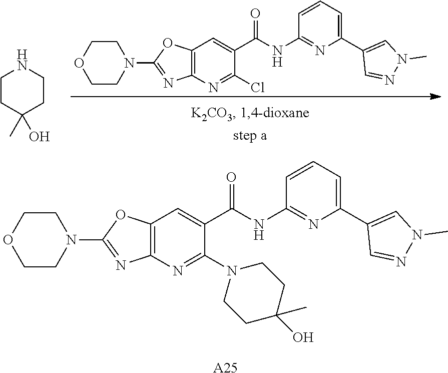

Step a. 5-(4-Hydroxy-4-methylpiperidin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (10 mg, 17%) as a white solid.

### Example 26, Method AC, AG

### Preparation of: N-(6-(1-Methyl-1H-pyrazol-4-yl)pyridin-2-yl)-2-morpholino-5-(1-oxa-7-azaspiro[3.5]nonan-7-yl)oxazolo[4,5-b]pyridine-6-carboxamide: (A26)

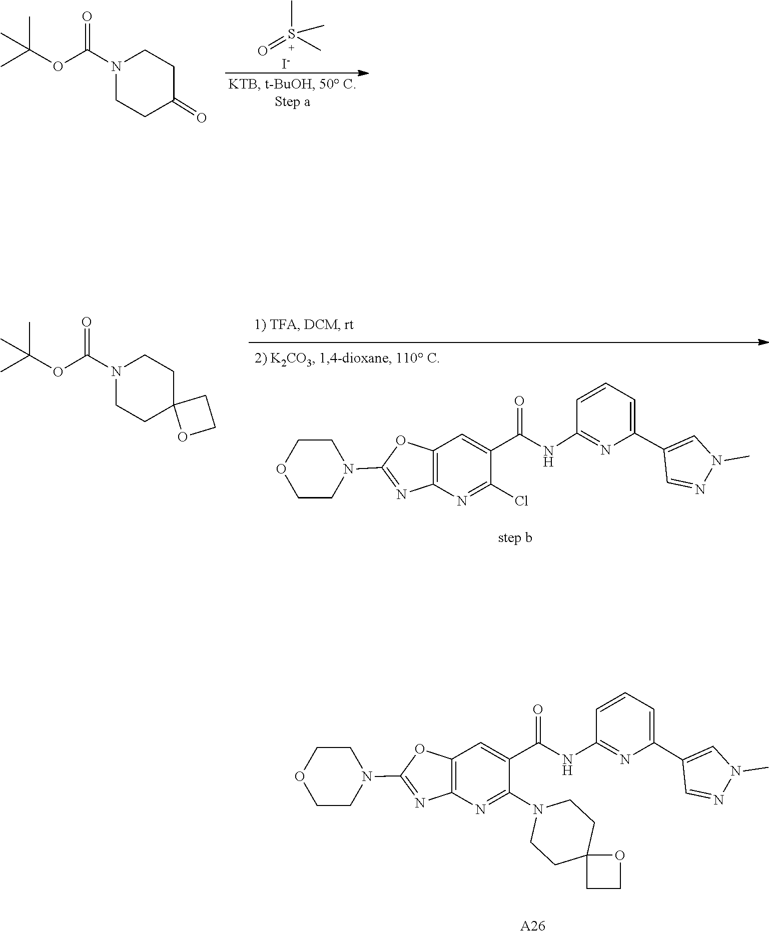

Step a. tert-Butyl 1-oxa-7-azaspiro[3.5]nonane-7-carboxylate: To a solution of trimethylsulfoxonium iodide (2.0 g, 10 mmol) in t-BuOH (70 mL) was added t-BuOK (2.8 g, 25 mmol). After being stirred for 1.5 h at 50° C., the mixture was added tert-butyl 4-oxopiperidine-1-carboxylate (5.5 g, 25 mmol) and stirred for 48 h at the same temperature. Cooling to room temperature, the mixture was diluted with EA (150 mL) and washed with saturated NaCl aqueous solution (50 mL×3). The organic layers were dried over Na₂SO₄, filtered and concentrated. The residue was purified by silica gel column chromatography (petroleum ether/ethyl acetate=10/1) to give the desired product (1.5 g, 66%) as a colorless oil. ¹H NMR (300 MHz, CDCl₃) δ 4.68-4.39 (m, 2H), 3.41 (s, 4H), 2.54-2.26 (m, 2H), 1.98-1.67 (m, 4H), 1.45 (s, 9H).

Step b. N-(6-(1-Methyl-1H-pyrazol-4-yl)pyridin-2-yl)-2-morpholino-5-(1-oxa-7-azaspiro[3.5]nonan-7-yl)oxazolo[4,5-b]pyridine-6-carboxamide: The procedure described in Example 1 was carried out to get the compound (20 mg, 27%) as a yellow solid.

### Example 27, Method AC

### Preparation of: (S)-5-(Hexahydropyrrolo[1,2-a]pyrazin-2(1H)-yl)-N-(6-(1-methyl-1H-pyrazol-4-yl)pyridin-2-yl)-2-morpholinooxazolo[4,5-b]pyridine-6-carboxamide: (A27)

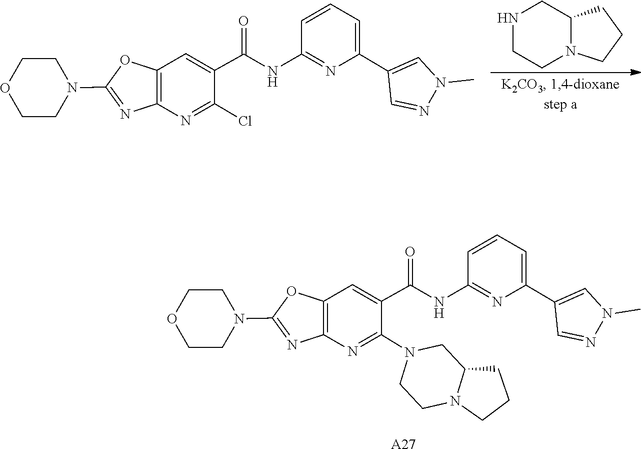

Step a. (S)-5-(Hexahydropyrrolo[1,2-a]pyrazin-2(1H)-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (14 mg, 38%) as a white solid.

### Example 28, Method AC

### Preparation of: 5-(3-Hydroxy-8-azabicyclo[3.2.1]octan-8-yl)-N-(6-(1-methyl-1H-pyrazol-4-yl)pyridin-2-yl)-2-morpholinooxazolo[4,5-b]pyridine-6-carboxamide: (A28)

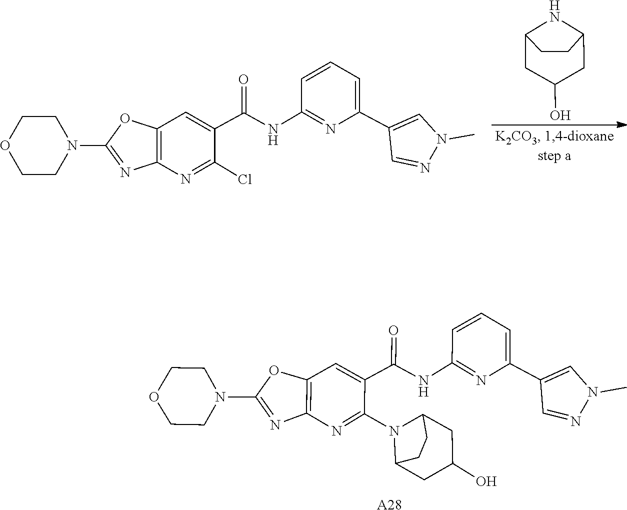

Step a. 5-(3-Hydroxy-8-azabicyclo[3.2.1]octan-8-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (18 mg, 30%) as a white solid.

### Example 29, Method AC, AP

### Preparation of: 5-(3-Aminopiperidin-1-yl)-N-(6-(1-methyl-1H-pyrazol-4-yl)pyridin-2-yl)-2-morpholinooxazolo[4,5-b]pyridine-6-carboxamide, Hydrochloride: (A29)

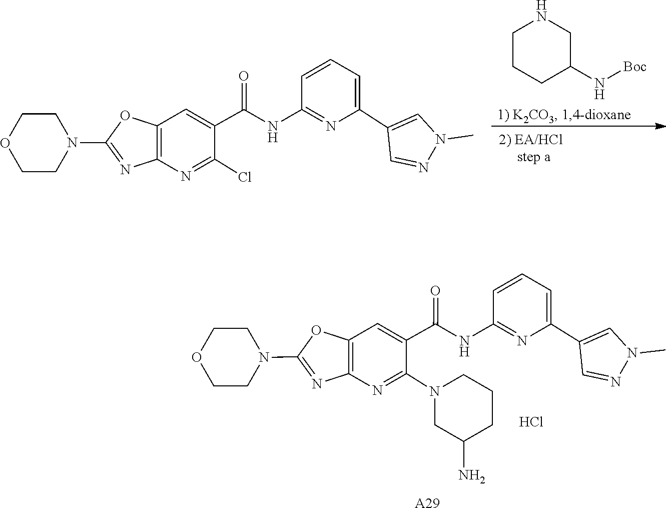 [SMILES: Cn1cc(-c2cccc(NC(=O)c3cc4oc(N5CCOCC5)nc4nc3Cl)n2)cn1 ; CC(C)(C)OC(=O)NC1CCCNC1 ; Cn1cc(-c2cccc(NC(=O)c3cc4oc(N5CCOCC5)nc4nc3N3CCCC(N)C3)n2)cn1 ; Cl ; "A29"]

Step a. 5-(3-Aminopiperidin-1-yl)-N-(6-(1-methyl-1H-pyrazol-4-yl)pyridin-2-yl)-2-morpholinooxazolo[4,5-b]pyridine-6-carboxamide Hydrochloride: The procedure described in Example 8 was carried out to get the compound (8 mg, 13%) as a yellow solid.

### Example 30, Method AC

### Preparation of: 5-(3,4-Dimethylpiperazin-1-yl)-N-(6-(1-methyl-1H-pyrazol-4-yl)pyridin-2-yl)-2-morpholinooxazolo[4,5-b]pyridine-6-carboxamide Hydrochloride: (A30)

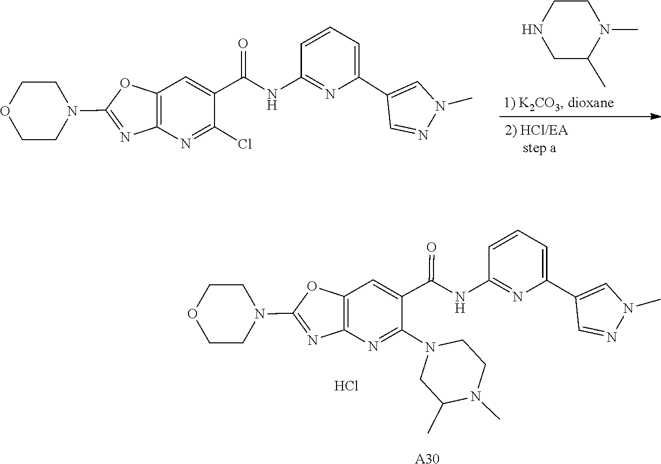

Step a. 5-(3,4-Dimethylpiperazin-1-yl)-N-(6-(1-methyl-1H-pyrazol-4-yl)pyridin-2-yl)-2-morpholinooxazolo[4,5-b]pyridine-6-carboxamide Hydrochloride: The procedure described in Example 1 was carried out to get the compound, which was added to the solution of 2.8 N HCV/EA (5 mL) and stirred at room temperature overnight. The mixture was filtered and dried to obtain the product (15 mg, 27%) as a yellow solid.

### Example 31, Method AC

### Preparation of: (S)-5-(3,4-Dimethylpiperazin-1-yl)-N-(6-(1-methyl-1H-pyrazol-4-yl)pyridin-2-yl)-2-morpholinooxazolo[4,5-b]pyridine-6-carboxamide Hydrochloride: (A31)

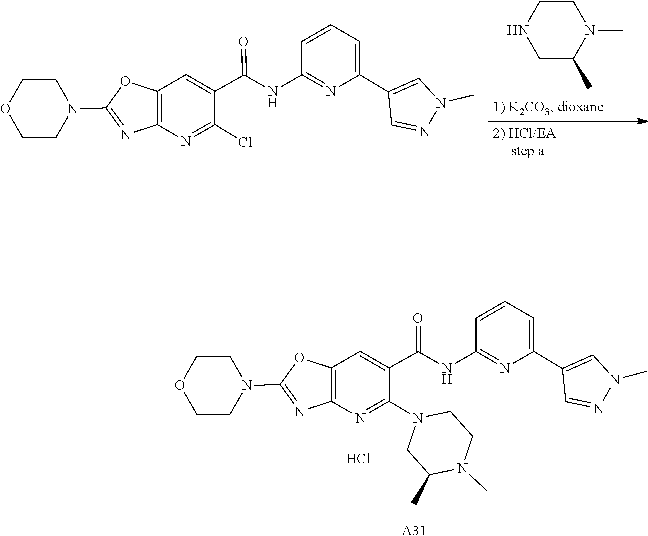

Step a. (S)-5-(3,4-Dimethylpiperazin-1-yl)-N-(6-(1-methyl-1H-pyrazol-4-yl)pyridin-2-yl)-2-morpholinooxazolo[4,5-b]pyridine-6-carboxamide Hydrochloride: The procedure described in Example 1 was carried out to get the compound, which was added to the solution of 2.8 N HCl/EA (5 mL) and stirred at room temperature overnight. The mixture was filtered and dried to obtain the product (19 mg, 32%) as a yellow solid.

### Example 32, Method AC

### Preparation of: 5-(3,5-Dimethylpiperazin-1-yl)-N-(6-(1-methyl-1H-pyrazol-4-yl)pyridin-2-yl)-2-morpholinooxazolo[4,5-b]pyridine-6-carboxamide: (A32)

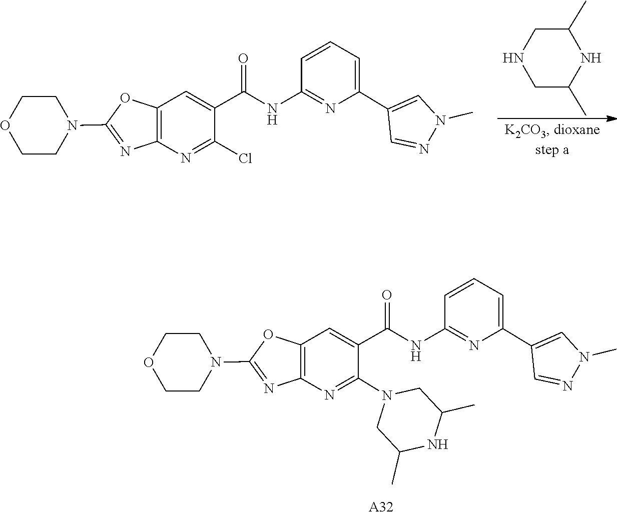

Step a. 5-(3,5-Dimethylpiperazin-1-yl)-N-(6-(1-methyl-1H-pyrazol-4-yl)pyridin-2-yl)-2-morpholinooxazolo[4,5-b]pyridine-6-carboxamide: The procedure described in Example 1 was carried out to get the compound (30 mg, 53%) as a white solid.

### Example 33, Method AH

### Preparation of: N-(6-(1-Methyl-1H-pyrazol-4-yl)pyridin-2-yl)-2-morpholino-5-(3,4,5-trimethylpiperazin-1-yl)oxazolo[4,5-b]pyridine-6-carboxamide: (A33)

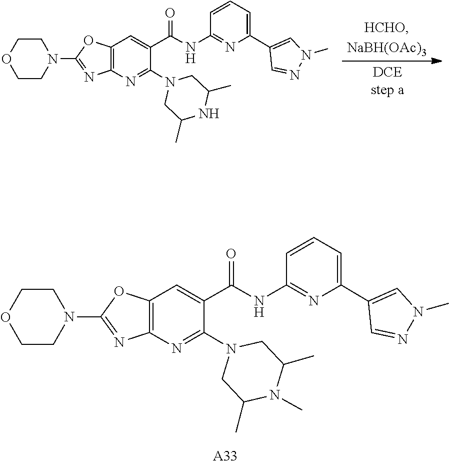

Step a. N-(6-(1-Methyl-1H-pyrazol-4-yl)pyridin-2-yl)-2-morpholino-5-(3,4,5-trimethylpiperazin-1-yl)oxazolo[4,5-b]pyridine-6-carboxamide: To a solution of 5-(3,5-dimethylpiperazin-1-yl)-N-(6-(1-methyl-1H-pyrazol-4-yl)pyridin-2-yl)-2-morpholinooxazolo[4,5-b]pyridine-6-carboxamide (120 mg, 0.23 mmol) and polyoxymethylene (POM) (70 mg, 2.3 mmol) in DCE (10 mL) was added NaBH(OAc)₃ (487 mg, 2.3 mmol) at 0° C. The reaction was stirred at room temperature overnight, and then quenched with saturated NH₄Cl aqueous solution (30 mL). The mixture was extracted with dichloromethane (30 mL×3). The combined organic layers were dried over Na₂SO₄, filtered and concentrated to give the desired product (65 mg, 53%) as a yellow solid.

### Example 34, Method AC

### Preparation of: 5-(4-(2-Methoxyethyl)piperazin-1-yl)-N-(6-(1-methyl-1H-pyrazol-4-yl)pyridin-2-yl)-2-morpholinooxazolo[4,5-b]pyridine-6-carboxamide Hydrochloride: (A34)

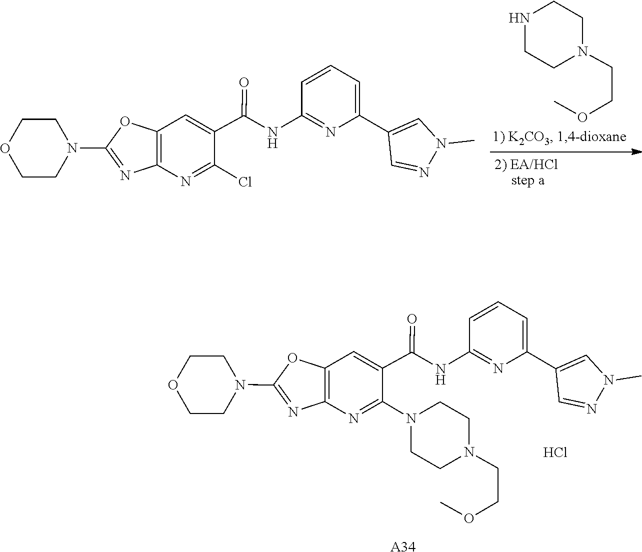

Step a. 5-(4-(2-Methoxyethyl)piperazin-1-yl)-N-(6-(1-methyl-1H-pyrazol-4-yl)pyridin-2-yl)-2-morpholinooxazolo[4,5-b]pyridine-6-carboxamide Hydrochloride: The procedure described in Example 1 was carried out to obtain the product, which was added to the solution of 2.8 N HCl/EA (5 mL) and stirred at room temperature overnight. The mixture was filtered and dried to get the compound (10 mg, 8%) as a yellow solid.

### Example 35, Method AC

### Preparation of: 5-(4-Cyclopropylpiperazin-1-yl)-N-(6-(1-methyl-1H-pyrazol-4-yl)pyridin-2-yl)-2-morpholinooxazolo[4,5-b]pyridine-6-carboxamide Hydrochloride: (A35)

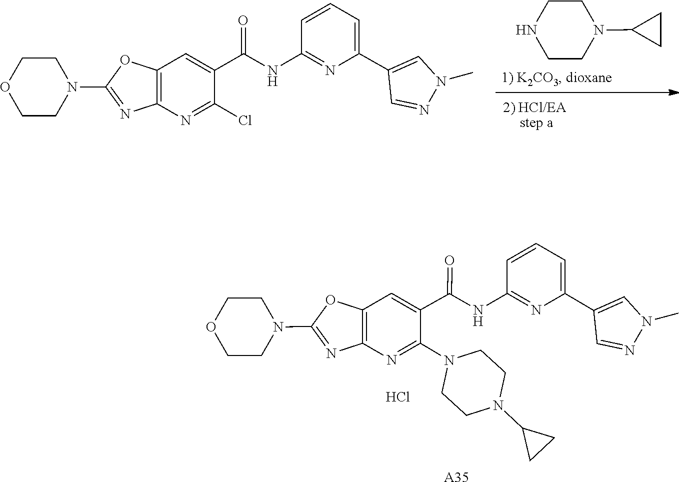 [SMILES: Cn1cc(-c2cccc(NC(=O)c3cc4oc(N5CCOCC5)nc4nc3Cl)n2)cn1 ; C1CN(C2CC2)CCN1 ; Cn1cc(-c2cccc(NC(=O)c3cc4oc(N5CCOCC5)nc4nc3N3CCN(C4CC4)CC3)n2)cn1 ; Cl ; "A35"]

Step a. 5-(4-Cyclopropylpiperazin-1-yl)-N-(6-(1-methyl-1H-pyrazol-4-yl)pyridin-2-yl)-2-morpholinooxazolo[4,5-b]pyridine-6-carboxamide Hydrochloride: The procedure described in Example 1 was carried out to obtain the product, which was added to the solution of 2.8 N HCl/EA (5 mL) and stirred at room temperature overnight. The mixture was filtered and dried to get the compound (30 mg, 48%) as a yellow solid.

### Example 36, Method AC

### Preparation of: 5-(4-(2-Hydroxyethyl)piperazin-1-yl)-N-(6-(1-methyl-1H-pyrazol-4-yl)pyridin-2-yl)-2-morpholinooxazolo[4,5-b]pyridine-6-carboxamide Hydrochloride: (A36)

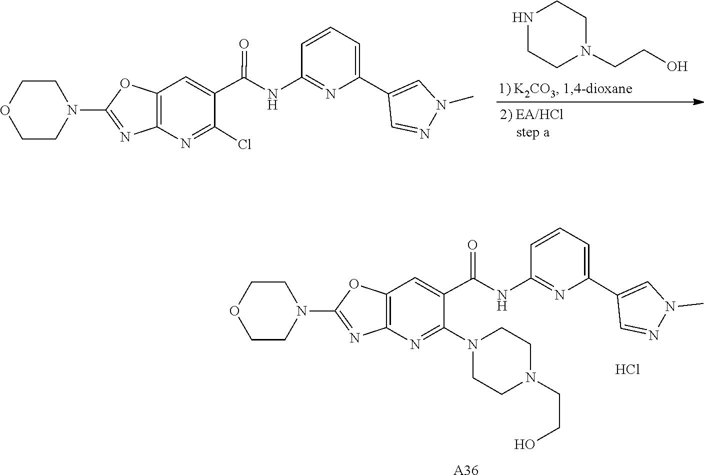 [SMILES: Cn1cc(-c2cccc(NC(=O)c3cc4oc(N5CCOCC5)nc4nc3Cl)n2)cn1 ; OCCN1CCNCC1 ; Cn1cc(-c2cccc(NC(=O)c3cc4oc(N5CCOCC5)nc4nc3N3CCN(CCO)CC3)n2)cn1 ; Cl ; "A36"]

Step a. 5-(4-(2-Hydroxyethyl)piperazin-1-yl)-N-(6-(1-methyl-1H-pyrazol-4-yl)pyridin-2-yl)-2-morpholinooxazolo[4,5-b]pyridine-6-carboxamide Hydrochloride: The procedure described in Example 1 was carried out to obtain the product, which was added to the solution of 2.8 N HCl/EA (5 mL) and stirred at room temperature overnight. The mixture was filtered and dried to get the compound (8 mg, 7%) as a yellow solid.

### Example 37, Method AC

### Preparation of: 5-(3-(Hydroxymethyl)-4-methylpiperazin-1-yl)-N-(6-(1-methyl-1H-pyrazol-4-yl)pyridin-2-yl)-2-morpholinooxazolo[4,5-b]pyridine-6-carboxamide Hydrochloride: (A37)

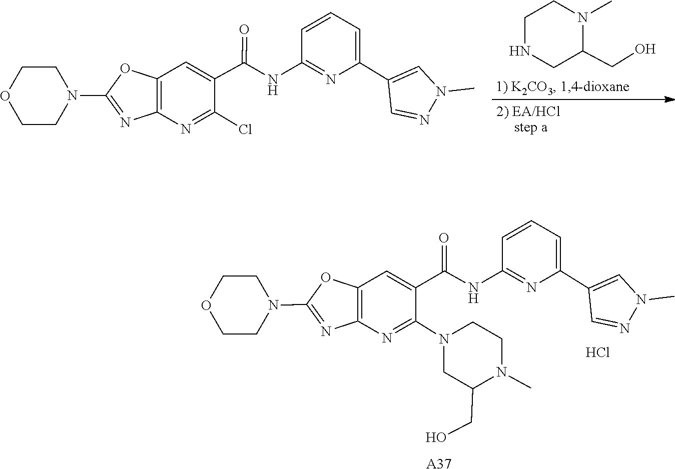

Step a. 5-(4-(2-Hydroxyethyl)piperazin-1-yl)-N-(6-(1-methyl-1H-pyrazol-4-yl)pyridin-2-yl)-2-morpholinooxazolo[4,5-b]pyridine-6-carboxamide Hydrochloride: The procedure described in Example 1 was carried out to obtain the product, which was added to the solution of 2.8 N HCl/EA (5 mL) and stirred at room temperature overnight. The mixture was filtered and dried to get the compound (10 mg, 7%) as a yellow solid.

### Example 38, Method AB, AC, AK

### Preparation of: (R)—N-(6-(Difluoromethyl)pyridin-2-yl)-5-(3-hydroxypyrrolidin-1-yl)-2-morpholinooxazolo[4,5-b]pyridine-6-carboxamide: (A38)

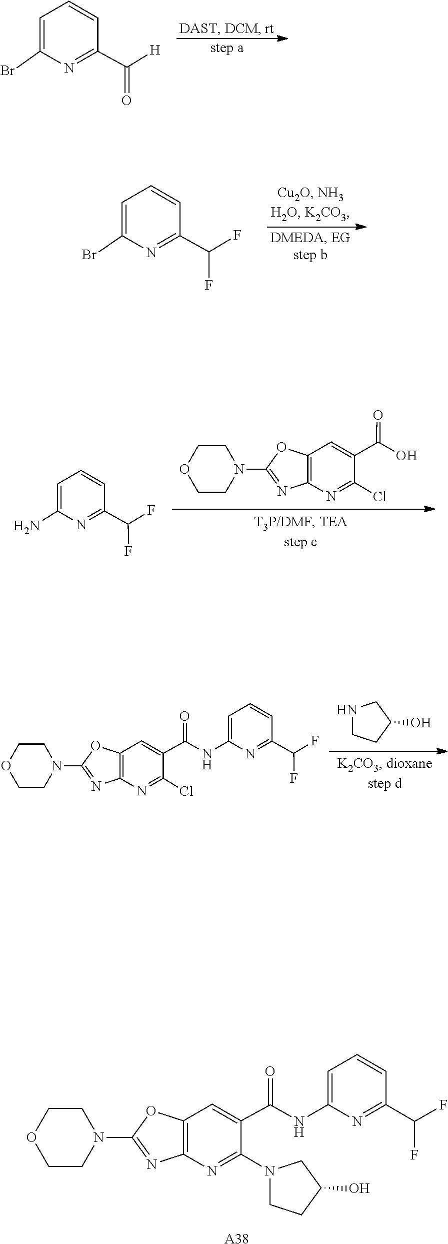

Step a. 2-Bromo-6-(difluoromethyl)pyridine: To a solution of 6-bromopicolinaldehyde (3.7 g, 20 mmol) in dry DCM (30 mL) was added DAST (3.9 g, 24 mmol) and the mixture was stirred at room temperature overnight. The mixture was quenched by saturated NaHCO₃ aqueous solution in ice water bath, and then extracted with DCM (20 mL×3). The combined organic layers were dried over Na₂SO₄, filtered and concentrated to give the desired product as a brown oil used for the next step directly.

Step b. 6-(Difluoromethyl)pyridin-2-amine: A suspension of 2-bromo-6-(difluoromethyl)pyridine (828 mg, 4.0 mmol), Cu₂O (29 mg, 0.2 mmol), K₂CO₃ (110 mg, 0.80 mmol) and N,N-dimethylethylenediamine (35 mg, 0.40 mmol) in a mixed solution (ammonia water/ethylene glycol=10 mL/8 mL) was stirred at the room temperature overnight. The mixture was diluted with DCM (20 mL) and washed with saturated NaCl aqueous solution (20 mL). The combined organic layers were dried over Na₂SO₄, filtrated and concentrated. The residue was purified by silica gel column chromatography to give the desired product (300 mg, 52%). ¹H NMR (300 MHz, CDCl₃) δ 7.58-7.48 (m, 1H), 6.94 (d, J=7.2 Hz, 1H), 6.57 (d, J=8.4 Hz, 1H), 6.42 (t, J=55.8 Hz, 1H), 4.58 (br s, 2H). LC-MS (m/z): 145.1 [M+H]⁺.

Step c. 5-Chloro-N-(6-(difluoromethyl)pyridin-2-yl)-2-morpholinooxazolo[4,5-b]pyridine-6-carboxamide:

The procedure described in Example 1 was carried out to obtain the product (25 mg, 6%) as a yellow solid. ¹H NMR (300 MHz, CDCl₃) δ 9.18 (s, 1H), 8.45 (d, J=8.1 Hz, 1H), 8.04-7.81 (m, 2H), 7.42 (d, J=7.5 Hz, 1H), 6.52 (t, J=55.8 Hz, 1H), 3.83 (s, 8H).

Step d. (R)—N-(6-(Difluoromethyl)pyridin-2-yl)-5-(3-hydroxypyrrolidin-1-yl)-2-morpholinooxazolo[4,5-b]pyridine-6-carboxamide: The procedure described in Example 1 was carried out to obtain the product (20 mg, 63%) as a white solid.

### Example 39, Method AB, AC

### Preparation of: (R)-5-(3-Hydroxypyrrolidin-1-yl)-2-morpholino-N-(pyrazolo[1,5-a]pyrimidin-3-yl)oxazolo[4,5-b]pyridine-6-carboxamide: (A39)

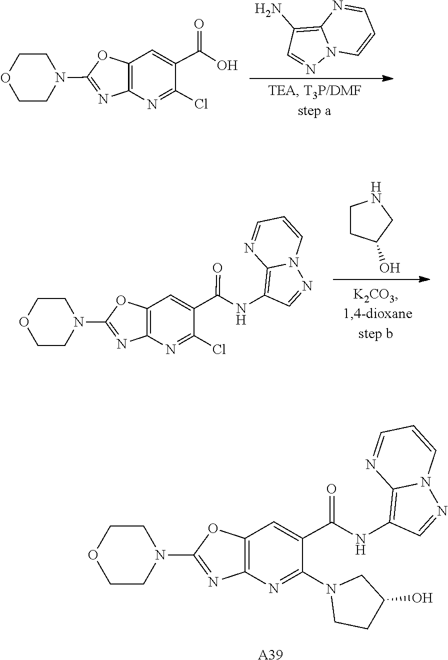 [SMILES: O=C(O)c1cc2oc(N3CCOCC3)nc2nc1Cl ; Nc1cnn2cccnc12 ; O=C(Nc1cnn2cccnc12)c1cc2oc(N3CCOCC3)nc2nc1Cl ; O[C@@H]1CCNC1 ; O=C(Nc1cnn2cccnc12)c1cc2oc(N3CCOCC3)nc2nc1N1CC[C@@H](O)C1 ; "A39"]

Step a. 5-Chloro-2-morpholino-N-(pyrazolo[1,5-a]pyrimidin-3-yl)oxazolo[4,5-b]pyridine-6-carboxamide: The procedure described in Example 1 was carried out to obtain the product (510 mg, 33%) as a white solid. ¹H NMR (300 MHz, DMSO-d₆) δ 10.81 (s, 1H), 9.05 (d, J=6.0 Hz, 1H), 8.65 (s, 1H), 8.53 (d, J=3.9 Hz, 1H), 8.03 (s, 1H), 7.04 (dd, J=6.9, 4.2 Hz, 1H), 3.80-3.72 (m, 4H), 3.71-3.64 (m, 4H).

Step b. (R)-5-(3-Hydroxypyrrolidin-1-yl)-2-morpholino-N-(pyrazolo[1,5-a]pyrimidin-3-yl)oxazolo[4,5-b]pyridine-6-carboxamide: The procedure described in Example 1 was carried out to get the compound (53 mg, 53%) as a white solid.

### Example 40, Method AB, AC

### Preparation of: (R)-5-(3-Hydroxypyrrolidin-1-yl)-N-(1-methyl-1H-pyrazol-4-yl)-2-morpholinooxazolo[4,5-b]pyridine-6-carboxamide: (A40)

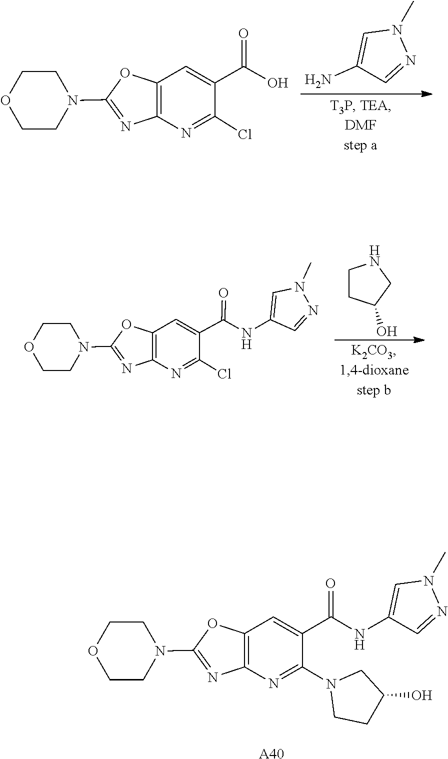

Step a. 5-Chloro-N-(1-methyl-1H-pyrazol-4-yl)-2-morpholinooxazolo[4,5-b]pyridine-6-carboxamide: The procedure described in Example 1 was carried out to obtain the product (240 mg, 35%) as a grey solid.

Step b. (R)-5-(3-Hydroxypyrrolidin-1-yl)-N-(1-methyl-1H-pyrazol-4-yl)-2-morpholinooxazolo[4,5-b]pyridine-6-carboxamide: The procedure described in Example 1 was carried out to get the compound (50 mg, 51%) as a white solid.

### Example 41, Method AB, AC

### Preparation of: 5-(4-Hydroxypiperidin-1-yl)-N-(6-methylpyridin-2-yl)-2-morpholinooxazolo[4,5-b]pyridine-6-carboxamide: (A41)

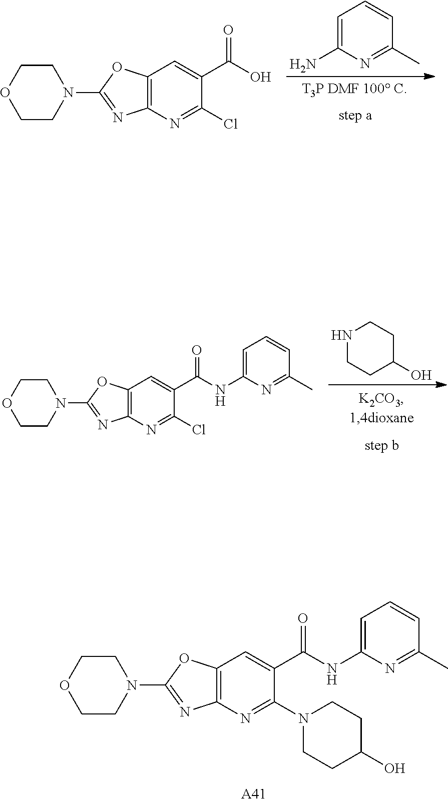

Step a. 5-Chloro-N-(6-methylpyridin-2-yl)-2-morpholinooxazolo[4,5-b]pyridine-6-carboxamide: The procedure described in Example 1 was carried out to obtain the product (40 mg, 28%) as a grey solid. ¹H NMR (300 MHz, DMSO-d₆) δ 10.98 (s, 1H), 8.08-7.94 (m, 2H), 7.79-7.66 (m, 1H), 7.04 (d, J=6.6 Hz, 1H), 3.86-3.72 (m, 4H), 3.72-3.62 (m, 4H), 2.48-2.31 (m, 3H). LC-MS (m/z): 374.1 [M+H]⁺.

Step b. 5-(4-Hydroxypiperidin-1-yl)-N-(6-methylpyridin-2-yl)-2-morpholinooxazolo[4,5-b]pyridine-6-carboxamide: The procedure described in Example 1 was carried out to get the compound (29 mg, 60%) as a white solid.

### Example 42, Method AB, AC

### Preparation of: N-(6-Cyanopyridin-2-yl)-5-(4-hydroxypiperidin-1-yl)-2-morpholinooxazolo[4,5-b]pyridine-6-carboxamide: (A42)

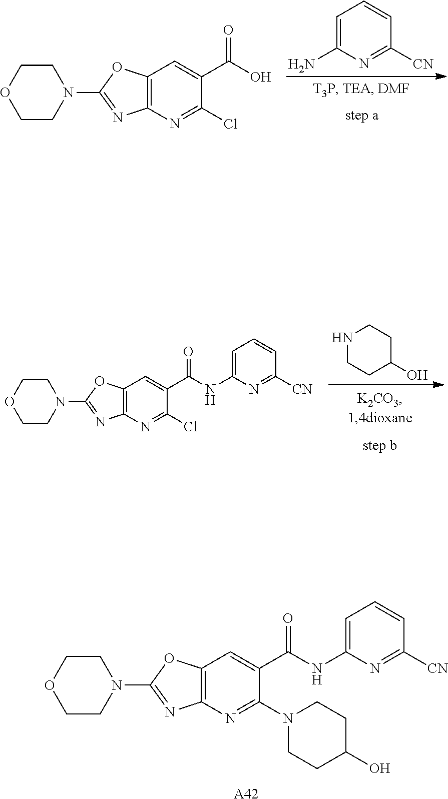

Step a. 5-Chloro-N-(6-cyanopyridin-2-yl)-2-morpholinooxazolo[4,5-b]pyridine-6-carboxamide: The procedure described in Example 1 was carried out to obtain the product (46 mg, 63%) as a grey solid.

Step b. N-(6-Cyanopyridin-2-yl)-5-(4-hydroxypiperidin-1-yl)-2-morpholinooxazolo[4,5-b]pyridine-6-carboxamide: The procedure described in Example 1 was carried out to get the compound (11 mg, 60%) as a white solid.

### Example 43, Method AB, AC

### Preparation of: 5-(4-Hydroxypiperidin-1-yl)-N-(6-methoxypyridin-2-yl)-2-morpholinooxazolo[4,5-b]pyridine-6-carboxamide: (A43)

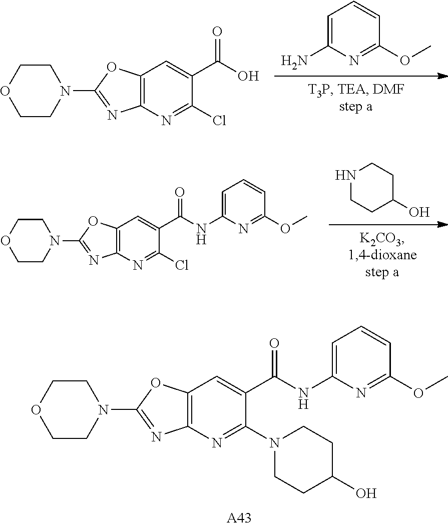

Step a. 5-Chloro-N-(6-methoxypyridin-2-yl)-2-morpholinooxazolo[4,5-b]pyridine-6-carboxamide: The procedure described in Example 1 was carried out to obtain the product (94 mg, 32%) as a yellow solid. ¹H NMR (300 MHz, DMSO-d₆) δ 10.90 (s, 1H), 8.08 (s, 1H), 7.85-7.68 (m, 2H), 6.65-6.53 (m, 1H), 3.83 (s, 3H), 3.79-3.74 (m, 4H), 3.74-3.68 (m, 4H). LC-MS (m/z): 390.1 [M+H]⁺.

Step b. 5-(4-Hydroxypiperidin-1-yl)-N-(6-methoxypyridin-2-yl)-2-morpholinooxazolo[4,5-b]pyridine-6-carboxamide: The procedure described in Example 1 was carried out to get the compound (28 mg, 48%) as a white solid.

### Example 44, Method AB, AC, A1

### Preparation of: N-(3-(Dimethylphosphoryl)phenyl)-5-(4-methoxypiperidin-1-yl)-2-morpholinooxazolo[4,5-b]pyridine-6-carboxamide: (A44)

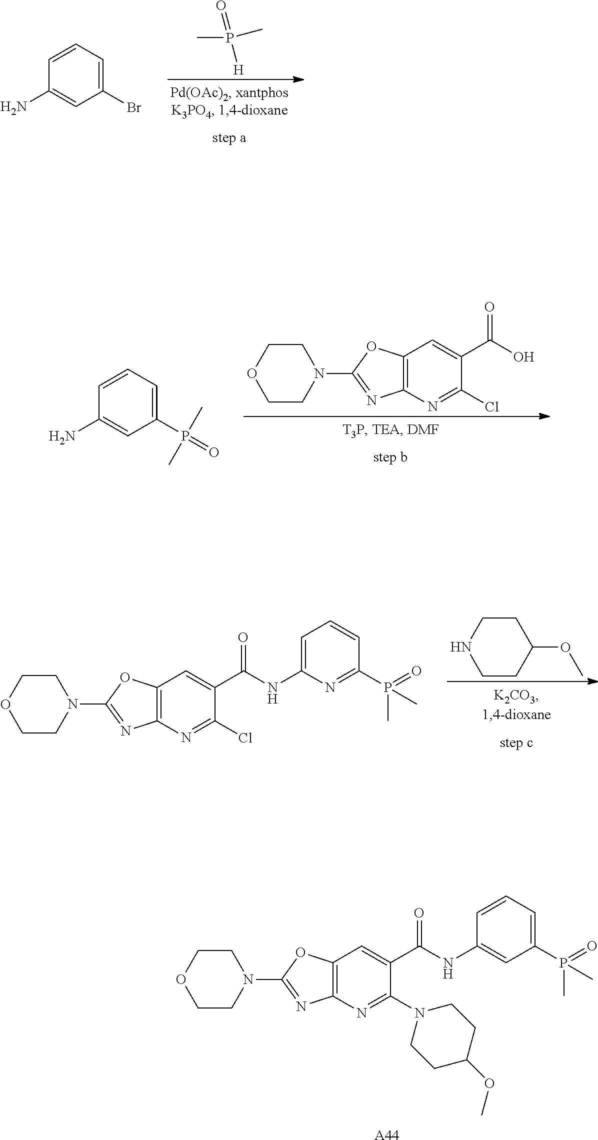

Step a. (3-Aminophenyl)dimethylphosphine oxide: A suspension of 3-bromoaniline (200 mg, 1.2 mmol), dimethylphosphine oxide (136 mg, 1.7 mmol), Pd(OAc)₂ (25 mg, 0.11 mmol), Xantphos (63 mg, 0.11 mmol) and K₃PO₄ (371 mg, 1.7 mmol) in dry 1,4-dioxane (10 mL) was stirred at 120° C. overnight. The mixture was filtered and the filtrate was concentrated. The residue was purified by silica gel column chromatography (DCM/MeOH=30/1) to give the desired product (80 mg, 41%) as a white solid. ¹H NMR (300 MHz, CDCl₃) δ 7.22 (s, 1H), 7.11 (d, J=12.6 Hz, 1H), 7.02-6.90 (m, 1H), 6.80 (d, J=8.4 Hz, 1H), 3.72 (s, 2H),1.69 (d, J=12.6 Hz, 6H). LC-MS (m/z): 170.1 [M+H]⁺.

Step b. 5-Chloro-N-(3-(dimethylphosphoryl)phenyl)-2-morpholinooxazolo[4,5-b]pyridine-6-carboxamide: The procedure described in Example 1 was carried out to get the desired product (120 mg, 26%) as a grey solid. ¹H NMR (300 MHz, CDCl₃) δ 10.16-10.11 (m, 1H), 8.24 (s, 1H), 8.04 (d, J=13.3 Hz, 1H), 7.85 (s, 1H), 7.53-7.43 (m, 1H), 7.29 (s, 1H), 3.93-3.69 (m, 8H), 1.61 (d, J=12.8 Hz, 6H). LC-MS (m/z): 435.0 [M+H]⁺.

Step c. N-(3-(Dimethylphosphoryl)phenyl)-5-(4-methoxypiperidin-1-yl)-2-morpholinooxazolo[4,5-b]pyridine-6-carboxamide: The procedure described in Example 1 was carried out to get the desired product (40 mg, 57%) as a white solid.

### Example 45, Method AB, AC

### Preparation of: 5-(4-Hydroxypiperidin-1-yl)-N-(2-methoxypyridin-3-yl)-2-morpholinooxazolo[4,5-b]pyridine-6-carboxamide: (A45)

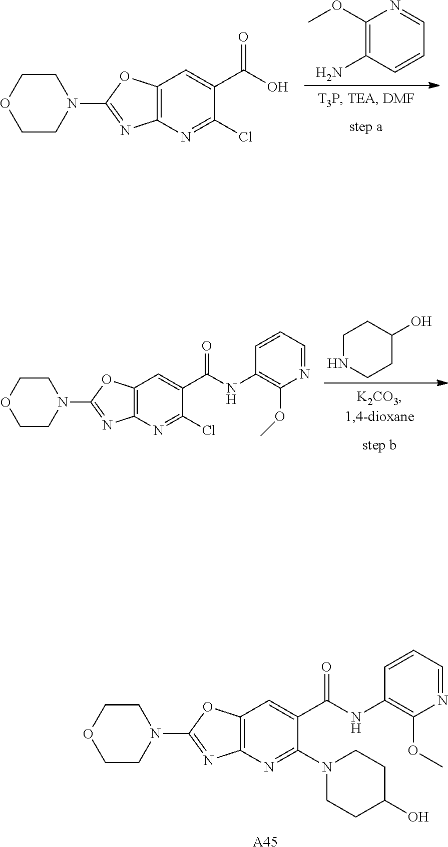

Step a. 5-Chloro-N-(2-methoxypyridin-3-yl)-2-morpholinooxazolo[4,5-b]pyridine-6-carboxamide: The procedure described in Example 1 was carried out to obtain the product (30 mg, 22%) as a yellow oil. ¹H NMR (300 MHz, CDCl₃) δ 9.22 (s, 1H), 8.73 (d, J=6.6 Hz, 1H), 8.08 (s, 1H), 7.91 (d, J=3.3 Hz, 1H), 7.00-6.90 (m, 1H), 4.05 (s, 3H), 3.83 (s, 8H). LC-MS (m/z): 390.1 [M+H]⁺.

Step b. 5-(4-Hydroxypiperidin-1-yl)-N-(2-methoxypyridin-3-yl)-2-morpholinooxazolo[4,5-b]pyridine-6-carboxamide: The procedure described in Example 1 was carried out to get the compound (17 mg, 58%) as a white solid.

### Example 46, Method AC

### Preparation of: 5-(4-Methoxypiperidin-1-yl)-N-(6-methoxypyridin-2-yl)-2-morpholinooxazolo[4,5-b]pyridine-6-carboxamide: (A46)

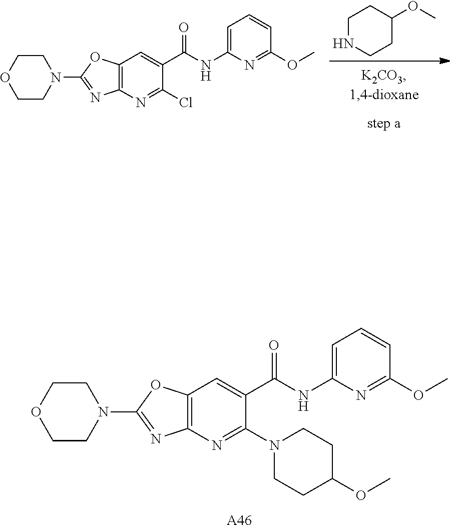

Step a. 5-(4-Methoxypiperidin-1-yl)-N-(6-methoxypyridin-2-yl)-2-morpholinooxazolo[4,5-b]pyridine-6-carboxamide: The procedure described in Example 1 was carried out to get the compound (25 mg, 48%) as a white solid.

### Example 47, Method AB, AC, AJ

### Preparation of: N-(2-Methoxy-3-(1-methyl-1H-pyrazol-4-yl)phenyl)-5-(4-methoxypiperidin-1-yl)-2-morpholinooxazolo[4,5-b]pyridine-6-carboxamide: (A47)

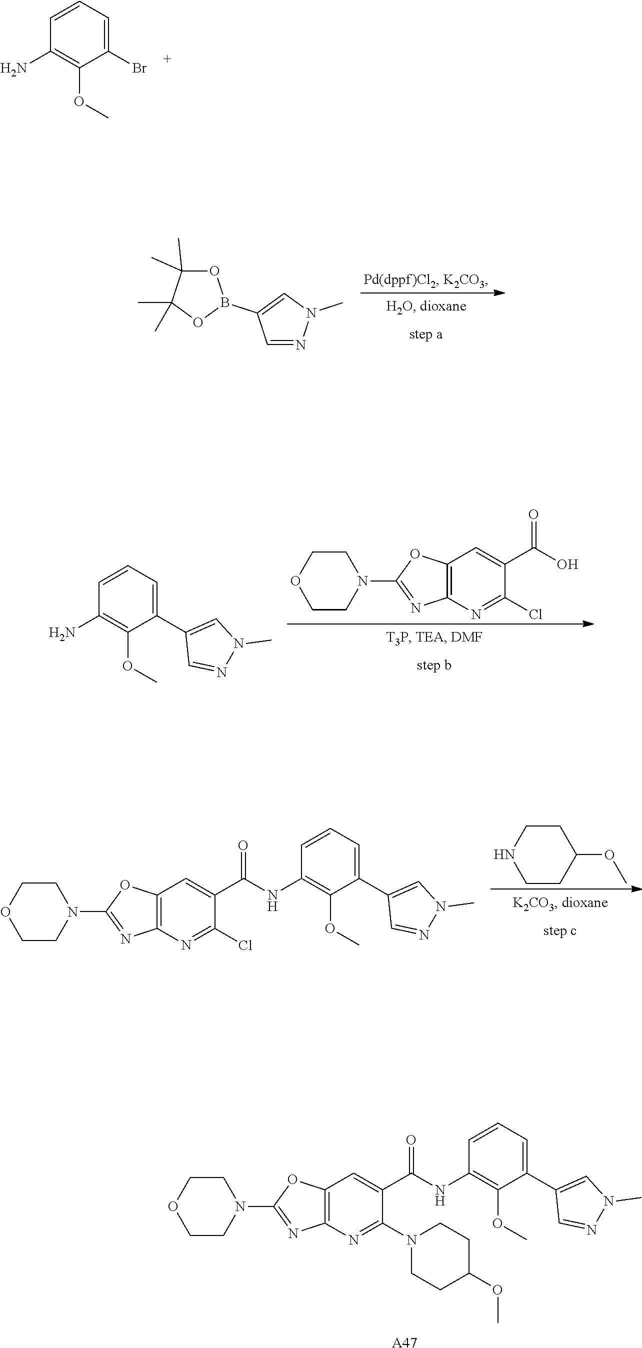

Step a. 2-Methoxy-3-(1-methyl-1H-pyrazol-4-yl)aniline: A suspension of 3-bromo-2-methoxyaniline (500 mg, 2.4 mmol), 1-methyl-4-(4,4,5,5-tetramethyl-1,3,2-dioxaborolan-2-yl)-1H-pyrazole (603 mg, 2.9 mmol), K₂CO₃ (684 mg, 4.9 mmol), Pd(dppf)Cl₂ (183 mg, 0.25 mmol) in a mixed solvent (1,4-dioxane/water=20 mL/2 mL) was stirred at 100° C. overnight under N₂ atmosphere. After cooling to room temperature, the mixture was concentrated in vacuum, and then the residue was purified by silica gel column chromatography (PE/EA=3/1) to give the desired product (380 mg, 70%) as a yellow solid.

Step b. 5-Chloro-N-(2-methoxy-3-(1-methyl-1H-pyrazol-4-yl)phenyl)-2-morpholinooxazolo[4,5-b]pyridine-6-carboxamide: The procedure described in Example 1 was carried out to get the compound (60 mg, 73%) as a white solid. ¹H NMR (300 MHz, CDCl₃) δ 9.49 (s, 1H), 8.42 (s, 1H), 8.16 (s, 1H), 7.87 (s, 1H), 7.80 (s, 1H), 7.22-7.12 (m, 2H), 3.98 (s, 3H), 3.90-3.80 (m, 8H), 3.67 (s, 3H).

Step c. N-(2-Methoxy-3-(1-methyl-1H-pyrazol-4-yl)phenyl)-5-(4-methoxypiperidin-1-yl)-2-morpholinooxazolo[4,5-b]pyridine-6-carboxamide: The procedure described in Example 1 was carried out to get the compound (13 mg, 18%) as a white solid.

### Example 48, Method AB, AC, AK

### Preparation of: N-(3-(2H-Tetrazol-5-yl)phenyl)-5-(4-methoxypiperidin-1-yl)-2-morpholinooxazolo[4,5-b]pyridine-6-carboxamide: (A48)

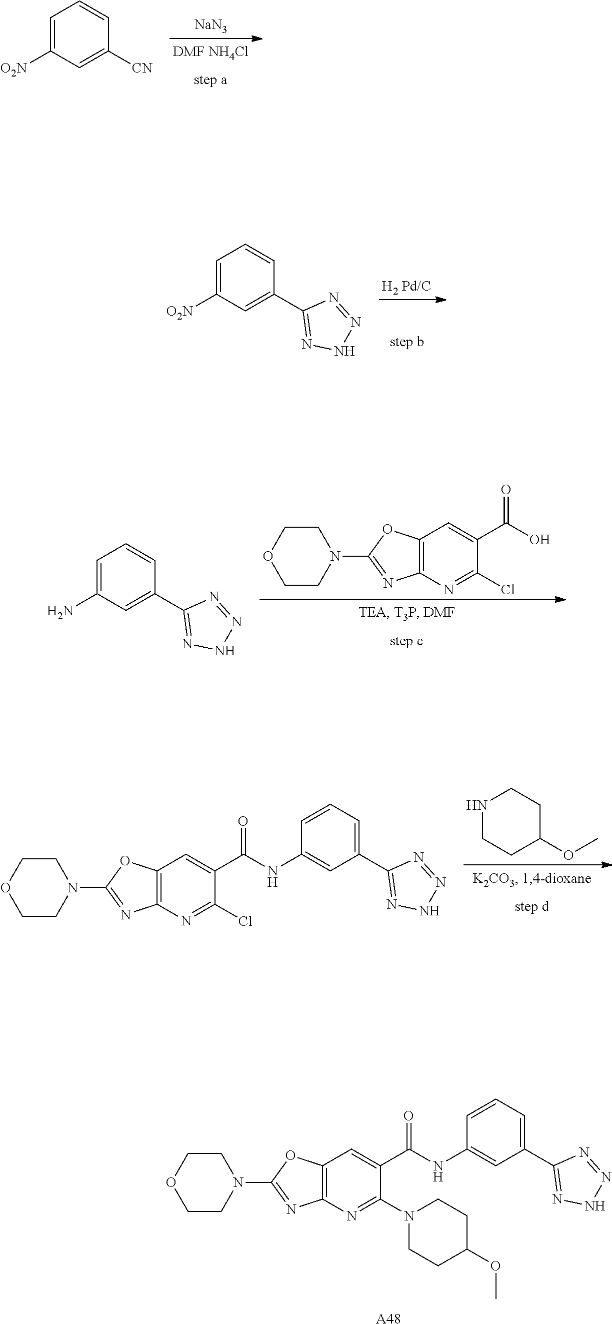

Step a. 5-(3-Nitrophenyl)-2H-tetrazole: To a solution of 3-nitrobenzonitrile (1.0 g, 6.8 mmol) in DMF (15 mL) was added NaN₃ (2.6 g, 40 mmol) and ammonium chloride (2.2 g, 40 mmol). The mixture was stirred at 120° C. overnight. After cooling to room temperature, the mixture was diluted with water (25 mL) and extracted with DCM (25 mL×3). The combined organic layers were dried over Na₂SO₄, filtrated and concentrated to give the desired product (620 mg, 50%) as a white solid. ¹H NMR (300 MHz, CDCl₃) δ 8.12 (s, 1H), 7.79-7.66 (m, 2H), 7.23-7.16 (m, 1H).

Step b. 3-(2H-Tetrazol-5-yl)aniline: A suspension of 5-(3-nitrophenyl)-2H-tetrazole (620 mg, 3.2 mmol) and 10% Pd/C (300 mg) in MeOH (50 mL) was stirred at room temperature under H₂ atmosphere overnight. The suspension was filtered and concentrated to give the desired product (430 mg, 82%). ¹H NMR (300 MHz, CDCl₃) δ 7.95 (s, 1H), 7.26 (s, 1H), 7.19 (d, J=7.2 Hz, 1H), 7.15-7.09 (m, 1H), 6.73 (d, J=7.2 Hz, 1H), 4.97 (s, 2H).

Step c. N-(3-(2H-Tetrazol-5-yl)phenyl)-5-chloro-2-morpholinooxazolo[4,5-b]pyridine-6-carboxamide: The procedure described in Example 1 was carried out to get the compound (51 mg, 19%) as a brown solid. ¹H NMR (300 MHz, DMSO-d₆) δ 10.82 (s, 1H), 8.55 (s, 1H), 8.14 (s, 1H), 7.82 (d, J=6.9 Hz, 1H), 7.77 (d, J=6.9 Hz, 1H), 7.68-7.54 (m, 1H), 3.85-3.71 (m, 8H).

Step d. N-(3-(2H-Tetrazol-5-yl)phenyl)-5-(4-methoxypiperidin-1-yl)-2-morpholinooxazolo[4,5-b]pyridine-6-carboxamide: The procedure described in Example 1 was carried out to get the compound (7 mg, 12%) as a white solid.

### Example 49, Method AC, AL

### Preparation of: (R)-5-(3-Hydroxypyrrolidin-1-yl)-N-(2-(2-methylpyridin-4-yl)oxazol-4-yl)-2-morpholinooxazolo[4,5-b]pyridine-6-carboxamide: (A49)

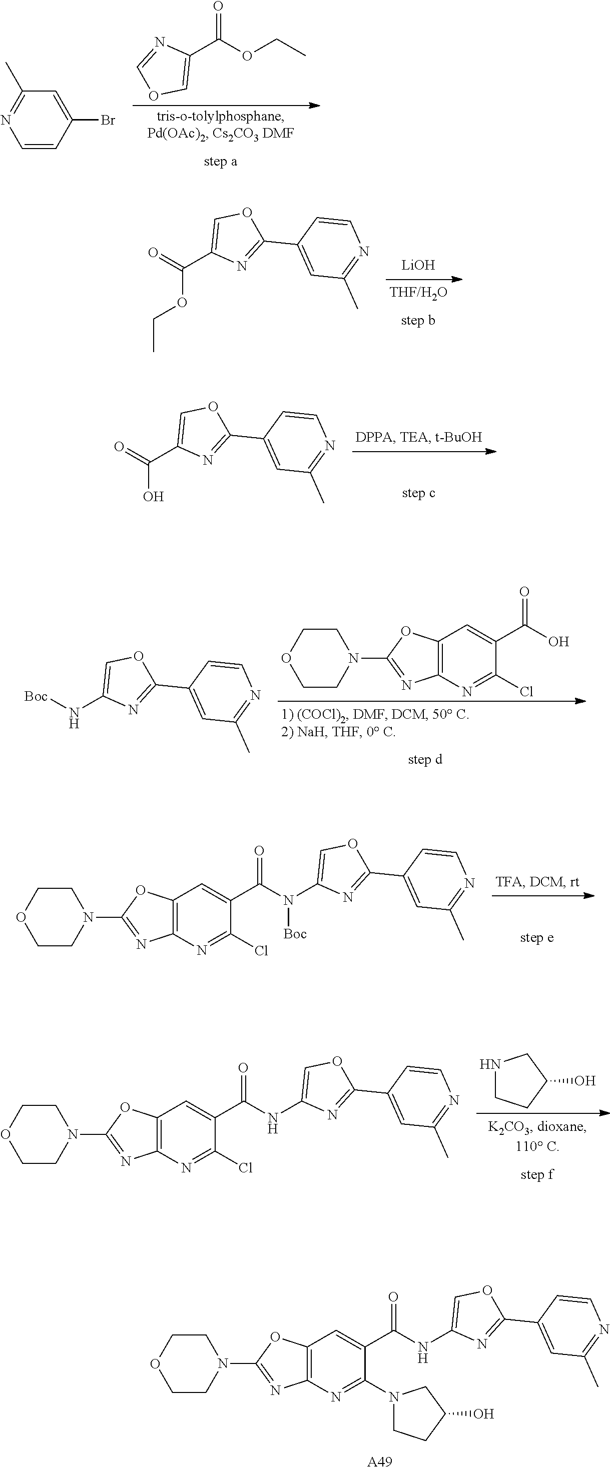

Step a. Ethyl 2-(2-methylpyridin-4-yl)oxazole-4-carboxylate: A suspension of 4-bromo-2-methylpyridine (7.5 g, 44 mmol), ethyl oxazole-4-carboxylate (6.2 g, 44 mmol), and tris-o-tolylphosphane (2.6 g, 8.7 mmol) in DMF (200 mL) was added Pd(OAc)₂ (500 mg, 4.4 mmol) and Cs₂CO₃ (15.6 g, 48 mmol). After being stirred at 70° C. overnight, the mixture was filtered and the filtrate was concentrated in vacuum to obtain the crude product that was used in the next step.

Step b. 2-(2-Methylpyridin-4-yl)oxazole-4-carboxylic acid: To a solution of the above crude in THF (50 mL) was added LiOH (2.1 g, 87 mmol) aqueous solution (10 mL). The mixture was stirred at room temperature overnight, and filtered. The filter cake was dissolved by water (20 mL) and adjusted pH to 4 with 1N HCl aqueous solution. The resulting precipitate was collected by filtration to obtain the product (2.3 g, 25%) as a yellow solid. ¹H NMR (300 MHz, DMSO-d₆) δ 8.93 (s, 1H), 8.64 (d, J=5.1 Hz, 1H), 7.81 (s, 1H), 7.72 (d, J=4.8 Hz, 1H), 2.57 (s, 3H). LC-MS (m/z): 205.1 [M+H]⁺.

Step c. tert-Butyl (2-(2-methylpyridin-4-yl)oxazol-4-yl)carbamate: To a solution of 2-(2-methylpyridin-4-yl)oxazole-4-carboxylic acid (510 mg, 2.5 mmol) and TEA (505 mg, 5.0 mmol) in t-BuOH (20 mL) was added DPPA (756 mg, 2.8 mmol) under N₂ atmosphere. After being stirred at 100° C. overnight, the mixture was cooled and concentrated in vacuum. The residue was purified by silica gel column (PE/AE=1/1) to get the desired product (230 mg, 33%) as a white solid. ¹H NMR (300 MHz, CDCl₃) δ 8.60 (d, J=5.1 Hz, 1H), 7.91 (s, 1H), 7.69 (s, 1H), 7.61 (d, J=4.8 Hz, 1H), 6.94 (s, 1H), 2.63 (s, 3H), 1.52 (s, 9H). LC-MS (m/z): 276.0 [M+H]⁺.

Step d. tert-Butyl (5-chloro-2-morpholinooxazolo[4,5-b]pyridine-6-carbonyl)(2-(2-methylpyridin-4-yl)oxazol-4-yl)carbamate: To a solution of 5-chloro-2-morpholinooxazolo[4,5-b]pyridine-6-carboxylic acid (500 mg, 4.3 mmol) in dry DCM (10 mL) was added catalytic amount of DMF and oxalyl chloride (1.6 g, 13 mmol). After being stirred for 1 h at 50° C., the reaction mixture was concentrated in vacuum to obtain the crude product. 60% NaH (142 mg, 3.6 mmol) was added to dry THF (25 mL), to above mixture were added tert-butyl (2-(2-methylpyridin-4-yl)oxazol-4-yl)carbamate (195 mg, 0.71 mmol) and the solution of above acyl chloride crude in THF (2 mL) at 0° C. under N₂ atmosphere. After being stirred for 1 h at room temperature, the mixture was quenched by adding acetic acid (1 mL) at −10° C. and concentrated in vacuum. The residue was purified by silica gel column (PE/AE=1/1) to obtain the desired product (150 mg, 39%) as a white solid. ¹H NMR (300 MHz, DMSO-d₆) δ 8.68-8.55 (m, 2H), 8.14 (s, 1H), 7.80 (s, 1H), 7.73-7.68 (m, 1H), 3.79-3.65 (m, 8H), 2.58 (s, 3H), 1.20 (s, 9H).

Step e. 5-Chloro-N-(2-(2-methylpyridin-4-yl)oxazol-4-yl)-2-morpholinooxazolo[4,5-b]pyridine-6-carboxamide: To a solution of tert-butyl (5-chloro-2-morpholinooxazolo[4,5-b]pyridine-6-carbonyl)(2-(2-methylpyridin-4-yl)oxazol-4-yl)carbamate (150 mg, 0.28 mmol) in DCM (5 mL) was added TFA (0.5 mL) and stirred for 5 h at room temperature. The mixture was concentrated in vacuum and the residue was adjusted pH to 8 with saturated NaHCO₃ aqueous solution. The resulting precipitate was collected by filtration and dried to obtain the product (90 mg, 73%) as a white solid. ¹H NMR (300 MHz, DMSO-d₆) δ 11.57 (s, 1H), 8.67 (d, J=5.1 Hz, 1H), 8.53 (s, 1H), 8.08 (s, 1H), 7.84 (s, 1H), 7.77 (d, J=4.8 Hz, 1H), 3.80-3.65 (m, 8H), 2.61 (s, 3H).

Step f. (R)-5-(3-Hydroxypyrrolidin-1-yl)-N-(2-(2-methylpyridin-4-yl)oxazol-4-yl)-2-morpholinooxazolo[4,5-b]pyridine-6-carboxamide: The procedure described in Example 1 was carried out to get the compound (15 mg, 38%) as a white solid.

### Example 50, Method AC

### Preparation of: 5-(4-Methoxypiperidin-1-yl)-N-(2-methoxypyridin-3-yl)-2-morpholinooxazolo[4,5-b]pyridine-6-carboxamide: (A50)

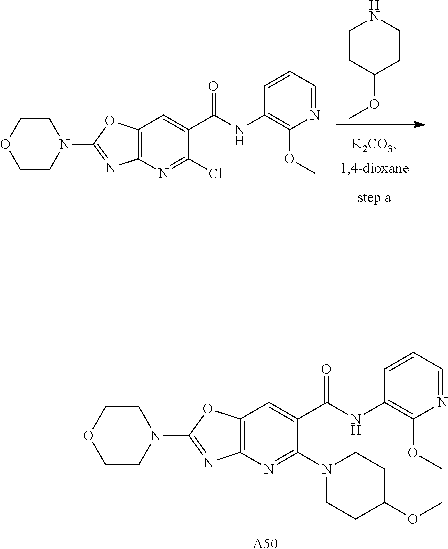

Step a. 5-(4-Methoxypiperidin-1-yl)-N-(2-methoxypyridin-3-yl)-2-morpholinooxazolo[4,5-b]pyridine-6-carboxamide: The procedure described in Example 1 was carried out to get the compound (15 mg, 38%) as a white solid.

### Example 51, Method AB, AM

### Preparation of: 5-(4-Methoxypiperidin-1-yl)-N-(6-(1-methyl-1H-pyrazol-4-yl)pyridin-2-yl)-2-(piperazin-1-yl)oxazolo[4,5-b]pyridine-6-carboxamide Hydrochloride: (A51)

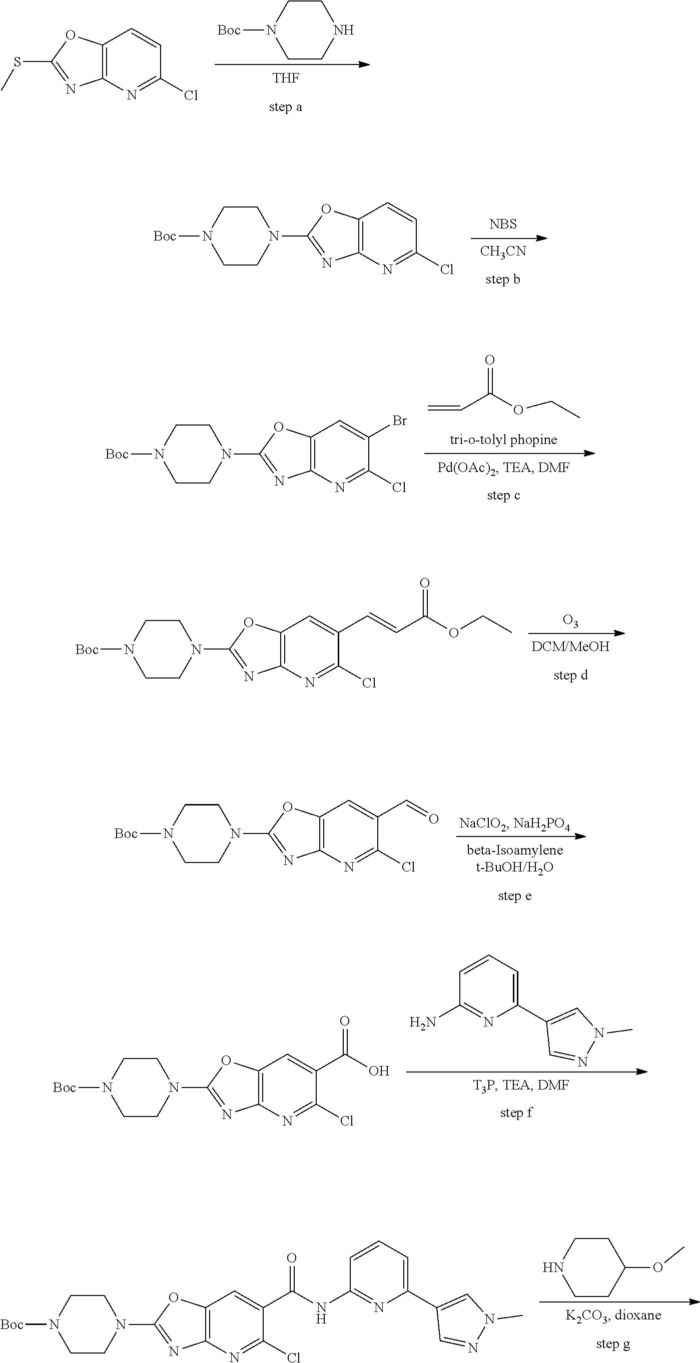

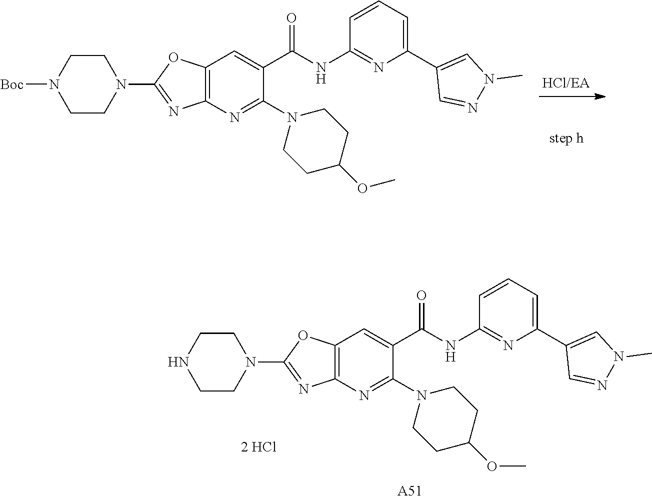

Step a. tert-Butyl 4-(5-chlorooxazolo[4,5-b]pyridin-2-yl)piperazine-1-carboxylate: The procedure described in Example 1 was carried out to get the desired compound (3.0 g, 72%) as a white solid. ¹H NMR (300 MHz, CDCl₃) δ 7.37 (d, J=8.1 Hz, 1H), 6.93 (d, J=8.1 Hz, 1H), 3.77-3.67 (m, 4H), 3.64-3.51 (m, 4H), 1.48 (s, 9H).

Step b. tert-Butyl 4-(6-bromo-5-chlorooxazolo[4,5-b]pyridin-2-yl)piperazine-1-carboxylate: The procedure described in Example 1 was carried out to get the desired compound (3.0 g, 83%) as a white solid. ¹H NMR (300 MHz, CDCl₃) δ 7.68 (s, 1H), 3.77-3.71 (m, 4H), 3.62-3.55 (m, 4H), 1.49 (s, 9H).

Step c. Tert-Butyl (E)-4-(5-chloro-6-(3-ethoxy-3-oxoprop-1-en-1-yl)oxazolo[4,5-b]pyridin-2-yl)piperazine-1-carboxylate: The procedure described in Example 1 was carried out to get the desired compound (460 mg, 46%) as a yellow solid. ¹H NMR (300 MHz, CDCl₃) δ 8.07 (d, J=15.9 Hz, 1H), 7.64 (s, 1H), 6.30 (d, J=15.9 Hz, 1H), 4.33-4.22 (m, 2H), 3.82-3.71 (m, 4H), 3.64-3.52 (m, 4H), 1.49 (s, 9H), 1.35 (t, J=5.7 Hz, 3H). LC-MS (m/z): 437.1 [M+H]⁺.

Step d. Tert-Butyl 4-(5-chloro-6-formyloxazolo[4,5-b]pyridin-2-yl)piperazine-1-carboxylate: The procedure described in Example 1 was carried out to get the desired compound (250 mg, 68%) as a yellow solid. ¹H NMR (300 MHz, CDCl₃) δ 10.37 (s, 1H), 7.91 (s, 1H), 3.88-3.73 (m, 4H), 3.66-3.52 (m, 4H), 1.48 (s, 9H). LC-MS (m/z): 367.1 [M+H]⁺.

Step e. 2-(4-(tert-Butoxycarbonyl)piperazin-1-yl)-5-chlorooxazolo[4,5-b]pyridine-6-carboxylic acid: The procedure described in Example 1 was carried out to get the desired compound (40 mg, 77%) as a white solid. ¹H NMR (300 MHz, DMSO-d₆) δ 8.14 (s, 1H), 3.78-3.63 (m, 4H), 3.60-3.46 (m, 4H), 1.44 (s, 9H). LC-MS (m/z): 383.1 [M+H]⁺.

Step f. tert-Butyl 4-(5-chloro-6-((6-(1-methyl-1H-pyrazol-4-yl)pyridin-2-yl)carbamoyl)oxazolo[4,5-b]pyridin-2-yl)piperazine-1-carboxylate: The procedure described in Example 1 was carried out to get the desired compound (10 mg, 18%) as a yellow solid. ¹H NMR (300 MHz, DMSO-d₆) δ 10.92 (s, 1H), 8.18 (s, 1H), 8.08 (s, 1H), 7.98-7.91 (m, 2H), 7.85-7.75 (m, 1H), 7.46-7.35 (m, 1H), 3.87 (s, 3H), 3.75-3.63 (m, 4H), 3.57-3.48 (m, 4H), 1.43 (s, 9H).

Step f. tert-Butyl 4-(5-(4-methoxypiperidin-1-yl)-6-((6-(1-methyl-1H-pyrazol-4-yl)pyridin-2-yl)carbamoyl)oxazolo[4,5-b]pyridin-2-yl)piperazine-1-carboxylate: The procedure described in Example 1 was carried out to get the desired compound (45 mg, 49%) as a white solid. ¹H NMR (300 MHz, DMSO-d₆) δ 13.46-13.31 (m, 1H), 8.35-8.20 (m, 2H), 8.10-7.99 (m, 2H), 7.78 (s, 1H), 7.39 (s, 1H), 3.87 (s, 3H), 3.76-3.63 (m, 9H), 3.25-3.13 (m, 5H), 3.10-2.93 (m, 2H), 2.25-1.98 (m, 4H), 1.43 (s, 9H). LC-MS (m/z): 618.3 [M+H]⁺.

Step f. 5-(4-Methoxypiperidin-1-yl)-N-(6-(1-methyl-1H-pyrazol-4-yl)pyridin-2-yl)-2-(piperazin-1-yl)oxazolo[4,5-b]pyridine-6-carboxamide Hydrochloride: A suspension of tert-butyl 4-(5-(4-methoxypiperidin-1-yl)-6-((6-(1-methyl-1H-pyrazol-4-yl)pyridin-2-yl)carbamoyl)oxazolo[4,5-b]pyridin-2-yl)piperazine-1-carboxylate (30 mg, 0.05 mmol) in 2.8 N HCl/EA (5 mL) was stirred at room temperature overnight. The mixture was concentrated and dried to get the compound (25 mg, 86%) as a yellow solid.

### Example 52, Method AO

### Preparation of: 5-(4-Methoxypiperidin-1-yl)-N-(6-(1-methyl-1H-pyrazol-4-yl)pyridin-2-yl)-2-(4-methylpiperazin-1-yl)oxazolo[4,5-b]pyridine-6-carboxamide: (A52)

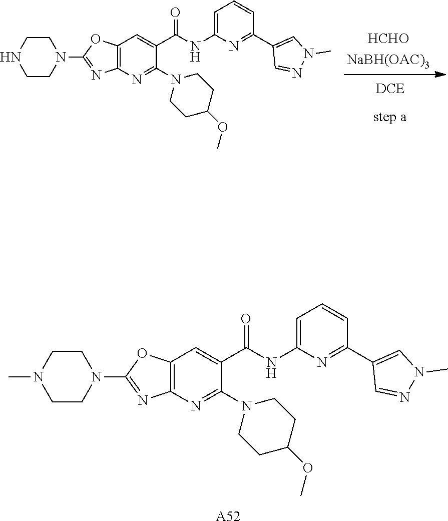

Step a. 5-(4-Methoxypiperidin-1-yl)-N-(6-(1-methyl-1H-pyrazol-4-yl)pyridin-2-yl)-2-(4-methylpiperazin-1-yl)oxazolo[4,5-b]pyridine-6-carboxamide: To a solution of5-(4-methoxypiperidin-1-yl)-N-(6-(1-methyl-1H-pyrazol-4-yl)pyridin-2-yl)-2-(piperazin-1-yl)oxazolo[4,5-b]pyridine-6-carboxamide (30 mg, 0.058 mmol) and POM (18 mg, 0.58 mmol) in DCE (2 mL) was added NaBH(OAc)₃ (124 mg, 0.58 mmol) at 0° C. The reaction was stirred at room temperature overnight, and then quenched with saturated NH₄Cl aqueous solution (30 mL). The mixture was extracted with dichloromethane (30 mL×3). The combined organic layers were dried over Na₂SO₄, filtered and concentrated. The residue was purified by silica gel column (DCM/MeOH=20/1) to give the desired product (6 mg, 20%) as a white solid.

### Example 53, Method AC

### Preparation of: 2,5-bis((R)-3-Hydroxypyrrolidin-1-yl)-N-(6-(1-methyl-1H-pyrazol-4-yl)pyridin-2-yl)oxazolo[4,5-b]pyridine-6-carboxamide: (A53)

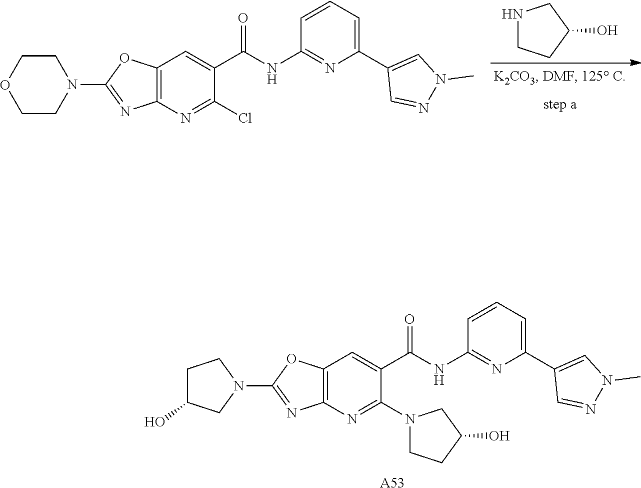

Step a. 2,5-bis((R)-3-Hydroxypyrrolidin-1-yl)-N-(6-(1-methyl-1H-pyrazol-4-yl)pyridin-2-yl)oxazolo[4,5-b]pyridine-6-carboxamide: The procedure described in Example 1 was carried out to get the by-product (20 mg, 37%) as a white solid.

### Example 54, Method AA, AB

### Preparation of: 5-Methoxy-N-(6-(1-methyl-1H-pyrazol-4-yl)pyridin-2-yl)-2-morpholinooxazolo[4,5-b]pyridine-6-carboxamide: (A54)

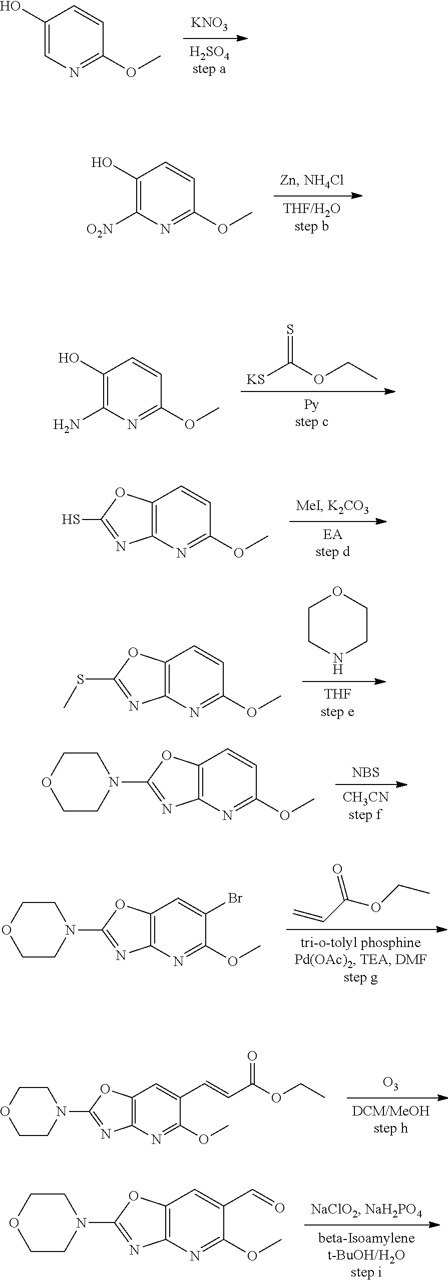

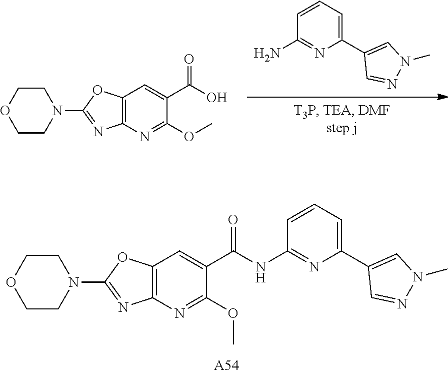

Step a. 6-Methoxy-2-nitropyridin-3-ol: The procedure described in Example 1 was carried out to obtain the product (5.0 g, 77%) as a yellow solid. H NMR (300 MHz, CDCl₃) δ 10.18 (s, 1H), 7.54 (d, J=9.0 Hz, 1H), 7.11 (d, J=9.0 Hz, 1H), 3.98 (s, 3H).

Step b. 2-Amino-6-methoxypyridin-3-ol: The procedure described in Example 1 was carried out to obtain the product (2.7 g, 82%) as a black solid. ¹H NMR (300 MHz, DMSO-d₆) δ 8.69 (s, 1H), 6.95-6.70 (m, 1H), 5.87-5.66 (m, 1H), 5.35 (s, 2H), 3.65 (s, 3H).

Step c. 5-Methoxyoxazolo[4,5-b]pyridine-2-thiol: The procedure described in Example 1 was carried out to obtain the product (1.0 g, 77%) as a yellow solid. ¹H NMR (300 MHz, CDCl₃) δ 7.49 (d, J=8.8 Hz, 1H), 6.60 (d, J=9.3 Hz, 1H), 3.92 (s, 3H). LC-MS (m/z): 183.1 [M+H]⁺.

Step d. 5-Methoxy-2-(methylthio)oxazolo[4,5-b]pyridine: The procedure described in Example 1 was carried out to obtain the product (1.8 g, 81%) as a yellow solid. ¹H NMR (300 MHz, CDCl₃) δ 7.81 (d, J=8.4 Hz, 1H), 6.82 (d, J=7.5 Hz, 1H), 4.19 (s, 3H), 2.98 (s, 3H).

Step e. 5-Methoxy-2-morpholinooxazolo[4,5-b]pyridine: The procedure described in Example 1 was carried out to obtain the product (700 mg, 58%) as a brown solid. ¹H NMR (300 MHz, CDCl₃) δ 7.39 (d, J=8.4 Hz, 1H), 6.37 (d, J=8.4 Hz, 1H), 3.96 (s, 3H), 3.84-3.79 (m, 4H), 3.74-3.68 (m, 4H).

Step f. 6-Bromo-5-methoxy-2-morpholinooxazolo[4,5-b]pyridine: The procedure described in Example 1 was carried out to obtain the product (90 mg, 68%) as a white solid. ¹H NMR (300 MHz, CDCl₃) δ 7.65 (s, 1H), 4.04 (s, 3H), 3.85-3.78 (m, 4H), 3.76-3.69 (m, 4H). LC-MS (m/z): 313.9 [M+H]⁺.

Step g. Ethyl (E)-3-(5-methoxy-2-morpholinooxazolo[4,5-b]pyridin-6-yl)acrylate: The procedure described in Example 1 was carried out to obtain the product (400 mg, 75%) as a yellow oil. ¹H NMR (300 MHz, CDCl₃) δ 7.91 (d, J=15.9 Hz, 1H), 7.57 (s, 1H), 6.42 (d, J=16.2 Hz, 1H), 4.26 (q, 7.2 Hz, 2H), 4.05 (s, 3H), 3.86-3.70 (m, 8H), 1.33 (t, J=7.2 Hz, 3H).

Step h. 5-Methoxy-2-morpholinooxazolo[4,5-b]pyridine-6-carbaldehyde: The procedure described in Example 1 was carried out to obtain the product (100 mg, 84%) as a yellow solid. ¹H NMR (300 MHz, CDCl₃) δ 10.29 (s, 1H), 7.87 (s, 1H), 4.09 (s, 3H), 3.82 (s, 8H).

Step i. 5-Methoxy-2-morpholinooxazolo[4,5-b]pyridine-6-carboxylic acid: The procedure described in Example 1 was carried out to obtain the product (70 mg, 74%) as a white solid. ¹H NMR (300 MHz, DMSO-d₆) δ 8.05 (s, 1H), 3.89 (s, 3H), 3.77-3.70 (m, 4H), 3.70-3.62 (m, 4H).

Step g. 5-Methoxy-N-(6-(1-methyl-1H-pyrazol-4-yl)pyridin-2-yl)-2-morpholinooxazolo[4,5-b]pyridine-6-carboxamide: The procedure described in Example 1 was carried out to obtain the product (16 mg, 26%) as a white solid.

### Example 55, Method AM

### Preparation of: N-(6-(1-Methyl-1H-pyrazol-4-yl)pyridin-2-yl)-2-(piperazin-1-yl)-5-(1-oxa-7-azaspiro[3.5]nonan-7-yl)oxazolo[4,5-b]pyridine-6-carboxamide: (A55)

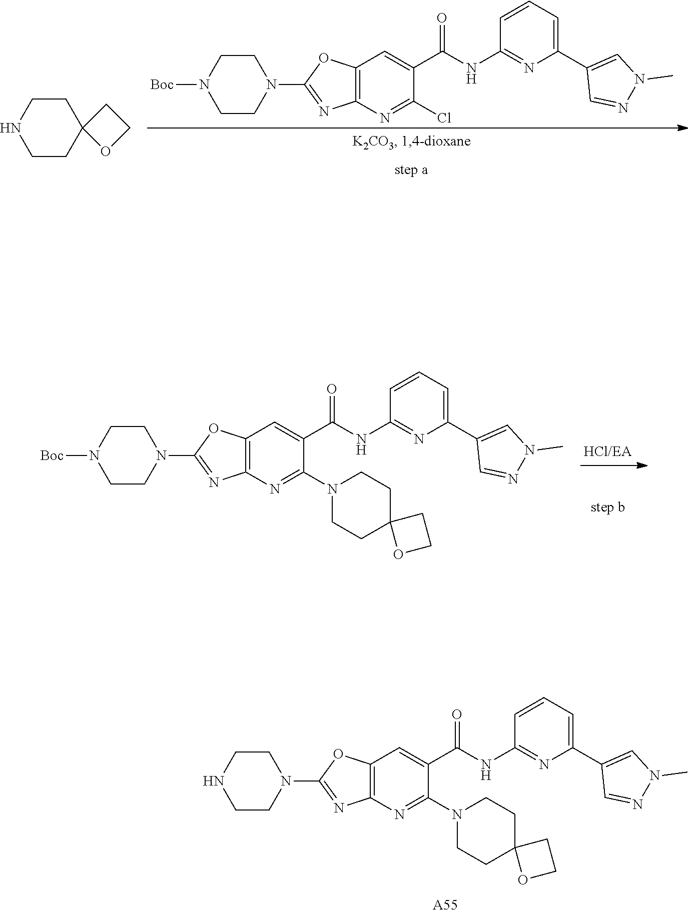 [SMILES: C1CC2(CCN1)CCO2 ; Cn1cc(-c2cccc(NC(=O)c3cc4oc(N5CCN(C(=O)OC(C)(C)C)CC5)nc4nc3Cl)n2)cn1 ; Cn1cc(-c2cccc(NC(=O)c3cc4oc(N5CCN(C(=O)OC(C)(C)C)CC5)nc4nc3N3CCC4(CCO4)CC3)n2)cn1 ; Cn1cc(-c2cccc(NC(=O)c3cc4oc(N5CCNCC5)nc4nc3N3CCC4(CCO4)CC3)n2)cn1 ; "A55"]

Step a. tert-Butyl 4-(6-((6-(1-methyl-1H-pyrazol-4-yl)pyridin-2-yl)carbamoyl)-5-(1-oxa-7-azaspiro[3.5]nonan-7-yl)oxazolo[4,5-b]pyridin-2-yl)piperazine-1-carboxylate: The procedure described in Example 1 was carried out to obtain the product (40 mg, 54%) as a yellow solid. ¹H NMR (300 MHz, DMSO-d₆) δ 12.40 (s, 1H), 8.22 (s, 1H), 8.13 (s, 1H), 8.05 (d, J=7.8 Hz, 1H), 7.92 (s, 1H), 7.78 (t, J=7.8 Hz, 1H), 7.37 (d, J=7.5 Hz, 1H), 5.65-5.56 (m, 1H), 4.52-4.43 (m, 1H), 3.89 (s, 3H), 3.80-3.61 (m, 6H), 3.58-3.44 (m, 6H), 3.26-3.18 (m, 2H), 2.45-2.35 (m, 2H), 2.27-2.15 (m, 2H), 1.41 (s, 9H). LC-MS (m/z): 629.9 [M+H]⁺.

Step b. N-(6-(1-Methyl-1H-pyrazol-4-yl)pyridin-2-yl)-2-(piperazin-1-yl)-5-(1-oxa-7-azaspiro[3.5]nonan-7-yl)oxazolo[4,5-b]pyridine-6-carboxamide: The procedure described in Example 51 was carried out to obtain the product (50 mg, 65%) as a white solid.

### Example 56, Method AM

### Preparation of: 5-(4-Hydroxy-4-methylpiperidin-1-yl)-N-(6-(1-methyl-1H-pyrazol-4-yl)pyridin-2-yl)-2-(piperazin-1-yl)oxazolo[4,5-b]pyridine-6-carboxamide: (A56)

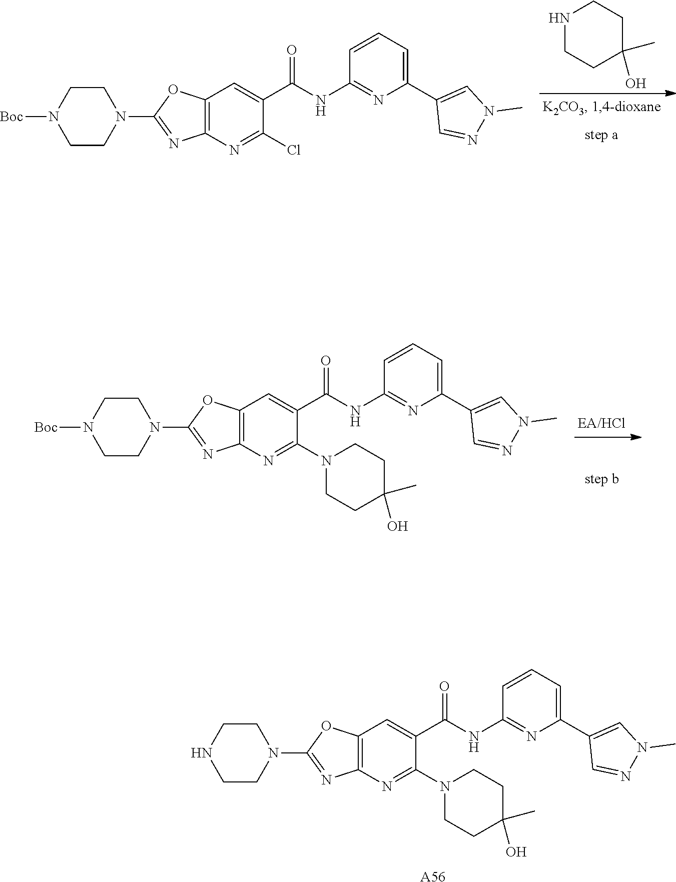

Step a. tert-Butyl 4-(5-(4-hydroxy-4-methylpiperidin-1-yl)-6-((6-(1-methyl-1H-pyrazol-4-yl)pyridin-2-yl)carbamoyl)oxazolo[4,5-b]pyridin-2-yl)piperazine-1-carboxylate: The procedure described in Example 1 was carried out to obtain the product (96 mg, 55%) as a white solid. ¹H NMR (300 MHz, DMSO-d₆) δ 13.17 (s, 1H), 8.36 (s, 1H), 8.26 (s, 1H), 8.08 (d, J=8.4 Hz, 1H), 8.01 (s, 1H), 7.79 (t, J=7.5 Hz, 1H), 7.38 (d, J=7.5 Hz, 1H), 4.60 (s, 1H), 3.87 (s, 3H), 3.75-3.66 (m, 4H), 3.57-3.47 (m, 4H), 3.28-3.20 (m, 2H), 3.07-2.94 (m, 2H), 1.89 (s, 3H), 1.44 (s, 9H), 1.33-1.17 (m, 4H). LC-MS (m/z): 617.8 [M+H]⁺.

Step b. 5-(4-Hydroxy-4-methylpiperidin-1-yl)-N-(6-(1-methyl-1H-pyrazol-4-yl)pyridin-2-yl)-2-(piperazin-1-yl)oxazolo[4,5-b]pyridine-6-carboxamide: The procedure described in Example 51 was carried out to obtain the product (38 mg, 49%) as a white solid.

### Example 57, Method AN

### Preparation of: 2-(4-(2-Hydroxyethyl)piperazin-1-yl)-5-(4-methoxypiperidin-1-yl)-N-(6-(1-methyl-1H-pyrazol-4-yl)pyridin-2-yl)oxazolo[4,5-b]pyridine-6-carboxamide: (A57)

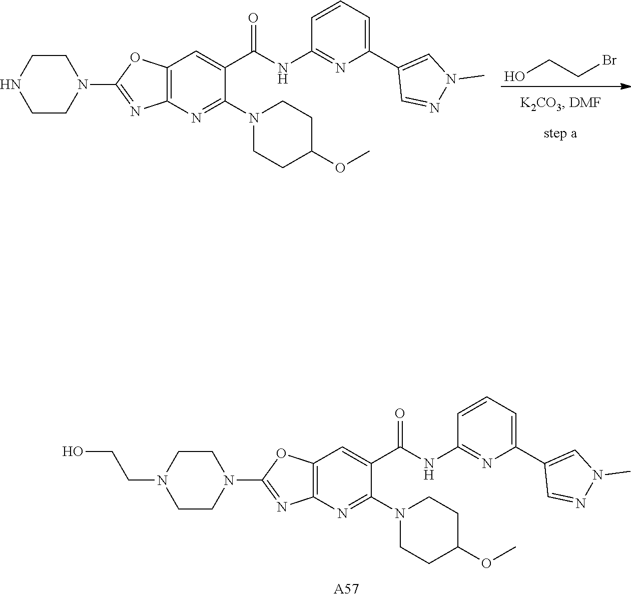

Step a. 2-(4-(2-Hydroxyethyl)piperazin-1-yl)-5-(4-methoxypiperidin-1-yl)-N-(6-(1-methyl-1H-pyrazol-4-yl)pyridin-2-yl)oxazolo[4,5-b]pyridine-6-carboxamide: To a solution of 5-(4-methoxypiperidin-1-yl)-N-(6-(1-methyl-1H-pyrazol-4-yl)pyridin-2-yl)-2-(piperazin-1-yl)oxazolo[4,5-b]pyridine-6-carboxamide (30 mg, 0.058 mmol) and 2-bromoethan-1-ol (11 mg, 0.087 mmol) in dry DMF (2 ml) was added K₂CO₃ (16 mg, 0.12 mmol), and then stirred at 100° C. overnight. The mixture was diluted by water (5 mL) and extracted with EA (10 mL×3). The combined organic layers were dried over Na₂SO₄, filtrated and concentrated and the residue was purified by silica gel column (DCM/MeOH=50/1) to give the desired product (5 mg, 15%) as a white solid.

### Example 58, Method AO

### Preparation of: N-(6-(1-Methyl-1H-pyrazol-4-yl)pyridin-2-yl)-2-(4-methylpiperazin-1-yl)-5-(1-oxa-7-azaspiro[3.5]nonan-7-yl)oxazolo[4,5-b]pyridine-6-carboxamide: (A58)

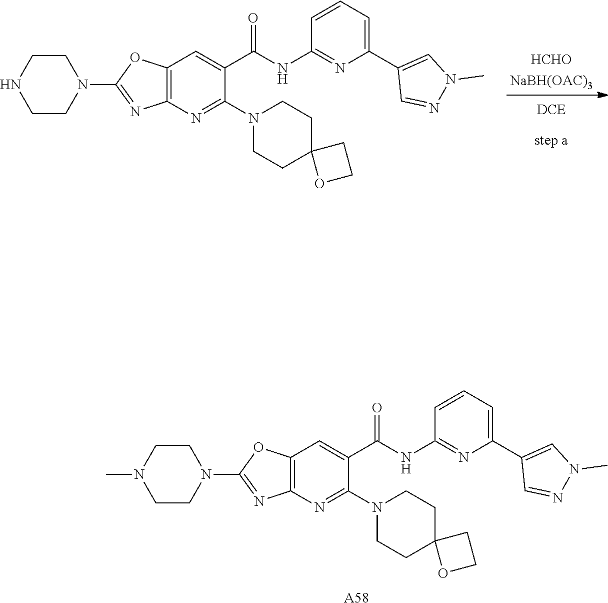

Step a. N-(6-(1-Methyl-1H-pyrazol-4-yl)pyridin-2-yl)-2-(4-methylpiperazin-1-yl)-5-(1-oxa-7-azaspiro[3.5]nonan-7-yl)oxazolo[4,5-b]pyridine-6-carboxamide: The procedure described in Example 52 was carried out to obtain the product (12 mg, 80%) as a white solid.

### Example 59, Method AO

### Preparation of: 5-(4-Hydroxy-4-methylpiperidin-1-yl)-N-(6-(1-methyl-1H-pyrazol-4-yl)pyridin-2-yl)-2-(4-methylpiperazin-1-yl)oxazolo[4,5-b]pyridine-6-carboxamide: (A59)

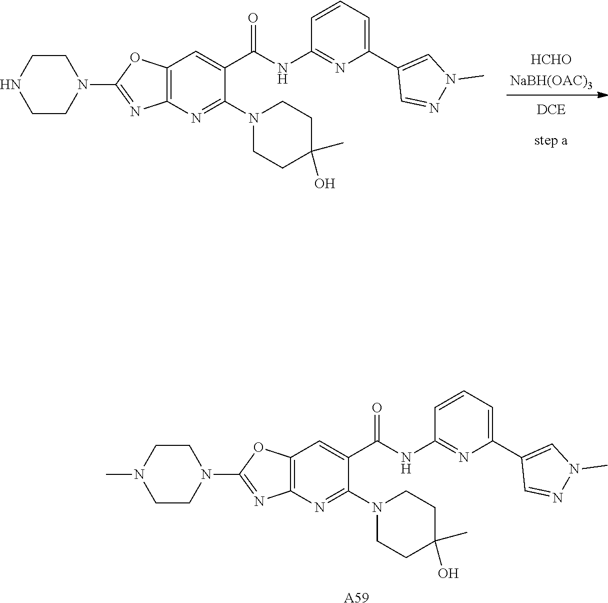

Step a. 5-(4-Hydroxy-4-methylpiperidin-1-yl)-N-(6-(1-methyl-1H-pyrazol-4-yl)pyridin-2-yl)-2-(4-methylpiperazin-1-yl)oxazolo[4,5-b]pyridine-6-carboxamide: The procedure described in Example 52 was carried out to obtain the product (6 mg, 40%) as a white solid.

### Example 60, Method BA, BB

### Preparation of: 6-(4-Methoxypiperidin-1-yl)-2,2-dimethyl-N-(6-(1-methyl-1H-pyrazol-4-yl)pyridin-2-yl)-2,3-dihydrofuro[2,3-b]pyridine-5-carboxamide: (B1)

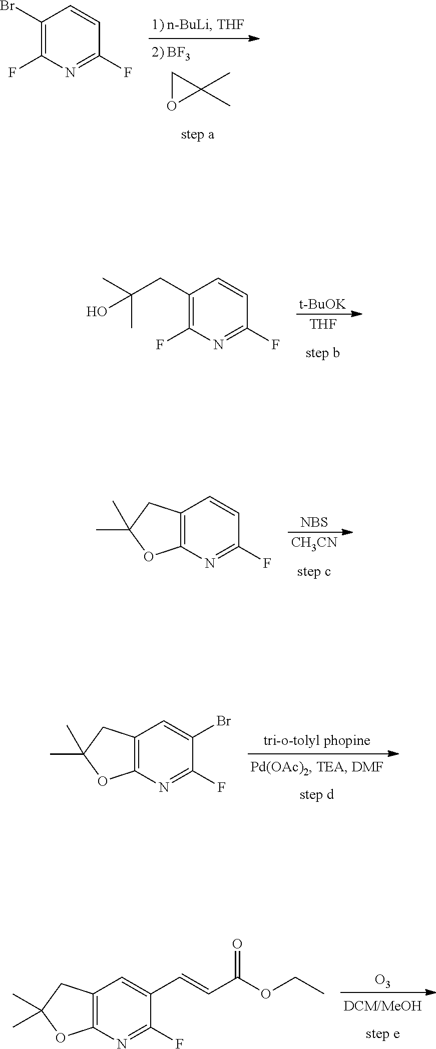

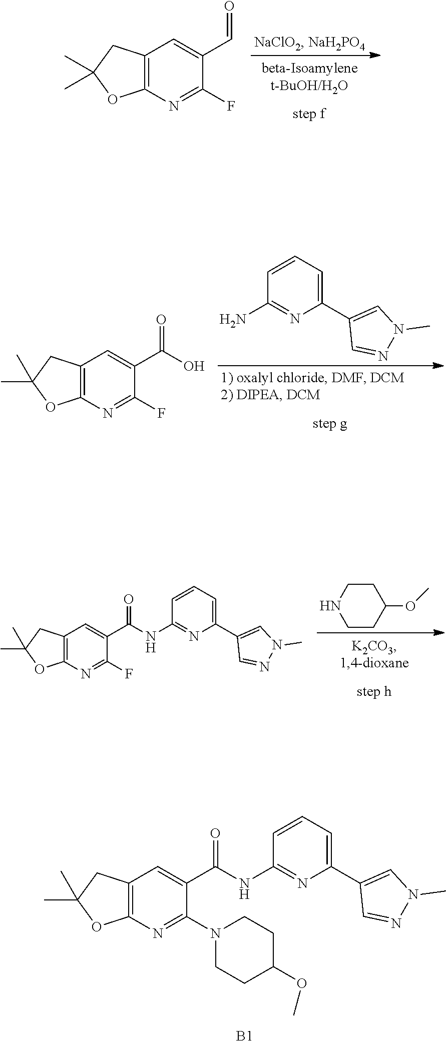

Step a. 1-(2,6-Difluoropyridin-3-yl)-2-methylpropan-2-ol: To a solution of 3-bromo-2,6-difluoropyridine (200 mg, 1.0 mmol) in dry THF (10 mL) was added n-BuLi (0.44 mL, 1.1 mmol) and the mixture was stirred for 0.5 h at −78° C. Then the mixture was added 2,2-dimethyloxirane (86 mg, 1.2 mmol) and boron trifluoride ether solution (0.15 mL, 1.2 mmol) slowly and stirred for 2 h at −78° C.

The reaction mixture was quenched by adding water (10 mL) and extracted with EA (20 mL×3). The combined organic layers were dried over Na₂SO₄, filtrated and concentrated and the residue was purified by silica gel column (PE/EA=4/1) to give the desired product (50 mg, 27%) as a yellow solid. ¹H NMR (300 MHz, CDCl₃) δ 7.82 (dd, J=16.5, 8.1 Hz, 1H), 6.79 (d, J=8.1 Hz, 1H), 2.78 (s, 2H), 1.26 (s, 6H).

Step b. 6-Fluoro-2,2-dimethyl-2,3-dihydrofuro[2,3-b]pyridine: To a solution of 1-(2,6-difluoropyridin-3-yl)-2-methylpropan-2-ol (40 mg, 0.21 mmol) in dry THF (10 mL) was added t-BuOK (47 mg, 0.42 mmol) and the mixture was stirred at 50° C. overnight. The mixture was diluted by EA (10 mL) and washed with water (10 mL×3). The combined organic layers were dried over Na₂SO₄, filtrated and concentrated and the residue was purified by silica gel column (PE/EA=5/1) to give the desired product (50 mg, 27%) as a colorlessoil. ¹H NMR (300 MHz, CDCl₃) δ 7.52-7.38 (m, 1H), 6.35 (d, J=6.9 Hz, 1H), 2.99 (s, 2H), 1.51 (s, 6H).

Step c. 5-Bromo-6-fluoro-2,2-dimethyl-2,3-dihydrofuro[2,3-b]pyridine: To a solution of 6-fluoro-2,2-dimethyl-2,3-dihydrofuro[2,3-b]pyridine (1.0 g, 6.0 mmol) in dry CH₃CN (100 mL) was added NBS (1.6 g, 9.0 mmol) and the mixture was stirred at room temperature overnight. The mixture was diluted by water (50 mL) and extracted with DCM (10 mL×3). The combined organic layers were dried over Na₂SO₄, filtrated and concentrated and the residue was purified by silica gel column (PE/EA=10/1) to give the desired product (300 mg, 21%) as a yellow solid. ¹H NMR (300 MHz, CDCl₃) δ 7.60 (d, J=8.4 Hz, 1H), 3.01 (s, 2H), 1.51 (s, 6H).

Step d. Ethyl (E)-3-(6-fluoro-2,2-dimethyl-2,3-dihydrofuro[2,3-b]pyridin-5-yl)acrylate: To a solution of 5-bromo-6-fluoro-2,2-dimethyl-2,3-dihydrofuro[2,3-b]pyridine (100 mg, 0.41 mmol) in dry DMF (2 mL) was added tri(o-tolyl)phosphine (36 mg, 0.12 mmol), palladium acetate (9.0 mg, 0.04 mmol), TEA(121 mg, 1.2 mmol) and ethyl acrylate (810 mg, 8.1 mmol). The mixture was refluxed for 2 h at 140° C., under N₂ and the solvent was removed in vacuum. The residue was purified by silica gel column chromatography(PE/EA=5/1) to give the desired product (50 mg, 46%) as a yellow solid. ¹H NMR (300 MHz, CDCl₃) δ 7.68 (d, J=16.5 Hz, 1H), 7.68 (s, 1H), 6.33 (d, J=16.2 Hz, 1H), 4.24 (q, J=7.2 Hz, 2H), 3.03 (s, 2H), 1.53 (s, 6H), 1.32 (t, J=7.2 Hz, 3H).

Step e. 6-Fluoro-2,2-dimethyl-2,3-dihydrofuro[2,3-b]pyridine-5-carbaldehyde: To a solution of ethyl (E)-3-(6-fluoro-2,2-dimethyl-2,3-dihydrofuro[2,3-b]pyridin-5-yl)acrylate (50 mg, 0.18 mmol) in DCM/MeOH (30 mL/3 mL) was bubbled with dry O₃ for 10 min at −78° C. The reaction was complete detected by TLC, and quenched by adding dimethyl sulfide (0.5 mL). The solvent was removed in vacuum to give the crude product (40 mg) as a yellow solid. ¹H NMR (300 MHz, CDCl₃) δ 10.14 (s, 1H), 8.01 (d, J=8.7 Hz, 1H), 3.06 (s, 2H), 1.57 (s, 6H).

Step f. 6-Fluoro-2,2-dimethyl-2,3-dihydrofuro[2,3-b]pyridine-5-carboxylic acid: To a solution of 6-fluoro-2,2-dimethyl-2,3-dihydrofuro[2,3-b]pyridine-5-carbaldehyde (50 mg, 0.25 mmol) in t-BuOH (5 mL) was added 2-Methyl-2-butene (175 mg, 2.5 mmol), sodium chlorite (112 mg, 1.2 mmol) and sodium dihydrogen phosphate dehydrate (390 mg, 2.5 mmol) aqueous solution (5 mL). After stirred for 2 h at room temperature, the mixture was adjusted pH to 2 with 1 N HCl aqueous solution and extracted with DCM (30 mL×3). The solvent was removed in vacuum to give the desired product (40 mg, 77%) as a white solid. ¹H NMR (300 MHz, DMSO-d₆) a 8.13 (d, J=9.3 Hz, 1H), 3.07 (s, 2H), 1.48 (s, 6H).

Step g. 6-Fluoro-2,2-dimethyl-N-(6-(1-methyl-1H-pyrazol-4-yl)pyridin-2-yl)-2,3-dihydrofuro[2,3-b]pyridine-5-carboxamide: To a solution of 6-fluoro-2,2-dimethyl-2,3-dihydrofuro[2,3-b]pyridine-5-carboxylic acid (477 mg, 2.3 mmol) in dry DCM (20 mL) was added catalytic amount of DMF and oxalyl chloride (2.8 g, 23 mmol). After being stirred for 6 h at 30° C., the reaction mixture was concentrated in vacuum to obtain the crude product. To another solution of 6-(1-methyl-1H-pyrazol-4-yl)pyridin-2-amine (470 mg, 2.7 mmol) and DIPEA (580 mg, 4.5 mmol) in dry DCM (20 mL) was added the solution of above crude in DCM (1 mL). The mixture was stirred for 3 h at the room temperature and quenched by saturated NaHCO₃ aqueous solution. The mixture was extracted with DCM (30 mL×3) and the combined organic layers were dried over Na₂SO₄, filtrated and concentrated and the residue was purified by silica gel column (DCM/MeOH=50/1) to give the desired product (450 mg, 54%) as a white solid. ¹H NMR (300 MHz, CDCl₃) δ 9.01 (d, J=13.8 Hz, 1H), 8.32 (d, J=9.6 Hz, 1H), 8.12 (d, J=8.1 Hz, 1H), 7.92 (d, J=3.6 Hz, 1H), 7.70 (t, J=7.8 Hz, 1H), 7.23 (d, J=7.8 Hz, 1H), 3.96 (s, 3H), 3.09 (s, 2H), 1.57 (s, 6H).

Step h. 6-(4-Methoxypiperidin-1-yl)-2,2-dimethyl-N-(6-(1-methyl-1H-pyrazol-4-yl)pyridin-2-yl)-2,3-dihydrofuro[2,3-b]pyridine-5-carboxamide: To a solution of 6-fluoro-2,2-dimethyl-N-(6-(1-methyl-1H-pyrazol-4-yl)pyridin-2-yl)-2,3-dihydrofuro[2,3-b]pyridine-5-carboxamide (55 mg, 0.15 mmol) in dry 1,4-dioxane (3 mL) was added K₂CO₃ (207 mg, 1.5 mmol) and 4-methoxypiperidine (172 mg, 1.5 mmol) and the mixture was stirred at 100° C. overnight. The mixture was concentrated in vacuum and the residue was purified by silica gel column (DCM/MeOH=50/1) to give the desired product (14 mg, 20%) as a white solid.

### Example 61, Method BB

### Preparation of: 2,2-Dimethyl-N-(6-(1-methyl-1H-pyrazol-4-yl)pyridin-2-yl)-6-(4-methylpiperazin-1-yl)-2,3-dihydrofuro[2,3-b]pyridine-5-carboxamide: (B2)

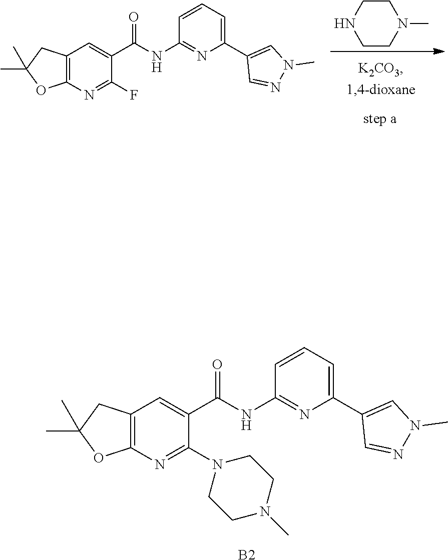

Step a. 2,2-Dimethyl-N-(6-(1-methyl-1H-pyrazol-4-yl)pyridin-2-yl)-6-(4-methylpiperazin-1-yl)-2,3-dihydrofuro[2,3-b]pyridine-5-carboxamide: The procedure described in Example 60 was carried to obtain the product (22 mg, 61%) as a white solid.

### Example 62, Method BB

### Preparation of: 6-(4-(2-Hydroxyethyl)piperazin-1-yl)-2,2-dimethyl-N-(6-(1-methyl-1H-pyrazol-4-yl)pyridin-2-yl)-2,3-dihydrofuro[2,3-b]pyridine-5-carboxamide: (B3)

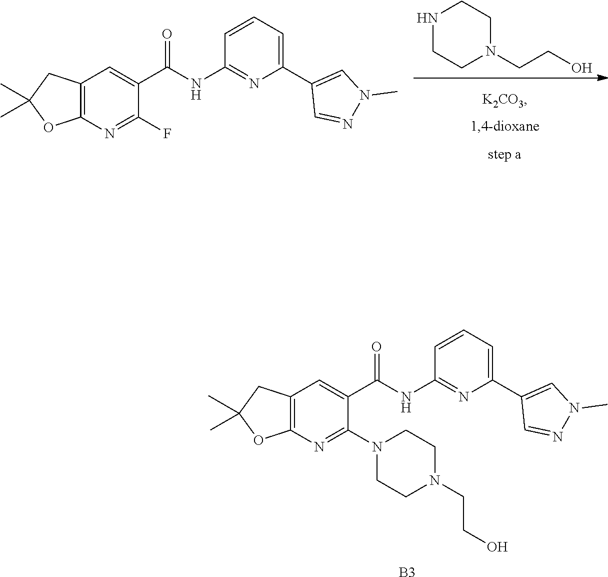

Step a. 6-(4-(2-Hydroxyethyl)piperazin-1-yl)-2,2-dimethyl-N-(6-(1-methyl-1H-pyrazol-4-yl)pyridin-2-yl)-2,3-dihydrofuro[2,3-b]pyridine-5-carboxamide: The procedure described in Example 60 was carried to obtain the product (21 mg, 54%) as a white solid.

### Example 63, Method BB

### Preparation of: 6-(4-Cyclopropylpiperazin-1-yl)-2,2-dimethyl-N-(6-(1-methyl-1H-pyrazol-4-yl)pyridin-2-yl)-2,3-dihydrofuro[2,3-b]pyridine-5-carboxamide: (B4)

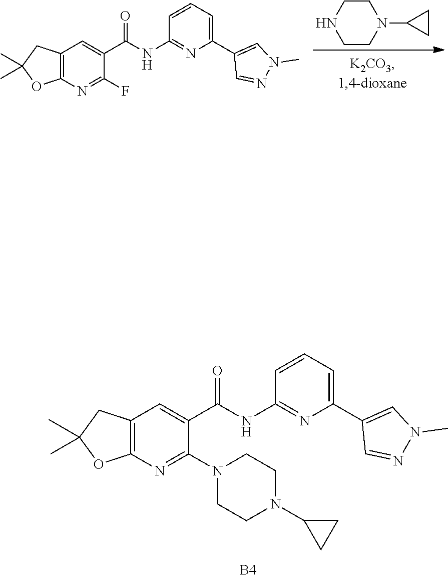

Step a. 6-(4-Cyclopropylpiperazin-1-yl)-2,2-dimethyl-N-(6-(1-methyl-1H-pyrazol-4-yl)pyridin-2-yl)-2,3-dihydrofuro[2,3-b]pyridine-5-carboxamide: The procedure described in Example 60 was carried to obtain the product (24 mg, 37%) as a white solid.

### Example 64, Method BB

### Preparation of: 6-(4-Hydroxypiperidin-1-yl)-2,2-dimethyl-N-(6-(1-methyl-1H-pyrazol-4-yl)pyridin-2-yl)-2,3-dihydrofuro[2,3-b]pyridine-5-carboxamide: (B5)

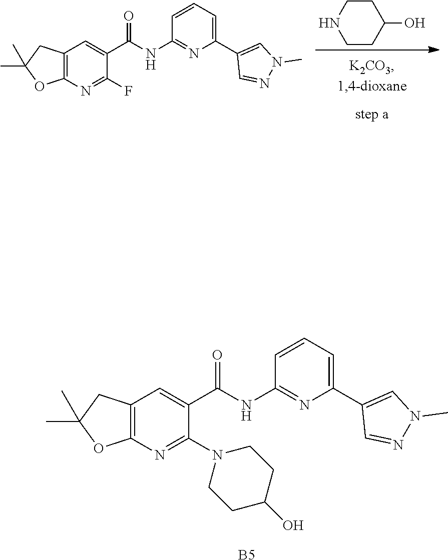

Step a. 6-(4-Hydroxypiperidin-1-yl)-2,2-dimethyl-N-(6-(1-methyl-1H-pyrazol-4-yl)pyridin-2-yl)-2,3-dihydrofuro[2,3-b]pyridine-5-carboxamide: The procedure described in Example 60 was carried to obtain the product (22 mg, 61%) as a white solid.

### Example 65, Method BB

### Preparation of: 2,2-Dimethyl-N-(6-(1-methyl-1H-pyrazol-4-yl)pyridin-2-yl)-6-(1-oxa-7-azaspiro[3.5]nonan-7-yl)-2,3-dihydrofuro[2,3-b]pyridine-5-carboxamide: (B6)

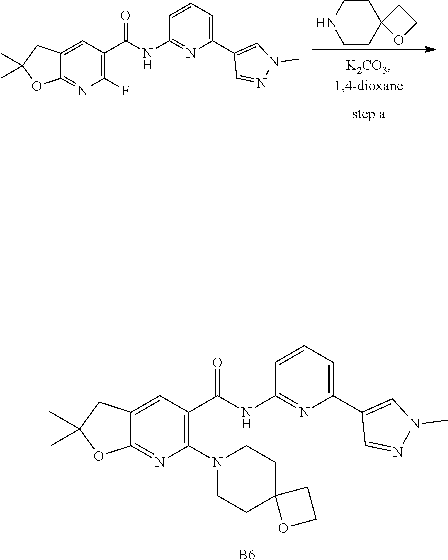

Step a. 2,2-Dimethyl-N-(6-(1-methyl-H-pyrazol-4-yl)pyridin-2-yl)-6-(1-oxa-7-azaspirol[3.5]nonan-7-yl)-2,3-dihydrofuro[2,3-b]pyridine-5-carboxamide: The procedure described in Example 60 was carried to obtain the product (17 mg, 38%) as a white solid.

### Example 66, Method BB

### Preparation of: 6-(4-Hydroxy-4-methylpiperidin-1-yl)-2,2-dimethyl-N-(6-(1-methyl-1H-pyrazol-4-yl)pyridin-2-yl)-2,3-dihydrofuro[2,3-b]pyridine-5-carboxamide: (B7)

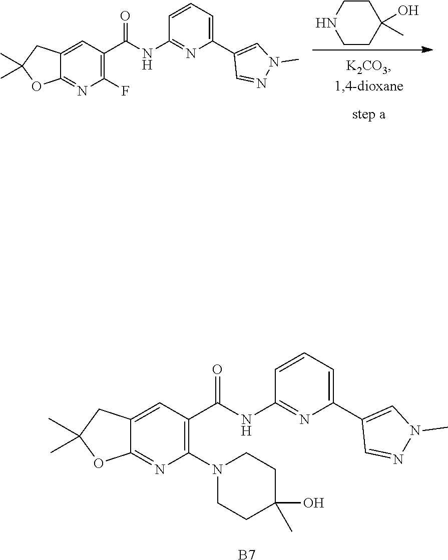

Step a. 6-(4-Hydroxy-4-methylpiperidin-1-yl)-2,2-dimethyl-N-(6-(1-methyl-1H-pyrazol-4-yl)pyridin-2-yl)-2,3-dihydrofuro[12,3-b]pyridine-5-carboxamide: The procedure described in Example 60 was carried to obtain the product (23 mg, 52%) as a white solid.

### Example 67, Method BB

### Preparation of: 2,2-Dimethyl-N-(6-(1-methyl-1H-pyrazol-4-yl)pyridin-2-yl)-6-(4,7-diazaspiro[2.5]octan-7-yl)-2,3-dihydrofurol[2,3-b]pyridine-5-carboxamide: (B8)

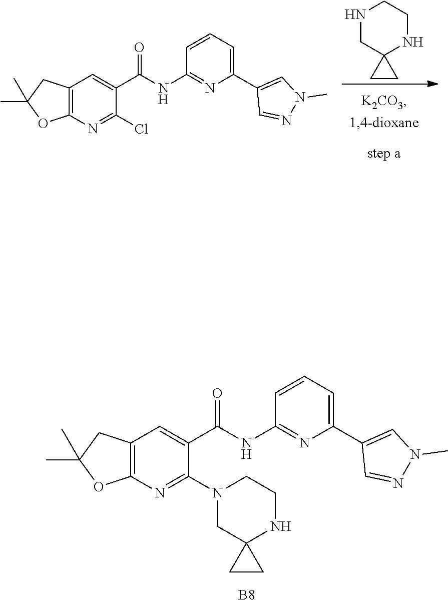

Step a. 2,2-Dimethyl-N-(6-(1-methyl-1H-pyrazol-4-yl)pyridin-2-yl)-6-(4,7-diazaspiro[2.5]octan-7-yl)-2,3-dihydrofuro[2,3-b]pyridine-5-carboxamide: The procedure described in Example 60 was carried to obtain the product (29 mg, 69%) as a white solid.

### Example 68, Method BB, BD

### Preparation of: N-(1-((1S,2R)-2-Fluorocyclopropyl)-2-oxo-1,2-dihydropyridin-3-yl)-6-(4-hydroxypiperidin-1-yl)-2,2-dimethyl-2,3-dihydrofuro[2,3-b]pyridine-5-carboxamide: (B9)

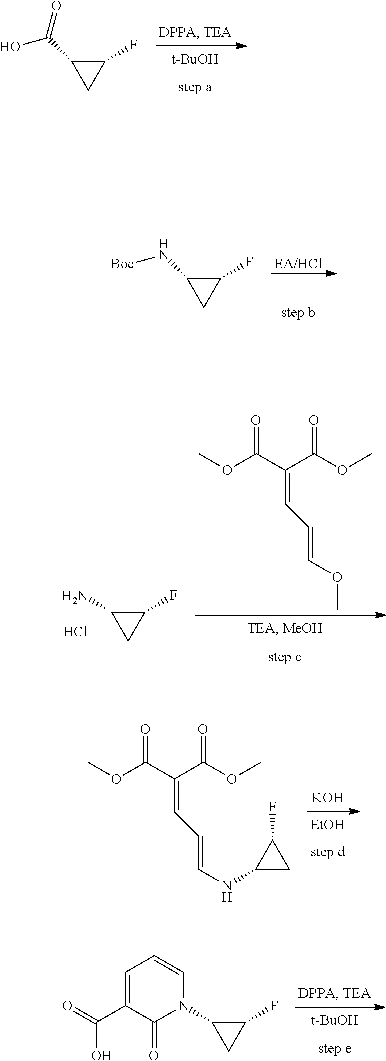

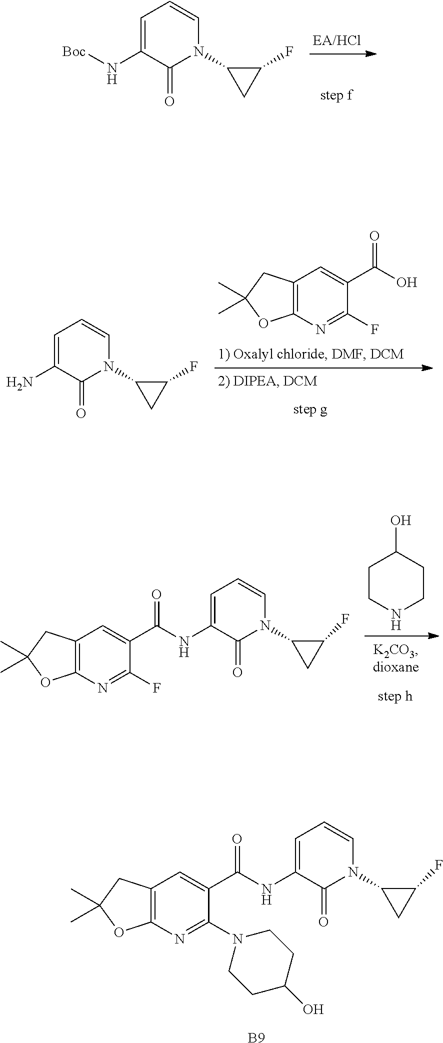

Step a. tert-Butyl ((1S,2R)-2-fluorocyclopropyl)carbamate: To a solution of (1R,2R)-2-fluorocyclopropane-1-carboxylic acid (2.8 g, 26.9 mmol) in t-BuOH (120 mL) was added TEA (8.1 g, 81 mmol) and DPPA (8.1 g, 30 mmol), and then stirred for 72 h at 90° C. under N₂. The mixture was diluted with saturated NaHCO₃ aqueous solution (50 mL) and extracted with EA (60 mL×2). The combined organic layers were dried over Na₂SO₄, filtrated and concentrated and the residue was purified by silica gel column (PE/EA=10/1) to give the desired product (3.4 g, 72%) as a green oil. ¹H NMR (300 MHz, CDCl₃) δ 4.83 (s, 1H), 4.58 (d, J=63.6 Hz, 1H), 2.62 (s, 1H), 1.45 (s, 9H), 1.10-0.82 (m, 2H).

Step b. (1S,2R)-2-Fluorocyclopropan-1-aminehydrochloride: To a solution of tert-butyl ((1S,2R)-2-fluorocyclopropyl)carbamate (2.1 g, 12 mmol) in EA (10 mL) was added 3 N HCl/EA solution (24 mL) and the mixture was stirred at room temperature overnight. The mixture was concentrated to get the desired product (1.3 g, 98%) as a white solid. ¹H NMR (300 MHz, DMSO-d₆) δ 8.69 (s, 2H), 4.88 (d, J=63.8 Hz, 1H), 2.69-2.54 (m, 1H), 1.27-1.01 (m, 2H).

Step c. Dimethyl 2-((E)-3-(((1S,2R)-2-fluorocyclopropyl)amino)allylidene)malonate: To a solution of (1S,2R)-2-fluorocyclopropan-1-aminehydrochloride (777 mg, 7.0 mmol) in dry methanol (12 mL) was added dimethyl (E)-2-(3-methoxyallylidene)malonate (2.1 g, 10 mmol) and TEA (2.1 g, 21 mmol), and then stirred for 3 h at room temperature under N₂ atmosphere. The mixture was concentrated in vacuum and the residue was purified by silica gel column (PE/EA=1/1) to give the desired product (1.1 g, 65%) as a yellow solid. ¹H NMR (300 MHz, CDCl₃) δ 7.64 (d, J=12.3 Hz, 1H), 7.10-6.99 (m, 1H), 6.39 (t, J=12.9 Hz, 1H), 5.09 (s, 1H), 4.72 (d, J=64.2 Hz, 1H), 3.80 (s, 3H), 3.75 (s, 3H), 2.60 (s, 1H), 1.27-1.16 (m, 1H), 1.07-0.91 (m, 1H).

Step d. 1-((1S,2R)-2-Fluorocyclopropyl)-2-oxo-1,2-dihydropyridine-3-carboxylic acid: To a solution of dimethyl 2-((E)-3-(((1S,2R)-2-fluorocyclopropyl)amino)allylidene)malonate (960 mg, 4 mmol) in EtOH (15 mL) was added KOH (360 mg, 6.4 mmol) at room temperature. After being stirred for 1 h at room temperature, the mixture was removed to reflux for 3 h at 90° C. The solvent was concentrated in vacuum and the residue was diluted with water (10 mL) and adjusted pH to 1 with 2N HCl aqueous solution. The resulting precipitate was filtered and dried to get the desired product (760 mg, 96%) as a yellow solid. ¹H NMR (300 MHz, CDCl₃) δ 14.07 (s, 1H), 8.53 (d, J=7.2 Hz, 1H), 7.70 (d, J=6.9 Hz, 1H), 6.57 (t, J=6.9 Hz, 1H), 4.97 (d, J=60.0 Hz, 1H), 3.47-3.32 (m, 1H), 1.71-1.53 (m, 2H).

Step e. tert-Butyl (1-((1S,2R)-2-fluorocyclopropyl)-2-oxo-1,2-dihydropyridin-3-yl)carbamate: To a solution of 1-((1S,2R)-2-fluorocyclopropyl)-2-oxo-1,2-dihydropyridine-3-carboxylic acid (760 mg, 3.9 mmol) in t-BuOH (25 mL) was added TEA (600 mg, 5.9 mmol) and DPPA (1.3 g, 4.6 mmol), and then stirred for 4 h at 90° C. under N₂. The mixture was diluted with saturated NaCl aqueous solution (15 mL) and extracted with EA (20 mL×2). The combined organic layers were dried over Na₂SO₄, filtrated and concentrated and the residue was purified by silica gel column (PE/EA=1/1) to give the desired product (480 mg, 48%) as a yellow solid. ¹H NMR (300 MHz, CDCl₃) δ 8.01-7.92 (m, 1H), 7.65 (s, 1H), 6.96 (d, J=6.3 Hz, 1H), 6.21 (t, J=7.2 Hz, 1H), 4.91 (d, J=64.5 Hz, 1H), 3.34-3.23 (m, 1H), 1.39-1.20 (m, 2H), 1.50 (s, 9H).

Step f. 3-Amino-1-((1S,2R)-2-fluorocyclopropyl)pyridin-2(1H)-one: To a solution of tert-butyl (1-((1S,2R)-2-fluorocyclopropyl)-2-oxo-1,2-dihydropyridin-3-yl)carbamate (450 mg, 1.6 mmol) in EA (2 mL) was added 3 N HCl/EA solution (12 mL) and the mixture was stirred for 3 h at room temperature. The mixture was concentrated in vacuum and the residue was adjusted pH to 8 with saturated NaHCO₃ aqueous solution and extracted with EA (20 mL×2). The combined organic layers were dried over Na₂SO₄, filtrated and concentrated to give the desired product (255 mg, 95%) as a yellowoil. ¹H NMR (300 MHz, CDCl₃) δ 6.77 (d, J=6.9 Hz, 1H), 6.52 (d, J=6.0 Hz, 1H), 6.06 (t, J=6.9 Hz, 1H), 4.89 (d, J=60.0 Hz, 1H), 4.21 (s, 2H), 3.34-3.21 (m, 1H), 1.50-1.35 (m, 2H).

Step g. 6-Fluoro-N-(1-((1S,2R)-2-fluorocyclopropyl)-2-oxo-1,2-dihydropyridin-3-yl)-2,2-dimethyl-2,3-dihydrofuro[2,3-b]pyridine-5-carboxamide: The procedure described in Example 60 was carried to get the desired product (100 mg, 56%) as a yellow solid. ¹H NMR (300 MHz, CDCl₃) δ 9.72 (s, 1H), 8.49 (s, 1H), 8.37-8.13 (m, 1H), 7.15-7.01 (m, 1H), 6.37-6.19 (m, 1H), 4.91 (d, J=60.4 Hz, 1H), 3.40-3.21 (m, 1H), 3.06 (s, 2H), 1.46-1.35 (m, 2H), 1.23 (s, 6H).

Step h. N-(1-((1S,2R)-2-Fluorocyclopropyl)-2-oxo-1,2-dihydropyridin-3-yl)-6-(4-hydroxypiperidin-1-yl)-2,2-dimethyl-2,3-dihydrofuro[2,3-b]pyridine-5-carboxamide: The procedure described in Example 60 was carried to get the desired product (69 mg, 58%) as a yellow solid.

### Example 69, Method BB, BE

### Preparation of: N-(1-Cyclopropyl-2-oxo-1,2-dihydropyridin-3-yl)-6-(4-hydroxypiperidin-1-yl)-2,2-dimethyl-2,3-dihydrofuro[2,3-b]pyridine-5-carboxamide: (B10)

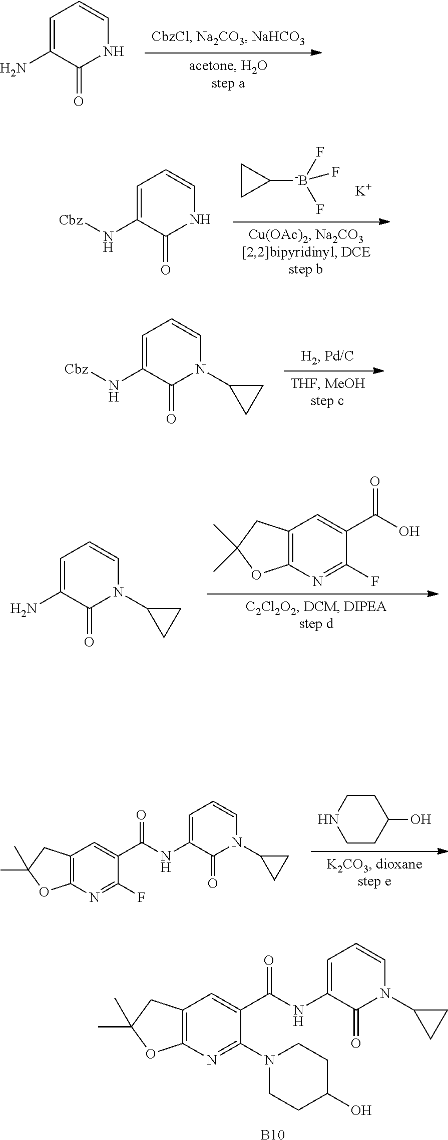

Step a. Benzyl (2-oxo-1,2-dihydropyridin-3-yl)carbamate: To a solution of 3-aminopyridin-2(1H)-one (1.2 g, 11 mmol) in water (150 mL) was added NaCO₃ (2.6 g, 24 mmol) and NaHCO₃ (1.0 g, 12 mmol), and then added acetone (20 mL) and Cbz-Cl (2.1 g, 12 mmol) at 0° C. The mixture was removed to room temperature and stirred for 4 h. The reaction was diluted with EA (100 mL) and washed with water (100 mL). The combined organic layers were dried over Na₂SO₄, filtrated and concentrated to give the desired product (2 g, 74%) as a white solid. ¹H NMR (300 MHz, DMSO-d₆) δ 11.92 (s, 1H), 8.31 (s, 1H), 7.83 (d, J=8.7 Hz, 1H), 7.47-7.25 (m, 5H), 7.07 (d, J=6.6 Hz, 1H), 6.23 (t, J=6.6 Hz, 1H), 5.15 (s, 2H). LC-MS (m/z): 244.9 [M+H]⁺.

Step b. Benzyl (1-cyclopropyl-2-oxo-1,2-dihydropyridin-3-yl)carbamate: A suspension of benzyl (2-oxo-1,2-dihydropyridin-3-yl)carbamate (1.4 g, 5.7 mmol), potassium cyclopropyltrifluoroborate (1.7 g, 11.5 mmol), copper acetate (1.2 g, 6.0 mmol), 2,2′-bipyridine (0.94 g, 6.0 mmol) and NaCO₃ (1.3 g, 13 mmol) in DCE (35 mL) was stirred for 15 h at 70° C. exposure to the air. The mixture was diluted with EA (100 mL) and washed with water (80 mL) and saturated brine solution (80 mL). The combined organic layers were dried over Na₂SO₄, filtrated and concentrated and the residue was purified by silica gel column (PE/EA=5/1) to give the desired product (327 mg, 20%) as a black solid. ¹H NMR (300 MHz, CDCl₃) δ 7.97 (s, 1H), 7.93-7.87 (m, 1H), 7.43-7.31 (m, 4H), 7.28-7.20 (m, 1H), 7.02-6.87 (m, 1H), 6.26-6.08 (m, 1H), 5.19 (s, 2H), 3.50-3.14 (m, 1H), 1.16-1.08 (m, 2H), 0.97-0.79 (m, 2H). LC-MS (m/z): 284.9 [M+H]⁺.

Step c. 3-Amino-1-cyclopropylpyridin-2(1H)-one: A suspension of benzyl (1-cyclopropyl-2-oxo-1,2-dihydropyridin-3-yl)carbamate (320 mg, 1.1 mmol) and 5% Pd/C (90 mg, 0.040 mmol) in MeOH (50 mL) and THF (15 mL) was stirred at room temperature under H₂ atmosphere overnight. The suspension was filtered and the filtrate concentrated to give the desired product (150 mg, 88%) as a grey oil. ¹H NMR (300 MHz, CDCl₃) δ 6.73 (d, J=6.3 Hz, 1H), 6.50 (d, J=7.5 Hz, 1H), 6.02 (t, J=6.3 Hz, 1H), 4.20 (s, 2H), 2.73-2.52 (m, 1H), 1.21-1.02 (m, 2H), 0.89-0.80 (m, 2H). LC-MS (m/z): 151.0 [M+H]⁺.

Step d. N-(1-Cyclopropyl-2-oxo-1,2-dihydropyridin-3-yl)-6-fluoro-2,2-dimethyl-2,3-dihydrofuro[2,3-b]pyridine-5-carboxamide: The procedure described in Example 60 was carried to get the desired product (60 mg, 39%) as a grey solid. 1H NMR (300 MHz, CDCl₃) δ 9.77 (s, 1H), 8.44 (s, 1H), 8.26 (d, J=7.5 Hz, 1H), 7.04 (d, J=10.2 Hz, 1H), 6.39-6.12 (m, 1H), 3.53-3.24 (m, 1H), 3.05 (s, 2H), 1.54 (s, 6H), 1.21-1.01 (m, 2H), 0.97-0.83 (m, 2H). LC-MS (m/z): 344.1 [M+H]⁺.

Step e. N-(1-Cyclopropyl-2-oxo-1,2-dihydropyridin-3-yl)-6-(4-hydroxypiperidin-1-yl)-2,2-dimethyl-2,3-dihydrofuro[2,3-b]pyridine-5-carboxamide: The procedure described in Example 60 was carried to get the desired product (16 mg, 19%) as a white solid.

### Example 70, Method BC

### Preparation of: 6-Methoxy-2,2-dimethyl-N-(6-(1-methyl-1H-pyrazol-4-yl)pyridin-2-yl)-2,3-dihydrofuro[2,3-b]pyridine-5-carboxamide: (B11)

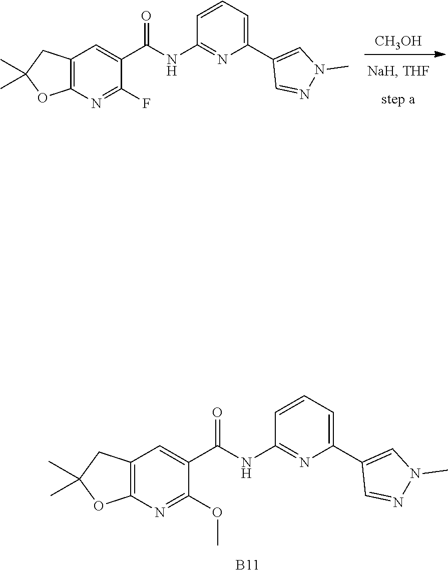

Step a. 6-Methoxy-2,2-dimethyl-N-(6-(1-methyl-1H-pyrazol-4-yl)pyridin-2-yl)-2,3-dihydrofuro[2,3-b]pyridine-5-carboxamide: To a solution of dry MeOH (87 mg, 2.7 mmol) in dry THF (5 mL) was added 60% NaH (108 mg, 2.7 mmol) slowly. After being stirred for 1 h at room temperature, the mixture was added 6-fluoro-2,2-dimethyl-N-(6-(1-methyl-1H-pyrazol-4-yl)pyridin-2-yl)-2,3-dihydrofuro[2,3-b]pyridine-5-carboxamide (50 mg, 0.14 mmol) and refluxed overnight. The reaction was diluted with water (10 mL) and extracted with EA (10 mL×3) and washed with saturated brine solution (20 mL). The combined organic layers were dried over Na₂SO₄, filtrated and concentrated and the residue was purified by silica gel column (DCM/MeOH=50/1) to give the desired product (20 mg, 39%) as a white solid.

### Example 71, Method BC

### Preparation of: 6-Ethoxy-2,2-dimethyl-N-(6-(1-methyl-1H-pyrazol-4-yl)pyridin-2-yl)-2,3-dihydrofuro[2,3-b]pyridine-5-carboxamide: (B12)

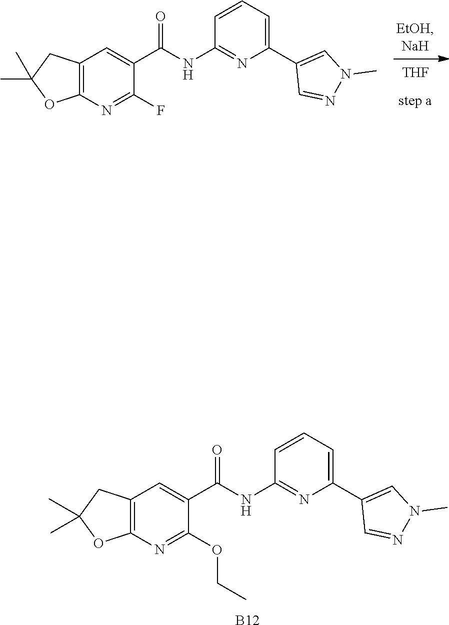

Step a. 6-Ethoxy-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (26 mg, 48%) as a white solid.

### Example 72, Method BC

### Preparation of: 6-Isopropoxy-2,2-dimethyl-N-(6-(1-methyl-1H-pyrazol-4-yl)pyridin-2-yl)-2,3-dihydrofuro[2,3-b]pyridine-5-carboxamide: (B13)

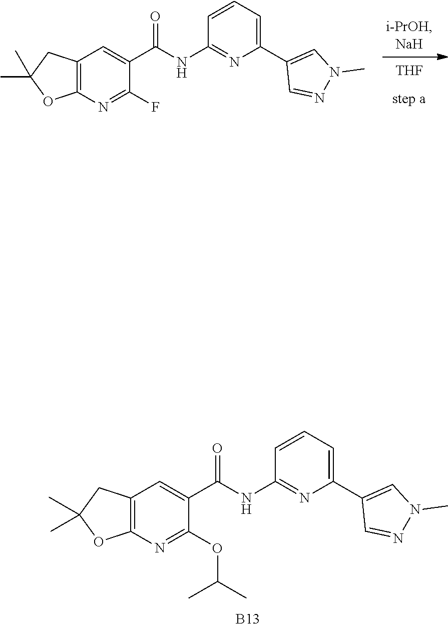

Step a. 6-Isopropoxy-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (26 mg, 47%) as a white solid.

### Example 73, Method BC

### Preparation of: 6-Cyclobutoxy-2,2-dimethyl-N-(6-(1-methyl-1H-pyrazol-4-yl)pyridin-2-yl)-2,3-dihydrofuro[2,3-b]pyridine-5-carboxamide: (B14)

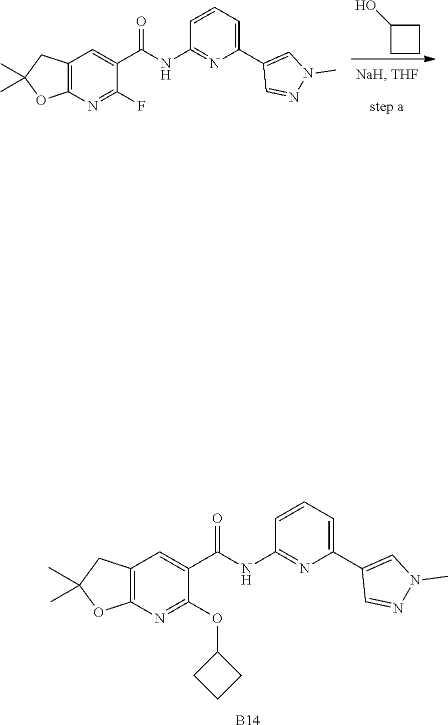

Step a. 6-Cyclobutoxy-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (28 mg, 52%) as a white solid.

### Example 74, Method BC

### Preparation of: 6-(Cyclopentyloxy)-2,2-dimethyl-N-(6-(1-methyl-1H-pyrazol-4-yl)pyridin-2-yl)-2,3-dihydrofuro[2,3-b]pyridine-5-carboxamide: (B15)

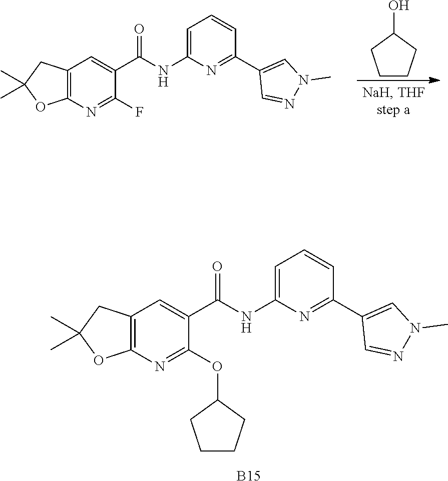

Step a. 6-(Cyclopentyloxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (16 mg, 27%) as a white solid.

### Example 75, Method BC

### Preparation of: 2,2-Dimethyl-N-(6-(1-methyl-1H-pyrazol-4-yl)pyridin-2-yl)-6-(oxetan-3-yloxy)-2,3-dihydrofuro[2,3-b]pyridine-5-carboxamide: (B16)

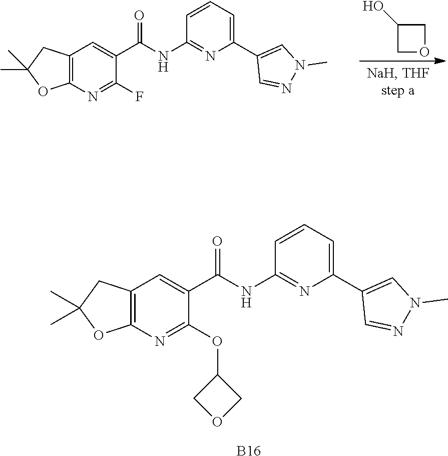

Step a. 2,2-Dimethyl-N-(6-(1-methyl-1H-pyrazol-4-yl)pyridin-2-yl)-6-(oxetan-3-yloxy)-2,3-dihydrofuro[2,3-b]pyridine-5-carboxamide: The procedure described in Example 70 was carried to get the desired product (20 mg, 35%) as a white solid.

### Example 76, Method BC

### Preparation of: (S)-2,2-Dimethyl-N-(6-(1-methyl-1H-pyrazol-4-yl)pyridin-2-yl)-6-((tetrahydrofuran-3-yl)oxy)-2,3-dihydrofuro[2,3-b]pyridine-5-carboxamide: (B17)

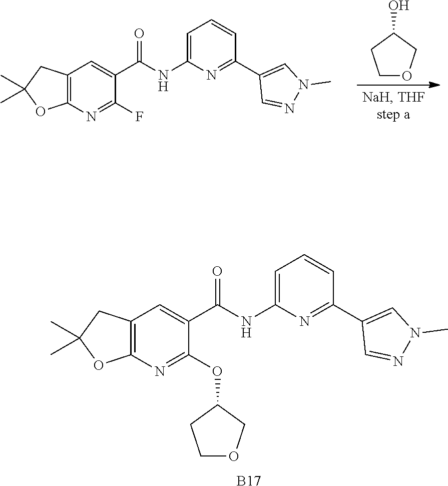

Step a. (S)-2,2-Dimethyl-N-(6-(1-methyl-1H-pyrazol-4-yl)pyridin-2-yl)-6-((tetrahydrofuran-3-yl)oxy)-2,3-dihydrofuro[2,3-b]pyridine-5-carboxamide: The procedure described in Example 70 was carried to get the desired product (12 mg, 20%) as a white solid.

### Example 77, Method BC

### Preparation of: 6-(2-Hydroxyethoxy)-2,2-dimethyl-N-(6-(1-methyl-1H-pyrazol-4-yl)pyridin-2-yl)-2,3-dihydrofuro[2,3-b]pyridine-5-carboxamide: (B18)

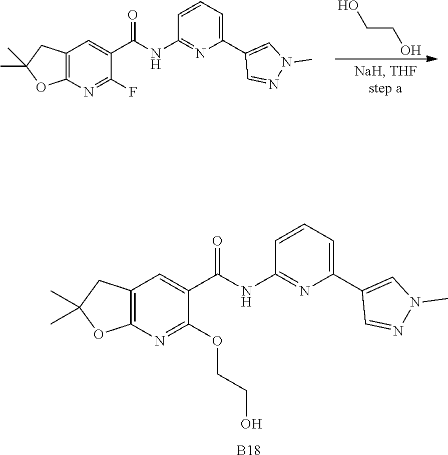

Step a. 6-(2-Hydroxyethoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (8 mg, 14%) as a white solid.

### Example 78, Method BC

### Preparation of: 6-(2-Methoxyethoxy)-2,2-dimethyl-N-(6-(1-methyl-1H-pyrazol-4-yl)pyridin-2-yl)-2,3-dihydrofuro[2,3-b]pyridine-5-carboxamide: (B19)

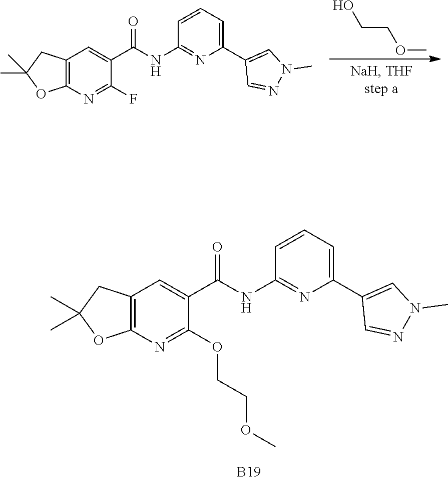

Step a. 6-(2-Methoxyethoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (23 mg, 40%) as a white solid.

### Example 79, Method BF

### Preparation of: 6-(Difluoromethoxy)-2,2-dimethyl-N-(6-(1-methyl-1H-pyrazol-4-yl)pyridin-2-yl)-2,3-dihydrofuro[2,3-b]pyridine-5-carboxamide: (B20)

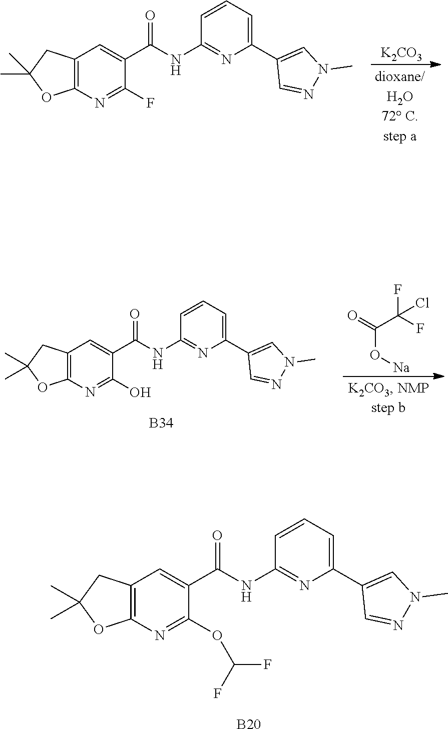

Step a. 6-Hydroxy-2,2-dimethyl-N-(6-(1-methyl-1H-pyrazol-4-yl)pyridin-2-yl)-2,3-dihydrofuro[2,3-b]pyridine-5-carboxamide: To a solution of 6-fluoro-2,2-dimethyl-N-(6-(1-methyl-1H-pyrazol-4-yl)pyridin-2-yl)-2,3-dihydrofuro[2,3-b]pyridine-5-carboxamide (350 mg, 0.95 mmol) in the mixed solution (1,4-dioxane 45 mL/water 45 mL) was added K₂CO₃ (1.3 g, 9.4 mmol), the mixture was stirred for 4 h at 72° C. After the reaction was complete, the mixture was concentrated in vacuum and the residue was purified by silica gel column. (DCM/MeOH=50/1) to give the desired product (170 mg, 49%) as a yellow solid.

Step b. 6-(Difluoromethoxy)-2,2-dimethyl-N-(6-(1-methyl-1H-pyrazol-4-yl)pyridin-2-yl)-2,3-dihydrofuro[2,3-b]pyridine-5-carboxamide: To a solution of 6-hydroxy-2,2-dimethyl-N-(6-(1-methyl-1H-pyrazol-4-yl)pyridin-2-yl)-2,3-dihydrofuro[2,3-b]pyridine-5-carboxamide (60 mg, 0.16 mmol) in NMP (9 mL) was added sodium 2-chloro-2,2-difluoroacetate (63 mg, 0.41 mmol) and K₂CO₃ (57 mg, 0.41 mmol). After being stirred for 15 h at 70° C., the mixture was quenched with water (15 mL) and extracted with EA (20 mL). The combined organic layers were dried over Na₂SO₄, filtrated and concentrated and the residue was purified by silica gel column (DCM/MeOH=50/1) to give the desired product (10 mg, 15%) as a white solid.

### Example 80, Method BC

### Preparation of: 2,2-Dimethyl-N-(6-(1-methyl-1H-pyrazol-4-yl)pyridin-2-yl)-6-((1-methylpyrrolidin-3-yl)oxy)-2,3-dihydrofuro[2,3-b]pyridine-5-carboxamide: (B21)

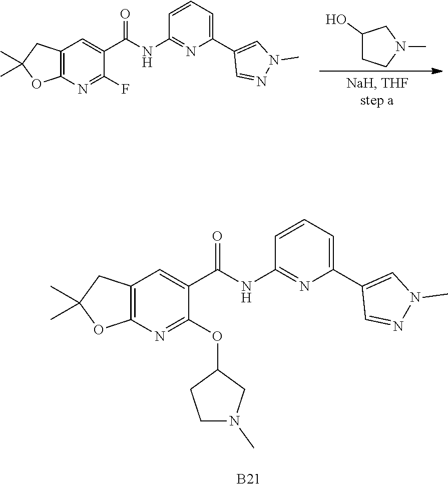

Step a. 2,2-Dimethyl-N-(6-(1-methyl-1H-pyrazol-4-yl)pyridin-2-yl)-6-((1-methylpyrrolidin-3-yl)oxy)-2,3-dihydrofuro[2,3-b]pyridine-5-carboxamide: The procedure described in Example 70 was carried to get the desired product (17 mg, 29%) as a white solid.

### Example 81, Method BC, BG

### Preparation of: 2,2-Dimethyl-N-(6-(1-methyl-1H-pyrazol-4-yl)pyridin-2-yl)-6-(pyrrolidin-3-yloxy)-2,3-dihydrofuro[2,3-b]pyridine-5-carboxamide: (B22)

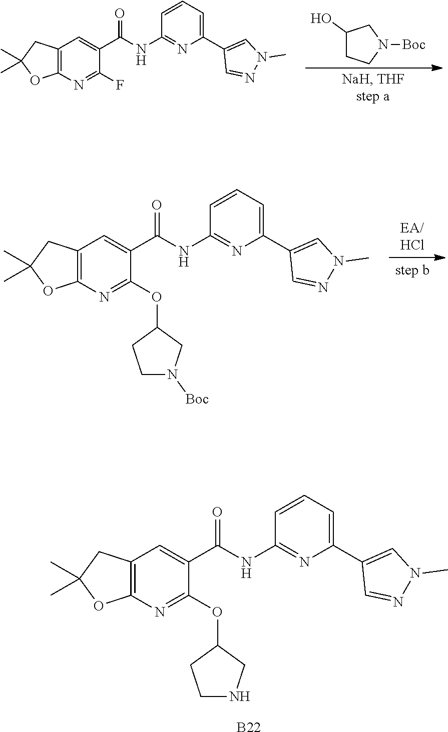

Step a. tert-Butyl 3-((2,2-dimethyl-5-((6-(1-methyl-1H-pyrazol-4-yl)pyridin-2-yl)carbamoyl)-2,3-dihydrofuro[2,3-b]pyridin-6-yl)oxy)pyrrolidine-1-carboxylate: The procedure described in Example 70 was carried to get the desired product (140 mg, 97%) as a white solid.

Step b. 2,2-Dimethyl-N-(6-(1-methyl-1H-pyrazol-4-yl)pyridin-2-yl)-6-(pyrrolidin-3-yloxy)-2,3-dihydrofuro[2,3-b]pyridine-5-carboxamide: To a solution of tert-butyl 3-((2,2-dimethyl-5-((6-(1-methyl-1H-pyrazol-4-yl)pyridin-2-yl)carbamoyl)-2,3-dihydrofuro[2,3-b]pyridin-6-yl)oxy)pyrrolidine-1-carboxylate (140 mg, 0.27 mmol) in EA (2 mL) was added 3 N HCl/EA (0.4 mL) and the mixture was stirred at room temperature overnight. The mixture was concentrated in vacuum and adjusted pH to 8 with saturated NaHCO₃ aqueous solution (5 mL), and then extracted with EA (6 mL×3). The combined organic layers were dried over Na₂SO₄, filtrated and concentrated and the residue was purified by silica gel column (DCM/MeOH=20/1) to give the desired product (37 mg, 32%) as a white solid.

### Example 82, Method BC

### Preparation of: 2,2-Dimethyl-N-(6-(1-methyl-1H-pyrazol-4-yl)pyridin-2-yl)-6-((tetrahydro-2H-pyran-4-yl)oxy)-2,3-dihydrofuro[2,3-b]pyridine-5-carboxamide: (B23)

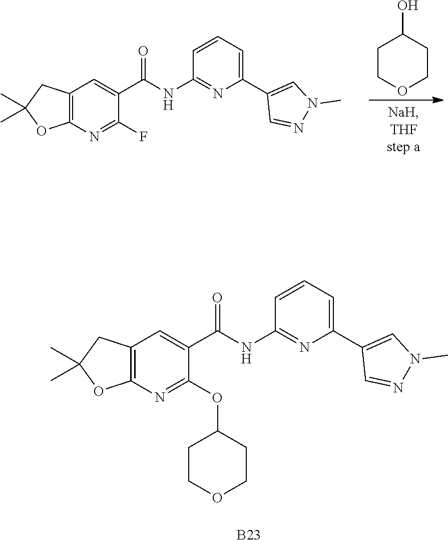

Step a. 2,2-Dimethyl-N-(6-(1-methyl-1H-pyrazol-4-yl)pyridin-2-yl)-6-((tetrahydro-2H-pyran-4-yl)oxy)-2,3-dihydrofuro[2,3-b]pyridine-5-carboxamide: The procedure described in Example 70 was carried to get the desired product (24 mg, 39%) as a white solid.

### Example 83, Method BC

### Preparation of: 6-(Cyclopropylmethoxy)-2,2-dimethyl-N-(6-(1-methyl-1H-pyrazol-4-yl)pyridin-2-yl)-2,3-dihydrofuro[2,3-b]pyridine-5-carboxamide: (B24)

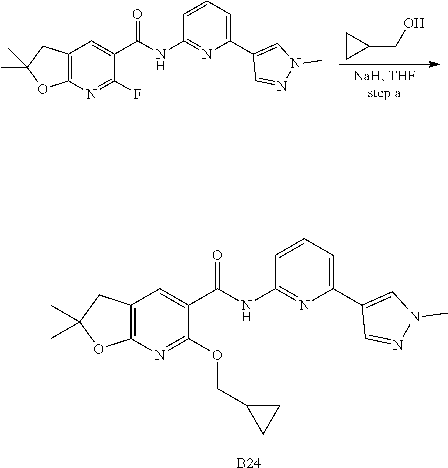

Step a. 6-(Cyclopropylmethoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (30 mg, 52%) as a white solid.

### Example 84, Method BC, BG

### Preparation of: 2,2-Dimethyl-N-(6-(1-methyl-1H-pyrazol-4-yl)pyridin-2-yl)-6-(2-(methylamino)ethoxy)-2,3-dihydrofuro[2,3-b]pyridine-5-carboxamide: (B25)

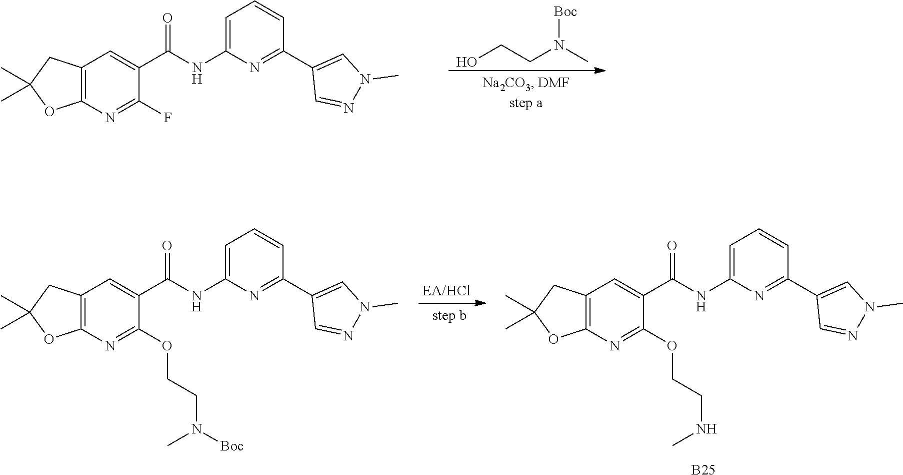

Step a. tert-Butyl (2-((2,2-dimethyl-5-((6-(1-methyl-1H-pyrazol-4-yl)pyridin-2-yl)carbamoyl)-2,3-dihydrofuro[2,3-b]pyridin-6-yl)oxy)ethyl)(methyl)carbamate: The procedure described in Example 70 was carried to get the desired product (35 mg, 49%) as a white solid. 1H NMR (300 MHz, DMSO-d₆) δ 10.21 (s, 1H), 8.26 (s, 1H), 8.21 (s, 1H), 8.02-7.93 (m, 2H), 7.77 (t, J=8.4 Hz, 1H), 7.38 (d, J=8.4 Hz, 1H), 4.67 (t, J=4.8 Hz, 2H), 3.89 (s, 3H), 3.68 (t, J=4.8 Hz, 2H), 3.06 (s, 2H), 1.99 (s, 3H), 1.48 (s, 6H), 1.17 (s, 9H). LC-MS (m/z): 522.9 [M+H]⁺.

Step b. 2,2-Dimethyl-N-(6-(1-methyl-1H-pyrazol-4-yl)pyridin-2-yl)-6-(2-(methylamino)ethoxy)-2,3-dihydrofuro[2,3-b]pyridine-5-carboxamide: The procedure described in Example 81 was carried to get the desired product (2 mg, 7%) as a white solid.

### Example 85, Method BC

### Preparation of: 6-(1-Cyclopropylethoxy)-2,2-dimethyl-N-(6-(1-methyl-1H-pyrazol-4-yl)pyridin-2-yl)-2,3-dihydrofuro[2,3-b]pyridine-5-carboxamide: (B26)

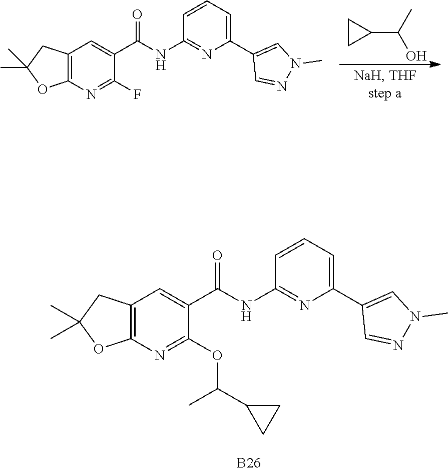

Step a.6-(1-cyclopropylethoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (7 mg, 12%) as a white solid.

### Example 86, Method BC, BH

### Preparation of: 6-(Cis-3-hydroxycyclobutoxy)-2,2-dimethyl-N-(6-(1-methyl-1H-pyrazol-4-yl)pyridin-2-yl)-2,3-dihydrofuro[2,3-b]pyridine-5-carboxamide: (B27)

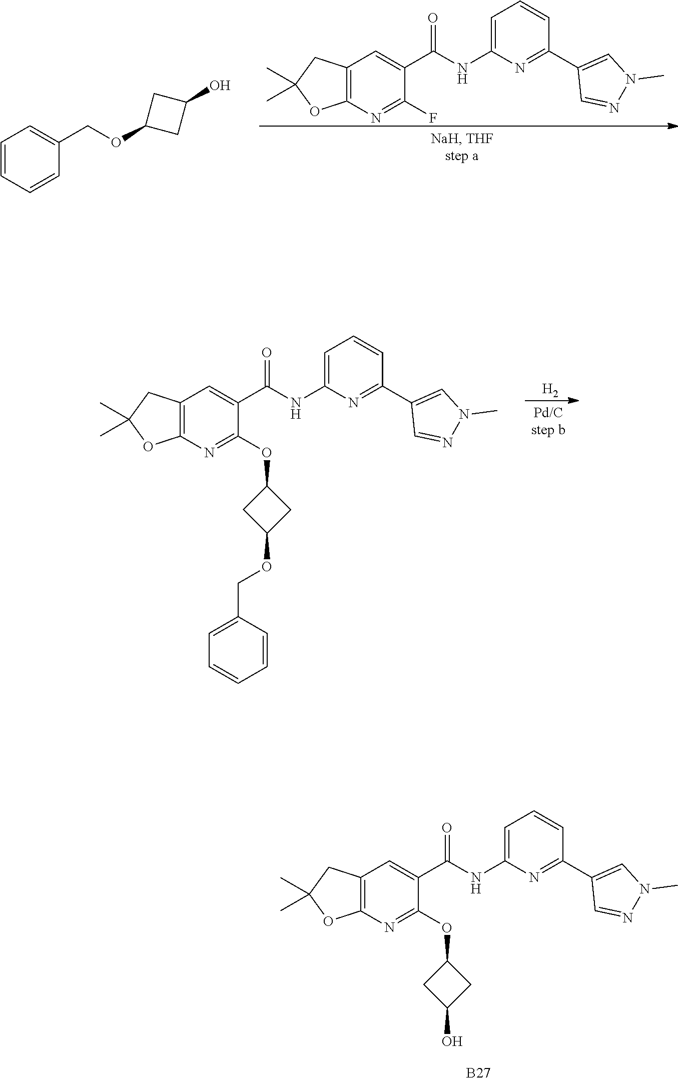

Step a. 6-(cis-3-(Benzyloxy)cyclobutoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the crude product (180 mg) as a colorless oil. ¹H NMR (300 MHz, DMSO-d₆) δ 10.52 (s, 1H), 8.29-8.16 (m, 2H), 8.06-7.93 (m, 2H), 7.80 (t, J=8.1 Hz, 1H), 7.46-7.21 (m, 6H), 5.07-4.99 (m, 1H), 4.47 (s, 2H), 3.93-3.87 (m, 1H), 3.82 (s, 3H), 3.05 (s, 2H), 3.01-2.90 (m, 2H), 2.34-2.19 (m, 2H), 1.47 (s, 6H). LC-MS (m/z): 525.8 [M+H]⁺.

Step b. 6-(cis-3-Hydroxycyclobutoxy)-2,2-dimethyl-N-(6-(1-methyl-1H-pyrazol-4-yl)pyridin-2-yl)-2,3-dihydrofuro[2,3-b]pyridine-5-carboxamide: A suspension of 6-(cis-3-(benzyloxy)cyclobutoxy)-2,2-dimethyl-N-(6-(1-methyl-1H-pyrazol-4-yl)pyridin-2-yl)-2,3-dihydrofuro[2,3-b]pyridine-5-carboxamide (180 mg, 0.34 mmol) and 5% Pd/C (30 mg) in MeOH (50 mL) was stirred at room temperature under H₂ atmosphere overnight. The suspension was filtered and concentrated to give the desired product (30 mg, 25%) as a white solid.

### Example 87, Method BC, BG

### Preparation of: 2,2-Dimethyl-N-(6-(1-methyl-1H-pyrazol-4-yl)pyridin-2-yl)-6-((2-oxo-1,3-oxazinan-5-yl)oxy)-2,3-dihydrofuro[2,3-b]pyridine-5-carboxamide: (B28)

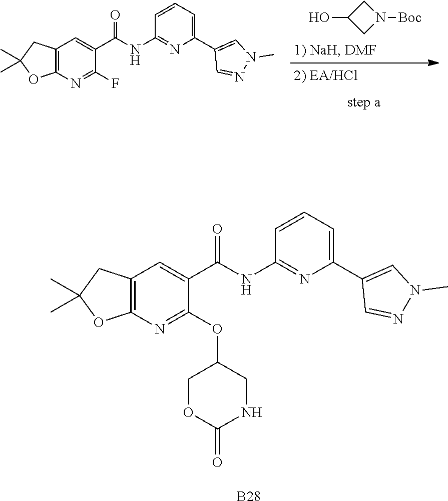

Step a. 2,2-Dimethyl-N-(6-(1-methyl-1H-pyrazol-4-yl)pyridin-2-yl)-6-((2-oxo-1,3-oxazinan-5-yl)oxy)-2,3-dihydrofuro[2,3-b]pyridine-5-carboxamide: The procedure described in Example 81 was carried to get the by-product (20 mg, 16%) as a white solid.

### Example 88, Method BC

### Preparation of: 6-Cyclopropoxy-2,2-dimethyl-N-(6-(1-methyl-1H-pyrazol-4-yl)pyridin-2-yl)-2,3-dihydrofuro[2,3-b]pyridine-5-carboxamide: (B29)

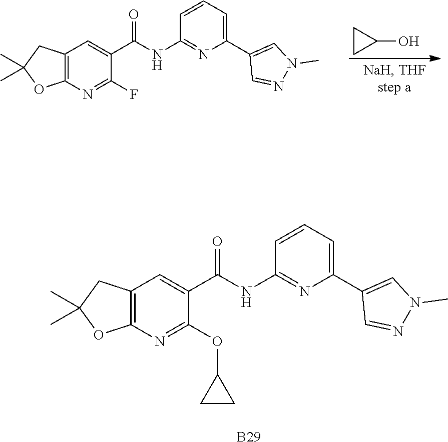

Step a. 6-Cyclopropoxy-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (27 mg, 39%) as a white solid.

### Example 89, Method BC, BH

### Preparation of: 6-(Azetidin-3-yloxy)-2,2-dimethyl-N-(6-(1-methyl-1H-pyrazol-4-yl)pyridin-2-yl)-2,3-dihydrofuro[2,3-b]pyridine-5-carboxamide: (B30)

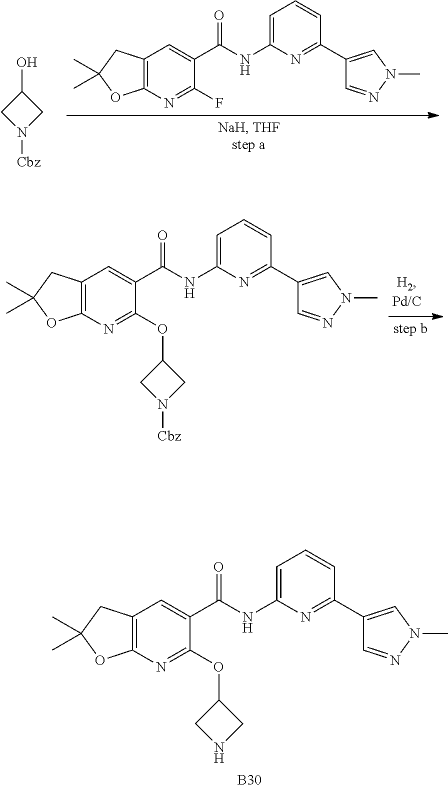

Step a. Benzyl 3-((2,2-dimethyl-5-((6-(1-methyl-1H-pyrazol-4-yl)pyridin-2-yl)carbamoyl)-2,3-dihydrofuro[2,3-b]pyridin-6-yl)oxy)azetidine-1-carboxylate: The procedure described in Example 70 was carried to get the desired product (560 mg, 31%) as a colorless oil. ¹H NMR (300 MHz, CDCl₃) δ 7.37-7.31 (m, 5H), 5.08 (s, 2H), 4.66-4.55 (m, 1H), 4.27-4.18 (m, 2H), 3.94-3.83 (m, 2H), 2.78 (d, J=4.5 Hz, 1H).

Step b. 6-(Azetidin-3-yloxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 86 was carried to get the desired product (75 mg, 71%) as a white solid.

### Example 90, Method BI

### Preparation of: 2,2-Dimethyl-N-(6-(1-methyl-1H-pyrazol-4-yl)pyridin-2-yl)-6-((1-methylazetidin-3-yl)oxy)-2,3-dihydrofuro[2,3-b]pyridine-5-carboxamide: (B31)

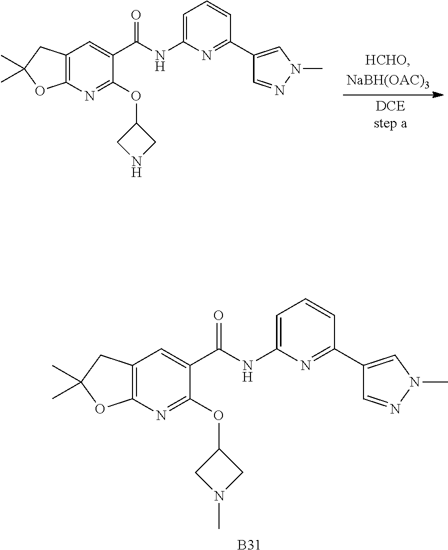

Step a. 2,2-Dimethyl-N-(6-(1-methyl-1H-pyrazol-4-yl)pyridin-2-yl)-6-((1-methylazetidin-3-yl)oxy)-2,3-dihydrofuro[2,3-b]pyridine-5-carboxamide: To a solution of 6-(azetidin-3-yloxy)-2,2-dimethyl-N-(6-(1-methyl-1H-pyrazol-4-yl)pyridin-2-yl)-2,3-dihydrofuro[2,3-b]pyridine-5-carboxamide (55 mg, 0.13 mmol) and POM (8.0 mg, 0.26 mmol) in DCE (10 mL) was added NaBH(OAc)₃ (41 mg, 0.20 mmol) at 0° C. The reaction was stirred at room temperature overnight, and then quenched with saturated NH₄Cl aqueous solution (30 mL). The mixture was extracted with dichloromethane (30 mL×3). The combined organic layers were dried over Na₂SO₄, filtered and concentrated. The residue was purified by silica gel column (DCM/MeOH=20/1) to give the desired product (8 mg, 14%) as a yellow solid.

### Example 91, Method BC

### Preparation of: 6-(2-(Dimethylamino)ethoxy)-2,2-dimethyl-N-(6-(1-methyl-1H-pyrazol-4-yl)pyridin-2-yl)-2,3-dihydrofuro[2,3-b]pyridine-5-carboxamide: (B32)

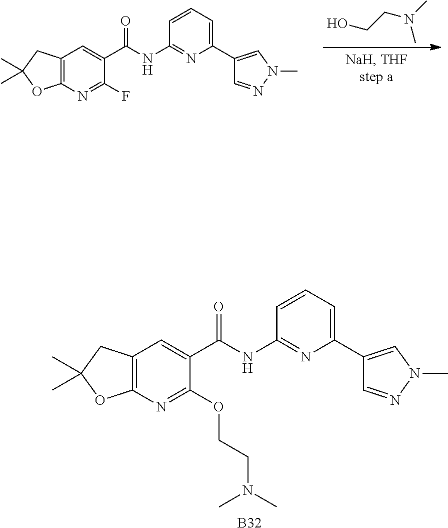

Step a. Benzyl 6-(2-(dimethylamino)ethoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (16 mg, 27%) as a white solid.

### Example 92, Method BC

### Preparation of: (R)-2,2-Dimethyl-N-(6-(1-methyl-1H-pyrazol-4-yl)pyridin-2-yl)-6-((tetrahydrofuran-3-yl)oxy)-2,3-dihydrofuro[2,3-b]pyridine-5-carboxamide: (B33)

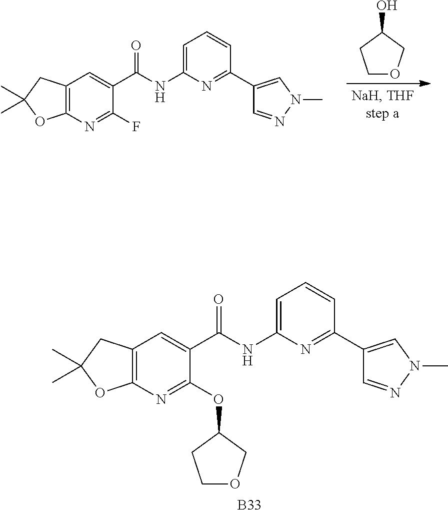

Step a. Benzyl (R)-2,2-dimethyl-N-(6-(1-methyl-1H-pyrazol-4-yl)pyridin-2-yl)-6-((tetrahydrofuran-3-yl)oxy)-2,3-dihydrofuro[2,3-b]pyridine-5-carboxamide: The procedure described in Example 70 was carried to get the desired product (29 mg, 49%) as a white solid.

### Example 93, Method BC, BJ

### Preparation of: 6-(3-Hydroxy-3-methylcyclobutoxy)-2,2-dimethyl-N-(6-(1-methyl-1H-pyrazol-4-yl)pyridin-2-yl)-2,3-dihydrofuro[2,3-b]pyridine-5-carboxamide: (B35)

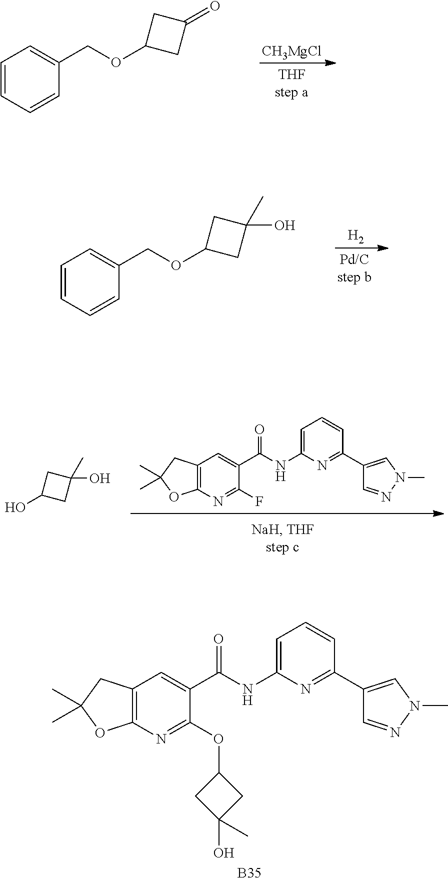

Step a. 3-(Benzyloxy)-1-methylcyclobutan-1-ol: To a solution of 3-(benzyloxy)-1-methylcyclobutan-1-ol (500 mg, 2.8 mmol) in dry THF (10 mL) was added 3 N methylmagnesium chloride (2.8 mL, 8.5 mmol) at −78° C. under N₂ atmosphere. After being stirred for 2 h at room temperature, the mixture was quenched by adding MeOH (5 mL) and concentrated in vacuum. The residue was purified by silica gel column (DCM/MeOH=20/1) to give the desired product (400 mg, 73%) as a yellow oil. ¹H NMR (300 MHz, DMSO-d₆) δ 7.31 (s, 5H), 5.00 (s, 1H), 4.33 (s, 2H), 3.69-3.61 (m, 1H), 2.30-2.20 (m, 2H), 1.98-1.88 (m, 2H), 1.14 (s, 3H). LC-MS (m/z): 214.9 [M+Na]⁺.

Step b.1-Methylcyclobutane-1,3-diol: A suspension of 3-(benzyloxy)-1-methylcyclobutan-1-ol (400 mg, 2.1 mmol) and 5% Pd/C (40 mg) in MeOH (20 mL) was stirred at room temperature under H₂ atmosphere overnight. The suspension was filtered and concentrated to give the desired product (110 mg, 52%) as a colorlessoil. ¹H NMR (300 MHz, DMSO-d₆) δ 4.89 (d, J=5.4 Hz, 1H), 4.83 (s, 1H), 3.74-3.64 (m, 1H), 2.23-2.13 (m, 2H), 1.90-1.79 (m, 2H), 1.12 (s, 3H).

Step c. 6-(3-Hydroxy-3-methylcyclobutoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (84 mg, 46%) as a white solid.

### Example 94, Method BC, BH, BK

### Preparation of: 6-(trans-3-Hydroxycyclobutoxy)-2,2-dimethyl-N-(6-(1-methyl-1H-pyrazol-4-yl)pyridin-2-yl)-2,3-dihydrofuro[2,3-b]pyridine-5-carboxamide: (B36)

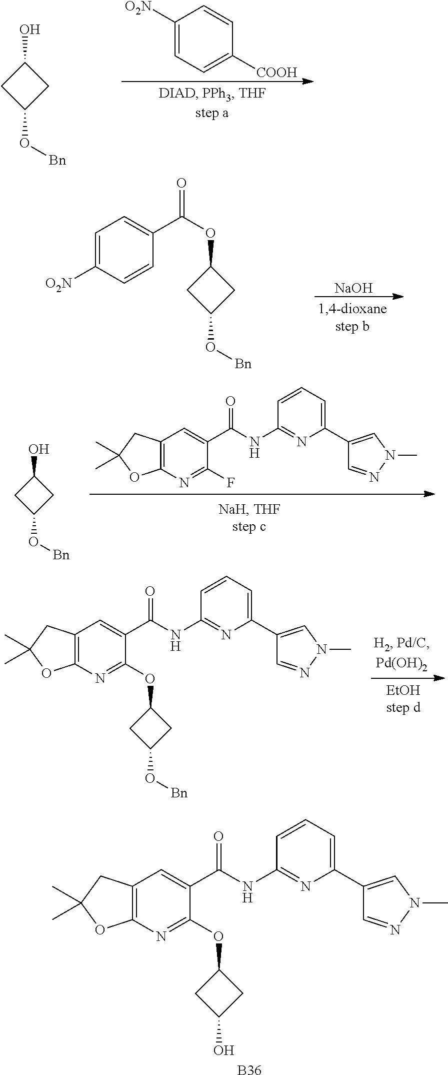

Step a. trans-3-(Benzyloxy)cyclobutyl 4-nitrobenzoate: To a solution of cis-3-(benzyloxy)cyclobutan-1-ol (100 mg, 0.56 mmol), 4-nitrobenzoic acid (187 mg, 1.1 mmol) and PPh₃ (440 mg, 1.7 mmol) in dry THF (10 mL) was added diisopropyl azodicarboxylate (DIAD) (339 mg, 1.7 mmol) slowly at 0° C. After being stirred at room temperature overnight, the solvent was concentrated in vacuum and the residue was purified by silica gel column (PE/EA=10/1) to give the desired product (168 mg, 91%) as a yellow solid. ¹H NMR (300 MHz, CDCl₃) δ 8.29 (d, J=8.8 Hz, 2H), 8.20 (d, J=8.3 Hz, 2H), 7.43-7.29 (m, 5H), 5.57-5.35 (m, 1H), 4.46 (s, 2H), 4.40-4.29 (m, 1H), 2.68-2.56 (m, 2H), 2.54-2.44 (m, 2H).

Step b. trans-3-(Benzyloxy)cyclobutan-1-ol: To a solution of trans-3-(benzyloxy)cyclobutyl 4-nitrobenzoate (168 mg, 0.51 mmol) in 1,4-dioxane (5 mL) was added 0.4 N NaOH aqueous solution (40 mg, 1.0 mmol). After being stirred for 1 h at room temperature, the mixture was acidified by AcOH (0.3 mL) and concentrated in vacuum. The residue was diluted with EA (20 mL) and washed with saturated NaHCO₃ aqueous solution (15 mL×3). The combined organic layers were dried over Na₂SO₄, filtered and concentrated to give the desired product (80 mg, 89%) as a yellow oil. ¹H NMR (300 MHz, CDCl₃) δ 7.40-7.28 (m, 5H), 4.64-4.50 (m, 1H), 4.41 (s, 2H), 4.35-4.23 (m, 1H), 2.50-2.30 (m, 2H), 2.24-2.11 (m, 2H), 1.75 (s, 1H).

Step c. 6-(trans-3-(Benzyloxy)cyclobutoxy)-2,2-dimethyl-N-(6-(1-methyl-1H-pyrazol-4-yl)pyridin-2-yl)-2,3-dihydrofuro[2,3-b]pyridine-5-carboxamide: The procedure described in Example 70 was carried to get the desired product (100 mg, 44%) as a white solid. ¹H NMR (300 MHz, CDCl₃) δ 10.55 (s, 1H), 8.35 (s, 1H), 8.18 (d, J=8.0 Hz, 1H), 7.97 (s, 1H), 7.82 (s, 1H), 7.70 (t, J=8.1 Hz, 1H), 7.38-7.31 (m, 5H), 7.21 (d, J=7.5 Hz, 1H), 5.70-5.57 (m, 1H), 4.61-4.52 (m, 1H), 4.47 (s, 2H), 3.93 (s, 3H), 3.05 (s, 2H), 2.82-2.61 (m, 4H), 1.59 (s, 6H).

Step d. 6-(trans-3-Hydroxycyclobutoxy)-2,2-dimethyl-N-(6-(1-methyl-1H-pyrazol-4-yl)pyridin-2-yl)-2,3-dihydrofuro[2,3-b]pyridine-5-carboxamide: To a suspension of 6-(trans-3-(benzyloxy)cyclobutoxy)-2,2-dimethyl-N-(6-(1-methyl-1H-pyrazol-4-yl)pyridin-2-yl)-2,3-dihydrofuro[2,3-b]pyridine-5-carboxamide (100 mg, 0.19 mmol), 5% Pd/C (20 mg) and Pd(OH)₂ (20 mg) in EtOH (20 mL) was added a drop of conc. HCl and the mixture was stirred at room temperature under H₂ atmosphere overnight. The suspension was filtered and concentrated to give the desired product (40 mg, 48%) as a colorless oil.

Table 1 shows a selection of the compounds prepared according to the methods discussed above in details and indicated in the third column of the table.

TABLE 1
Selected compounds (A1-A59 and B1-B36) of the present disclosure.

| NO. | Structure | method | ¹HNMR & LC-MS |
|---|---|---|---|
| A1 | 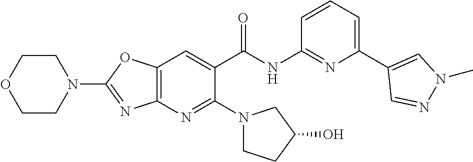 [SMILES: Cn1cc(-c2cccc(NC(=O)c3cc4oc(N5CCOCC5)nc4nc3N3CC[C@@H](O)C3)n2)cn1] | AA, AB, AC | ¹H NMR (300 MHZ, DMSO-d₆) δ 10.67 (s, 1H), 8.20 (s, 1H), 8.00-7.88 (m, 2H), 7.82-7.71 (m, 2H), 7.40-7.30 (m, 1H), 4.93-4.83 (m, 1H), 4.36-4.24 (m, 1H), 3.88 (s, 3H), 3.79-3.68 (m, 4H), 3.68-3.59 (m, 4H), 3.58-3.45 (m, 2H), 3.12-3.02 (m, 1H), 2.04-1.70 (m, 3H). LC-MS (m/z): 491.2 [M + H]⁺. |
| A2 | 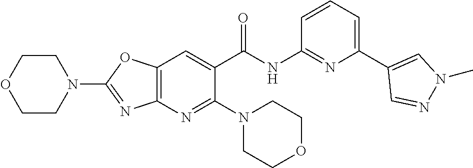 [SMILES: Cn1cc(-c2cccc(NC(=O)c3cc4oc(N5CCOCC5)nc4nc3N3CCOCC3)n2)cn1] | AC | ¹H NMR (300 MHz, DMSO-d₆) δ 12.83 (s, 1H), 8.25 (s, 1H), 8.24 (s, 1H), 8.05 (d, J = 7.5 Hz, 1H), 7.96 (s, 1H), 7.85- 7.77 (m, 1H), 7.39 (d, J = 7.2 Hz, 1H), 4.06-3.94 (m, 4H), 3.94 (s, 3H), 3.80- 3.72 (m, 4H), 3.73-3.67 (m, 4H), 3.18-3.08 (m, 4H). LC-MS (m/z): 491.2 [M + H]⁺. |
| A3 | 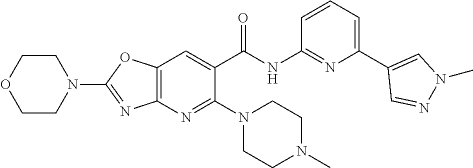 [SMILES: CN1CCN(c2nc3nc(N4CCOCC4)oc3cc2C(=O)Nc2cccc(-c3cnn(C)c3)n2)CC1] | AC | ¹H NMR (300 MHZ, DMSO-d₆) δ 12.65 (s, 1H), 8.28 (s, 1H), 8.26 (s, 1H), 8.11-8.05 (m, 2H), 7.79 (d, J = 8.4 Hz, 1H), 7.39 (d, J = 7.5 Hz, 1H), 3.91 (s, 3H), 3.76-3.73 (m, 4H), 3.72- 3.68 (m, 4H), 3.18-3.09 (m, 4H), 2.77-2.70 (m, 4H), 2.33-2.27 (m, 3H). LC-MS (m/z): 504.2 [M + H]⁺. |
| A4 | 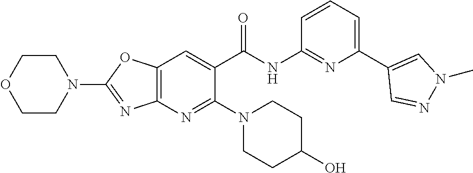 [SMILES: Cn1cc(-c2cccc(NC(=O)c3cc4oc(N5CCOCC5)nc4nc3N3CCC(O)CC3)n2)cn1] | AC | ¹H NMR (300 MHZ, DMSO-d₆) δ 13.50 (s, 1H), 8.42 (s, 1H), 8.27 (s, 1H), 8.05 (s, 1H), 8.02 (s, 1H), 7.83- 7.74 (m, 1H), 7.39 (d, J = 7.5 Hz, 1H), 5.16 (s, 1H), 4.08-3.98 (m, 1H), 3.86 (s, 3H), 3.77-3.71 (m, 4H), 3.71- 3.66 (m, 4H), 3.25-3.14 (m, 2H), 3.06- 2.94 (m, 2H), 2.13-1.96 (m, 4H). LC-MS (m/z): 505.2 [M + H]⁺. |
| A5 | 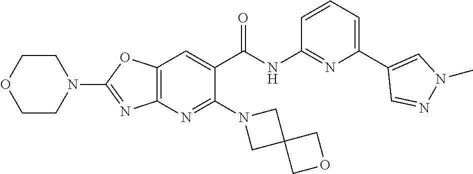 [SMILES: Cn1cc(-c2cccc(NC(=O)c3cc4oc(N5CCOCC5)nc4nc3N3CC4(COC4)C3)n2)cn1] | AC | ¹H NMR (300 MHZ, DMSO-d₆) δ 10.45 (s, 1H), 8.21 (s, 1H), 7.99-7.92 (m, 2H), 7.83-7.75 (m, 2H), 7.39 (d, J = 7.5 Hz, 1H), 4.71-4.63 (m, 4H), 4.10-4.03 (m, 4H), 3.88 (s, 3H), 3.74- 3.69 (m, 4H), 3.65-3.61 (m, 4H). LC-MS (m/z): 503.2 [M + H]⁺. |
| A6 | 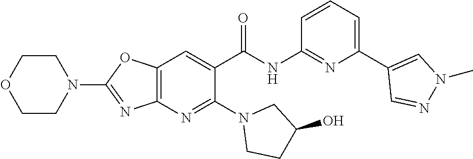 [SMILES: Cn1cc(-c2cccc(NC(=O)c3cc4oc(N5CCOCC5)nc4nc3N3CC[C@H](O)C3)n2)cn1] | AC | ¹H NMR (300 MHZ, DMSO-d₆) δ 10.67 (s, 1H), 8.20 (s, 1H), 8.02-7.90 (m, 2H), 7.82-7.70 (m, 2H), 7.36 (d, J = 7.5 Hz, 1H), 4.93 (s, 1H), 4.34- 4.27 (m, 1H), 3.88 (s, 3H), 3.75-3.68 (m, 4H), 3.66-3.59 (m, 4H), 3.57- 3.53 (m, 2H), 3.14-2.99 (m, 2H), 1.93- 1.75 (m, 2H). LC-MS (m/z): 491.1 [M + H]⁺. |
| A7 | 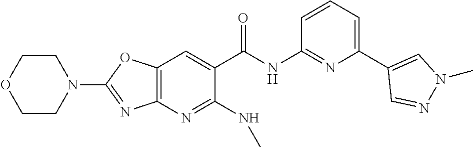 [SMILES: CNc1nc2nc(N3CCOCC3)oc2cc1C(=O)Nc1cccc(-c2cnn(C)c2)n1] | AC | ¹H NMR (300 MHZ, DMSO-d₆) δ 10.14 (s, 1H), 8.40 (s, 1H), 8.31 (s, 1H), 8.23 (s, 1H), 7.99 (s, 1H), 7.80- 7.72 (m, 2H), 7.40-7.32 (m, 1H), 3.89 (s, 3H), 3.78-3.71 (m, 4H), 3.70- 3.62 (m, 4H), 2.92 (d, J = 4.2 Hz, 3H). LC-MS (m/z): 435.1 [M + H]⁺. |
| A8 | 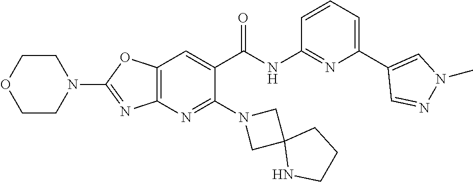 [SMILES: Cn1cc(-c2cccc(NC(=O)c3cc4oc(N5CCOCC5)nc4nc3N3CC4(CCCN4)C3)n2)cn1] | AC, AD, AP | ¹H NMR (300 MHZ, DMSO-d₆) δ 10.53 (s, 1H), 8.23 (s, 1H), 7.99-7.85 (m, 3H), 7.83-7.71 (m, 1H), 7.43- 7.32 (m, 1H), 4.27-4.12 (m, 2H), 3.99- 3.91 (m, 2H), 3.88 (s, 3H), 3.76- 3.69 (m, 4H), 3.67-3.57 (m, 4H), 3.16- 3.01 (m, 2H), 2.16-2.05 (m, 2H), 1.90-1.76 (m, 2H). LC-MS (m/z): 516.2 [M + H]⁺. |
| A9 | 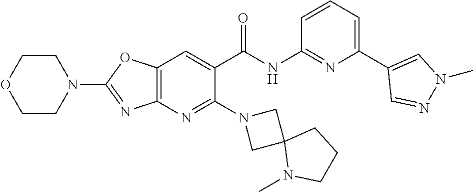 [SMILES: CN1CCCC12CN(c1nc3nc(N4CCOCC4)oc3cc1C(=O)Nc1cccc(-c3cnn(C)c3)n1)C2] | AC, AE | ¹H NMR (300 MHZ, DMSO-d₆) δ 10.39 (s, 1H), 8.20 (s, 1H), 7.99-7.91 (m, 2H), 7.85 (s, 1H), 7.82-7.72 (m, 1H), 7.42-7.33 (m, 1H), 4.13-3.99 (m, 2H), 3.88 (s, 3H), 3.79-3.69 (m, 6H), 3.69-3.57 (m, 4H), 2.56 (s, 2H), 2.36 (s, 3H), 2.13-1.93 (m, 2H), 1.71- 1.54 (m, 2H). LC-MS (m/z): 530.2 [M + H]⁺. |
| A10 | 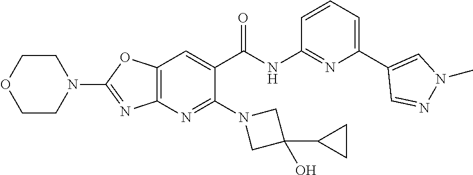 [SMILES: Cn1cc(-c2cccc(NC(=O)c3cc4oc(N5CCOCC5)nc4nc3N3CC(O)(C4CC4)C3)n2)cn1] | AC | ¹H NMR (300 MHZ, DMSO-d₆) δ 10.40 (s, 1H), 8.20 (s, 1H), 8.00-7.90 (m, 2H), 7.85-7.75 (m, 2H), 7.38 (d, J = 7.2 Hz, 1H), 5.42 (s, 1H), 3.88 (s, 3H), 3.80-3.69 (m, 8H), 3.68-3.59 (m, 4H), 1.27-1.15 (m, 1H), 0.40- 0.25 (m, 4H). LC-MS (m/z): 517.2 [M+ H]⁺. |
| A11 | 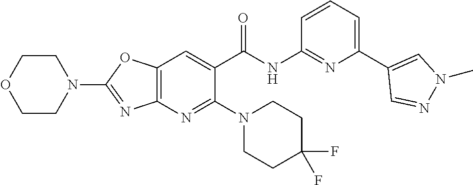 [SMILES: Cn1cc(-c2cccc(NC(=O)c3cc4oc(N5CCOCC5)nc4nc3N3CCC(F)(F)CC3)n2)cn1] | AC | ¹H NMR (300 MHZ, DMSO-d₆) δ 12.83 (s, 1H), 8.28 (s, 1H), 8.16 (s, 1H), 8.06 (d, J = 7.5 Hz, 1H), 7.93 (s, 1H), 7.86-7.78 (m, 1H), 7.41 (d, J = 7.2 Hz, 1H), 3.87 (s, 3H), 3.78-3.73 (m, 4H), 3.72-3.68 (m, 4H), 3.27- 3.23 (m, 4H), 2.47-2.37 (m, 4H). LC- MS (m/z): 525.2 [M + H]⁺. |
| A12 | 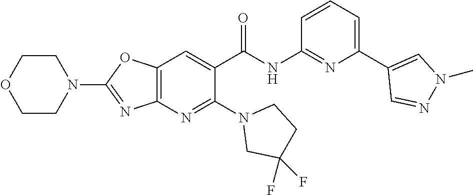 [SMILES: Cn1cc(-c2cccc(NC(=O)c3cc4oc(N5CCOCC5)nc4nc3N3CCC(F)(F)C3)n2)cn1] | AC | ¹H NMR (300 MHZ, DMSO-d₆) δ 10.92 (s, 1H), 8.20 (s, 1H), 7.99-7.89 (m, 3H), 7.82-7.75 (m, 1H), 7.38 (d, J = 7.5 Hz, 1H), 3.88 (s, 3H), 3.75- 3.70 (m, 4H), 3.69-3.62 (m, 4H), 3.60- 3.54 (m, 2H), 3.26-3.23 (m, 2H), 2.45-2.41 (m, 2H). LC-MS (m/z): 511.1 [M + H]⁺. |
| A13 | 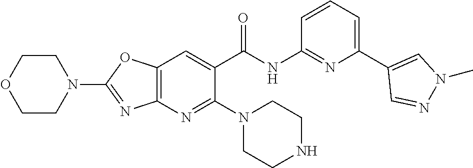 [SMILES: Cn1cc(-c2cccc(NC(=O)c3cc4oc(N5CCOCC5)nc4nc3N3CCNCC3)n2)cn1] | AC | ¹H NMR (300 MHZ, DMSO-d₆) δ 12.65 (s, 1H), 8.25 (s, 1H), 8.23 (s, 1H), 8.06 (d, J = 9.0 Hz, 1H), 8.00 (s, 1H), 7.85-7.77 (m, 1H), 7.41 (d, J = 7.2 Hz, 1H), 3.92 (s, 3H), 3.77-3.72 (m, 4H), 3.72-3.66 (m, 4H), 3.30- 3.25 (m, 4H), 3.23-3.15 (m, 4H). LC- MS (m/z): 490.2 [M + H]⁺. |
| A14 | 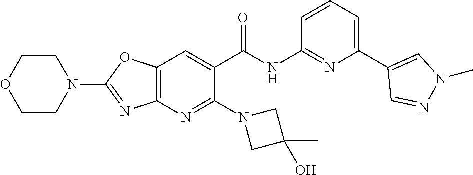 [SMILES: Cn1cc(-c2cccc(NC(=O)c3cc4oc(N5CCOCC5)nc4nc3N3CC(C)(O)C3)n2)cn1] | AC | ¹H NMR (300 MHZ, DMSO-d₆) δ 10.50 (s, 1H), 8.21 (s, 1H), 7.96 (s, 1H), 7.96-7.92 (m, 1H), 7.83 (s, 1H), 7.81-7.75 (m, 1H), 7.38 (d, J = 7.2 Hz, 1H), 5.48 (s, 1H), 3.88 (s, 3H), 3.83-3.75 (m, 4H), 3.76-3.70 (m, 4H), 3.68-3.62 (m, 4H), 1.42 (s, 3H). LC-MS (m/z): 491.1 [M + H]⁺. |
| A15 | 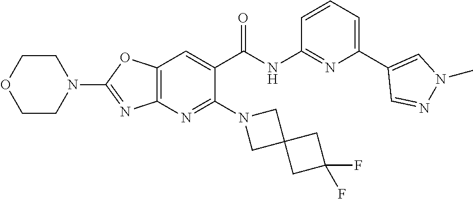 [SMILES: Cn1cc(-c2cccc(NC(=O)c3cc4oc(N5CCOCC5)nc4nc3N3CC4(C3)CC(F)(F)C4)n2)cn1] | AC | ¹H NMR (300 MHZ, DMSO-d₆) δ 10.50 (s, 1H), 8.25 (s, 1H), 8.03-7.95 (m, 2H), 7.87 (s, 1H), 7.84-7.78 (m, 1H), 7.42 (d, J = 7.2 Hz, 1H), 4.10- 4.01 (m, 4H), 3.93 (s, 3H), 3.81-3.73 (m, 4H), 3.71-3.62 (m, 4H), 2.91- 2.80 (m, 4H). LC-MS (m/z): 537.2 [M + H]⁺. |
| A16 | 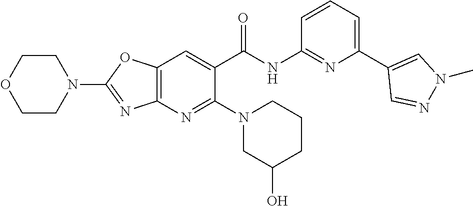 [SMILES: Cn1cc(-c2cccc(NC(=O)c3cc4oc(N5CCOCC5)nc4nc3N3CCCC(O)C3)n2)cn1] | AC | ¹H NMR (300 MHZ, DMSO-d₆) δ 12.98 (s, 1H), 8.26 (s, 1H), 8.20 (s, 1H), 8.05 (d, J = 9.3 Hz, 1H), 7.95 (s, 1H), 7.83-7.75 (m, 1H), 7.38 (d, J = 7.8 Hz, 1H), 5.07-4.99 (m, 1H), 4.14- 4.06 (m, 1H), 3.90 (s, 3H), 3.82- 3.73 (m, 4H), 3.73-3.66 (m, 4H), 3.13- 3.06 (m, 1H), 2.92-2.83 (m, 1H), 2.80-2.65 (m, 2H), 2.05-1.88 (m, 3H), 1.43-1.33 (m, 1H). LC-MS (m/z): 505.2 [M + H]⁺. |
| A17 | 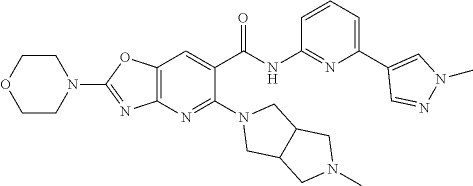 [SMILES: CN1CC2CN(c3nc4nc(N5CCOCC5)oc4cc3C(=O)Nc3cccc(-c4cnn(C)c4)n3)CC2C1] | AC | ¹H NMR (300 MHZ, DMSO-d₆) δ 11.30 (s, 1H), 8.21 (s, 1H), 8.01-7.88 (m, 2H), 7.84-7.73 (m, 2H), 7.43- 7.31 (m, 1H), 3.88 (s, 3H), 3.78-3.70 (m, 4H), 3.69-3.62 (m, 4H), 3.26- 3.11 (m,4H), 3.00-2.85 (m, 2H), 2.81- 2.57 (m, 4H), 2.29 (s, 3H). LC-MS (m/z): 530.2 [M + H]⁺. |
| A18 | 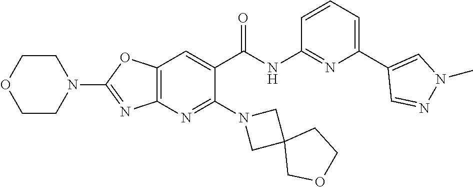 [SMILES: Cn1cc(-c2cccc(NC(=O)c3cc4oc(N5CCOCC5)nc4nc3N3CC4(CCOC4)C3)n2)cn1] | AC, AF | ¹H NMR (300 MHZ, CDCl₃) δ 10.20 (s, 1H), 8.23-8.10 (m, 1H), 8.04 (s, 1H), 7.96 (s, 1H), 7.89 (s, 1H), 7.76- 7.65 (m, 1H), 7.24-7.17 (m, 1H), 4.10 (s, 3H), 4.02-3.93 (m, 5H), 3.89- 3.76 (m, 11H), 2.22 (t, J = 7.1 Hz, 2H). LC-MS (m/z): 517.2 [M + H]⁺ |
| A19 | 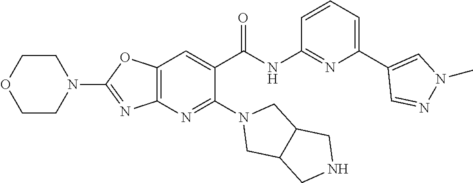 [SMILES: Cn1cc(-c2cccc(NC(=O)c3cc4oc(N5CCOCC5)nc4nc3N3CC4CNCC4C3)n2)cn1] | AC, AP | ¹H NMR (300 MHZ, DMSO-d₆) δ 10.88 (s, 1H), 8.22 (s, 1H), 7.99-7.94 (m, 2H), 7.90 (s, 1H), 7.84-7.73 (m, 1H), 7.41-7.36 (m, 1H), 3.89 (s, 3H), 3.77-3.70 (m, 4H), 3.69-3.61 (m, 4H), 3.56-3.44 (m, 4H), 3.34-3.27 (m, 2H), 3.10-2.99 (m, 4H). LC-MS (m/z): 516.2 [M + H]⁺. |
| A20 | 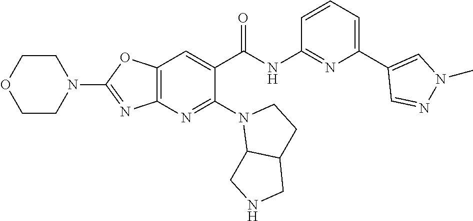 [SMILES: Cn1cc(-c2cccc(NC(=O)c3cc4oc(N5CCOCC5)nc4nc3N3CCC4CNCC43)n2)cn1] | AC, AP | ¹H NMR (300 MHZ, DMSO-d₆) δ 12.09 (s, 1H), 8.21 (s, 1H), 8.08-8.01 (m, 2H), 8.00-7.94 (m, 1H), 7.83- 7.76 (m, 1H), 7.38 (d, J = 6.9 Hz, 1H), 4.57-4.52 (m, 1H), 3.89 (s, 3H), 3.76- 3.71 (m, 4H), 3.70-3.65 (m, 4H), 3.09-3.01 (m, 2H), 2.84-2.74 (m, 4H), 2.64-2.55 (m, 2H), 2.12-2.07 (m, 1H), 1.77-1.70 (m, 1H). LC-MS (m/z): 516.3 [M + H]⁺. |
| A21 | 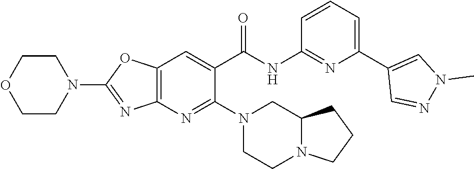 [SMILES: Cn1cc(-c2cccc(NC(=O)c3cc4oc(N5CCOCC5)nc4nc3N3CCN4CCC[C@@H]4C3)n2)cn1] | AC | ¹H NMR (300 MHZ, DMSO-d₆) δ 12.63 (s, 1H), 8.28-8.19 (m, 2H), 8.05 (d, J = 8.4 Hz, 1H), 8.00 (s, 1H), 7.83-7.75 (m, 1H), 7.38 (d, J = 7.8 Hz, 1H), 3.89 (s, 3H), 3.78-3.72 (m, 4H), 3.71-3.66 (m, 4H), 3.23-3.10 (m, 4H), 3.04-2.75 (m, 4H), 1.88- 1.71 (m, 3H), 1.61-1.37 (m, 2H). LC- MS (m/z): 530.3 [M + H]⁺. |
| A22 | 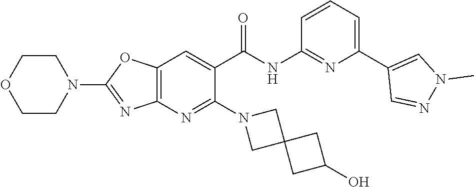 [SMILES: Cn1cc(-c2cccc(NC(=O)c3cc4oc(N5CCOCC5)nc4nc3N3CC4(CC(O)C4)C3)n2)cn1] | AC | ¹H NMR (300 MHZ, DMSO-d₆) δ 10.42 (s, 1H), 8.21 (s, 1H), 7.99-7.89 (m, 2H), 7.85-7.75 (m, 2H), 7.38 (d, J = 7.2 Hz, 1H), 5.03-4.98 (m, 1H), 3.90-3.87 (m, 4H)., 3.86-3.81 (m, 4H), 3.74-3.71 (m, 4H), 3.65-3.62 (m, 4H), 2.46-2.35 (m, 2H), 2.02- 1.91 (m, 2H). LC-MS (m/z): 517.2 [M + H]⁺. |
| A23 | 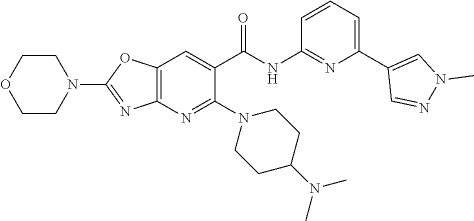 [SMILES: CN(C)C1CCN(c2nc3nc(N4CCOCC4)oc3cc2C(=O)Nc2cccc(-c3cnn(C)c3)n2)CC1] | AC | ¹H NMR (300 MHz, DMSO-d₆) δ 12.61-12.38 (m, 1H), 8.39 (s, 1H), 8.27 (s, 1H), 8.13 (d, J = 8.4 Hz, 1H), 8.07 (s, 1H), 7.90-7.80 (m, 1H), 7.44 (d, J = 7.6 Hz, 1H), 3.92 (s, 3H), 3.83- 3.70 (m, 8H), 3.38-3.29 (m, 3H), 3.14- 2.94 (m, 2H), 2.55 (s, 6H), 2.20- 2.10 (m, 2H), 2.06-1.94 (m, 2H). LC- MS (m/z): 532.2 [M + H]⁺. |
| A24 | 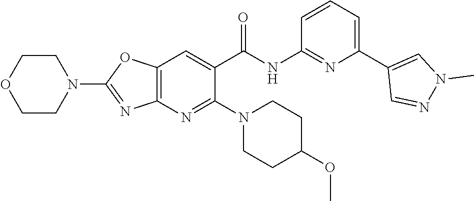 [SMILES: COC1CCN(c2nc3nc(N4CCOCC4)oc3cc2C(=O)Nc2cccc(-c3cnn(C)c3)n2)CC1] | AC | ¹H NMR (300 MHZ, DMSO-d₆) δ 13.50-13.36 (m, 1H), 8.29-8.25 (m, 2H), 8.07-8.02 (m, 2H), 7.83-7.75 (m, 1H), 7.39 (d, J = 7.2 Hz, 1H), 3.87 (s, 3H), 3.76-3.65 (m, 8H), 3.38 (s, 3H), 3.26-3.15 (m, 3H), 3.06-2.93 (m, 2H), 2.23-2.12 (m, 2H), 2.09- 1.98 (m, 2H). LC-MS (m/z): 519.3 [M + H]⁺. |
| A25 | 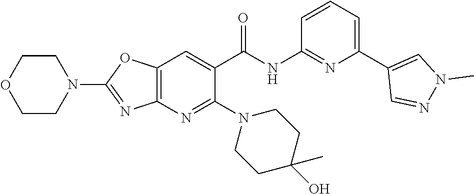 [SMILES: Cn1cc(-c2cccc(NC(=O)c3cc4oc(N5CCOCC5)nc4nc3N3CCC(C)(O)CC3)n2)cn1] | AC | ¹H NMR (300 MHZ, DMSO-d₆) δ 13.20-13.12 (m, 1H), 8.36 (s, 1H), 8.27 (s, 1H), 8.12-8.05 (m, 1H), 8.00 (s, 1H), 7.79 (s, 1H), 7.38 (d, J = 7.2 Hz, 1H), 4.61 (s, 1H), 3.87 (s, 3H), 3.79-3.67 (m, 8H), 3.28-3.20 (m, 2H), 3.04-2.93 (m, 2H), 1.95-1.82 (m, 4H), 1.26 (s, 3H). LC-MS (m/z): 519.2 [M + H]⁺. |
| A26 | 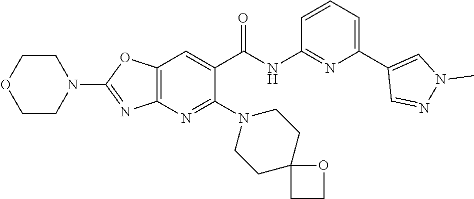 [SMILES: Cn1cc(-c2cccc(NC(=O)c3cc4oc(N5CCOCC5)nc4nc3N3CCC4(CCO4)CC3)n2)cn1] | AC, AG | ¹H NMR (300 MHZ, DMSO-d₆) δ 12.42 (s, 1H), 8.22 (s, 1H), 8.13 (s, 1H), 8.09-7.98 (m, 1H), 7.92 (s, 1H), 7.84-7.70 (m, 1H), 7.42-7.31 (m, 1H), 5.67-5.52 (m, 1H), 4.59-4.41 (m, 1H), 3.89 (s, 3H), 3.80-3.63 (m, 8H), 3.61- 3.49 (m, 4H), 3.25-3.14 (m, 2H), 2.43- 2.32 (m, 2H), 2.27-2.13 (m, 2H). |
| A27 | 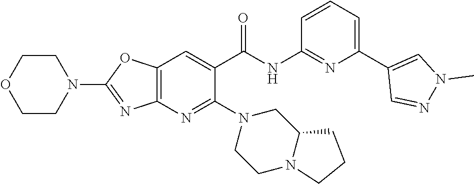 [SMILES: Cn1cc(-c2cccc(NC(=O)c3cc4oc(N5CCOCC5)nc4nc3N3CCN4CCC[C@H]4C3)n2)cn1] | AC | ¹H NMR (300 MHZ, DMSO-d₆) δ 12.76 (s, 1H), 8.28-8.23 (m, 2H), 8.07 (d, J = 8.1 Hz, 1H), 8.01 (s, 1H), 7.80 (t, J = 7.8 Hz, 1H), 7.41-7.35 (m, 1H), 3.89 (s, 3H), 3.78-3.67 (m, 8H), 3.19-3.03 (m, 4H), 2.98-2.85 (m, 2H), 2.83-2.61 (m, 2H), 2.32- 2.17 (m, 1H), 1.90-1.65 (m, 3H), 1.46- 1.31 (m, 1H). LC-MS (m/z): 530.3 [M + H]⁺. |
| A28 | 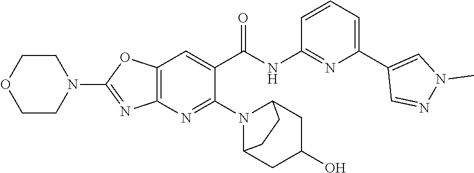 [SMILES: Cn1cc(-c2cccc(NC(=O)c3cc4oc(N5CCOCC5)nc4nc3N3C4CCC3CC(O)C4)n2)cn1] | AC | ¹H NMR (300 MHz, DMSO-d₆) δ 11.52 (s, 1H), 8.18 (s, 1H), 8.11-8.01 (m, 2H), 7.88 (s, 1H), 7.81-7.75 (m, 1H), 7.36 (d, J = 6.6 Hz, 1H), 4.58- 4.52 (m, 1H), 4.04-3.93 (m, 2H), 3.88 (s, 3H), 3.78-3.70 (m, 4H), 3.70- 3.62 (m, 4H), 2.45-2.37 (m, 2H), 2.21- 2.13 (m, 2H), 1.95-1.85 (m, 2H), 1.81-1.72 (m, 2H). LC-MS (m/z): 531.2 [M + H]⁺. |
| A29 | 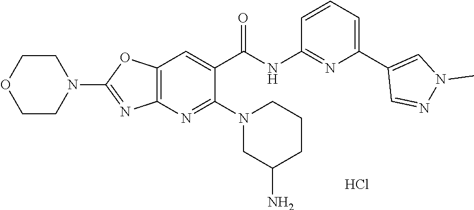 [SMILES: Cn1cc(-c2cccc(NC(=O)c3cc4oc(N5CCOCC5)nc4nc3N3CCCC(N)C3)n2)cn1 ; Cl] | AC, AP | ¹H NMR (300 MHZ, DMSO-d₆) δ 12.57 (s, 1H), 8.34-8.28 (m, 2H), 8.26 (s, 1H), 8.20 (s, 1H), 8.06 (d, J = 7.5 Hz, 1H), 7.95 (s, 1H), 7.87-7.79 (m, 1H), 7.41 (d, J = 7.5 Hz, 1H), 3.93 (s, 3H), 3.80-3.67 (m, 8H), 3.24- 3.17 (m, 1H), 3.07-3.00 (m, 1H), 2.85- 2.69 (m, 2H), 2.31-2.23 (m, 1H), 2.15-2.05 (m, 1H), 2.02-1.95 (m, 2H), 1.62-1.55 (m, 1H). |
| A30 | 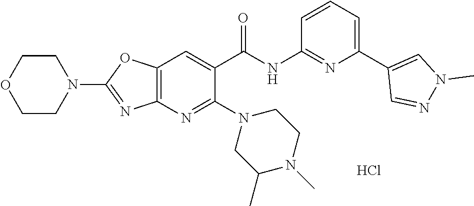 [SMILES: CC1CN(c2nc3nc(N4CCOCC4)oc3cc2C(=O)Nc2cccc(-c3cnn(C)c3)n2)CCN1C ; Cl] | AC | ¹H NMR (300 MHZ, DMSO-d₆) δ 11.81 (s, 1H), 11.57 (s, 1H), 8.36 (s, 1H), 8.20 (s, 1H), 8.08-7.97 (m, 2H), 7.87 (t, J = 7.8 Hz, 1H), 7.45 (d, J = 7.5 Hz, 1H), 3.96 (s, 1H), 3.92 (s, 3H), 3.73-3.65 (m, 8H), 3.60-3.46 (m, 4H), 3.43-3.26 (m, 2H), 2.78 (s, 3H), 1.38 (d, J = 5.4 Hz, 3H). |
| A31 | 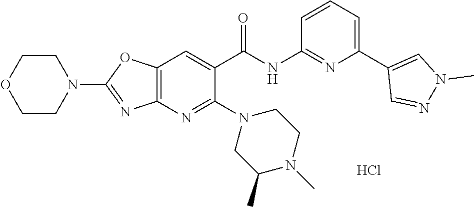 [SMILES: C[C@H]1CN(c2nc3nc(N4CCOCC4)oc3cc2C(=O)Nc2cccc(-c3cnn(C)c3)n2)CCN1C ; Cl] | AC | ¹H NMR (300 MHZ, DMSO-d₆) δ 11.80 (s, 1H), 11.43 (s, 1H), 8.35 (s, 1H), 8.21 (s, 1H), 8.09-7.98 (m, 2H), 7.87 (t, J = 7.8 Hz, 1H), 7.45 (d, J = 7.5 Hz, 1H), 3.95 (s, 1H), 3.92 (s, 3H), 3.84-3.81 (m, 8H), 3.60-3.47 (m, 4H), 3.41-3.27 (m, 2H), 2.79 (s, 3H), 1.38 (d, J = 5.4 Hz, 3H). |
| A32 | 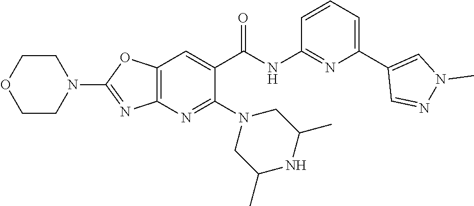 [SMILES: CC1CN(c2nc3nc(N4CCOCC4)oc3cc2C(=O)Nc2cccc(-c3cnn(C)c3)n2)CC(C)N1] | AC | ¹H NMR (300 MHz, DMSO-d₆) δ 13.10 (s, 1H), 8.26 (s, 1H), 8.16 (s, 1H), 8.02 (d, J = 8.1 Hz, 1H), 7.94 (s, 1H), 7.79 (t, J = 7.8 Hz, 1H), 7.38 (d, J = 7.8 Hz, 1H), 3.91 (s, 3H), 3.80-3.62 (m, 8H), 3.53-3.42 (m, 2H), 3.06 (d, J = 11.1 Hz, 2H), 2.60-2.52 (m, 2H), 1.01 (d, J = 6.0 Hz, 6H). |
| A33 | 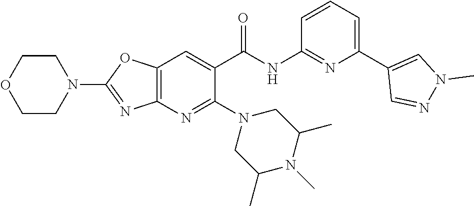 [SMILES: CC1CN(c2nc3nc(N4CCOCC4)oc3cc2C(=O)Nc2cccc(-c3cnn(C)c3)n2)CC(C)N1C] | AH | ¹H NMR (300 MHz, DMSO-d₆) δ 12.34 (s, 1H), 8.27 (s, 1H), 8.24 (s, 1H), 8.06 (d, J = 7.8 Hz, 1H), 8.02 (s, 1H), 7.81 (t, J = 7.8 Hz, 1H), 7.40 (d, J = 7.5 Hz, 1H), 3.90 (s, 3H), 3.79-3.68 (m, 8H), 3.54-3.46 (m, 4H), 3.23 (s, 3H), 3.08-2.89 (m, 2H), 1.16 (s, 6H). |
| A34 | 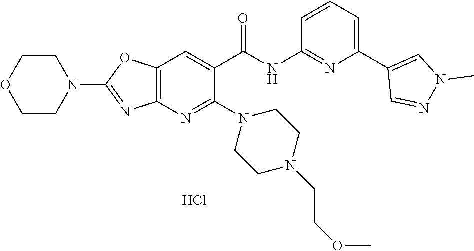 [SMILES: COCCN1CCN(c2nc3nc(N4CCOCC4)oc3cc2C(=O)Nc2cccc(-c3cnn(C)c3)n2)CC1 ; Cl] | AC | ¹H NMR (300 MHZ, DMSO-d₆) δ 11.74 (s, 1H), 11.28 (s, 1H), 8.33 (s, 1H), 8.19 (s, 1H), 8.11-7.98 (m, 2H), 7.91-7.80 (m, 1H), 7.44 (d, J = 7.5 Hz, 1H), 4.26-4.09 (m, 11H), 3.93- 3.88 (m, 2H), 3.81-3.72 (m, 4H), 3.72- 3.62 (m, 4H), 3.52 (s, 3H), 3.38- 3.27 (m, 2H). |
| A35 | 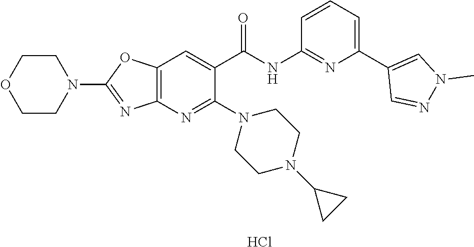 [SMILES: Cn1cc(-c2cccc(NC(=O)c3cc4oc(N5CCOCC5)nc4nc3N3CCN(C4CC4)CC3)n2)cn1 ; Cl] | AC | ¹H NMR (300 MHZ, DMSO-d₆) δ 11.68 (s, 1H), 11.50 (s, 1H), 8.38 (s, 1H), 8.17 (s, 1H), 8.11-7.97 (m, 2H), 7.93-7.77 (m, 1H), 7.51-7.39 (m, 1H), 3.89 (s, 3H), 3.82-3.66 (m, 8H), 3.64-3.38 (m, 8H), 2.77-2.65 (m, 1H), 1.32-1.15 (m, 2H), 0.85-0.70 (m, 2H). |
| A36 | 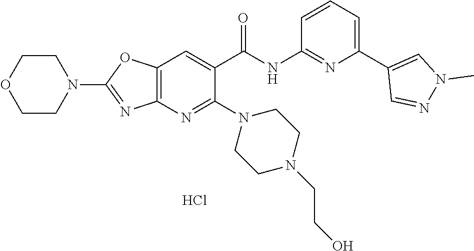 [SMILES: Cn1cc(-c2cccc(NC(=O)c3cc4oc(N5CCOCC5)nc4nc3N3CCN(CCO)CC3)n2)cn1 ; Cl] | AC | ¹H NMR (300 MHZ, DMSO-d₆) δ 11.77 (s, 1H), 10.89 (s, 1H), 8.32 (s, 1H), 8.20 (s, 1H), 8.11-7.94 (m, 2H), 7.93-7.80 (m, 1H), 7.43 (d, J = 7.5 Hz, 1H), 3.91 (s, 3H), 3.86-3.78 (m, 8H), 3.69-3.60 (m, 4H), 3.58-3.46 (m, 4H), 3.42-3.29 (m, 2H), 3.25- 3.13 (m, 2H). |
| A37 | 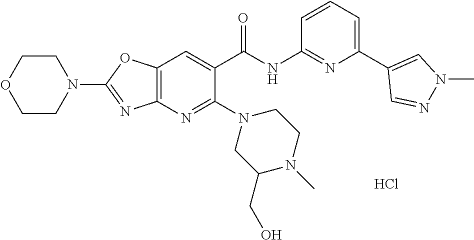 [SMILES: CN1CCN(c2nc3nc(N4CCOCC4)oc3cc2C(=O)Nc2cccc(-c3cnn(C)c3)n2)CC1CO ; Cl] | AC | ¹H NMR (300 MHZ, DMSO-d₆) δ 11.83 (s, 1H), 10.50 (s, 1H), 8.31 (s, 1H), 8.22 (s, 1H), 8.15-7.98 (m, 2H), 7.90-7.80 (m, 1H), 7.52-7.39 (m, 1H), 3.93 (s, 3H), 3.72-3.63 (m, 16H), 3.47-3.36 (m, 3H), 2.91-2.83 (m, 2H). |
| A38 | 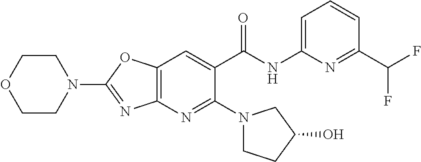 [SMILES: O=C(Nc1cccc(C(F)F)n1)c1cc2oc(N3CCOCC3)nc2nc1N1CC[C@@H](O)C1] | AB, AC, AK | ¹H NMR (300 MHZ, DMSO-d₆) δ 11.07 (s, 1H), 8.32 (d, J = 7.8 Hz, 1H), 8.07-7.93 (m, 1H), 7.75 (s, 1H), 7.41 (d, J = 7.2 Hz, 1H), 6.89 (t, J = 55.2 Hz, 1H), 4.92-4.80 (m, 1H), 4.33-4.22 (m, 1H), 3.79-3.67 (m, 4H), 3.67-3.59 (m, 4H), 3.59-3.51 (m, 1H), 3.50-3.42 (m, 1H), 3.06-2.97 (m, 1H), 2.00-1.68 (m, 3H). LC-MS (m/z): 461.4 [M + H]⁺. |
| A39 | 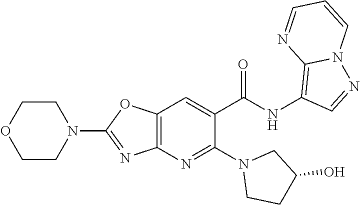 [SMILES: O=C(Nc1cnn2cccnc12)c1cc2oc(N3CCOCC3)nc2nc1N1CC[C@@H](O)C1] | AB, AC | ¹H NMR (300 MHZ, DMSO-d₆) δ 10.48 (s, 1H), 9.04 (d, J = 3.9 Hz, 1H), 8.60 (s, 1H), 8.54-8.48 (m, 1H), 7.75 (s, 1H), 7.06-6.99 (m, 1H), 4.88 (s, 1H), 4.33-4.25 (m, 1H), 3.77-3.69 (m, 4H), 3.68-3.61 (m, 4H), 3.58- 3.54 (m, 2H), 3.18-3.03 (m, 2H), 1.99- 1.72 (m, 2H). LC-MS (m/z): 451.1 [M + H]+. Purity: 97%. |
| A40 | 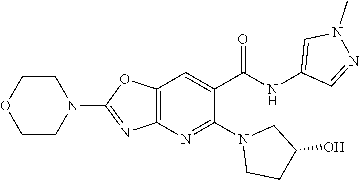 [SMILES: Cn1cc(NC(=O)c2cc3oc(N4CCOCC4)nc3nc2N2CC[C@@H](O)C2)cn1] | AB, AC | ¹H NMR (300 MHZ, DMSO-d₆) δ 10.65 (s, 1H), 7.61 (s, 1H), 7.56 (s, 1H), 6.53 (s, 1H), 4.89 (s, 1H), 4.31- 4.23 (m, 1H), 3.75 (s, 3H), 3.73-3.67 (m, 4H), 3.65-3.58 (m, 4H), 3.54- 3.49 (m, 2H), 3.06-2.99 (m, 2H), 1.90- 1.74 (m, 2H). LC-MS (m/z): 414.2 [M + H]⁺. |
| A41 | 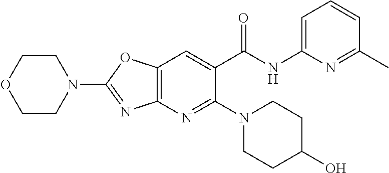 [SMILES: Cc1cccc(NC(=O)c2cc3oc(N4CCOCC4)nc3nc2N2CCC(O)CC2)n1] | AB, AC | ¹H NMR (300 MHZ, DMSO-d₆) δ 12.82 (s, 1H), 8.20 (s, 1H), 8.03 (s, 1H), 7.72 (s, 1H), 6.99 (s, 1H), 4.78 (s, 1H), 3.75-3.68 (m, 9H), 3.25-3.12 (m, 2H), 3.00-2.88 (m, 2H), 2.44- 2.34 (m, 3H), 2.02-1.73 (m, 4H). LC- MS (m/z): 439.2 [M + H]⁺. |
| A42 | 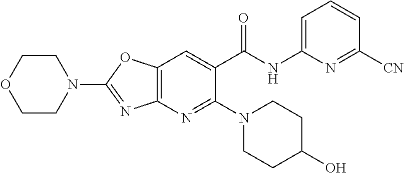 [SMILES: N#Cc1cccc(NC(=O)c2cc3oc(N4CCOCC4)nc3nc2N2CCC(O)CC2)n1] | AB, AC | ¹H NMR (300 MHZ, DMSO-d₆) δ 13.14 (s, 1H), 8.55 (d, J = 8.7 Hz, 1H), 8.18 (s, 1H), 8.13-8.02 (m, 1H), 7.76 (d, J = 7.2 Hz, 1H), 4.81 (s, 1H), 3.82- 3.62 (m, 10H), 3.26-3.18 (m, 1H), 3.05-2.83 (m, 2H), 2.05-1.82 (m, 2H), 1.81-1.58 (m, 2H). LC-MS (m/z): 450.1 [M + H]⁺. |
| A43 | 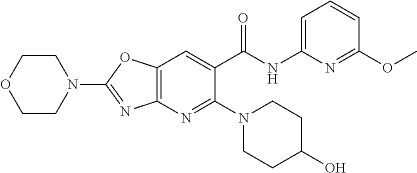 [SMILES: COc1cccc(NC(=O)c2cc3oc(N4CCOCC4)nc3nc2N2CCC(O)CC2)n1] | AB, AC | ¹H NMR (300 MHZ, DMSO-d₆) δ 13.22 (s, 1H), 8.25 (s, 1H), 7.85 (d, J = 7.5 Hz, 1H), 7.77-7.69 (m, 1H), 6.55 (d, J = 7.2 Hz, 1H), 4.84 (s, 1H), 3.87 (s, 3H), 3.78-3.64 (m, 8H), 3.25- 3.13 (m, 3H), 3.04-2.86 (m, 2H), 2.06- 1.79 (m, 4H). LC-MS (m/z): 455.2 [M + H]⁺. |
| A44 | 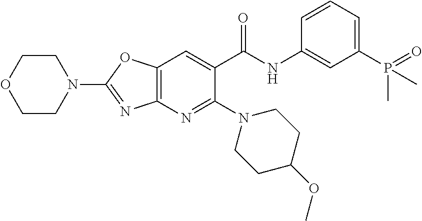 [SMILES: COC1CCN(c2nc3nc(N4CCOCC4)oc3cc2C(=O)Nc2cccc(P(C)(C)=O)c2)CC1] | AB, AC, AI | ¹H NMR (300 MHZ, DMSO-d₆) δ 11.52 (s, 1H), 8.17 (d, J = 11.4 Hz, 1H), 8.03 (s, 1H), 7.83 (s, 1H), 7.54- 7.39 (m, 2H), 3.78-3.71 (m, 4H), 3.69- 3.64 (m, 4H), 3.28-3.19 (m, 6H), 3.03-2.89 (m, 2H), 2.04-1.86 (m, 2H), 1.65 (d, J = 13.2 Hz, 6H), 1.60- 1.53 (m, 2H). LC-MS (m/z): 514.2 [M + H]⁺. |
| A45 | 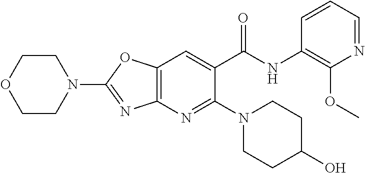 [SMILES: COc1ncccc1NC(=O)c1cc2oc(N3CCOCC3)nc2nc1N1CCC(O)CC1] | AB, AC | ¹H NMR (300 MHZ, DMSO-d₆) δ 12.09 (s, 1H), 8.83 (d, J = 7.7 Hz, 1H), 8.21 (s, 1H), 7.89 (d, J = 4.5 Hz, 1H), 7.12-6.95 (m, 1H), 4.86 (s, 1H), 4.03 (s, 3H), 3.81-3.63 (m, 8H), 3.25- 3.10 (m, 3H), 3.06-2.86 (m, 2H), 1.94- 1.86 (m, 2H), 1.81-1.66 (m, 2H). LC-MS (m/z): 455.1 [M + H]⁺. |
| A46 | 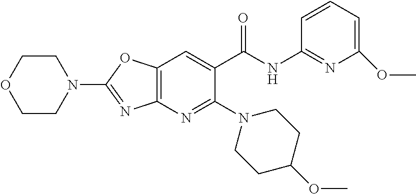 [SMILES: COc1cccc(NC(=O)c2cc3oc(N4CCOCC4)nc3nc2N2CCC(OC)CC2)n1] | AC | ¹H NMR (300 MHZ, DMSO-d₆) δ 13.21 (s, 1H), 8.26 (s, 1H), 7.84 (d, J = 7.9 Hz, 1H), 7.73 (t, J = 7.5 Hz, 1H), 6.55 (d, J = 8.1 Hz, 1H), 3.84 (s, 3H), 3.76-3.68 (m, 9H), 3.28 (s, 3H), 3.23- 3.16 (m, 2H), 3.02-2.93 (m, 2H), 2.11 (s, 2H), 2.02-1.85 (m, 2H). LC- MS (m/z): 469.2 [M + H]⁺. |
| A47 | 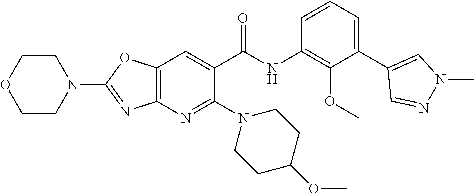 [SMILES: COc1c(NC(=O)c2cc3oc(N4CCOCC4)nc3nc2N2CCC(OC)CC2)cccc1-c1cnn(C)c1] | AB, AC, AJ | ¹H NMR (300 MHZ, DMSO-d₆) δ 12.11 (s, 1H), 8.30 (d, J = 7.7 Hz, 1H), 8.21 (s, 1H), 8.12 (s, 1H), 7.90 (s, 1H), 7.32 (d, J = 8.9 Hz, 1H), 7.20-7.10 (m, 1H), 3.91 (s, 3H), 3.79-3.64 (m, 8H), 3.57 (s, 3H), 3.45-3.40 (m, 3H), 3.27 (s, 3H), 3.09-2.94 (m, 2H), 2.06- 1.94 (m, 2H), 1.85-1.68 (m, 2H). |
| A48 | 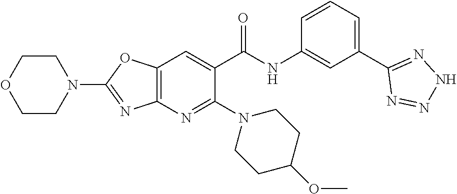 [SMILES: COC1CCN(c2nc3nc(N4CCOCC4)oc3cc2C(=O)Nc2cccc(-c3nn[nH]n3)c2)CC1] | AB, AC, AK | ¹H NMR (300 MHZ, DMSO-d₆) δ 11.43 (s, 1H), 8.54 (s, 1H), 8.04 (s, 1H), 7.81-7.61 (m, 2H), 7.51-7.42 (m, 1H), 3.82-3.63 (m, 8H), 3.51- 3.45 (m, 2H), 3.40-3.31 (m, 4H), 3.04- 2.92 (m, 2H), 1.95 (s, 2H), 1.62 (s, 2H). |
| A49 | 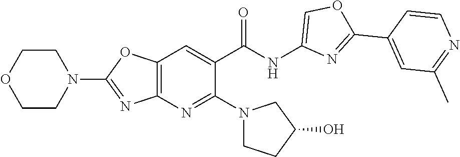 [SMILES: Cc1cc(-c2nc(NC(=O)c3cc4oc(N5CCOCC5)nc4nc3N3CC[C@@H](O)C3)co2)ccn1] | AC, AL | ¹H NMR (300 MHZ, DMSO-d₆) δ 11.21 (s, 1H), 8.62 (d, J = 5.1 Hz, 1H), 8.41 (s, 1H), 7.75-7.67 (m, 2H), 7.67- 7.60 (m, 1H), 4.86 (s, 1H), 4.31-4.20 (m, 1H), 3.80-3.66 (m, 4H), 3.66-3.57 (m, 4H), 3.57-3.50 (m, 1H), 3.49-3.41 (m, 1H), 3.05-2.89 (m, 1H), 2.56 (s, 3H), 2.00-1.67 (m, 3H). LC-MS (m/z): 490.4 [M − H]⁻. |
| A50 | 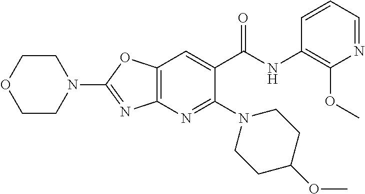 [SMILES: COc1ncccc1NC(=O)c1cc2oc(N3CCOCC3)nc2nc1N1CCC(OC)CC1] | AC | ¹H NMR (300 MHZ, DMSO-d₆) δ 12.09 (s, 1H), 8.84 (d, J = 7.8 Hz, 1H), 8.22 (s, 1H), 7.91-7.85 (m, 1H), 7.09- 6.97 (m, 1H), 4.00 (s, 3H), 3.77- 3.72 (m, 4H), 3.71-3.66 (m, 4H), 3.30 (s, 3H), 3.25-3.22 (m, 1H), 3.21- 3.14 (m, 2H), 3.00-2.89 (m, 2H), 2.11- 1.98 (m, 2H), 1.85-1.70 (m, 2H). |
| A51 | 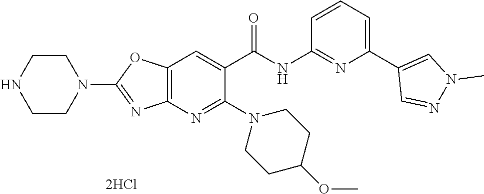 [SMILES: COC1CCN(c2nc3nc(N4CCNCC4)oc3cc2C(=O)Nc2cccc(-c3cnn(C)c3)n2)CC1] | AB, AM | ¹H NMR (300 MHZ, DMSO-d₆) δ 13.37 (s, 1H), 9.69-9.44 (m, 2H), 8.46-8.21 (m, 2H), 8.11-7.93 (m, 2H), 7.80 (s, 1H), 7.41 (s, 1H), 4.00- 3.92 (m, 4H), 3.88 (s, 3H), 3.38 (s, 3H), 3.31-3.17 (m, 7H), 3.05-2.89 (m, 2H), 2.23-1.96 (m, 4H). |
| A52 | 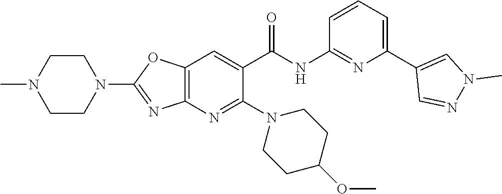 | AO | ¹H NMR (300 MHZ, DMSO-d₆) δ 13.42 (s, 1H), 8.41-8.18 (m, 2H), 8.09-7.99 (m, 2H), 7.86-7.72 (m, 1H), 7.40 (d, J = 6.9 Hz, 1H), 3.88 (s, 3H), 3.75-3.66 (m, 7H), 3.23-3.18 (m, 4H), 3.06-2.94 (m, 5H), 2.25 (s, 3H), 2.17-2.01 (m, 4H). |
| A53 | 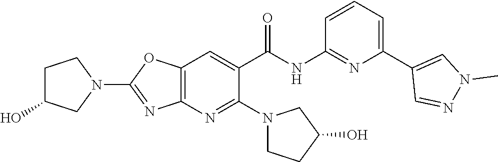 [SMILES: Cn1cc(-c2cccc(NC(=O)c3cc4oc(N5CC[C@@H](O)C5)nc4nc3N3CC[C@@H](O)C3)n2)cn1] | AC | ¹H NMR (300 MHZ, DMSO-d₆) δ 10.65 (s, 1H), 8.20 (s, 1H), 8.11-7.86 (m, 2H), 7.86-7.61 (m, 2H), 7.49-7.23 (m, 1H), 5.16 (s, 1H), 4.89 (s, 1H), 4.52-4.41 (m, 2H), 3.88 (s, 3H), 3.78- 3.40 (m, 8H), 3.17-2.94 (m, 1H), 2.17- 1.70 (m, 3H). LC-MS (m/z): 491.2 [M + H]⁺. |
| A54 | 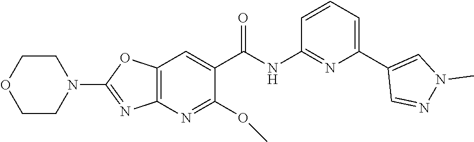 [SMILES: COc1nc2nc(N3CCOCC3)oc2cc1C(=O)Nc1cccc(-c2cnn(C)c2)n1] | AA, AB | ¹H NMR (300 MHZ, DMSO-d₆) δ 10.38 (s, 1H), 8.32-8.17 (m, 2H), 8.10-7.92 (m, 2H), 7.79 (s, 1H), 7.41 (s, 1H), 4.15 (s, 3H), 3.90 (s, 3H), 3.79- 3.64 (m, 8H). LC-MS (m/z): 436.1 [M + H]⁺. |
| A55 | 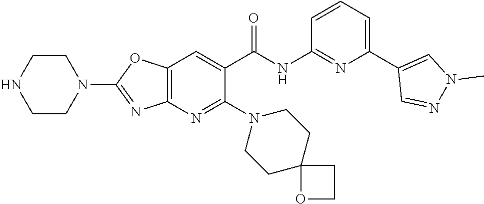 [SMILES: Cn1cc(-c2cccc(NC(=O)c3cc4oc(N5CCNCC5)nc4nc3N3CCC4(CCO4)CC3)n2)cn1] | AM | ¹H NMR (300 MHZ, DMSO-d₆) δ 12.45 (s, 1H), 8.21 (s, 1H), 8.13 (s, 1H), 8.06 (d, J = 7.8 Hz, 1H), 7.92 (s, 1H), 7.77 (t, J = 7.8 Hz, 1H), 7.37 (d, J = 7.2 Hz, 1H), 5.66-5.56 (m, 1H), 4.55-4.43 (m, 1H), 3.89 (s, 3H), 3.72- 3.62 (m, 6H), 3.57-3.49 (m, 2H), 3.24-3.17 (m, 3H), 2.93-2.84 (m, 4H), 8 2.43-2.35 (m, 2H), 2.25-2.17 (m, 2H). LC-MS (m/z): 529.8 [M + H]⁺. |
| A56 | 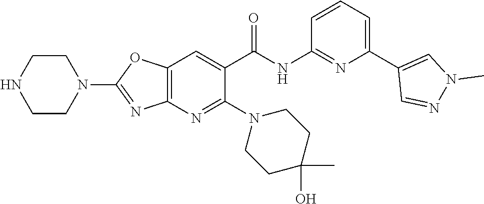 [SMILES: Cn1cc(-c2cccc(NC(=O)c3cc4oc(N5CCNCC5)nc4nc3N3CCC(C)(O)CC3)n2)cn1] | AM | ¹H NMR (300 MHZ, DMSO-d₆) δ 13.19 (s, 1H), 8.36 (s, 1H), 8.26 (s, 1H), 8.08 (d, J = 7.8 Hz, 1H), 8.00 (s, 1H), 7.79 (t, J = 7.5 Hz, 1H), 7.38 (d, J = 7.5 Hz, 1H), 4.62 (s, 1H), 3.87 (s, 3H), 3.76-3.68 (m, 4H), 3.19-3.13 (m, 2H), 3.05-2.93 (m, 7H), 1.96- 1.82 (m, 4H), 1.26 (s, 3H). LC-MS (m/z): 517.8 [M + H]⁺. |
| A57 | 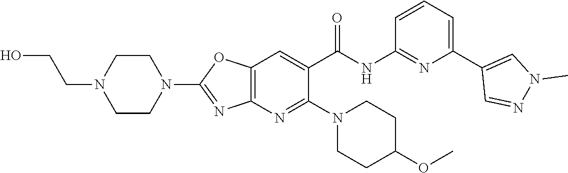 [SMILES: COC1CCN(c2nc3nc(N4CCN(CCO)CC4)oc3cc2C(=O)Nc2cccc(-c3cnn(C)c3)n2)CC1] | AN | ¹H NMR (300 MHZ, DMSO-d₆) δ 13.42 (s, 1H), 8.29-8.16 (m, 2H), 8.08-7.96 (m, 2H), 7.82-7.75 (m, 1H), 7.39 (d, J = 7.8 Hz, 1H), 4.49 (s, 1H), 3.88 (s, 3H), 3.72-3.65 (m, 4H), 3.57-3.50 (m, 5H), 3.45 (s, 3H), 3.24- 3.18 (m, 2H), 3.06-2.96 (m, 2H), 2.61-2.54 (m, 4H), 2.25-2.01 (m, 4H). LC-MS (m/z): 561.8 [M + H]⁺. |
| A58 | 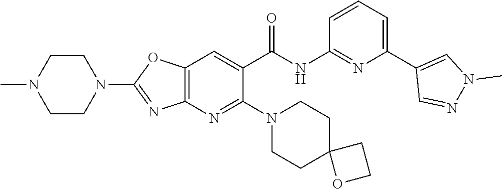 | AO | ¹H NMR (300 MHZ, DMSO-d₆) δ 12.43 (s, 1H), 8.22 (s, 1H), 8.13 (s, 1H), 8.09-7.98 (m, 1H), 7.92 (s, 1H), 7.83-7.71 (m, 1H), 7.37 (d, J = 7.5 Hz, 1H), 5.67-5.54 (m, 1H), 4.58- 4.44 (m, 1H), 3.89 (s, 3H), 3.81-3.63 (m, 6H), 3.60-3.47 (m, 4H), 3.25- 3.18 (m, 2H), 2.61-2.54 (m, 2H), 2.43- 2.36 (m, 2H), 2.30 (s, 3H), 2.24- 2.14 (m, 2H).LC-MS (m/z): 543.8 [M + H]⁺. |
| A59 | 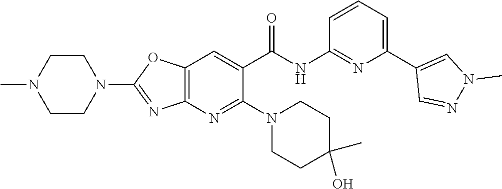 | AO | 1H NMR (300 MHZ, DMSO-d₆) δ 13.18 (s, 1H), 8.35 (s, 1H), 8.25 (s, 1H), 8.08 (d, J = 8.3 Hz, 1H), 8.00 (s, 1H), 7.84-7.73 (m, 1H), 7.38 (d, J = 7.8 Hz, 1H), 4.60 (s, 1H), 3.87 (s, 3H), 3.77-3.58 (m, 4H), 3.51-3.41 (m, 2H), 3.25-3.18 (m, 2H), 3.11-2.91 (m, 4H), 2.29 (s, 3H), 1.96-1.79 (m, 4H), 1.25 (s, 3H). LC-MS (m/z): 531.8 [M + H]+. |
| B1 | 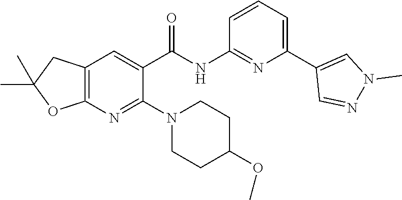 | BA, BB | ¹H NMR (300 MHZ, DMSO-d₆) δ 12.71 (s, 1H), 8.25 (s, 1H), 8.22 (s, 1H), 8.07-7.97 (m, 2H), 7.78 (t, J = 7.5 Hz, 1H), 7.44-7.27 (m, 1H), 3.87 (s, 3H), 3.55-3.47 (m, 4H), 3.26- 3.16 (m, 2H), 3.08 (s, 2H), 2.99-2.87 (m, 2H), 2.19-2.08 (m, 2H), 2.06- 1.94 (m, 2H), 1.47 (s, 6H). |
| B2 | 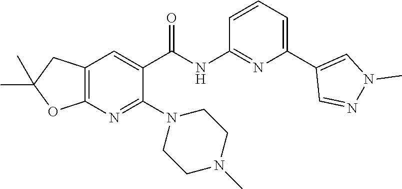 | BB | ¹H NMR (300 MHz, DMSO-d₆) δ 11.96 (s, 1H), 8.26 (s, 1H), 8.16 (s, 1H), 8.70-7.95 (m, 2H), 7.82-7.75 (t, J = 7.5 Hz, 1H), 7.38 (d, J = 7.5 Hz, 1H), 3.89 (s, 3H), 3.73 (s, 2H), 3.15- 2.95 (m, 4H), 2.75-2.55 (m, 4H), 2.27 (s, 3H), 1.47 (s, 6H). LC-MS (m/z): 447.8 [M + H]⁺. |
| B3 | 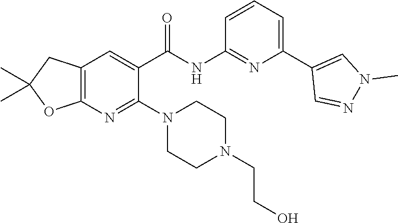 | BB | ¹H NMR (300 MHZ, DMSO-d₆) δ 12.01 (s, 1H), 8.27 (s, 1H), 8.16 (s, 1H), 8 8.10-7.99 (m, 2H), 7.85-7.74 (m, 1H), 7.44-7.35 (m, 1H), 4.48 (s, 1H), 3.89 (s, 3H), 3.61-3.45 (m, 4H), 3.21-2.99 (m, 6H), 2.87-2.70 (m, 4H), 1.57-1.38 (m, 6H). LC-MS (m/z): 477.8 [M + H]⁺. |
| B4 | 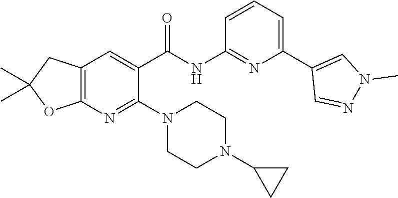 | BB | ¹H NMR (300 MHZ, DMSO-d₆) δ 11.99 (s, 1H), 8.27 (s, 1H), 8.15 (s, 1H), 8.08-7.98 (m, 2H), 7.79 (s, 1H), 7.46-7.28 (m, 1H), 3.87 (s, 3H), 3.11- 2.95 (m, 6H), 2.93-2.80 (m, 4H), 1.72-1.60 (m, 1H), 1.46 (s, 6H), 0.54- 0.42 (m, 2H), 0.38-0.28 (m, 2H). LC-MS (m/z): 473.9 [M + H]⁺. |
| B5 | 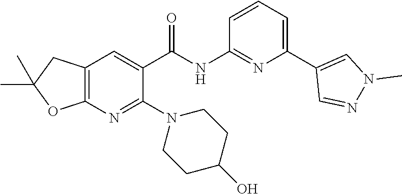 | BB | ¹H NMR (300 MHZ, DMSO-d₆) δ 12.80 (s, 1H), 8.40 (s, 1H), 8.22 (s, 1H), 8.05 (s, 1H), 8.02 (d, J = 7.8 Hz, 1H), 7.78 (t, J = 8.1 Hz, 1H), 7.38 (d, J = 7.5 Hz, 1H), 5.11-5.06 (m, 1H), 3.86 (s, 3H), 3.74 (s, 2H), 3.25- 3.15(m, 2H), 3.08 (s, 1H), 3.02-2.85 (m, 2H), 2.05-1.85 (m, 4H), 1.47 (s, 6H). LC-MS (m/z): 448.8 [M + H]⁺. |
| B6 | 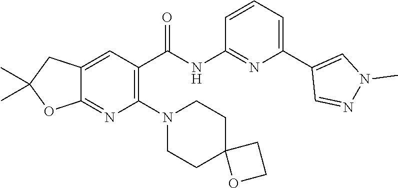 | BB | ¹H NMR (300 MHZ, DMSO-d₆) δ 11.46 (s, 1H), 8.13 (s, 1H), 8.06 (s, 1H), 8.01 (d, J = 8.4 Hz, 1H), 7.92 (s, 1H), 7.77 (t, J = 7.8 Hz, 1H), 7.36 (d, J = 7.5 Hz, 1H), 5.60-5.50 (m, 1H), 4.48-4.36 (m, 1H), 3.89 (s, 3H), 3.70- 3.60 (m, 2H), 3.59-3.45 (m, 2H), 3.25-3.20 (m, 2H), 3.04 (s, 2H), 2.35- 2.25 (m, 2H), 2.23-2.12 (m, 2H), 1.52-1.38 (m, 6H).LC-MS (m/z): 474.9 [M + H]⁺. |
| B7 | 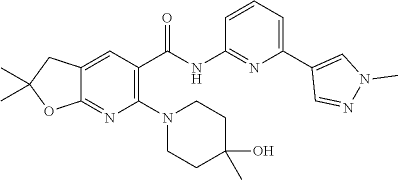 | BB | ¹H NMR (300 MHZ, DMSO-d₆) δ 12.44 (s, 1H), 8.33 (s, 1H), 8.19 (s, 1H), 8.04 (d, J = 8.4 Hz, 1H), 7.99 (s, 1H), 7.78 (t, J = 7.5 Hz, 1H), 7.37 (d, J = 7.5 Hz, 1H), 4.54 (s, 1H), 3.87 (s, 3H), 3.26-3.13 (m, 2H), 3.07 (s, 2H), 3.03-2.93 (m, 2H), 1.92-1.72 (m, 4H), 1.53-1.40 (m, 6H), 1.22 (s, 3H). LC-MS (m/z): 462.9 [M + H]⁺ |
| B8 | 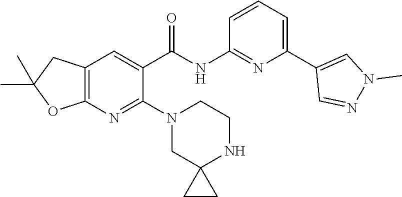 | BB | ¹H NMR (300 MHZ, DMSO-d₆) δ 12.11 (s, 1H), 8.22 (s, 1H), 8.17 (s, 1H), 8.06 (d, J = 6.6 Hz, 1H), 7.98 (s, 1H), 7.81-7.72 (m, 1H), 7.37 (d, J = 6.6 Hz, 1H), 3.89 (s, 3H), 3.21-3.07 (m, 6H), 3.01 (s, 2H), 1.57-1.39 (m, 6H), 0.67-0.48 (m, 4H). LC-MS (m/z): 459.8 [M + H]⁺. |
| B9 | 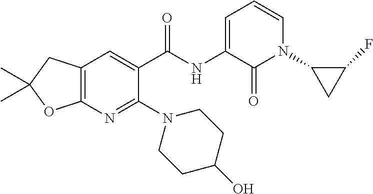 [SMILES: CC1(C)Cc2cc(C(=O)Nc3cccn([C@H]4C[C@H]4F)c3=O)c(N3CCC(O)CC3)nc2O1] | BB, BD | ¹H NMR (300 MHz, DMSO-d₆) δ 11.49 (s, 1H), 8.45 (d, J = 6.0 Hz, 1H), 8.05 (s, 1H), 7.37 (d, J = 6.6 Hz, 1H), 6.33-6.26 (m, 1H), 5.01 (d, J = 64.8 Hz, 1H), 4.67-4.57 (m, 1H), 3.76- 3.62 (m, 1H), 3.25-3.16 (m, 3H), 3.03 (s, 2H), 2.95-2.81 (m, 2H), 1.93- 1.84 (m, 2H), 1.78-1.60 (m, 2H), 1.45 (s, 6H), 1.28-1.14 (m, 2H). LC-MS (m/z): 442.8 [M + H]⁺. |
| B10 | 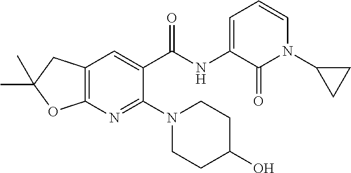 [SMILES: CC1(C)Cc2cc(C(=O)Nc3cccn(C4CC4)c3=O)c(N3CCC(O)CC3)nc2O1] | BB, BE | ¹H NMR (300 MHZ, DMSO-d₆) δ 11.44 (s, 1H), 8.41 (d, J = 6.6 Hz, 1H), 8.04 (s, 1H), 7.26 (d, J = 6.0 Hz, 1H), 6.39-6.15 (m, 1H), 4.70-4.55 (m, 1H), 3.73-3.61 (m, 1H), 3.49-3.42 (m, 1H), 3.28-3.14 (m, 2H), 3.03 (s, 2H), 2.94-2.82 (m, 2H), 2.01-1.79 (m, 2H), 1.77-1.62 (m, 2H), 1.45 (s, 6H), 1.11-0.93 (m, 2H), 0.96-0.83 (m, 2H). LC-MS (m/z): 424.9 [M + H]⁺. |
| B11 | 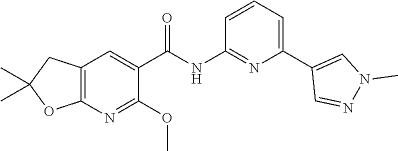 | BC | ¹H NMR (300 MHZ, DMSO-d₆) δ 10.20 (s, 1H), 8.28 (s, 1H), 8.21 (s, 1H), 8.07-7.96 (m, 2H), 7.79 (t, J = 7.8 Hz, 1H), 7.40 (d, J = 7.2 Hz, 1H), 4.09 (s, 3H), 3.90 (s, 3H), 3.06 (s, 2H), 1.48 (s, 6H). LC-MS (m/z): 379.9 [M + H]⁺. |
| B12 | 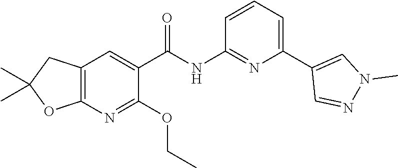 | BC | ¹H NMR (300 MHZ, DMSO-d₆) δ 10.45 (s, 1H), 8.22 (s, 1H), 8.21 (s, 1H), 8.02 (d, J = 8.1 Hz, 1H), 7.94 (s, 1H), 7.77 (t, J = 7.8 Hz, 1H), 7.38 (d, J = 7.5 Hz, 1H), 4.51 (q, J = 6.6 Hz, 2H), 3.89 (s, 3H), 3.06 (s, 2H), 1.55 (t, J = 6.6 Hz, 3H), 1.48 (s, 6H). LC-MS (m/z): 393.8 [M + H]⁺. |
| B13 | 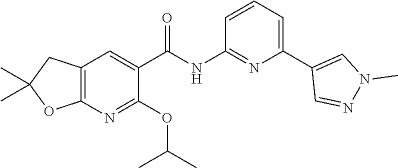 | BC | ¹H NMR (300 MHZ, DMSO-d₆) δ 10.56 (s, 1H), 8.24 (s, 1H), 8.20 (s, 1H), 8.02 (d, J = 6.3 Hz, 1H), 7.93 (s, 1H), 7.79 (t, J = 4.8 Hz, 1H), 7.38 (d, J = 7.8 Hz, 1H), 5.46-5.29 (m, 1H), 3.90 (s, 3H), 3.05 (s, 2H), 1.66-1.34 (m, 12H). LC-MS (m/z): 407.9 [M + H]⁺. |
| B14 | 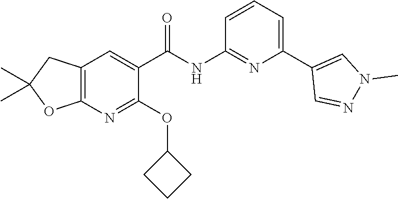 | BC | ¹H NMR (300 MHZ, DMSO-d₆) δ 10.51 (s, 1H), 8.22 (s, 1H), 8.21 (s, 1H), 8.02 (d, J = 8.1 Hz, 1H), 7.93 (s, 1H), 7.80 (t, J = 7.8 Hz, 1H), 7.42- 7.36 (m, 1H), 5.35-5.22 (m, 1H), 3.89 (s, 3H), 3.04 (s, 2H), 2.63-2.53 (m, 2H), 2.36-2.26 (m, 2H), 1.99-1.86 (m, 1H), 1.82-1.68 (m, 1H), 1.47 (s, 6H). LC-MS (m/z): 419.9 [M + H]⁺. |
| B15 | 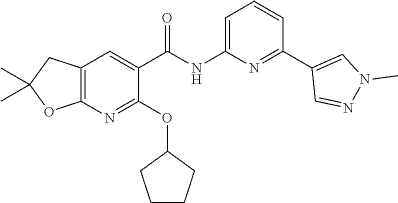 | BC | ¹H NMR (300 MHZ, DMSO-d₆) δ 10.43 (s, 1H), 8.23 (s, 1H), 8.18 (s, 1H), 8.04 (d, J = 8.1 Hz, 1H), 7.92 (s, 1H), 7.79 (t, J = 8.1 Hz, 1H), 7.38 (d, J = 6.9 Hz, 1H), 5.67-5.61 (m, 1H), 3.89 (s, 3H), 3.05 (s, 2H), 2.09-1.88 (m, 6H), 1.78-1.63 (m, 2H), 1.48 (s, 6H). LC-MS (m/z): 433.9 [M + H]⁺. |
| B16 | 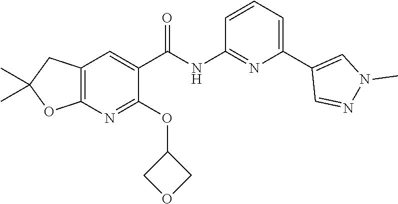 | BC | ¹H NMR (300 MHZ, DMSO-d₆) δ 10.36 (s, 1H), 8.22 (s, 1H), 8.21 (s, 1H), 8.02 (d, J = 7.5 Hz, 1H), 7.94 (s, 1H), 7.81 (t, J = 4.8 Hz, 1H), 7.40 (d, J = 7.8 Hz, 1H), 5.77-5.61 (m, 1H), 5.10-4.91 (m, 2H), 4.82-4.69 (m, 2H), 3.89 (s, 3H), 3.06 (s, 2H), 1.47 (s, 6H). LC-MS (m/z): 421.8 [M + H]⁺. |
| B17 | 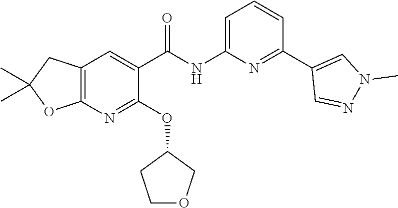 | BC | ¹H NMR (300 MHZ, DMSO-d₆) δ 10.39 (s, 1H), 8.23 (s, 1H), 8.20 (s, 1H), 8.00 (d, J = 8.1 Hz, 1H), 7.95 (s, 1H), 7.79 (t, J = 8.1 Hz, 1H), 7.38 (d, J = 7.8 Hz, 1H), 5.78-5.70 (m, 1H), 4.17-3.92 (m, 4H), 3.89 (s, 3H), 3.06 (s, 2H), 2.39-2.19 (m, 2H), 1.48 (s, 6H). LC-MS (m/z): 435.8 [M + H]⁺. |
| B18 | 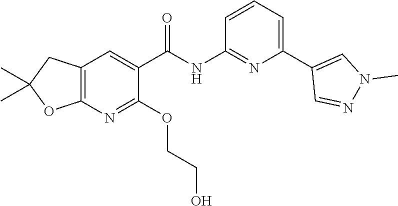 | BC | ¹H NMR (300 MHZ, DMSO-d₆) δ 10.46 (s, 1H), 8.24 (s, 1H), 8.22 (s, 1H), 8.02 (s, 1H), 8.01 (d, J = 6.0 Hz, 1H), 7.78 (t, J = 7.8 Hz, 1H), 7.38 (d, J = 7.5 Hz, 1H), 5.04-4.91 (m, 1H), 4.55-4.45 (m, 2H), 4.12-3.74 (m, 5H), 3.06 (s, 2H), 1.48 (s, 6H). LC-MS (m/z): 409.8 [M + H]⁺. |
| B19 | 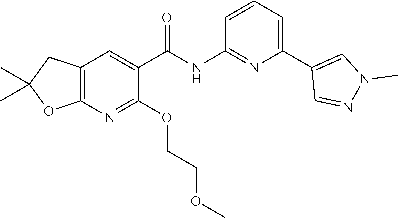 | BC | ¹H NMR (300 MHZ, DMSO-d₆) δ 10.32 (s, 1H), 8.24 (s, 1H), 8.23 (s, 1H), 8.02 (d, J = 5.7 Hz, 1H), 7.99 (s, 1H), 7.79 (t, J = 7.8 Hz, 1H), 7.38 (d, J = 7.8 Hz, 1H), 4.63-4.54 (m, 2H), 3.92-3.81 (m, 5H), 3.30 (s, 3H), 3.06 (s, 2H), 1.48 (s, 6H). LC-MS (m/z): 423.9 [M + H]⁺. |
| B20 | 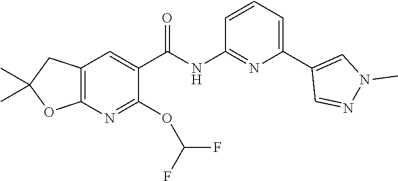 | BF | ¹H NMR (300 MHZ, DMSO-d₆) δ 10.22 (s, 1H), 8.18 (s, 1H), 8.06 (s, 1H), 7.99-7.93 (m, 1H), 7.93 (s, 1H), 7.80 (t, J = 8.1 Hz, 1H), 7.75 (d, J = 71.7 Hz, 1H), 7.40 (d, J = 7.8 Hz, 1H), 3.89 (s, 3H), 3.09 (s, 2H), 1.49 (s, 6H). LC-MS (m/z): 415.8 [M + H]⁺. |
| B21 | 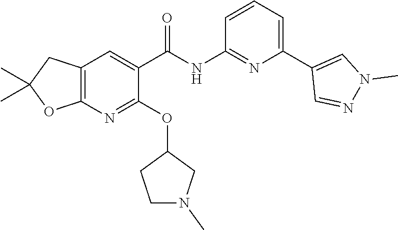 | BC | ¹H NMR (300 MHZ, CDCl₃) δ 9.93 (s, 1H), 8.31 (s, 1H), 8.15 (d, J = 8.1 Hz, 1H), 7.85 (s, 1H), 7.70 (t, J = 7.8 Hz, 1H), 7.24 (s, 1H), 7.22-7.18 (m, 1H), 5.93-5.85 (m, 1H), 3.99 (s, 3H), 3.06 (s, 2H), 2.91 (s, 3H), 2.75-2.62 (m, 2H), 2.18-2.15 (m, 2H), 1.86-1.75 (m, 2H), 1.56 (s, 6H). LC-MS (m/z): 448.9 [M + H]⁺. |
| B22 | 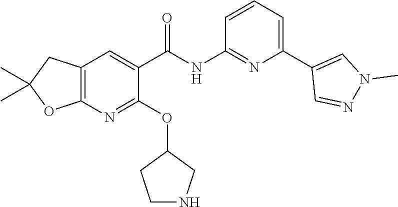 | BC, BG | ¹H NMR (300 MHZ, DMSO-d₆) δ 10.20 (s, 1H), 8.27-8.17 (m, 2H), 8.02 (d, J = 7.5 Hz, 1H), 7.95 (s, 1H), 7.88-7.75 (m, 1H), 7.40 (d, J = 6.9 Hz, 1H), 5.68-5.52 (m, 1H), 3.91 (s, 3H), 3.28-3.18 (m, 5H), 3.07 (s, 2H), 2.43-2.36 (m, 2H), 1.49 (s, 6H). LC- MS (m/z): 434.9 [M + H]⁺. |
| B23 | 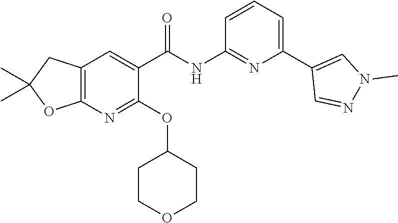 | BC | ¹H NMR (300 MHZ, DMSO-d₆) δ 10.46 (s, 1H), 8.25 (s, 1H), 8.20 (s, 1H), 8.05 (d, J = 8.1 Hz, 1H), 7.95 (s, 1H), 7.87-7.72 (m, 1H), 7.40 (d, J = 7.5 Hz, 1H), 5.63-5.46 (m, 1H), 4.15- 3.97 (m, 2H), 3.88 (s, 3H), 3.76- 3.62 (m, 2H), 3.06 (s, 2H), 2.18-2.05 (m, 2H), 1.94-1.77 (m, 2H), 1.48 (s, 6H). LC-MS (m/z): 449.8 [M + H]⁺. |
| B24 | 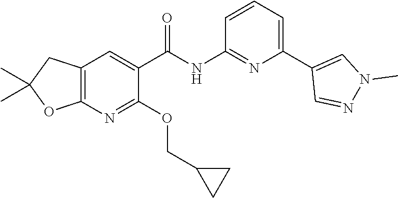 | BC | ¹H NMR (300 MHZ, DMSO-d₆) δ 10.47 (s, 1H), 8.24 (s, 1H), 8.20 (s, 1H), 8.05 (d, J = 8.1 Hz, 1H), 7.94 (s, 1H), 7.79 (t, J = 7.8 Hz, 1H), 7.38 (d, J = 7.5 Hz, 1H), 4.34 (d, J = 4.2 Hz, 2H), 3.88 (s, 3H), 3.05 (s, 2H), 1.47 (s, 6H), 1.45-1.37 (m, 1H), 0.75-0.60 (m, 2H), 0.59-0.47 (m, 2H). LC-MS (m/z): 419.8 [M + H]⁺. |
| B25 | 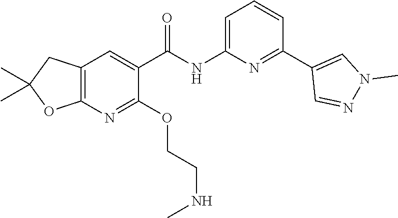 | BC, BG | ¹H NMR (300 MHZ, DMSO-d₆) δ 10.31 (s, 1H), 8.26 (s, 1H), 8.17 (s, 1H), 8.06-7.93 (m, 2H), 7.79 (t, J = 8.4 Hz, 1H), 7.38 (d, J = 7.5 Hz, 1H), 4.63 (t, J = 4.5 Hz, 2H), 3.89 (s, 3H), 3.58 (t, J = 4.5 Hz, 2H), 3.06 (s, 2H), 2.54 (s, 3H), 1.48 (s, 6H). LC-MS (m/z): 422.9 [M + H]⁺. |
| B26 | 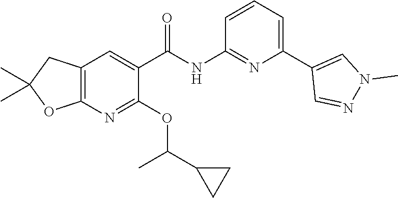 | BC | ¹H NMR (300 MHz, DMSO-d₆) δ 10.57 (s, 1H), 8.24 (s, 1H), 8.17 (s, 1H), 8.04 (d, J = 7.8 Hz, 1H), 7.91 (s, 1H), 7.79 (t, J = 7.8 Hz, 1H), 7.38 (d, J = 7.5 Hz, 1H), 5.00-4.79 (m, 1H), 3.89 (s, 3H), 3.05 (s, 2H), 1.51-1.47 (m, 9H), 1.40-1.30 (m, 1H), 0.75- 0.32 (m, 4H). LC-MS (m/z): 433.8 [M + H]⁺. |
| B27 | 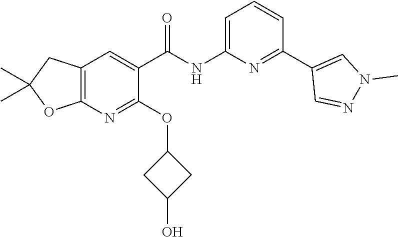 | BC, BH | ¹H NMR (300 MHZ, DMSO-d₆) δ 10.54 (s, 1H), 8.26 (s, 1H), 8.21 (s, 1H), 8.08-7.91 (m, 2H), 7.81 (t, J = 7.8 Hz, 1H), 7.40 (d, J = 7.5 Hz, 1H), 5.45-5.35 (m, 1H), 4.96-4.86 (m, 1H), 4.04-3.94 (m, 1H), 3.88 (s, 3H), 3.05 (s, 2H), 2.99-2.87 (m, 2H), 2.26- 2.12 (m, 2H), 1.47 (s, 6H). LC-MS (m/z): 435.8 [M + H]⁺. |
| B28 | 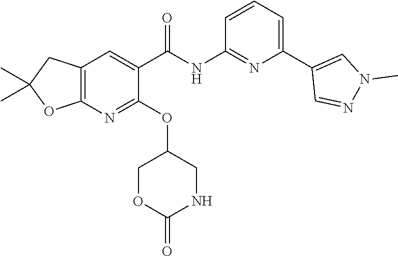 | BC, BG | ¹H NMR (300 MHZ, DMSO-d₆) δ 12.10 (s, 1H), 8.40 (s, 1H), 8.24 (s, 1H), 8.08 (d, J = 8.1 Hz, 1H), 7.95 (s, 1H), 7.75 (t, J = 7.8 Hz, 1H), 7.65 (s, 1H), 7.35 (s, 1H), 5.00-4.84 (m, 1H), 4.29-4.20 (m, 2H), 3.89 (s, 3H), 3.74- 3.45 (m, 2H), 3.07 (s, 2H), 1.56 (s, 6H). |
| B29 | 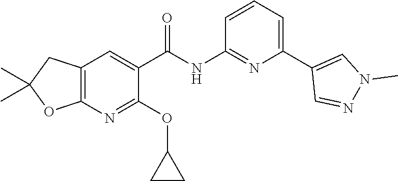 | BC | ¹H NMR (300 MHZ, DMSO-d₆) δ 10.14 (s, 1H), 8.29-8.17 (m, 2H), 7.99 (d, J = 8.4 Hz, 1H), 7.94 (s, 1H), 7.85-7.73 (m, 1H), 7.38 (d, J = 7.1 Hz, 1H), 4.56-4.45 (m, 1H), 3.91 (s, 3H), 3.07 (s, 2H), 1.49 (s, 6H), 1.03- 0.86 (m, 4H). LC-MS (m/z): 405.8 [M + H]⁺. |
| B30 | 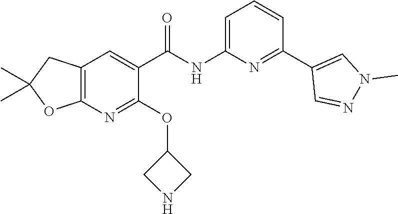 | BC, BH | ¹H NMR (300 MHz, DMSO-d₆) δ 10.36 (s, 1H), 8.13 (s, 1H), 8.07 (s, 1H), 7.95-7.87 (m, 2H), 7.72 (t, J = 8.1 Hz, 1H), 7.30 (d, J = 7.8 Hz, 1H), 5.38-5.23 (m, 1H), 3.90-3.81 (m, 2H), 3.78 (s, 3H), 3.56-3.43 (m, 2H), 3.41-3.38 (m, 1H), 3.03 (s, 2H), 1.47 (s, 6H). LC-MS (m/z): 420.9 [M + H]⁺. |
| B31 | 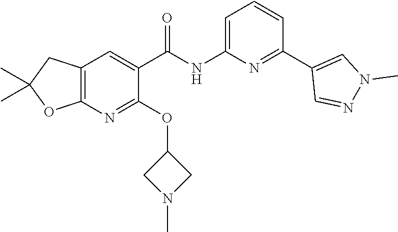 | BI | ¹H NMR (300 MHz, DMSO-d₆) δ 10.41 (s, 1H), 8.25-8.19 (m, 2H), 8.01 (d, J = 6.6 Hz, 1H), 7.96 (s, 1H), 7.81 (t, J = 7.1 Hz, 1H), 7.40 (d, J = 7.1 Hz, 1H), 5.33-5.23 (m, 1H), 3.89 (s, 3H), 3.83-3.73 (m, 2H), 3.25- 3.15 (m, 2H), 3.05 (s, 2H), 2.35 (s, 3H), 1.47 (s, 6H). LC-MS (m/z): 434.9 [M + H]⁺. |
| B32 | 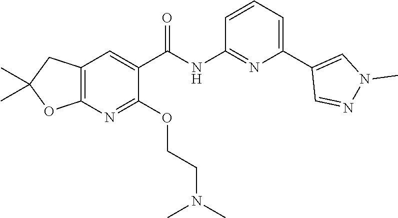 | BC | ¹H NMR (300 MHz, CDCl₃) 8 10.43 (s, 1H), 8.33 (s, 1H), 8.15 (d, J = 8.7 Hz, 1H), 7.97 (s, 1H), 7.86 (s, 1H), 7.68 (t, J = 7.5 Hz, 1H), 7.18 (d, J = 7.8 Hz, 1H), 4.66 (s, 2H), 3.96 (s, 3H), 3.11-3.02 (m, 2H), 3.02-2.92 (m, 2H), 2.39 (s, 6H), 1.58 (s, 6H). |
| B33 | 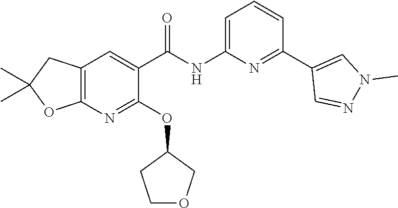 | BC | ¹H NMR (300 MHZ, DMSO-d₆) δ 10.38 (s, 1H), 8.23 (s, 1H), 8.20 (s, 1H), 8.00 (d, J = 8.4 Hz, 1H), 7.95 (s, 1H), 7.79 (t, J = 7.5 Hz, 1H), 7.38 (d, J = 6.9 Hz, 1H), 5.78-5.69 (m, 1H), 4.22-3.91 (m, 4H), 3.89 (s, 3H), 3.06 (s, 2H), 2.33-2.17 (m, 2H), 1.48 (s, 6H). LC-MS (m/z): 435.8 [M + H]⁺. |
| B34 | 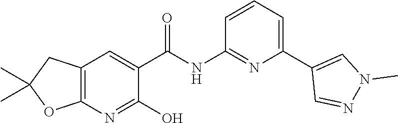 | BF | ¹H NMR (300 MHZ, DMSO-d₆) δ 12.16 (s, 1H), 8.28 (s, 1H), 8.22 (s, 1H), 8.07 (d, J = 5.1 Hz, 1H), 7.94 (s, 1H), 7.73 (t, J = 8.1 Hz, 1H), 7.32 (d, J = 7.5 Hz, 1H), 3.88 (s, 3H), 2.96 (s, 2H), 1.51 (s, 6H). |
| B35 | 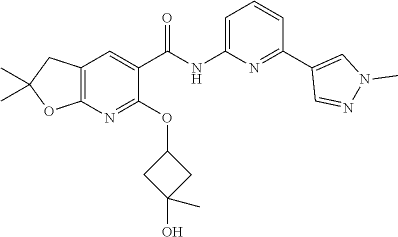 | BC, BJ | ¹H NMR (300 MHZ, DMSO-d₆) δ 10.50 (s, 1H), 8.26 (s, 1H), 8.22 (s, 1H), 8.06-7.94 (m, 2H), 7.80 (t, J = 7.8 Hz, 1H), 7.39 (d, J = 7.5 Hz, 1H), 5.35 (s, 1H), 5.04-4.95 (m, 1H), 3.89 (s, 3H), 3.05 (s, 2H), 2.69-2.57 (m, 2H), 2.42-2.31 (m, 2H), 1.47 (s, 6H), 1.33 (s, 3H). LC-MS (m/z): 449.8 [M + H]⁺. |
| B36 | 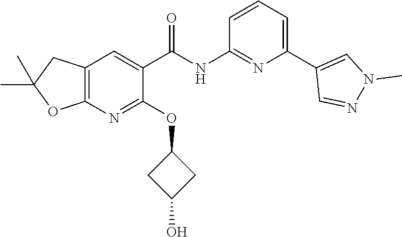 | BC, BH, BK | ¹H NMR (300 MHZ, DMSO-d₆) δ 10.50 (s, 1H), 8.26-8.16 (m, 2H), 8.05-7.98 (m, 1H), 7.94 (s, 1H), 7.84- 7.76 (m, 1H), 7.44-7.34 (m, 1H), 5.51-5.42 (m, 1H), 5.38-5.28 (m, 1H), 4.67-4.56 (m, 1H), 3.88 (s, 3H), 3.05 (s, 2H), 2.68-2.55 (m, 4H), 1.47 (s, 6H). |

### Example 95. Screening the Compounds Against IRAK4 with ATP

Prepare Ix kinase base buffer and stop buffer for testing kinases: (1) base buffer: 50 mM HEPES (pH 7.5), 0.0015% Brij-35; (2) stop buffer: 100 mM HEPES (pH 7.5), 0.015% Brij-35, 0.2% Coating Reagent #3, 50 mM EDTA. Then prepare compounds for testing: Dilute the compound to 50× of the final desired highest inhibitor concentration in reaction by 100% DMSO. Transfer 100 μL of this compound dilution to a well in a 96-well plate (source plate).

Serially dilute the compounds by 3-fold for a total of 10 points. Transfer 10 μL of the compound from the source plate to a new 96-well plate (intermediate plate) and add 90 μL of Ix kinase buffer. Shake the mixture on the intermediate plate for 10 min. Transfer 5 μL of each well from the 96-well intermediate plate to a 384-well plate in duplicates. Add 10 μL of 2.5× enzyme solution to each well of the 384-well assay plate and incubate at room temperature for 10 min. Add 10 μL, of 2.5×FAM-labeled peptide and ATP solution and incubate at 28° C. for a period of time. Finally stop the reaction by adding 30 μL of stop buffer and collect data on Caliper to obtain the IC₅₀.

TABLE 2
Results of selected compounds of IC₅₀ against IRAK4 kinase

| | IC₅₀ | | IC₅₀ | | IC₅₀ | | IC₅₀ |
|---|---|---|---|---|---|---|---|
| | (nM) | | (nM) | | (nM) | | (nM) |
| | (10 μM | | (10 μM | | (10 μM | | (10 μM |
| NO. | ATP) | NO. | ATP) | NO. | ATP) | NO. | ATP) |
| A1 | 405 | A2 | 45 | A3 | 43 | A4 | 11 |
| A6 | 194 | A10 | 122 | A11 | 22 | A12 | 33 |
| A13 | 304 | A14 | 164 | A15 | 244 | A16 | 31 |
| A18 | 221 | A21 | 26 | A22 | 188 | A23 | 425 |
| A24 | 4.1 | A25 | 8.6 | A26 | 4.2 | A27 | 81 |
| A28 | 80 | A30 | 125 | A34 | 40 | A35 | 8.9 |
| A36 | 17 | A37 | 462 | A42 | 87 | A43 | 137 |
| A45 | 131 | A46 | 179 | A49 | >6000 | A50 | 98 |
| A51 | 7.0 | A52 | 5.1 | A54 | 21 | A55 | 5.9 |
| A56 | 7.8 | A57 | 5.1 | A58 | 4.9 | A59 | 6.6 |
| B1 | 4.5 | B2 | 54 | B3 | 31 | B4 | 7.5 |
| B5 | 7.6 | B6 | 5.9 | B7 | 11 | B8 | 224 |
| B9 | 69 | B10 | 479 | B11 | 50 | B12 | 9.2 |
| B13 | 6.2 | B14 | 6.1 | B15 | 5.6 | B16 | 11 |
| B17 | 6.5 | B18 | 22 | B19 | 18 | B20 | 27 |
| B21 | 75 | B22 | 354 | B23 | 11 | B24 | 11 |
| B26 | 13 | B27 | 7.3 | B28 | 248 | B29 | 6.2 |
| B30 | 170 | B31 | 176 | B32 | 236 | B33 | 8.9 |
| B34 | 243 | B35 | 6.8 | B36 | 4.5 | CA4948 | 115 |

CA4948 (emavusertib):

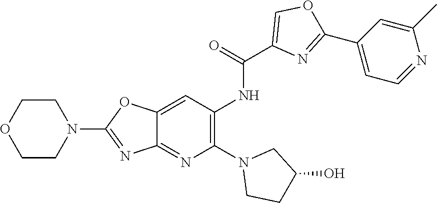 [SMILES: Cc1cc(-c2nc(C(=O)Nc3cc4oc(N5CCOCC5)nc4nc3N3CC[C@@H](O)C3)co2)ccn1]

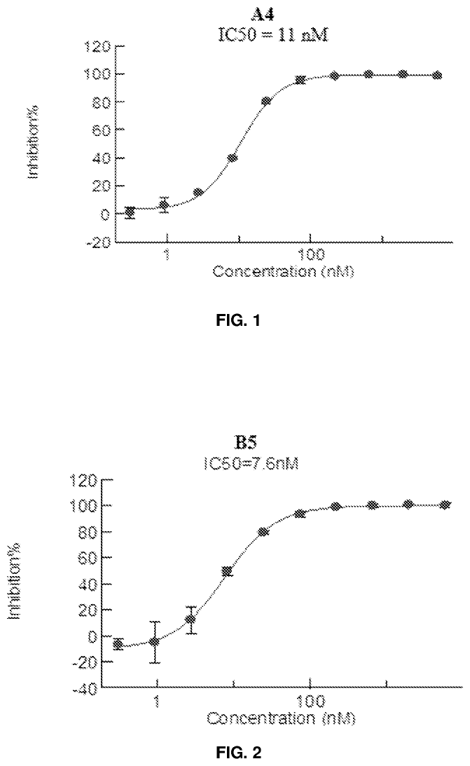

The results of the enzyme activity experiments showed that the compounds of the present disclosure displayed the unexpected inhibitory effect on IRAK4 kinase. As shown, the activities of Compounds A24, A26, B1, and B36 are more than 25 times better than that of the positive control compound CA4948 (see, ACS Med. Chem. Lett. 2020, 11, 2374-2381). The activities of Compounds A4, A25, A35, A51-A52, A55-A59, B4-B7, B12-B17, B23-B24, B26-B27, B29, B33, and B35 are more than 10 times better than that of the control compound CA4948. The activities of Compounds A11, A36, A54, and B18-B19 are more than 5 times better than that of CA4948. FIG. 1 shows the experimental results of the inhibition of IRAK4 kinase by Compound A4. FIG. 2 shows the experimental results of the inhibition of IRAK4 kinase by Compound B5.

### Example 96. Kinase Binding Ability

The Equilibrium dissociation constant reflected the test compound's kinase binding (K_{d}). The KINOMEscan, a site-directed competition binding assay, was used to measure interactions between test compounds and specific kinases. The kinase-tagged T7 phage strains were derived from the BL21 strain's E. coli host. T7 phage was used to infect E. coli, which was then incubated until lysis occurred. The remaining kinases were produced in HEK-293 cells and tagged with DNA. To create affinity resins for kinase, streptavidin-coated magnetic beads were treated with biotinylated small molecule ligands. The assay consists of three components: a DNA-tagged kinase, an immobilized ligand, and the test compound. An immobilized ligand could compete with the compound binding the kinase active site. The affinity beads were washed with wash buffer and re-suspended in elution buffer following reactions in a polypropylene 384-well plate. qPCR of the kinase-DNA tag was used to assess the test compound's ability to bind with the selected kinase. The assay was carried out in accordance with the manufacturer's instructions (Eurofins DiscoverX Corporation).

Binding constants (K_{d}) were calculated with a standard dose-response curve using the Hill equation:

$$Response = Background + \frac{Signal - Background}{1 + ( K \text{?} / Dos \text{?} )}$$

$$\text{?} \text{indicates text missing or illegible when filed}$$

TABLE 3
K_{d} between IRAK4 and selected compounds

| Cmpd. NO. | K_{d} (nM) |
|---|---|
| A1 | 29 |
| A4 | 0.59 |
| B5 | 0.69 |

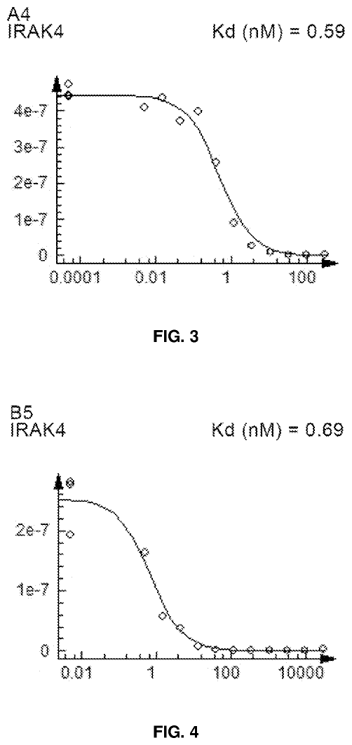

The results of the binding assay showed that Compounds A1, A4, and B5 all have high binding affinities to IRAK4; and that the K_{d} of Compounds A4 and B5 with IRAK4 is less than 1 nM. FIG. 3 shows the experimental results of the kinase binding assay for Compound A4, while FIG. 4 shows the experimental results of the kinase binding assay for Compound B5.

### Example 97. Assessment of Metabolic Stability in Microsomes

The appropriate concentrations of microsomes working solutions (0.56 mg/mL) were prepared in potassium phosphate buffer. The test compounds were incubated with Human, SD Rat, CD-1 Mouse and Beagle Dog liver microsomes for 10 min at 37° C. with constant shaking. The reaction was initiated by the addition of nicotinamide adenine dinucleotide phosphate (NADPH) cofactor after a 10 min pre-incubation. At 5, 15, 30, 45, and 60 min, the samples were taken and placed into the stop solution (Cold acetonitrile containing 200 ng/mL tolbutamide and 200 ng/mL labetalol as internal standards). All sampling plates were shaken for 10 min, then centrifuged at 4000 rpm for 20 minutes at 4° C. Each supernatant of bioanalysis plate was sealed and shaken for 10 minutes prior to LC-MS analysis.

TABLE 4
Metabolic stability of selected compounds
with four species liver microsomes

| | Human | | Rat | | Mouse | | Dog | |
|---|---|---|---|---|---|---|---|---|
| Cmpd. | microsome | | microsome | | microsome | | microsome | |
| NO. | (t_{1/2}) | | (t_{1/2}) | | (t_{1/2}) | | (t_{1/2}) | |
| A1 | >145 | min | >145 | min | >145 | min | >145 | min |
| A4 | >145 | min | 92 | min | >145 | min | 102 | min |
| A24 | 117 | min | 76 | min | 89 | min | 68 | min |
| B5 | >145 | min | >145 | min | 71 | min | >145 | min |

The results of the metabolic stability experiments showed that (1) Compounds A1 and A4 have good metabolic stability in the liver microsomes of human, rat, mouse and dog; that (2) Compound A24 has good metabolism in the liver microsomes of human and mouse, and moderate metabolic stability in liver microsomes of rat and dog; and that (3) Compound B5 has good metabolic stability in liver microsomes of human, rat and dog, and moderate metabolic stability in mice liver microsomes.

### Example 98. Pharmacokinetics Study in ICR Mice

Solution used:10% DMSO+10% Solutol+80% (20% Hp-β-CD)

Single dose pharmacokinetic parameters were determined at Medicilon using male ICR mice (3 rats per group, 2 groups). Groups were dosed i.v. (2 mg/kg) or p.o. (10 mg/kg) and blood samples were taken via submandibular or saphenous vein at the following time points post dosing: 0.083 (i.v. only), 0.25, 0.5, 1, 2, 4, 6 (p.o. only), 8 and 24 h. An aliquot of 50 μL of blood plasma was protein precipitated with 250 μL internal standard solution (200 ng/mL tolbutamide in MeOH), the mixture was vortex-mixed for 1 min and centrifuged at 14,000 rpm for 5 min. The concentration of the test compound in the plasma samples was determined using LC-MS/MS with the peak areas compared to a calibration curve determined using 5-5000 ng/mL test compound in ICR mice plasma. The results are shown in Table 5. Area under the curve (AUC) is the definite integral of the plasma concentration of a drug as a function of time from time zero to 24 hours. Cₘₐₓ represents peak plasma concentration of the drug; Tₘₐₓ represents the time to reach Cₘₐₓ; T_{1/2} represents elimination half-life or the time taken for Cₘₐₓ to drop in half; Cl represents clearance; V_{d} represents apparent volume of distribution; F represents bioavailability; p.o. represents oral administration; and i.v. represents intravenous injection.

TABLE 5
PK profiles of selected compounds in ICR mice

| | | |
|---|---|---|
| Cmpd. NO. | A4 | B5 |

| | | | |
|---|---|---|---|
| i.v.: 2 mg/kg | Cl (mL/kg/min) | 8.1 | 13.6 |
| | V_{d} (L/kg) | 0.98 | 3.6 |
| | AUC(ng · h/mL) | 4051 | 2452 |
| | T_{1/2} (h) | 1.4 | 3.1 |
| p.o.: 10 mg/kg | Cₘₐₓ (ng/mL) | 8584 | 1457 |
| | Tₘₐₓ (h) | 1.17 | 2.0 |
| | AUC (ng · h/mL) | 26417 | 9992 |
| | F (%) | 130 | 91 |

The results of in vivo pharmacokinetic experiments showed that when administered intravenously to mice, compounds A4 and B5 showed a low clearance rate and a good half-life. These results indicate that compounds A4 and B5 were well metabolized in mice. After oral administration, these compounds showed extremely high oral exposure and bioavailability. These results indicate that compounds A4 and B5 are well absorbed orally in mice.

1. A compound of Formula (I): 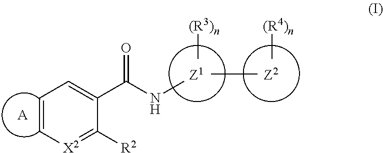
or a pharmaceutically acceptable salt, ester, solvate, prodrug, isotope-labeled derivative, or isomer thereof, wherein:
ring A is selected from the group consisting of: 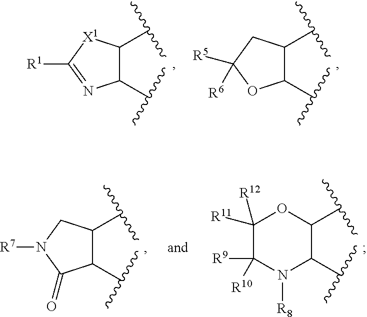
each n is independently 0, 1, 2, or 3;
X¹ is O or S;
X² is N or CH;
if present, R¹ is hydrogen, C₁₋₆ alkyl, C₃₋₆ cycloalkyl, C₁₋₂ alkylene-C₃₋₆ cycloalkyl, —NH(C₁₋₆ alkyl), —N(C₁₋₆ alkyl)₂, —NH(C₃₋₆ cycloalkyl), —N(C₃₋₆ cycloalkyl)₂, —OC₁₋₆ alkyl, 3-8 membered heterocyclyl, 6-10 membered bridged biheterocyclyl, 5-12 membered spiro biheterocyclyl, 6-12 membered fused biheterocyclyl, C₆₋₁₀ aryl, or 5-10 membered heteroaryl, wherein each of C₁₋₆ alkyl, C₃₋₆ cycloalkyl, aryl, heteroaryl, heterocyclyl, bridged biheterocyclyl, spiro biheterocyclyl, and fused biheterocyclyl are substituted with 1, 2 or 3 R^{a} independently; if present, each of heterocyclyl, bridged biheterocyclyl, spiro biheterocyclyl, fused biheterocyclyl, and heteroaryl comprises one or more heteroatoms independently selected from the group consisting of O, S, NH, N, P(═O), S(═O) and S(═O)₂;
R² is hydrogen, C₁₋₆ alkyl, C₃₋₆ cycloalkyl, C₁₋₂ alkylene-C₃₋₆ cycloalkyl, —NH(C₁₋₆ alkyl), —N(C₁₋₆ alkyl)₂, —NH(C₃₋₆ cycloalkyl), —N(C₃₋₆ cycloalkyl)₂, hydroxy, —OC₁₋₆ alkyl, —OC₃₋₆ cycloalkyl, O-heterocyclyl wherein the heterocyclyl is a 3-8 membered heterocyclyl, 3-8 membered heterocyclyl, 6-10 membered bridged biheterocyclyl, 5-12 membered spiro biheterocyclyl, 6-12 membered fused biheterocyclyl, C₆₋₁₀ aryl, or 5-10 membered heteroaryl, wherein each of C₁₋₆ alkyl, C₃₋₆ cycloalkyl, aryl, heteroaryl, heterocyclyl, bridged biheterocyclyl, spiro biheterocyclyl, and fused biheterocyclyl are substituted with 1, 2 or 3 R^{b} independently; if present, each of heterocyclyl, bridged biheterocyclyl, spiro biheterocyclyl, fused biheterocyclyl, and heteroaryl comprises one or more heteroatoms independently selected from the group consisting of O, S, NH, N, P(═O), S(═O) and S(═O)₂; preferably, R² is hydrogen, C₁₋₆ alkyl, C₃₋₆ cycloalkyl, C₁₋₂ alkylene-C₃₋₆ cycloalkyl, —NH(C₁₋₆ alkyl), —N(C₁₋₆ alkyl)₂, —NH(C₃₋₆ cycloalkyl), —N(C₃₋₆ cycloalkyl)₂, —OC₁₋₆ alkyl, 3-8 membered heterocyclyl, 6-10 membered bridged biheterocyclyl, 5-12 membered spiro biheterocyclyl, 6-12 membered fused biheterocyclyl, C₆₋₁₀ aryl, or 5-10 membered heteroaryl, wherein each of C₁₋₆ alkyl, C₃₋₆ cycloalkyl, aryl, heteroaryl, heterocyclyl, bridged biheterocyclyl, spiro biheterocyclyl, and fused biheterocyclyl are substituted with 1, 2 or 3 R^{b} independently; if present, each of heterocyclyl, bridged biheterocyclyl, spiro biheterocyclyl, fused biheterocyclyl, and heteroaryl comprises one or more heteroatoms independently selected from the group consisting of O, S, NH, N, P(═O), S(═O) and S(═O)₂;
if present, each R⁵ and R⁶ is independently hydrogen, deuterium, C₁₋₆ alkyl, C₃₋₆ cycloalkyl, or C₁₋₂ alkylene-C₃₋₆ cycloalkyl, wherein each C₁₋₆ alkyl and C₃₋₆ cycloalkyl is substituted with 1, 2, or 3 R^{c} independently; or R⁵ and R⁶, together with the carbon atoms they bound with, form a saturated 5- or 6-membered spiro heterocycle comprising one or more heteroatoms independently selected from the group consisting of oxygen, nitrogen, and sulfur, wherein the saturated 5- or 6-membered spiro heterocycle is substituted with 1, 2, or 3 R^{c};
If present, R⁷ is hydrogen, C₁₋₆ alkyl, C₃₋₆ cycloalkyl, C₁₋₂ alkylene-C₃₋₆ cycloalkyl, or 5-or 6-membered heterocyclyl comprising one or more heteroatoms independently selected from the group consisting of oxygen, nitrogen, and sulfur, wherein each C₁₋₆ alkyl and C₃₋₆ cycloalkyl is substituted with 1, 2, or 3 R^{c} independently;
if present, R⁸ is hydrogen, C₁₋₆ alkyl, C₃₋₆ cycloalkyl, C₁₋₂ alkylene-C₃₋₆ cycloalkyl, or 5-6 membered heterocyclyl comprising one or more heteroatoms independently selected from the group consisting of oxygen, nitrogen, and sulfur; each R⁹, R¹⁰, R¹¹, and R¹² is independently hydrogen, deuterium, C₁₋₆ alkyl, C₃₋₆ cycloalkyl, C₁₋₂ alkylene-C₃₋₆ cycloalkyl, or 5-6 membered heterocyclyl comprising one or more heteroatoms independently selected from the group consisting of oxygen, nitrogen, and sulfur; wherein each C₁₋₆ alkyl, C₃₋₆ cycloalkyl, and heterocyclyl is substituted with 1, 2, or 3 R^{c} independently; or R⁹ and R¹⁰, together with the carbon they bound with, form a carbonyl; ring Z¹ is selected from the group consisting of:
(1) 5-6 membered heteroaryl comprising 1, 2, or 3 heteroatoms independently selected from the group consisting of oxygen, nitrogen, and sulfur;
(2) phenyl;
(3) 5-6 membered unsaturated or saturated heterocyclyl comprising 1 or 2 heteroatoms independently selected from the group consisting of oxygen and nitrogen;
(4) 7-10 membered fused bicyclic heterocyclyl comprising 1, 2, or 3 heteroatoms independently selected from the group consisting of oxygen, nitrogen, and sulfur; and
(5) unsaturated or saturated C₃₋₆ cycloalkyl;
ring Z² is absent, or ring Z² is selected from the group consisting of:
(1) the group consisting of oxygen, nitrogen, and sulfur;
(2) phenyl;
(3) 5-6 membered unsaturated or saturated heterocyclyl comprising 1 or 2 heteroatoms independently selected from the group consisting of oxygen and nitrogen; and
(4) unsaturated or saturated C₃₋₆ cycloalkyl;
wherein when ring Z² is absent, R^{d} replaces ring Z² and R^{d} connected with ring Z¹;
if present, each R^{a} is independently hydrogen, deuterium, fluorine, chlorine, bromine, iodine, hydroxy, amino, methylamino, dimethylamino, cyano, methyl, deuterated methyl, methoxy, deuterated methoxy, ethyl, cyclopropyl, tert-butoxycarbonyl, carbamoyl, C₁₋₂ alkylene-hydroxy, C₁₋₂ alkylene-methoxy, or C₁₋₂ alkylene-deuterated methoxy;
if present, each R^{b} is independently hydrogen, deuterium, fluorine, chlorine, bromine, iodine, hydroxy, amino, methylamino, dimethylamino, cyano, C₁₋₃ alkyl, deuterated methyl, methoxy, deuterated methoxy, cyclopropyl, C₁₂ alkylene-hydroxy, C₁ 2 alkylene-methoxy, or C₁₂ alkylene-deuterated methoxy; or two R^{b} on any non-adjacent carbons, together with atoms attached thereto, form a ring; or two R^{b} on the same carbon, together with the carbon attached thereto, form a carbonyl if present, each R^{c} is independently hydrogen, deuterium, fluorine, chlorine, bromine, iodine, hydroxy, amino, methylamino, dimethylamino, cyano, methyl, deuterated methyl, methoxy, or deuterated methoxy;
if present, each R^{d} is independently hydrogen, deuterium, methyl, deuterated methyl, ethyl, cyclopropyl, C₁₋₂ alkylene-hydroxy, C₁₋₂ alkylene-methoxy, C₁₋₂ alkylene-deuterated methoxy, trifluoromethyl, trifluoromethoxy, difluoromethyl, or difluoromethoxy; and
if present, each R³ and R⁴ is independently hydrogen, deuterium, fluorine, chlorine, bromine, iodine, hydroxy, amino, methylamino, dimethylamino, nitro, cyano, methyl, deuterated methyl, methoxy, deuterated methoxy, ethyl, cyclopropyl, trifluoromethyl, trifluoromethoxy, difluoromethyl, difluoromethoxy, or dimethylphosphinyl.

2. The compound of claim 1, or a pharmaceutically acceptable salt, ester, solvate, prodrug, isotope-labeled derivative, or isomer thereof, wherein the compound is formula (I-A): 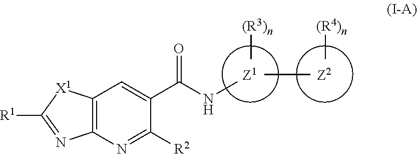
wherein n, X¹, Z¹, Z², R¹, R², R³, and R⁴ are defined according to claim 1.

3. The compound of claim 1, or a pharmaceutically acceptable salt, ester, solvate, prodrug, isotope-labeled derivative, or isomer thereof, wherein the compound is Formula (I-B): 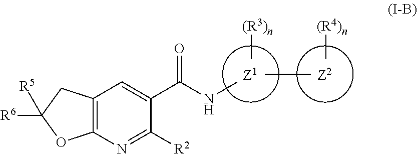
wherein n, Z¹, Z², R², R³, R⁴, R⁵, and R⁶ are defined according to claim 1.

4. The compound of claim 1, or a pharmaceutically acceptable salt, ester, solvate, prodrug, isotope-labeled derivative, or isomer thereof, wherein the compound is formula (I-C): 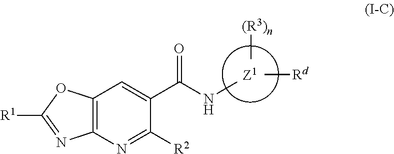
wherein n, R¹, R², R³, R^{d}, and Z¹ are defined according to claim 1.

5. The compound of claim 1, or a pharmaceutically acceptable salt, ester, solvate, prodrug, isotope-labeled derivative, or isomer thereof, wherein the compound is formula (I-D): 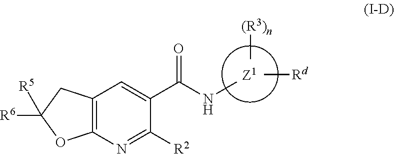
wherein n, R², R³, R⁵, R⁶, R^{d}, and Z¹ are defined according to claim 1.

6. The compound of claim 1, or a pharmaceutically acceptable salt, ester, solvate, prodrug, isotope-labeled derivative, or isomer thereof, wherein:
ring Z¹ is selected from the group consisting of:
(1) 5-6 membered heteroaryl comprising 1, 2, or 3 heteroatoms independently selected from the group consisting of oxygen, nitrogen, and sulfur;
(2) phenyl; and
(3) 7-10 membered fused bicyclic heterocyclyl comprising 1, 2, or 3 heteroatoms independently selected from the group consisting of oxygen, nitrogen, and sulfur.

7. The compound of claim 1, or a pharmaceutically acceptable salt, ester, solvate, prodrug, isotope-labeled derivative, or isomer thereof, wherein:
(1) when ring Z² is present 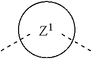
is selected from the group consisting of: 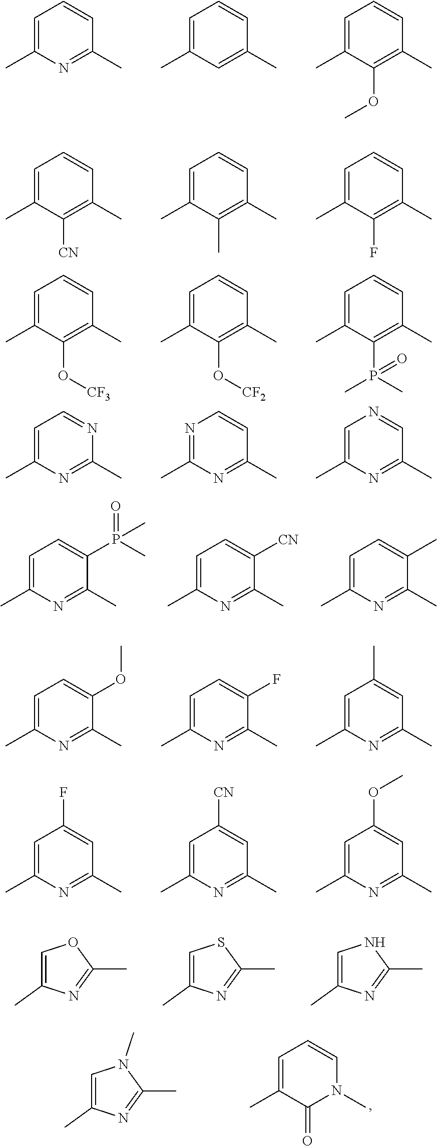
preferably selected from the group consisting of: 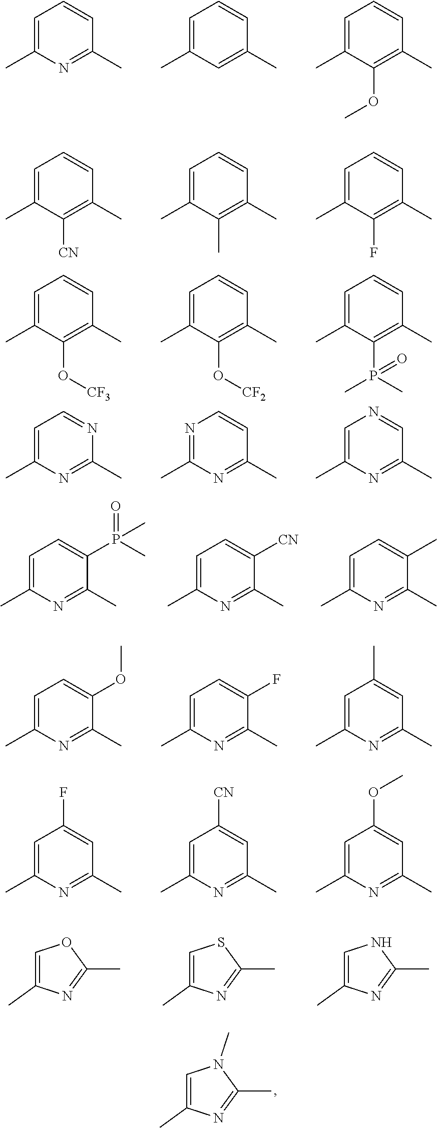
or preferably selected from the group consisting of: 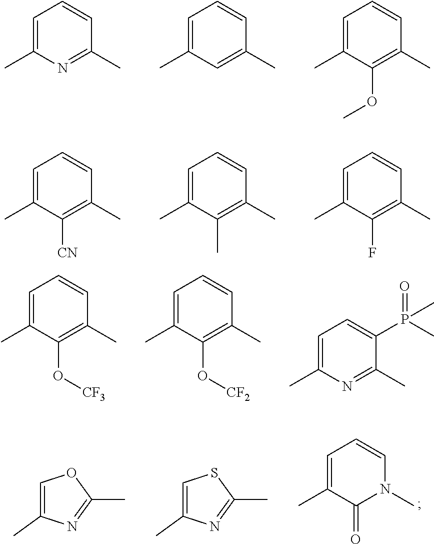
(2) when Z² is absent, 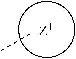
is selected from the group consisting of: 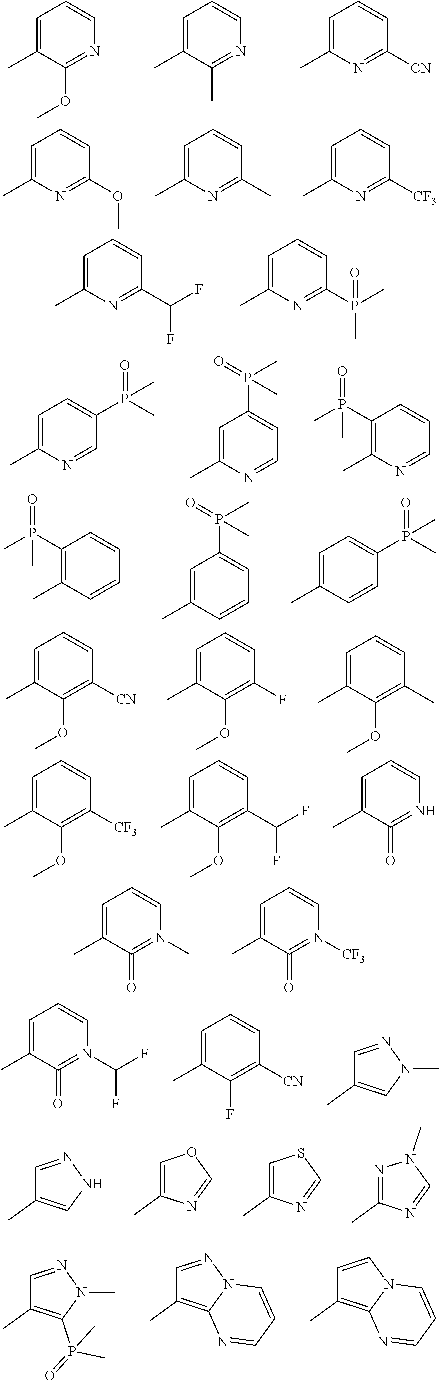 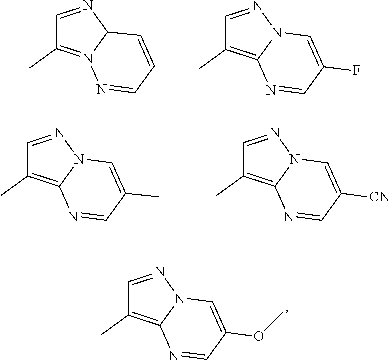
preferably selected from the group consisting of:

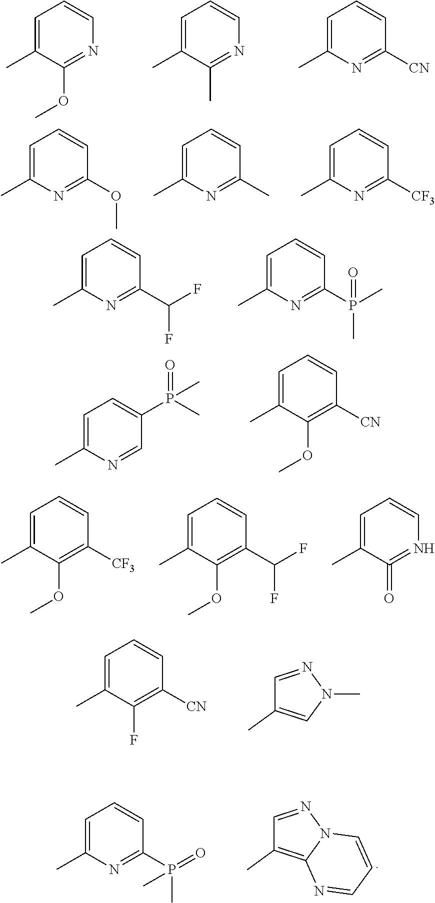

8. The compound of claim 1, or a pharmaceutically acceptable salt, ester, solvate, prodrug, isotope-labeled derivative, or isomer thereof, wherein:
R¹ is selected from the group consisting of: 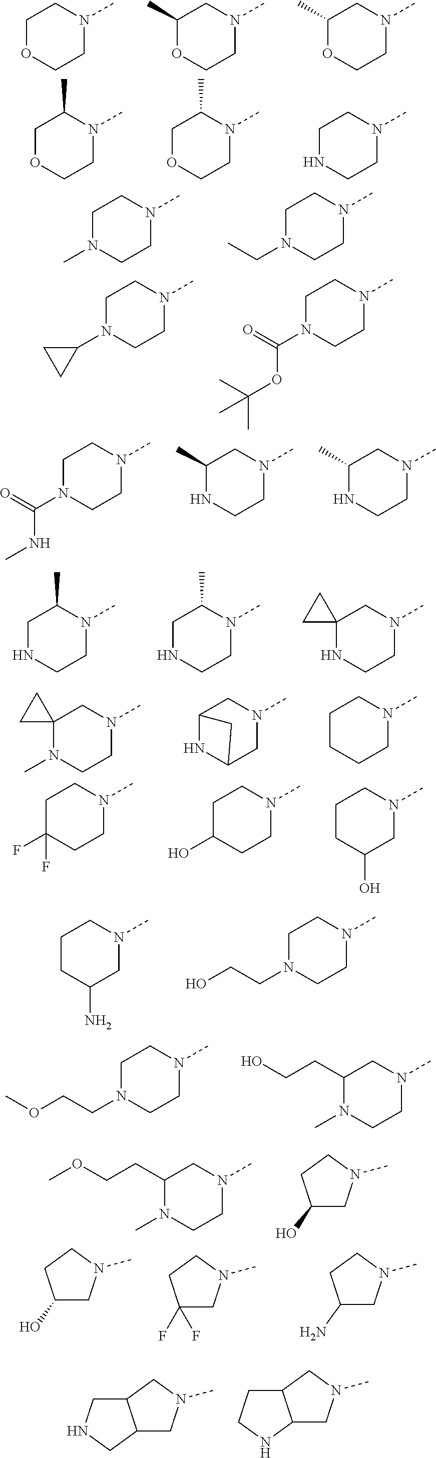 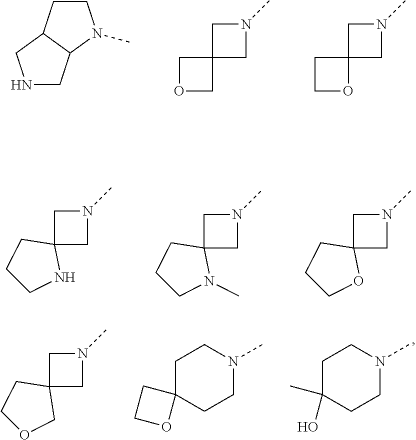
preferably selected from the group consisting of:

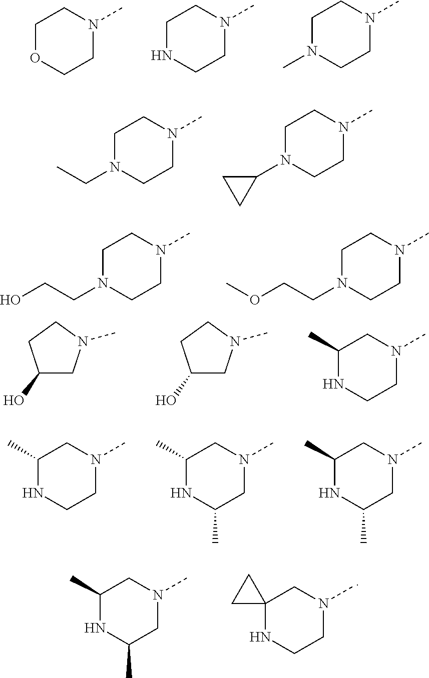

9. The compound of claim 1, or a pharmaceutically acceptable salt, ester, solvate, prodrug, isotope-labeled derivative, or isomer thereof, wherein:
(1) when ring Z² is present, 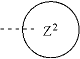
is selected from the group consisting of: 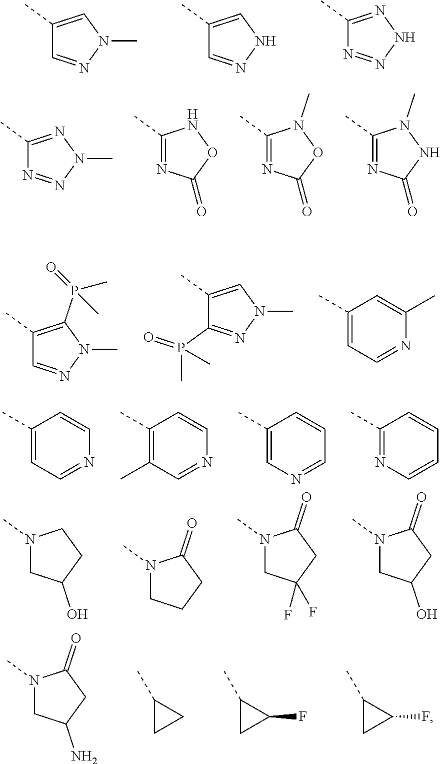
preferably selected from the group consisting of: 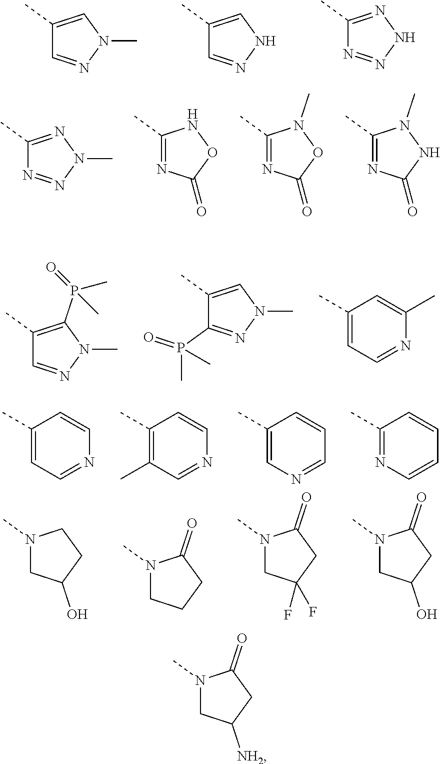
or preferably selected from the group consisting of: 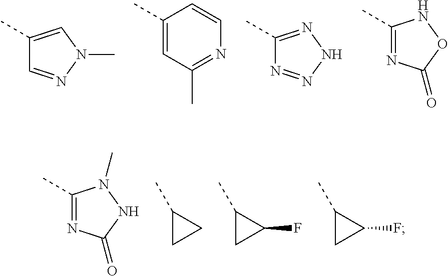
(2) when ring Z² is absent, R^{d} replaces ring Z², and R^{d} connected with ring Z¹, and R^{d} is according to claim 1.

10. The compound of claim 1, or a pharmaceutically acceptable salt, ester, solvate, prodrug, isotope-labeled derivative, or isomer thereof, wherein:
R² is —NH(C₁₋₆ alkyl), —N(C₁₋₆ alkyl)₂, —NH(C₃₋₆ cycloalkyl), —N(C₃₋₆ cycloalkyl)₂, hydroxy, —OC₁₋₆ alkyl, —OC₃₋₆ cycloalkyl, O-heterocyclyl wherein the heterocyclyl is a 3-8 membered heterocyclyl, 3-8 membered heterocyclyl, 6-10 membered bridged biheterocyclyl, 5-12 membered spiro biheterocyclyl, 6-12 membered fused biheterocyclyl, wherein each of C₁₋₆ alkyl, C₃₋₆ cycloalkyl, heterocyclyl, spiro biheterocyclyl, and fused biheterocyclyl are substituted with 1, 2 or 3 R^{b} independently, and R^{b} is according to claim 1; preferably R² is —NH(C₁₋₆ alkyl), —N(C₁₋₆ alkyl)₂, —NH(C₃₋₆ cycloalkyl), —N(C₃₋₆ cycloalkyl)₂, —OC₁₋₆ alkyl, 3-8 membered heterocyclyl, 5-12 membered spiro biheterocyclyl, and 6-12 membered fused biheterocyclyl, wherein each of C₁₋₆ alkyl, C₃₋₆ cycloalkyl, heterocyclyl, spiro biheterocyclyl, and fused biheterocyclyl are substituted with 1, 2 or 3 R^{b} independently, and R^{b} is according to claim 1.

11. The compound of claim 1, or a pharmaceutically acceptable salt, ester, solvate, prodrug, isotope-labeled derivative, or isomer thereof, wherein:
R² is selected from the group consisting of: 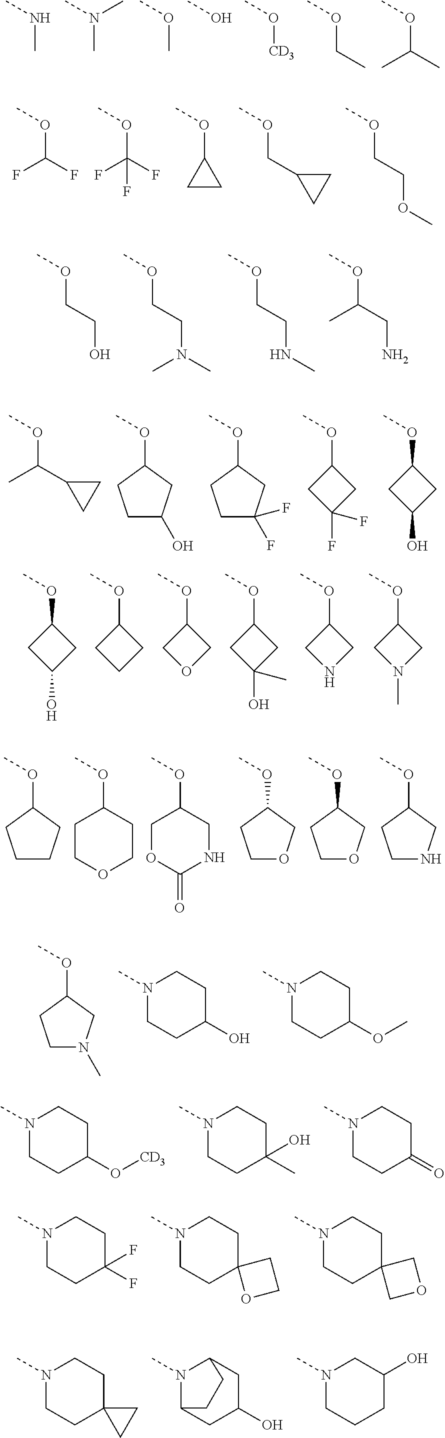 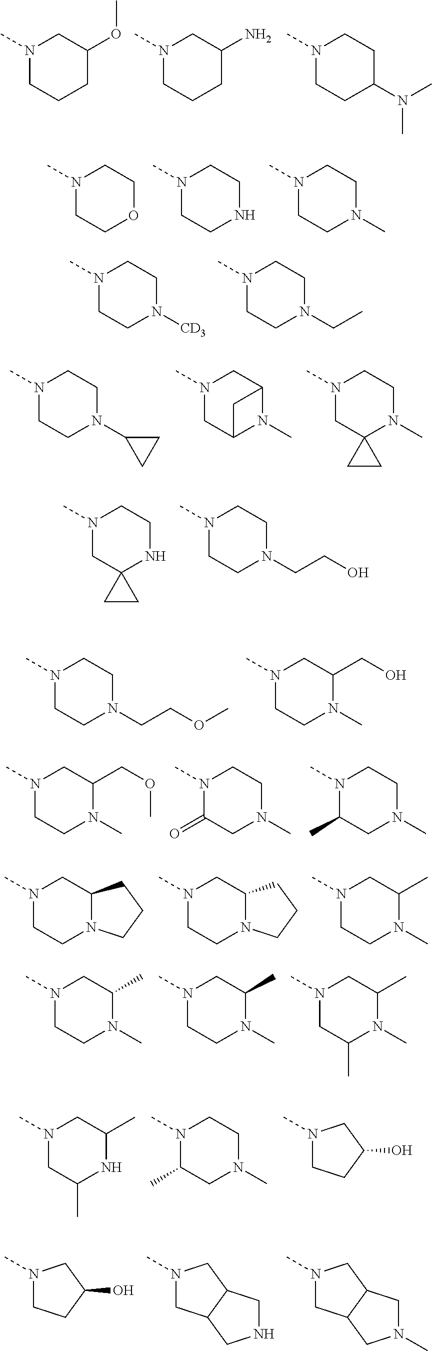 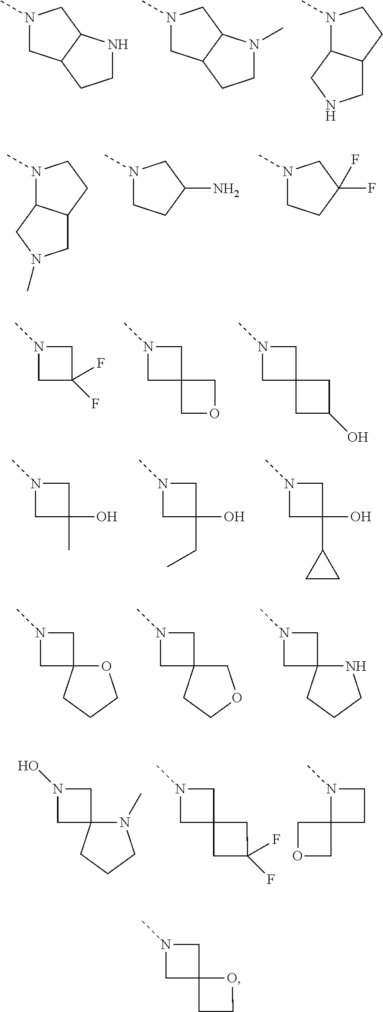
preferably selected from the group consisting of: 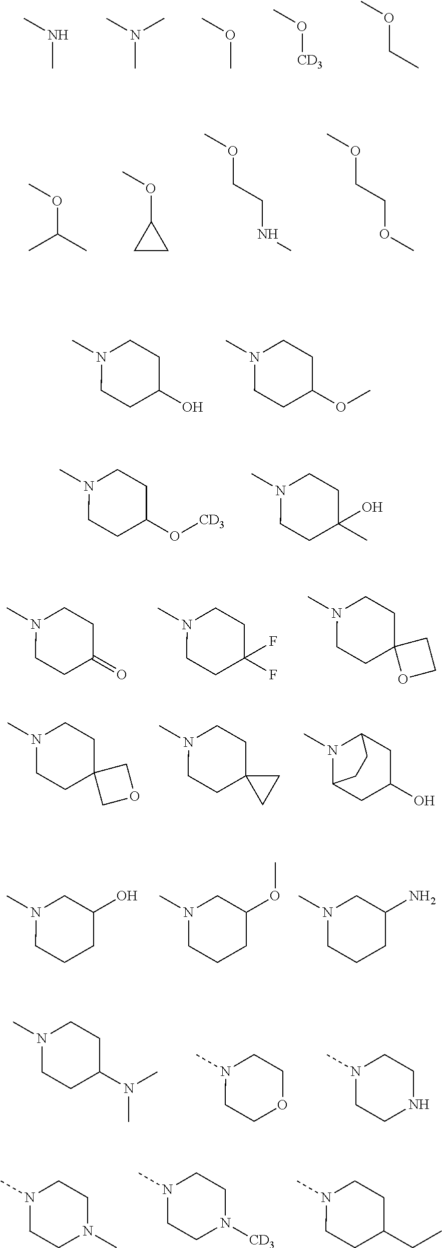 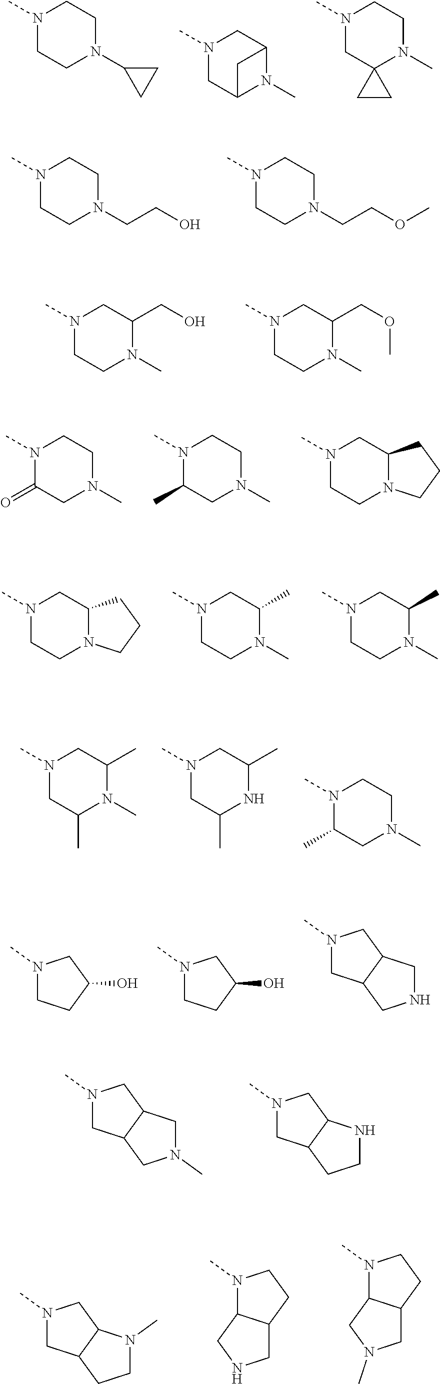 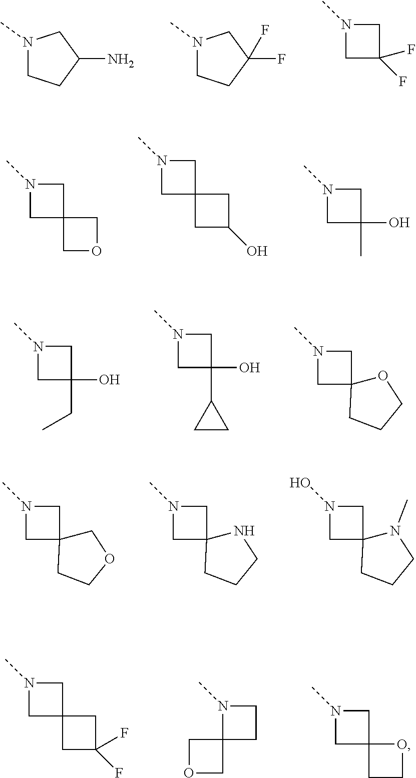
or preferably selected from the group consisting of:

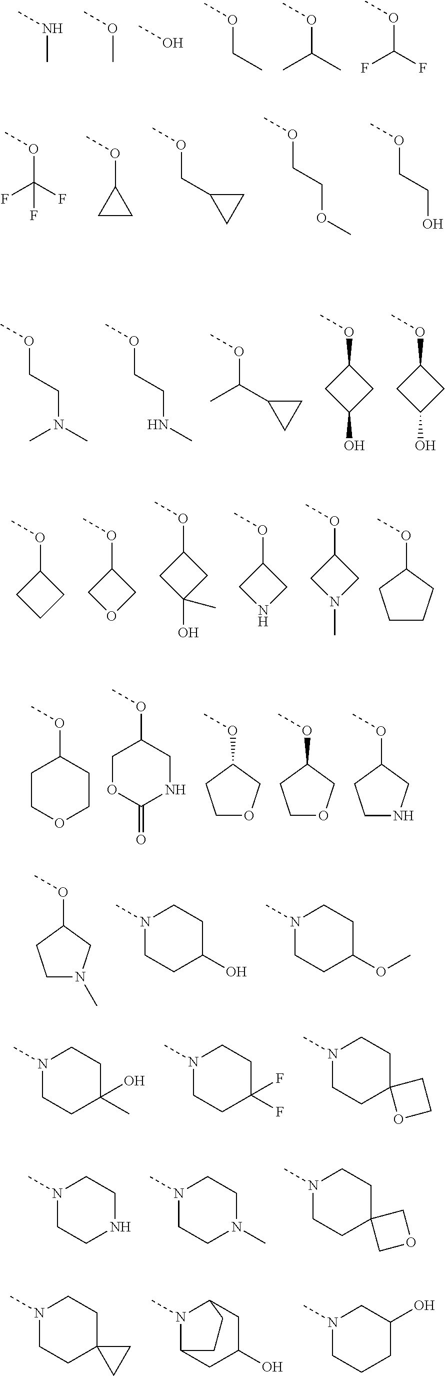

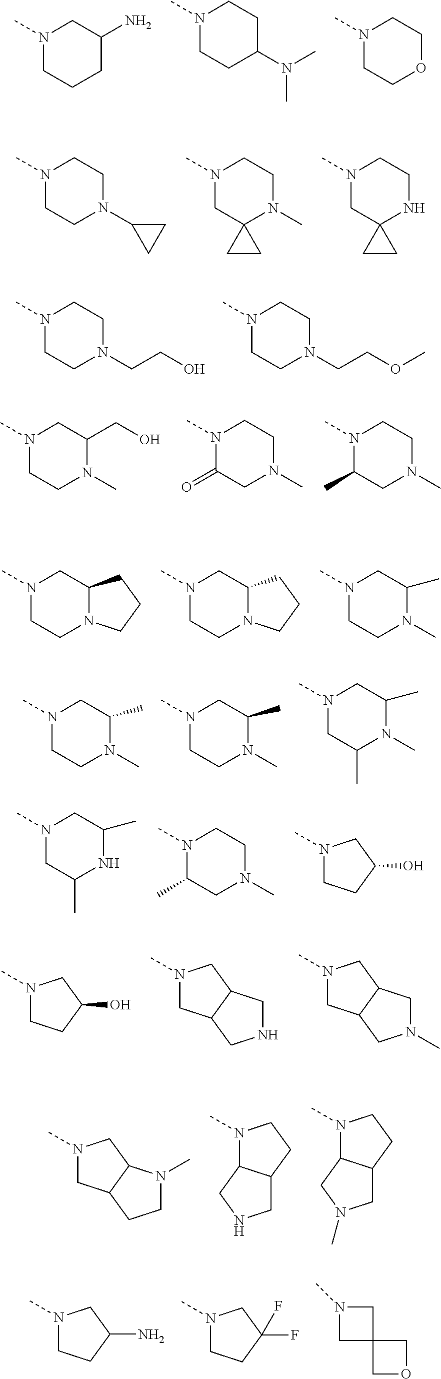

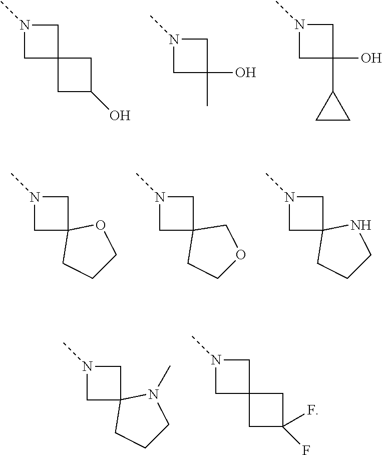

12. A compound of Formula (II): 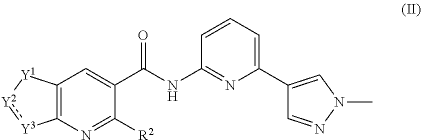
or a pharmaceutically acceptable salt, ester, solvate, prodrug, isotope-labeled derivative, or isomer thereof, wherein: 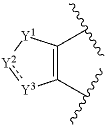
is selected from the group consisting of: 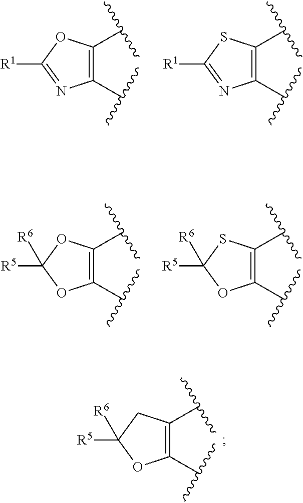
R¹ is selected from the group consisting of: 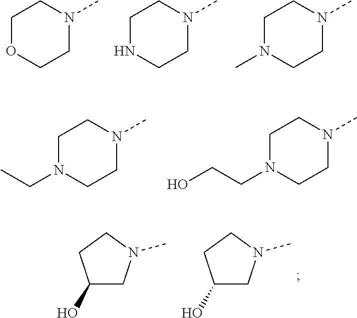
R² is selected from the group consisting of: 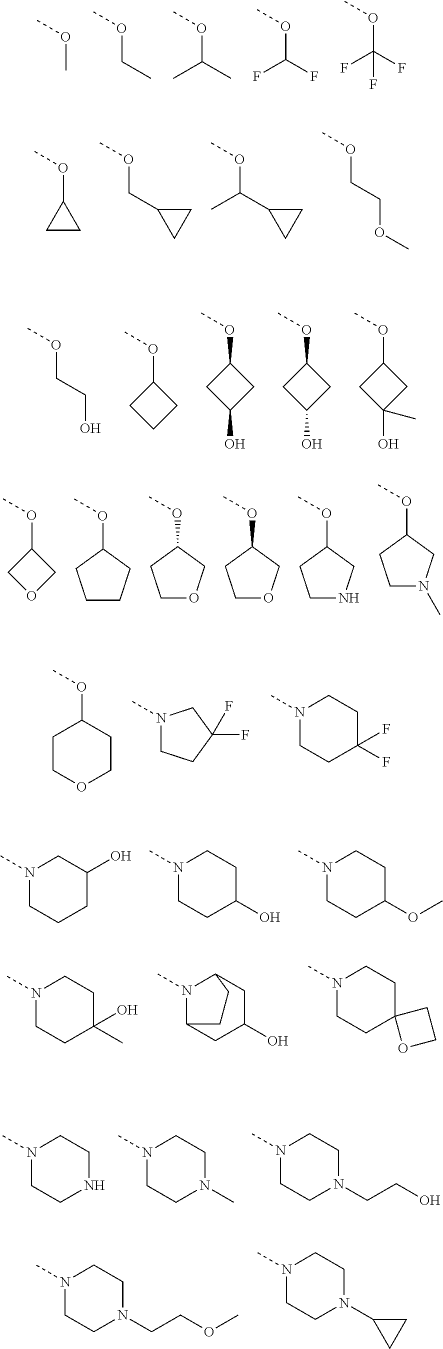 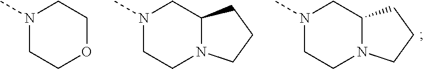
each of R⁵ and R⁶ is independently C₁₋₆ alkyl;
preferably, 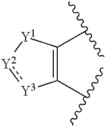
is selected from the group consisting of: 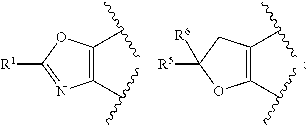
R¹ is selected from the group consisting of: 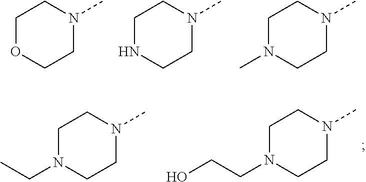
R² is selected from the group consisting of: 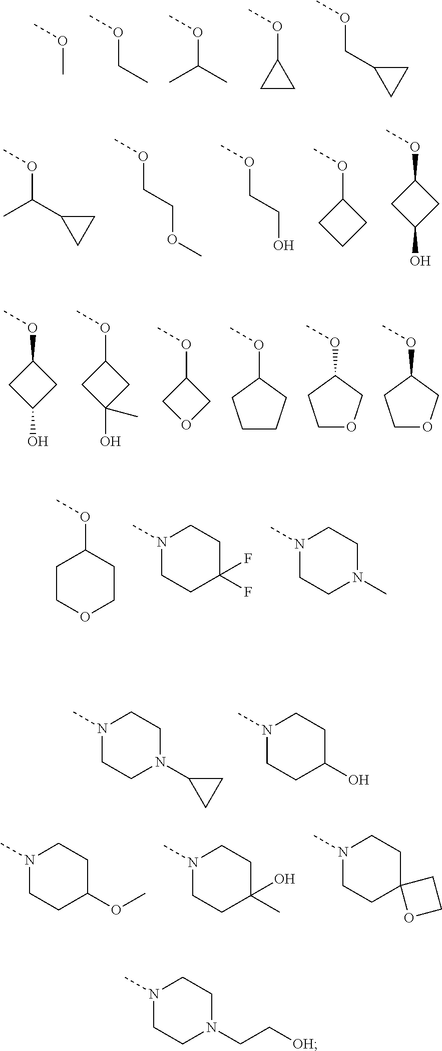
and each of R⁵ and R⁶ is methyl.

13. A compound or a pharmaceutically acceptable salt, ester, solvate, prodrug, isotope-labeled derivative, or isomer thereof, wherein the compound is selected from the group consisting of:

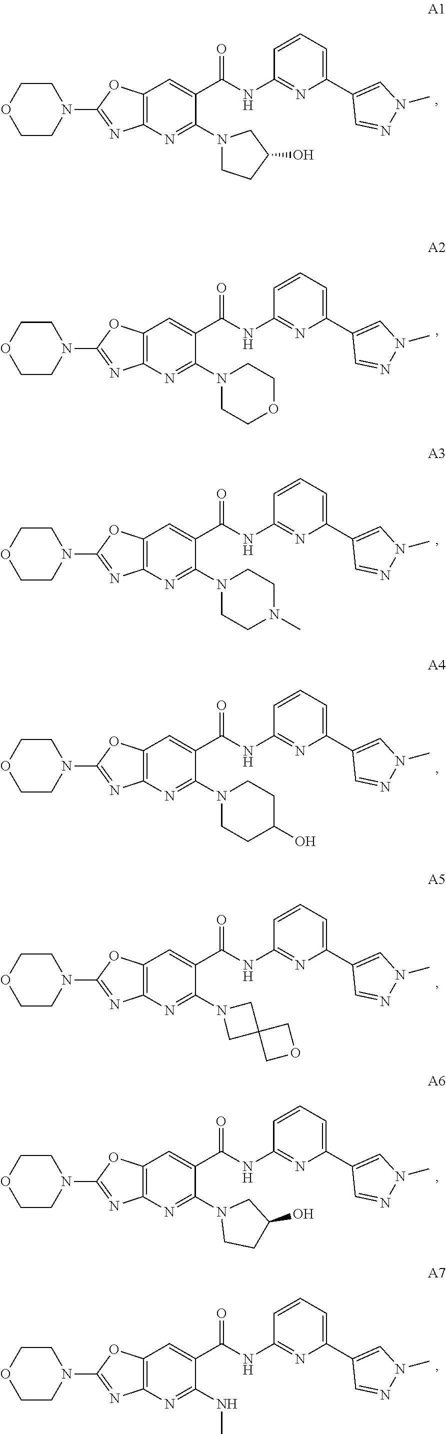

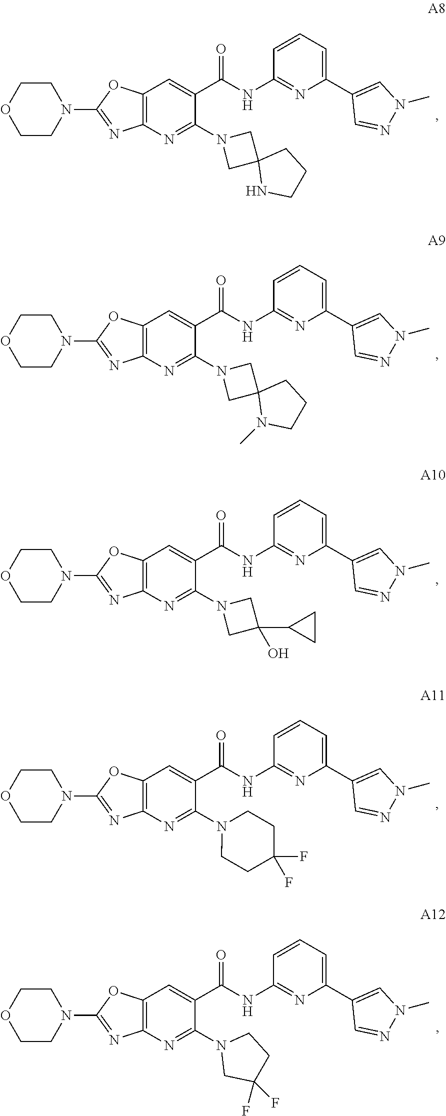 [SMILES: Cn1cc(-c2cccc(NC(=O)c3cc4oc(N5CCOCC5)nc4nc3N3CC4(CCCN4)C3)n2)cn1 ; CN1CCCC12CN(c1nc3nc(N4CCOCC4)oc3cc1C(=O)Nc1cccc(-c3cnn(C)c3)n1)C2 ; Cn1cc(-c2cccc(NC(=O)c3cc4oc(N5CCOCC5)nc4nc3N3CC(O)(C4CC4)C3)n2)cn1 ; Cn1cc(-c2cccc(NC(=O)c3cc4oc(N5CCOCC5)nc4nc3N3CCC(F)(F)CC3)n2)cn1 ; Cn1cc(-c2cccc(NC(=O)c3cc4oc(N5CCOCC5)nc4nc3N3CCC(F)(F)C3)n2)cn1]

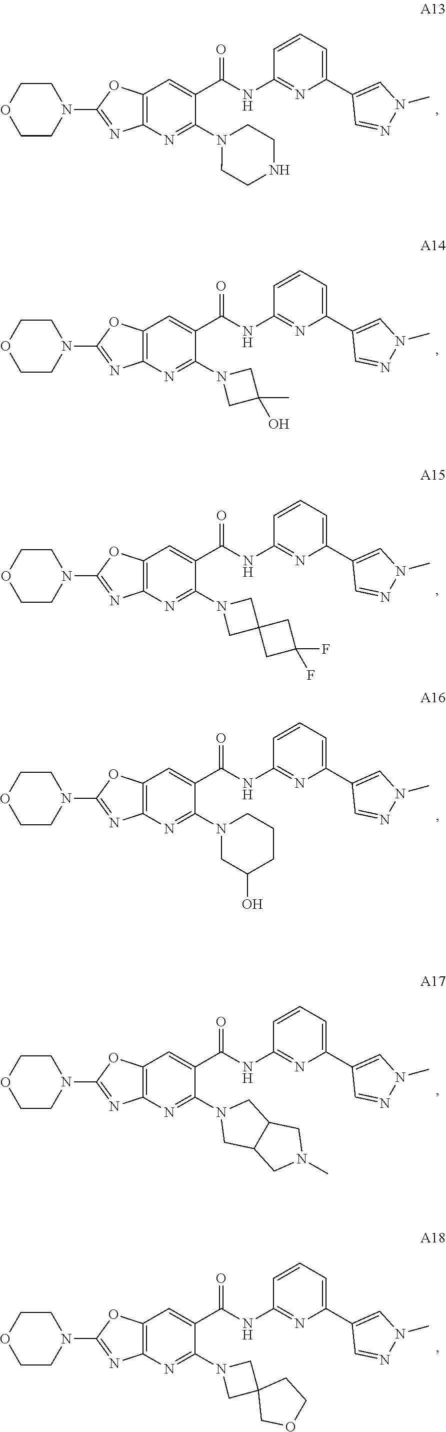 [SMILES: Cn1cc(-c2cccc(NC(=O)c3cc4oc(N5CCOCC5)nc4nc3N3CCNCC3)n2)cn1 ; Cn1cc(-c2cccc(NC(=O)c3cc4oc(N5CCOCC5)nc4nc3N3CC(C)(O)C3)n2)cn1 ; Cn1cc(-c2cccc(NC(=O)c3cc4oc(N5CCOCC5)nc4nc3N3CC4(C3)CC(F)(F)C4)n2)cn1 ; Cn1cc(-c2cccc(NC(=O)c3cc4oc(N5CCOCC5)nc4nc3N3CCCC(O)C3)n2)cn1 ; CN1CC2CN(c3nc4nc(N5CCOCC5)oc4cc3C(=O)Nc3cccc(-c4cnn(C)c4)n3)CC2C1 ; Cn1cc(-c2cccc(NC(=O)c3cc4oc(N5CCOCC5)nc4nc3N3CC4(CCOC4)C3)n2)cn1]

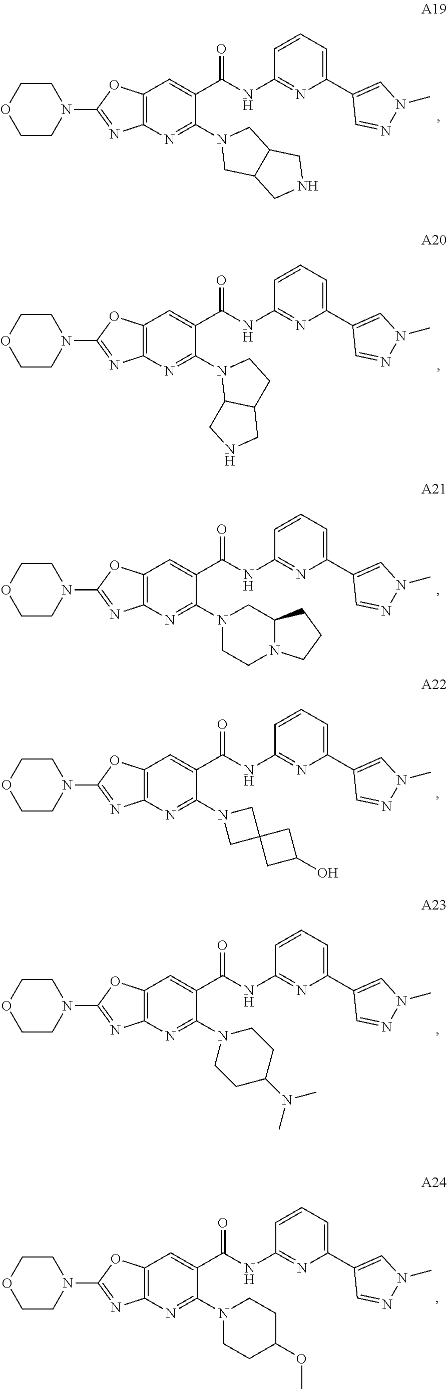 [SMILES: Cn1cc(-c2cccc(NC(=O)c3cc4oc(N5CCOCC5)nc4nc3N3CC4CNCC4C3)n2)cn1 ; Cn1cc(-c2cccc(NC(=O)c3cc4oc(N5CCOCC5)nc4nc3N3CCC4CNCC43)n2)cn1 ; Cn1cc(-c2cccc(NC(=O)c3cc4oc(N5CCOCC5)nc4nc3N3CCN4CCC[C@@H]4C3)n2)cn1 ; Cn1cc(-c2cccc(NC(=O)c3cc4oc(N5CCOCC5)nc4nc3N3CC4(CC(O)C4)C3)n2)cn1 ; CN(C)C1CCN(c2nc3nc(N4CCOCC4)oc3cc2C(=O)Nc2cccc(-c3cnn(C)c3)n2)CC1 ; COC1CCN(c2nc3nc(N4CCOCC4)oc3cc2C(=O)Nc2cccc(-c3cnn(C)c3)n2)CC1]

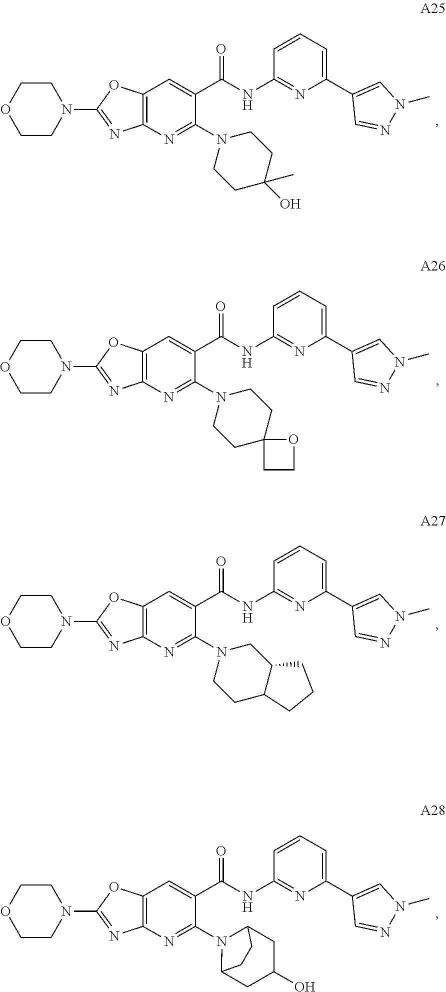

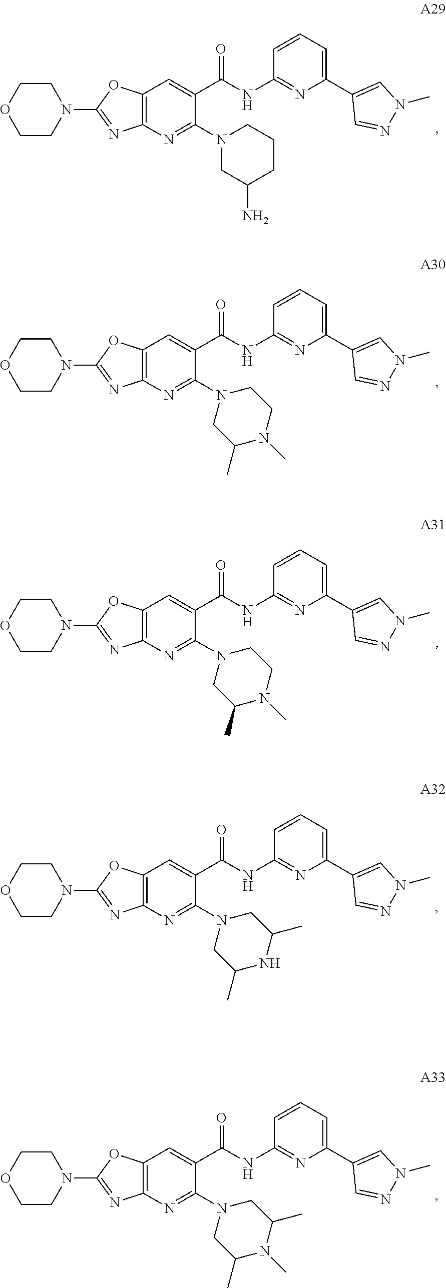 [SMILES: Cn1cc(-c2cccc(NC(=O)c3cc4oc(N5CCOCC5)nc4nc3N3CCCC(N)C3)n2)cn1 ; CC1CN(c2nc3nc(N4CCOCC4)oc3cc2C(=O)Nc2cccc(-c3cnn(C)c3)n2)CCN1C ; C[C@H]1CN(c2nc3nc(N4CCOCC4)oc3cc2C(=O)Nc2cccc(-c3cnn(C)c3)n2)CCN1C ; CC1CN(c2nc3nc(N4CCOCC4)oc3cc2C(=O)Nc2cccc(-c3cnn(C)c3)n2)CC(C)N1 ; CC1CN(c2nc3nc(N4CCOCC4)oc3cc2C(=O)Nc2cccc(-c3cnn(C)c3)n2)CC(C)N1C]

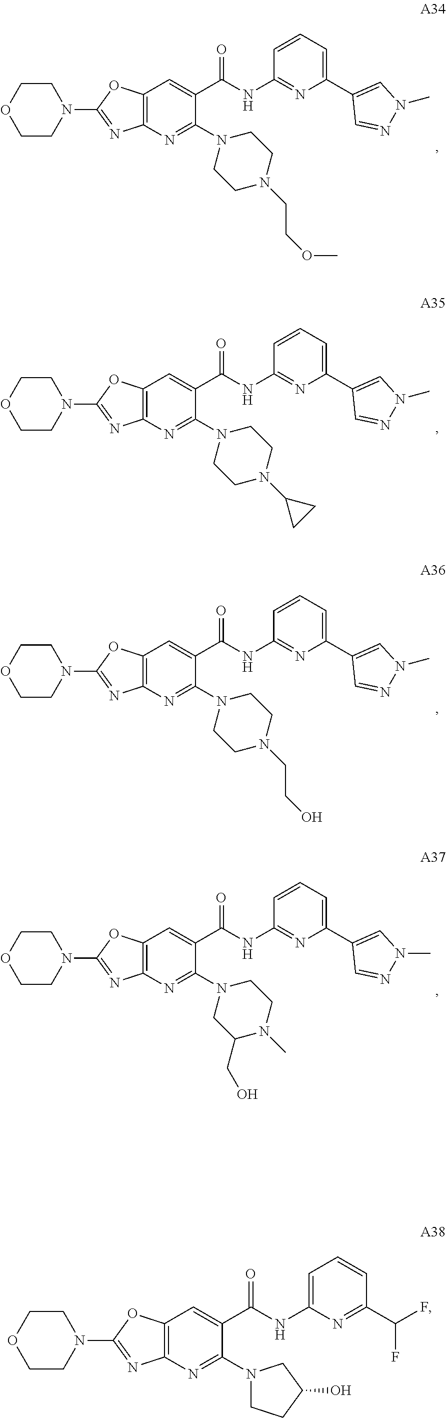 [SMILES: COCCN1CCN(c2nc3nc(N4CCOCC4)oc3cc2C(=O)Nc2cccc(-c3cnn(C)c3)n2)CC1 ; Cn1cc(-c2cccc(NC(=O)c3cc4oc(N5CCOCC5)nc4nc3N3CCN(C4CC4)CC3)n2)cn1 ; Cn1cc(-c2cccc(NC(=O)c3cc4oc(N5CCOCC5)nc4nc3N3CCN(CCO)CC3)n2)cn1 ; CN1CCN(c2nc3nc(N4CCOCC4)oc3cc2C(=O)Nc2cccc(-c3cnn(C)c3)n2)CC1CO ; O=C(Nc1cccc(C(F)F)n1)c1cc2oc(N3CCOCC3)nc2nc1N1CC[C@@H](O)C1]

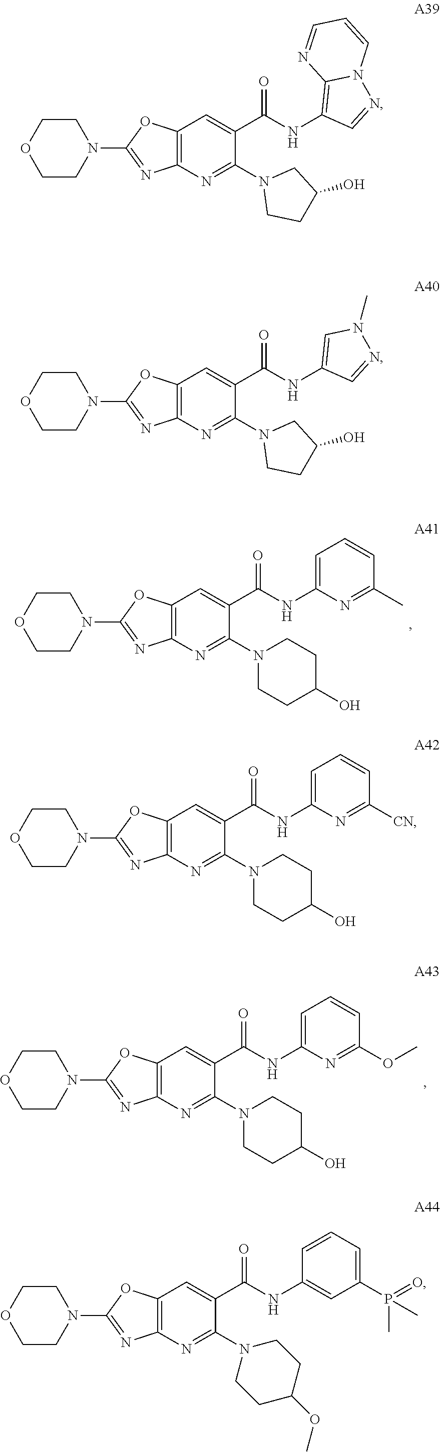 [SMILES: O=C(Nc1cnn2cccnc12)c1cc2oc(N3CCOCC3)nc2nc1N1CC[C@@H](O)C1 ; Cn1cc(NC(=O)c2cc3oc(N4CCOCC4)nc3nc2N2CC[C@@H](O)C2)cn1 ; Cc1cccc(NC(=O)c2cc3oc(N4CCOCC4)nc3nc2N2CCC(O)CC2)n1 ; N#Cc1cccc(NC(=O)c2cc3oc(N4CCOCC4)nc3nc2N2CCC(O)CC2)n1 ; COc1cccc(NC(=O)c2cc3oc(N4CCOCC4)nc3nc2N2CCC(O)CC2)n1 ; COC1CCN(c2nc3nc(N4CCOCC4)oc3cc2C(=O)Nc2cccc(P(C)(C)=O)c2)CC1]

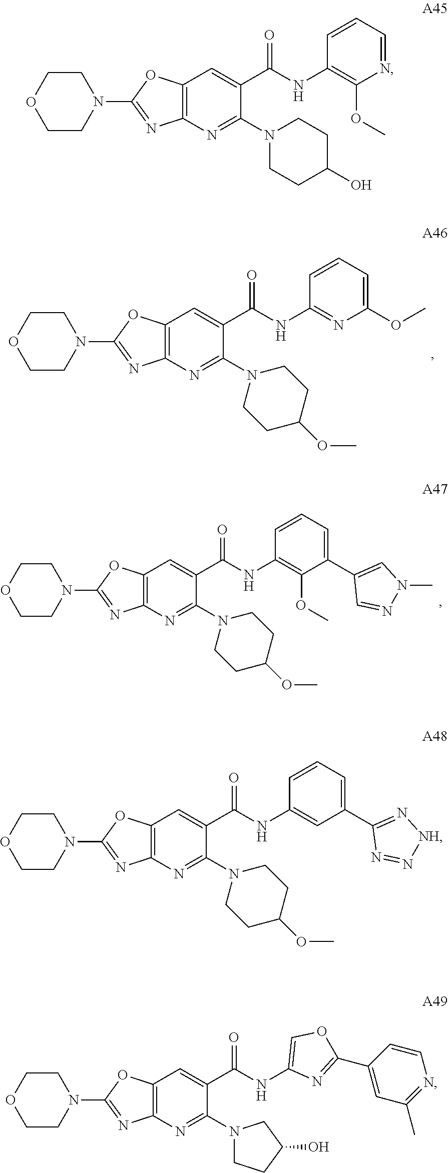 [SMILES: COc1ncccc1NC(=O)c1cc2oc(N3CCOCC3)nc2nc1N1CCC(O)CC1 ; COc1cccc(NC(=O)c2cc3oc(N4CCOCC4)nc3nc2N2CCC(OC)CC2)n1 ; COc1c(NC(=O)c2cc3oc(N4CCOCC4)nc3nc2N2CCC(OC)CC2)cccc1-c1cnn(C)c1 ; COC1CCN(c2nc3nc(N4CCOCC4)oc3cc2C(=O)Nc2cccc(-c3nn[nH]n3)c2)CC1 ; Cc1cc(-c2nc(NC(=O)c3cc4oc(N5CCOCC5)nc4nc3N3CC[C@@H](O)C3)co2)ccn1]

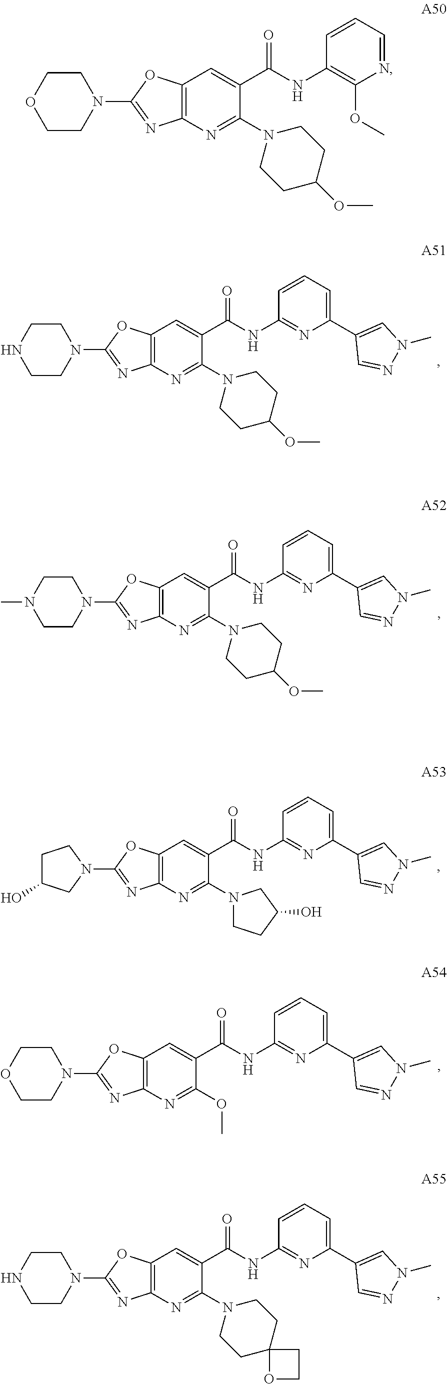

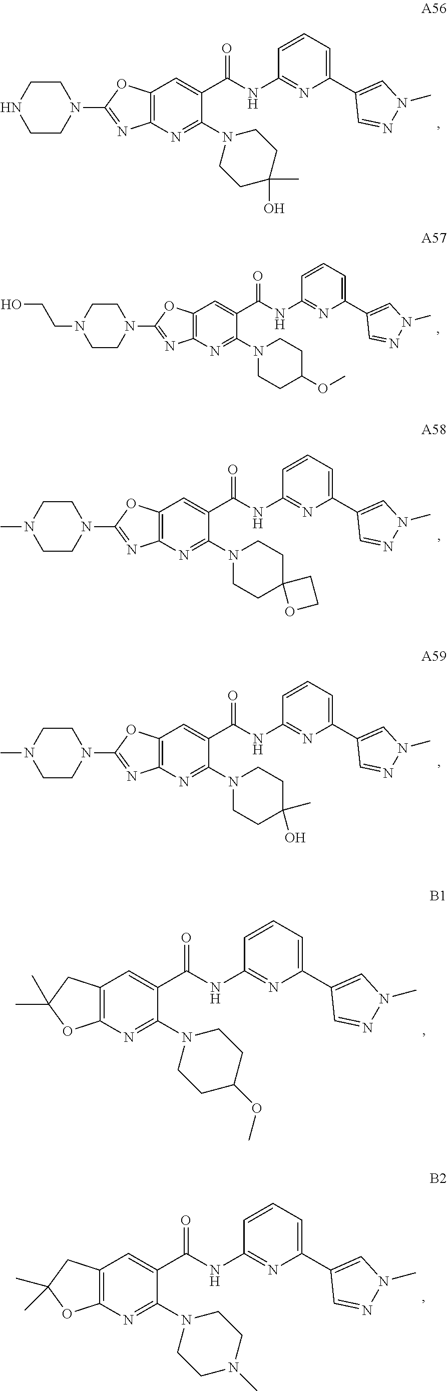

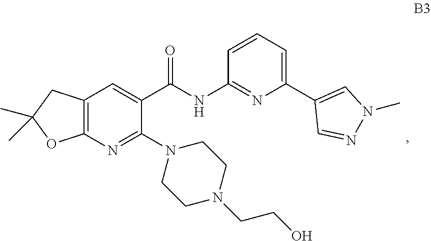

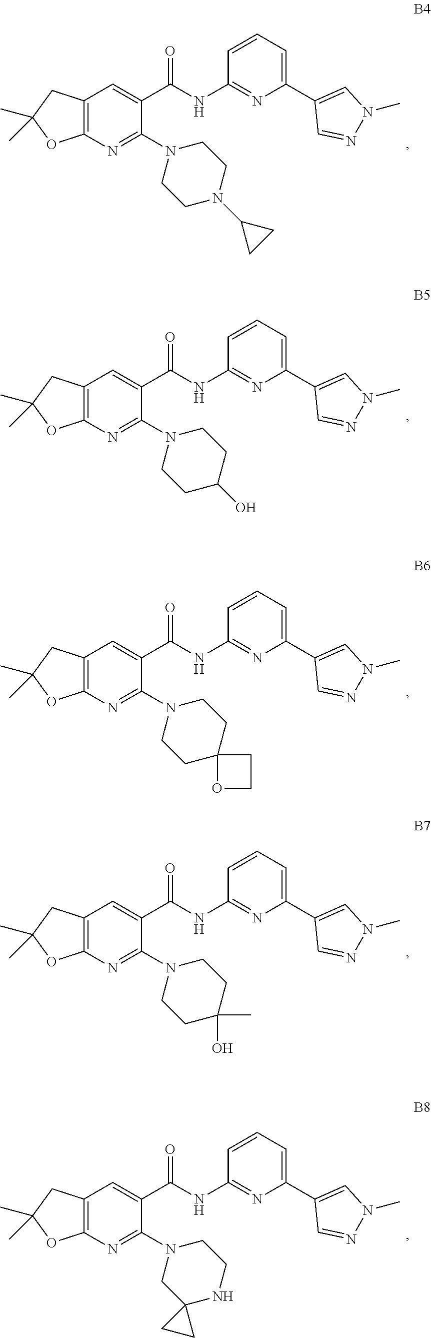

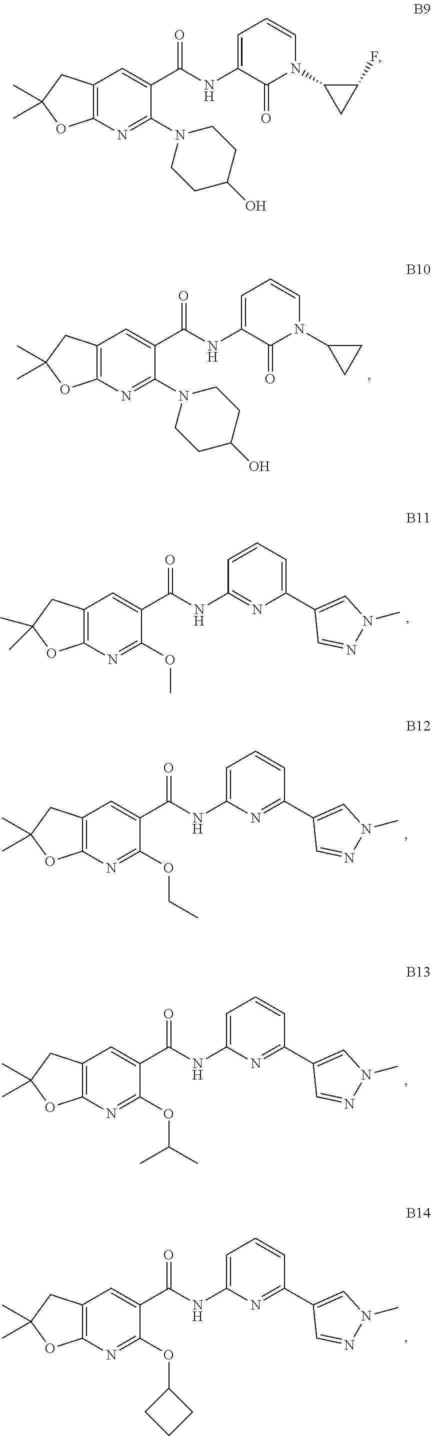

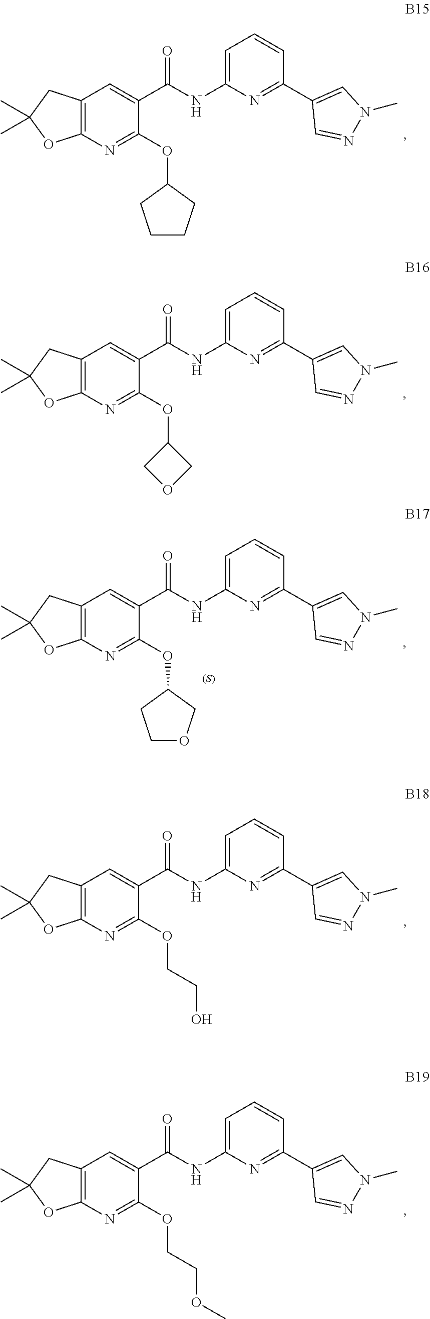

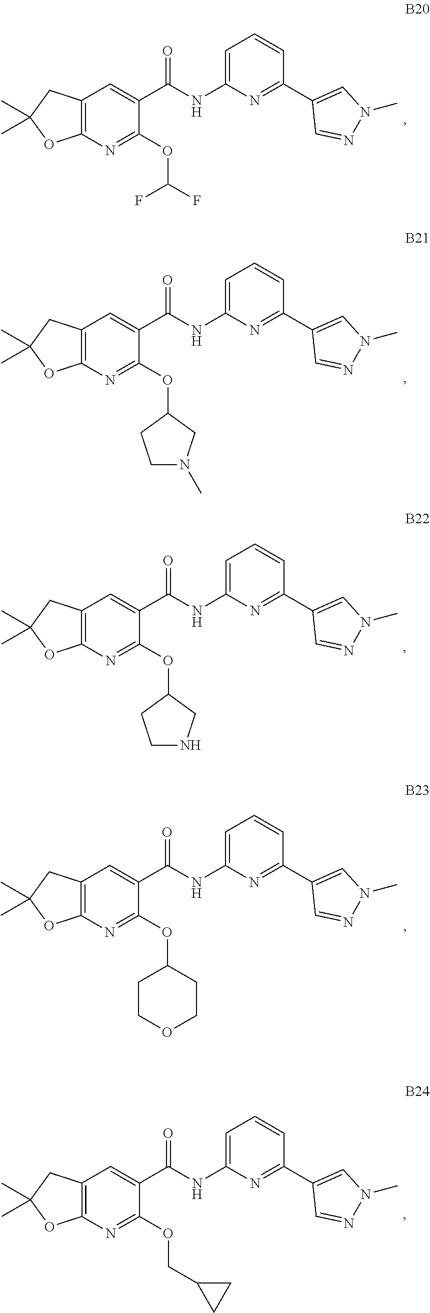

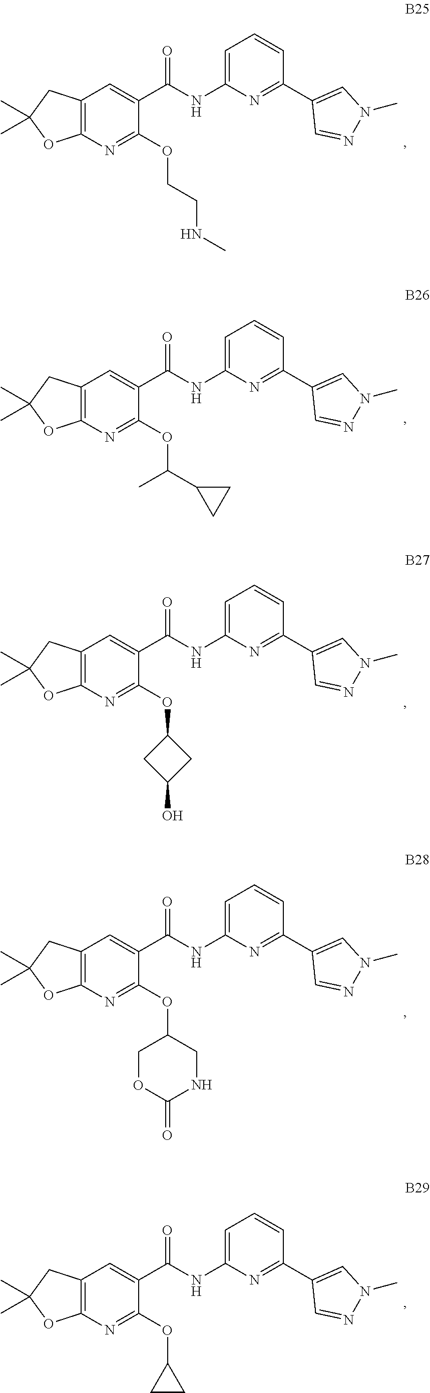

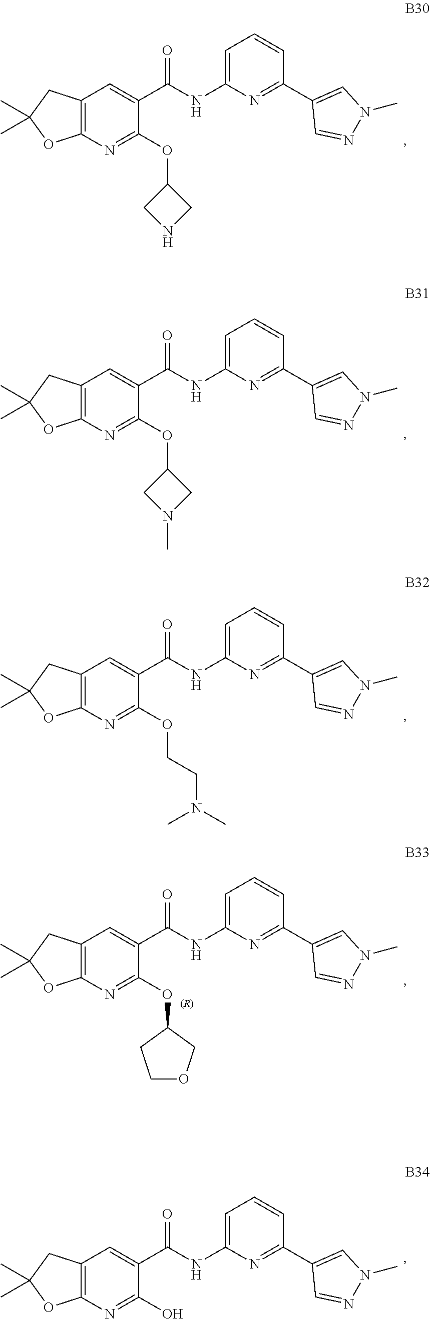

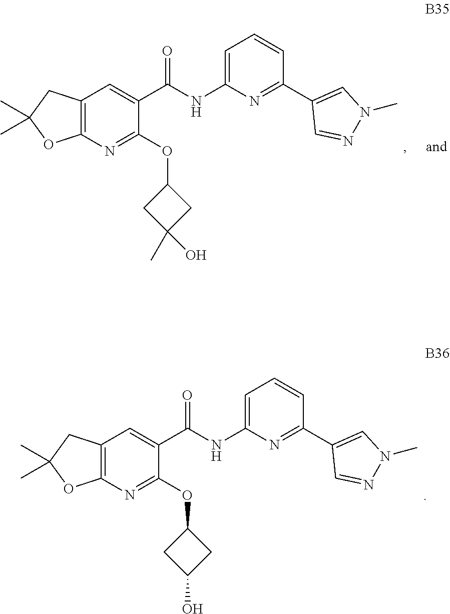

14. A pharmaceutical composition comprising a therapeutically effective amount of the compound of claim 1 or a pharmaceutically acceptable salt, ester, solvate, prodrug, isotope-labeled derivative, or isomer thereof, and a pharmaceutically acceptable carrier.

15. A composition comprising:
(i) the compound of claim 1 or a pharmaceutically acceptable salt, ester, solvate, prodrug, isotope-labeled derivative, or isomer thereof; and
(ii) one or more additional therapeutic agent, wherein the one or more additional therapeutic agent is an anti-neurodegenerative agent, an anti-inflammatory agent, and/or an anti-cancer agent.

16. A method for treating a disease or disorder in a subject, the method comprising administering to the subject a therapeutically effective amount of the compound of claim 1, or a pharmaceutically acceptable salt, ester, solvate, prodrug, isotope-labeled derivative, or isomer thereof, wherein the disease or disorder is associated with interleukin-1 receptor-associated kinase 4 (IRAK4).

17. The method of claim 16, where the disease or disorder is uveitis, dermatitis, acute lung injury, type II diabetes, arthritis, ulcerative colitis, Crohn's disease, early-onset inflammatory bowel disease, extraintestinal inflammatory bowel disease, ischemia/reperfusion injury in organ transplant, nonalcoholic fatty liver disease, autoimmune hepatitis, asthma, endometriosis, psoriasis, systemic lupus erythematosus, sarcoidosis, Wegener's granulomatosis, pulmonary fibrosis, renal fibrosis, hepatic fibrosis, myocardiale infarction, hypersensitivity pneumonitis, interstitial lung disease, ankylosing spondylitis, sclerosis, systemic sclerosis, polymyositis, rheumatoid arthritis, myasthenia gravis, juvenile onset diabetes mellitus, glomerulonephritis, autoimmune thyroiditis, graft rejection, Blau syndrome, scleroderma, stomatitis, retinitis pigmentosa, proliferative vitreoretinopathy, Best's yolk macula degeneration, eczema, urticaria, vasculitis, eosinophilic fasciitis, wet and dry age-related macular degeneration, diabetic retinopathy, retinopathy of prematurity, diabetic macular inflammation, retinal vein occlusion, cystic macular edema, glaucoma, Parkinson's disease, Alzheimer's disease, Huntington's disease, breast cancer, lung cancer, bladder cancer, pancreatic cancer, liver cancer, head and neck squamous cell carcinoma, thyroid carcinoma, sarcoma, osteosarcoma, desmoid, melanoma, prostate cancer, colorectal cancer, ovarian cancer, cervical cancer, esophageal cancer, gastric cancer, myeloma, lymphoma, mantle cell lymphoma, cutaneous T-cell lymphoma, chronic and nonprogressive anemia, idiopathic or essential thrombocythemia, leukemia, acute leukemia, chronic leukemia, lymphocytic leukemia, myelogenous leukemia, myelodysplastic syndrome, myeloproliferative disorder, brain tumor, astrocytoma, medulloblastoma, Schwann cell tumor, primary neuroectodermal tumor, or pituitary tumor.

18. The method of claim 16, where the disease or disorder is lymphoma, endometriosis, psoriasis, systemic lupus erythematosus, multiple sclerosis, or rheumatoid arthritis.

19. The method of claim 17, wherein the lymphoma is primary central nervous system lymphoma or diffuse large B-cell lymphoma with MYD88 L265P mutation.
